(19) United States
(12) Patent Application Publication
(10) Pub. No.: US 2025/0152748 A1
(43) Pub. Date: May 15, 2025

(54) HYDRODYNAMIC GENE DELIVERY

(71) Applicant: Johns Hopkins University, Baltimore, MD (US)

(72) Inventors: Robert L. Kruse, Baltimore, MD (US); Vivek Kumbhari, Baltimore, MD (US); Yuting Huang, Baltimore, MD (US); Florin M. Selaru, Baltimore, MD (US)

(21) Appl. No.: 18/284,040

(22) PCT Filed: Mar. 25, 2022
(86) PCT No.: PCT/US22/21984

§ 371 Date: Sep. 25, 2023

Related U.S. Application Data
(60) Provisional application No. 63/165,944, filed on Mar. 25, 2021.

(51) Int. Cl. A61K 48/00; A61B 6/00; A61K 31/10; A61K 31/155; A61K 31/24; A61K 31/245; A61K 38/17; A61K 38/31; A61M 25/10

(52) U.S. Cl. CPC A61K 48/0075; A61B 6/481; A61K 31/10; A61K 31/155; A61K 31/24; A61K 31/245; A61K 38/1709; A61K 38/31; A61K 48/0058; A61M 25/10; A61M 2025/1052; A61M 2210/1071

(57) ABSTRACT

Hydrodynamic injection of nucleic acid or protein for in vivo gene therapy to biliary duct, liver, pancreas, and kidney of a subject.

### CROSS-REFERENCE TO RELATED APPLICATION

This application claims the benefit of and priority to U.S. Provisional Application No. 63/165,944, filed Mar. 25, 2021, the entire contents of which are hereby incorporated by reference.

### BACKGROUND

The liver, pancreas and kidneys are affected in many acquired and inherited gene disorders. Devastating single gene disorders such as alpha-1 antitrypsin deficiency, cystic fibrosis, and many others could theoretically be treated by inserting a corrected copy of the defective gene into affected cells. This presents an opportunity for the application of organ-targeted gene therapy, where the replacement of single gene has been shown to have a significant clinical impact. However, a technically simple, free of significant complications, organ-specific method to deliver gene therapy into the liver, pancreas, or kidney does not currently exist. The lack of such a method is a major drawback to the effective treatment of millions of patients, many of whom are children. Previous attempts at treatment of some of these disorders highlighted the potentially catastrophic side effects associated with the delivery vehicle, as well as with the method of delivery. There is need for the development of a clinical-grade, simple, safe, and efficient in vivo nucleic acid delivery system.

### SUMMARY

The present disclosure provides methods and systems for intra-biliary delivery of nucleic acid and protein via hydrodynamic administration.

In one aspect, methods and systems are provided to treat or prevent kidney, liver, or pancreas disease by genetic therapy. In particular embodiments, methods are provided to treat or prevent hemophilia A, hemophilia B, alpha-1 antitrypsin deficiency, familial hypercholesterolemia, progressive familial intrahepatic cholestasis, hereditary hemochromatosis, Wilson's disease, Crigler-Najjar Syndrome, methymalonic academia, phenylketonuria, and/or ornithine transcarbamylase deficiency, and other diseases and disorders as disclosed herein by genetic therapy.

More particularly, in one preferred aspect, methods and systems are provided for treating a subject comprising: administering with a catheter an effective amount of a nucleic acid and/or protein solution at high-fluid pressure through the subject's biliary tree into the subject's liver, wherein the catheter delivery portion is advanced through the biliary tract upstream of the cystic duct and into the common hepatic duct.

In one embodiment, the catheter tip is positioned at an extrahepatic location during delivery of the nucleic acids. In another embodiment, the catheter tip is positioned within an intrahepatic position within the liver parenchyma during delivery of the nucleic acids. In a further embodiment, the catheter tip is placed within or proximate to the liver parenchyma. In a yet further embodiment, the catheter is positioned whereby the catheter balloon is inflated proximate to or downstream of the liver hilum within the common hepatic duct.

The administered nucleic acid and/or protein solution suitably increases pressure within the liver. Suitably, the catheter balloon is inflated inside the liver parenchyma to prevent retrograde flow of fluid into the common hepatic duct.

In a further aspect, methods and systems are provided that may comprise: administering to the subject's nucleic acid and/or protein solution biliary tree, liver, kidney, or pancreas, 1) a first fluid composition that does not contain nucleic acid and/or protein and thereafter 2) an effective amount of a nucleic acid and/or protein solution. In one embodiment, suitably, the first fluid injection serves to clear bile, urine, or pancreatic exocrine secretions from the biliary system, ureters and renal pelvis, or pancreatic ductal system, before the injection of the nucleic acid and/or protein solution.

In a further aspect, methods and systems are provided that may comprise: administering a radiocontrast agent through the subject's biliary tree, liver, kidney or pancreas to verify catheter placement after balloon inflation, and thereafter administering an effective amount of a nucleic acid and/or protein solution at high-fluid pressure through the subject's biliary tree, liver, kidney or pancreas. Suitably, the catheter position is selected and/or verified with visualization of the administered radiocontrast agent. Suitably, the radiocontrast agent is administered through the subject's biliary tree into the subject's liver, through the subject's pancreatic ducts into the subject's pancreas, and/or through the subject's ureters into the subject's kidneys.

In an additional aspect, methods and systems are provided for placing the catheter into the common hepatic duct for biliary hydrodynamic injection, wherein the catheter placement includes the use of endoscopic retrograde cholangiopancreatography (ERCP) in order to insert a catheter into the common hepatic duct at a location specified herein, or alternatively, the subject's pancreatic duct.

In each of such aspects, in at least certain embodiments, preferably the catheter is selectively advanced into the right hepatic bile duct whereby the nucleic acids is administered within right liver lobes.

In each of such aspects, in at least certain embodiments, preferably the catheter is selectively advanced into the left hepatic bile duct whereby the nucleic acids is administered within left liver lobes.

In each of such aspects, in at least certain embodiments, preferably one or more pharmacologic agents is injected into the pancreatic duct prior to or after the endoscopic retrograde cholanglopancreatography procedure suitably to decrease the frequency and severity of post-procedure pancreatitis. Exemplary pharmacologic agents include one or more agents that inhibit pancreatic digestive enzymes. Suitable pharmacologic agents for administration may comprise gabexate mesilate, nafamostat mesylate, ulinastatin, Camostat mesylate, Aprotinin, Pefabloc, Trasylol, and Urinary Trypsin Inhibitor, or enzyme suppressive agents, including somatostatin. Additional suitable pharmacologic agents comprise may include one or more agents that suppress the immune system such as corticosteroids, tacrolimus, or sirolimus.

In an additional aspect, methods and systems are provided for placing a catheter into the common hepatic duct for biliary hydrodynamic injection, comprising: (a) advancing an endoscope/echoendoscope into the small intestine or stomach; (b) inserting a needle through the small intestine or stomach wall and into a bile duct or gallbladder; (c) optionally injecting fluid into a bile duct or the gallbladder suitably in order to increase its diameter to allow easier entry of a guidewire and/or catheter; (d) passing a guidewire through the needle into a bile duct or gallbladder; (e) advancing a catheter over the wire into the common hepatic duct and administering nucleic acid and/or protein via the catheter.

Suitably in such methods and systems, sonographic guidance is used to locate the common bile duct, common hepatic duct, right hepatic duct, left hepatic duct, small intrahepaticducts, or gallbladder. A contrast agent also suitably may be injected to opacity the biliary tree. Exemplary contrast agents include carbon dioxide or an isosmolar non-ionic contrast agent

Suitably in such methods and systems the catheter ls localized into the right or left common hepatic duct for nucleic acid and/or protein injection. Suitably in such methods the bile duct accessed initially is upstream of the common hepatic duct in the liver.

In certain embodiments of such methods and systems, an upstream branch of the common hepatic duct is first accessed, and a catheter is advanced antegrade to bile flow to mediate positioning in common hepatic duct or kept within the specific bile duct branch. In further embodiments, a catheter is configured whereby the open exit of the injection port where the nucleic acid and/or protein solution exits is downstream from the occlusion balloon (the side facing the intrahepatic biliary tree), mediating transfection to a target area. In yet additional embodiments, a second balloon is utilized that occludes the catheter insertion site of the biliary tree. Preferably, the second balloon is adjustable along the length of the catheter such that it could be positioned or inflated at the target location of needle entry into the biliary tree.

In certain embodiments, an upstream branch of the common hepatic duct is first accessed, the injection opening in the catheter is closer to a second proximal catheter balloon, such that fluid flows in the antegrade direction, but is stopped by a distal first catheter balloon in the common hepatic duct thereby restricting fluid into the intrahepatic ductal system.

In certain embodiments, a stent is positioned in the opening created by the needle, in order to allow for repeated endoscopic accession of the bile duct system over time for additional hydrodynamic injections.

In a further aspect, methods and systems are provided for placing a catheter into the common hepatic duct for biliary hydrodynamic injection, the methods suitably comprising: (a) using ultrasound, computed tomography or another imaging modality for percutaneous needle placement directly from the skin into the gallbladder or into the bile ducts of the liver; (b) injecting a contrast agent into the gallbladder and/or bile duct to opacify the biliary system; (c) advancing a wire though the needle into the common hepatic duct from the initial point of entry; (d) advancing a catheter over the wire into the common hepatic duct and administering nucleic acid and/or protein.

In one embodiment, an upstream branch of the common hepatic duct is first accessed by percutaneous route, and the catheter is be advanced antegrade to bile flow to mediate positioning in common hepatic duct or kept within the specific bile duct branch. Suitably, a catheter is used that is configured such that the open exit of the injection port where the nucleic acid and/or protein solution exits is downstream from the occlusion balloon (the side facing the intrahepatic biliary tree) mediating transfection to that specific area. In certain preferred embodiments, a catheter is used that comprises a second balloon such that it occludes the catheter insertion site of the biliary tree or gallbladder. In additional preferred embodiments, a catheter is used that comprises a second balloon that is adjustable along the length of the catheter such that it could be inflated at a target location.

In certain embodiments of the present methods and systems, including to decrease risk of pancreatitis for example with patients at high risk, the present methods may be favored over endoscopic retrograde cholangiopancreatography for catheter placement into the common hepatic duct prior to gene injection.

In a further embodiment, methods and systems are provided for biliary hydrodynamic injection into the liver, wherein a guidewire used for catheter placement into the common hepatic duct is kept in during hydrodynamic injection for quality assurance of catheter placement and stability during injection.

In a yet further embodiment, methods and systems are provided for biliary hydrodynamic injection into the liver, wherein a guidewire is selectively advanced into the right or left common hepatic ducts and kept in during the hydrodynamic injection for quality assurance of catheter placement and stability during injection.

In certain preferred embodiments of the present methods and systems, the nucleic acid and/or protein solution is administered through the subject's biliary tree into the subject's liver.

In additional preferred embodiments of the present methods and systems, the subject's cells are transfected with the administered nucleic acid or protein.

In yet additional preferred embodiments of the present methods and systems, the methods may further comprise electing an amount of nucleic acid or protein to be administered to the subject based on the subject liver weight.

In additional preferred embodiments of the present methods and systems, the nucleic acid comprises DNA administered in an amount of at least 1 mg of DNA per kilogram of the subject's total liver tissue weight.

In further preferred embodiments of the present methods and systems, the nucleic acid comprises RNA administered in amount of at least 1 mg of RNA per kilogram of the subject's total liver tissue weight.

In additional preferred embodiments of the present methods and systems, the nucleic acid is administered as a fluid composition in a total volume amount of 30 mL or greater per kilogram of the subject's total liver tissue weight.

In further preferred embodiments of the present methods and systems, the nucleic acid is administered as a fluid composition in a total volume amount of 100 mL or greater per kilogram of the subject's total liver tissue weight.

In additional preferred embodiments of the present methods and systems, hydrodynamic injection results in the loss of volume injected through escape into the vascular system, such that the biliary system is not closed and not restricted by the volume injected.

In further preferred embodiments of the present methods and systems, the volume of nucleic acid or protein solution injected correlates with the subject's liver weight.

In a particular aspect, methods and systems are provided for determining liver weight of individual patients for nucleic acid, protein, and/or volume dosing purposes, comprising: calculating a subject's deal body weight, or calculating a subject's liver weight from computed tomography scan or magnetic resonance imaging, and thereafter conducting a biliary hydrodynamic procedure.

In additional preferred embodiments of the present methods and systems, the flow rate is independent of the subject's liver weight.

In yet additional preferred embodiments of the present methods and systems, the nucleic acid is administered as a fluid composition at an injection flow rate equal to or greater than 2 mL/sec and does not result in bile duct rupture.

In further preferred embodiments of the present methods and systems, the nucleic acid is administered as a fluid composition at an injection flow rate equal to or greater than 5 mL/sec and does not result in bile duct rupture.

In additional preferred embodiments of the present methods and systems, the nucleic acid is administered as a fluid composition at an injection flow rate equal to or greater than 10 mL/sec and does not result in bile duct rupture.

In further preferred embodiments of the present methods and systems, a non-nucleic acid solution is administered at a flow rate of 1 mL/sec or less, and at a volume greater than 20 mL prior to nucleic acid injection in order to clear biliary substances from the system.

In additional preferred embodiments of the present methods and systems, a first non-nucleic acid composition is administered prior to the nucleic acids or proteins. Preferably, the first non-nucleic acid composition is administered in an amount approximately equal to the subject's native biliary volume. Also preferred is where the non-nucleic acid solution comprises normal saline solution, Dextrose 5% in Water, lactate ringer's solution, and phosphate buffered solution.

In additional preferred embodiments of the present methods and systems, liver enzymes of the subject, such as aspartate aminotransferase, alanine aminotransferase, lactate dehydrogenase, gamma-glutamyl transferase, and alkaline phosphatase of the subject are monitored during and/or subsequent to the administering.

In further preferred embodiments of the present methods and systems, the flow rate is adjusted according to the degree of liver damage designated for the injection.

In yet further preferred embodiments of the present methods and systems, the flow rate of the nucleic acid or protein solution is >5 mL/second to yield serum or plasma ALT or AST level of the subject of at least 100 U/L within 5 minutes to 5 days following administering of the nucleic acid solution.

In additional preferred embodiments of the present methods and systems, the flow rate of the nucleic acid or protein solution is >5 mL/second to yield serum or plasma ALT or AST level of the subject of at least 200 U/L within 5 minutes to 5 days following administering of the nucleic acid solution.

In further preferred embodiments of the present methods and systems, the flow rate of the nucleic acid or protein solution is equal to or less than 5 mL/sec to avoid induction of liver enzyme elevation from the injection.

In additional preferred embodiments of the present methods and systems, one or more immunosuppressant or anti-inflammatory agents, such as cyclophosphamide, cyclosporin, tacrolimus, sirolumus, mycophenolate mofetil, dexamethasone and/or prednisone are administered to the subject prior to, during, or after biliary hydrodynamic injection suitably to reduce inflammation from the injection or immune responses against transgene.

In a further aspect, methods are provided for targeting nucleic acid and/or protein transfection of specific hepatocytes within the liver tissue by varying the flow rate of biliary hydrodynamic injection.

In certain preferred embodiments of such methods and systems, a higher flow rate is used equal to or above 4 mL/sec targets hepatocytes preferentially at the lobular borders (zone 1), at portal triads, and near large vessels. In additional embodiments, suitably a lower flow rate equal to or lower than 4 mL/sec targets hepatocytes preferentially around the central vein of the lobular (zone 3) and throughout lobules. In further embodiments, suitably at least two different flow rates are employed during biliary hydrodynamic injection, above and below 4 mL/sec, in order to maximize the gene delivery to an increased proportion of hepatocytes within the entire hepatic lobule and liver.

In additional preferred embodiments of the present methods and systems, including for gene delivery into cholangiocytes, nucleic acid and/or protein solution may be injected at elevated pressure through the biliary system, preferably thereby allowing nucleic acids and/or proteins to enter directly cholangiocytes bordering the bile ducts.

In further preferred embodiments of the present methods and systems, including for gene delivery into endothelial cells of the liver, nucleic acid and/or protein solution may be injected at elevated pressure through the biliary system, preferably thereby yielding elevated pressure in portal triads and fluid solution escaping from bile ducts into endothelial cells.

In additional preferred embodiments of the present methods and systems, including for gene delivery into fibroblasts of the liver, nucleic acid and/or protein solution may be injected at elevated pressure through the biliary system, preferably thereby yielding elevated pressure in portal triads and fluid solution escaping from bile ducts into fibroblasts.

In further preferred embodiments of the present methods and systems, including for gene delivery into neurons of the liver, nucleic acid and/or protein solution may be injected at elevated pressure through the biliary system, preferably thereby yielding elevated pressure in portal triads and fluid solution escaping from bile ducts into neurons.

In additional preferred embodiments of the present methods and systems, including for gene delivery into smooth muscle cells of the liver, nucleic acid and/or protein solution may be injected at elevated pressure through the biliary system, preferably thereby yielding elevated pressure in portal triads and fluid solution escaping from bile ducts into smooth muscle cells.

In further preferred embodiments of the present methods and systems, including for gene delivery into hepatocytes, nucleic acid and/or protein solution may be injected at elevated pressure through the biliary system, preferably thereby allowing nucleic acid and/or proteins to enter directly into hepatocytes bordering the canaliculi and bile ducts.

In additional preferred embodiments of the present methods and systems, including for targeting expression into specific cell-types within the liver during biliary hydrodynamic injection, DNA molecules that contain cell-type specific promoters may be injected to target expression for specific cell types. Suitably, cholangiocytes are targeted for expression with cytokeratin-19 promoter and cytokeratin-18 promoter. In another embodiment, hepatocytes suitably are targeted for expression with alpha-1 antitrypsin promoter, thyroxine binding globulin promoter, albumin promoter, HBV core promoter, or hemopexin promoter. In a further embodiment, suitably endothelial cells are targeted for expression with intercellular adhesion molecule-2 (ICAM-2) promoter, fms-like tyrosine kinase-1 (Flt-1) promoter, vascular endothelial cadherin promoter, or von Willenbrand Factor (vWF) promoter. In a yet further embodiment, fibroblasts are suitably targeted for expression with COLIJA1 promoter, COL1A2 promoter, FGF10 promoter, Fsp1 promoter, GFAP promoter, NG2 promoter, or PDGFR promoter. In a yet further embodiment, smooth muscle cells of the liver are suitably targeted for expression with muscle creatine kinase promoter. In a still further embodiment, neurons of the liver are suitably targeted for expression with synapsin I promoter, calcium/calmodulin-dependent protein kinase II promoter, tubulin alpha I promoter, neuron-specific enolase promoter and platelet-derived growth factor beta chain promoter.

In another embodiment, methods are provided for targeting expression into multiple cell-types within the liver during biliary hydrodynamic injection, comprising injecting DNA molecules that promoters active in two or more of hepatocytes, cholangiocytes, endothelial cells, or fibroblasts. In such methods, suitably, promoters used to target two or more cell types in the liver include cytomegalovirus promoter, EF1alpha promoter, SV40 promoter, ubiquitin B, GAPDH, beta-actin, or PGK-1 promoter.

In a further aspect, methods are provided for nucleic acid and/or protein delivery into cells of the pancreas, comprising: (a) placing a catheter through the major duodenal papilla into the main pancreatic duct, distal to the portion of where the pancreatic duct the fuses with the common bile duct; (b) removing fluid residing in the pancreatic duct removing digestive enzymes from the ductal lumen; (c) injecting contrast into the pancreatic duct, in order to confirm correct placement; (d) inflating a balloon in the catheter near the entrance to the pancreatic duct past the common bile duct to prevent retrograde flow of fluid; and (e) injecting a composition comprising DNA, RNA, oligonucleotides or proteins.

In a further aspect, methods are provided for nucleic acid and/or protein delivery into cells of the pancreas, comprising: (a) placing a catheter through the minor duodenal papilla into the accessory or dorsal pancreatic duct, with option to advance farther into the main pancreatic duct; (b) removing fluid residing in the pancreatic duct removing digestive enzymes from the ductal lumen; (c) injecting contrast into the pancreatic duct, in order to confirm correct placement; (d) inflating a balloon in the catheter at the fusion of the dorsal and ventral pancreatic ducts to prevent retrograde flow of fluid; and (e) injecting a composition comprising DNA, RNA, oligonucleotides or proteins.

In such methods, endoscopic retrograde cholangiopancreatography (ERCP)-mediated placement of a catheter into the pancreatic duct may be utilized. Preferably, fluid residing in the pancreatic duct is removed by suction.

In such methods, preferably, the catheter used for injection has at least three ports for balloon air, for pressure catheter insertion, and for DNA injection. Preferred catheters may further comprise guidewire and an injection port, such that the pressure catheter could be inserted through either port depending on size, and the DNA solution could be inserted through either port depending on size. Preferably, a guidewire is selectively advanced into the pancreatic duct and kept in during the hydrodynamic injection for quality assurance of the targeted depth of catheter and maintenance of stability of the catheter during injection.

In such methods, preferably injection parameters for pancreatic injection correspond to the weight of the pancreas. Preferably, the volume such of the nucleic acid or protein composition injected into the pancreas is at minimum 10 mL, more optimally 20 mL, or can exceed 30 or 40 mL in volume, such that volume escapes from the pancreatic ducts into the parenchymal tissue.

Suitably, the flow rate for administered fluid(s) (such as the nucleic acid or protein composition) for the procedure exceeds 1 mL/sec and more preferably 2 mL/sec and in other embodiments 3 mL/sec, or 4 mL/sec. Suitably, the injection procedure is monitored by intraductal pressure, in order to ensure pancreatic function. Preferably, the optimal ductal pressure for pancreatic gene delivery is greater than 50 mmHg, greater than 75 mmHg, greater than 100 mmHg, greater than 150 mmHg, or greater than 200 mmHg.

In such methods, preferably, a DNA composition is primed into the circuit tubing between the power injector and the distal end of the catheter tip. Preferably, the DNA composition is injected with a double-barrel power injector, such that a non-DNA composition chases the DNA solution through the circuit such that there is no DNA composition remaining.

In such methods, preferably, the amylase and lipase levels are trended for degree of pancreatic injury.

In such methods, preferably, the catheter is advanced farther into the pancreatic duct to deliver gene specifically into the distal portions of the pancreas.

In such methods, preferably the solution corresponds to normal saline, phosphate buffer solution, or dextrose 5% water.

In such methods, preferably additional pharmacological agents are added to the nucleic acid and/or protein solution for delivery into cells of the pancreas. For example, the pharmacologic agents may serve to prevent or ameliorate the development of pancreatitis post-injection. Suitable pharmacologic agents suppress the immune system and may include for example cyclophosphamide, cyclosporin, tacrolimus, sirolumus, mycophenolate mofetil, dexamethasone and/or prednisone.

The pharmacologic agents also may inhibit pancreatic digestive enzymes. Suitable pharmacologic agents may include gabexate mesilate, nafamostat mesylate, ulinastatin, Camostat mesylate, Aprotinin, Pefabloc, Trasylol, and Urinary Trypsin Inhibitor, or enzyme suppressive agents, including somatostatin.

In such methods, preferably a solution with pharmacologic agents inhibiting pancreatitis is administered at a flow rate (<1 mL/sec) after the hydrodynamic injection is completed and before repeat fluoroscopy.

In such methods, preferably the pancreatic tissue targeted is a tumor, suitably such that the catheter is placed within close proximity to the site of the tumor, for example where the administered nucleic acid or protein can contact the tumor, or where the catheter tip contacts or is within 0.1, 0.2, 0.3, 0.4 or 0.5 from the tumor at the time of administering nucleic acid or protein.

In such methods, the nucleic acid suitably may be circular DNA, such as plasmid DNA or minicircle DNA, or linear DNA, such as close-ended DNA.

In such methods, the macromolecule suitably may be one or more of mRNA, small interfering RNA, antisense RNA, ribozymes, or proteins.

In such methods, in certain embodiments, the pancreas has a disease pathology that the nucleic acid and/or injection will attempt to treat, including cancer, cystic fibrosis, type 1 diabetes, type 2 diabetes, autoimmune pancreatitis, and rare monogenic pancreatic disorders.

In such methods, in certain embodiments, including for gene delivery into pancreatic ductal epithelial cells, nucleic acid and/or solution may be at elevated pressure through the pancreatic ductal system, preferably thereby allowing nucleic acids and/or proteins to enter directly pancreatic ductal epithelial cells bordering the pancreatic ducts.

In such methods, in certain embodiments, including for gene delivery into acinar cells of the pancreas, nucleic acid and/or protein solution may be injected at elevated pressure through the pancreatic ductal system, preferably thereby allowing fluid solution to escape the ductal system into acinar cells.

In such methods, in certain embodiments, including for gene delivery into islet cells of the pancreas, nucleic acid and/or protein solution may be injected at elevated pressure through the pancreatic ductal system, preferably thereby allowing fluid solution to escape the ductal system into islet cells.

In such methods, in certain embodiments, including for gene delivery into endothelial cells of the pancreas, nucleic acid and/or protein solution may be injected at elevated pressure through the pancreatic ductal system, preferably thereby allowing fluid solution to escape the ductal system into endothelial cells.

In such methods, in certain embodiments, including for gene delivery into neurons of the pancreas, nucleic acid and/or protein solution may be injected at elevated pressure through the pancreatic ductal system, preferably thereby allowing fluid solution to escape the ductal system into neurons.

In such methods, in certain embodiments, including for targeting expression into specific cell-types within the pancreas during pancreatic ductal hydrodynamic injection, DNA molecules that contain cell-type specific promoters may be injected preferably thereby to target expression for specific cell types. In one embodiment, preferably pancreatic acinar cells are targeted for expression with chymotrypsin-like elastase-1 promoter, Ptf1a promoter, or Amy2a promoter. In another embodiment, preferably pancreatic ductal epithelial cells are targeted for expression withSox9 promoter, Hnf1b promoter, Krt19 promoter, and Muc 1 promoter. In another embodiment, preferably pancreatic islet cells are targeted for expression with Insulin promoter, glucagon promoter, somatostatin promoter, ghrelin promoter, or neurogenin-3 promoter. In another embodiment, endothelial cells of the pancreas are targeted for expression with intercellular adhesion molecule-2 (ICAM-2) promoter, fms-like tyrosine kinase-1 (Flt-1) promoter, vascular endothelial cadherin promoter, or von Willenbrand Factor (vWF) promoter. In another embodiment, neurons of the pancreas are targeted for expression with synapsin I promoter, calcium/calmodulin-dependent protein kinase II promoter, tubulin alpha I promoter, neuron-specific enolase promoter and platelet-derived growth factor beta chain promoter.

In a further aspect, methods are provided for targeting expression into multiple cell-types within the pancreas during pancreatic ductal hydrodynamic injection, comprising injecting DNA molecules that promoters active in two or more of pancreatic acinar, ductal epithelial, or islet cells. In one embodiment of such methods, promoters used to target two or more cell types in the pancreas include cytomegalovirus promoter, EFlalpha promoter, SV40 promoter, ubiquitin B promoter, GAPDH promoter, beta-actin promoter, or PGK-1 promoter.

In a further aspect, methods are provided for nucleic acid and/or protein delivery into the different cell types of the kidney, comprising: (a) obtaining a cystoscope and advancing through the urethra and into the bladder; (b) advancing a catheter through the cystoscope and into the right or left ureter; (c) advancing the catheter to the distal end of the ureter and entrance to the renal pelvis, preferably using injection of contrast and fluoroscopy to confirm the location of the catheter; (d) suction of fluid through the catheter to drain the renal pelvis of urine alleviating potential toxicity; (e) opening of a balloon to seal at the ureter, preferably to prevent antegrade flow of solution during hydrodynamic injection; (f) injection of contrast and imaging with fluoroscopy to confirm balloon seal, optionally followed by removal of contrast; (g) priming of catheter circuit from power injector to distal tip with DNA solution; (h) injecting fluid containing nucleic acids and/or proteins.

In preferred embodiments, such methods may further comprise one or more of the following: (i) use of a guidewire in certain embodiments to facilitate canulation of the ureteral orifice, followed by insertion of the catheter; (ii) injecting of fluid containing nucleic acids and/or proteins with a power-injector at high speed and high pressure, monitoring injection with a pressure catheter for quality assurance of injection goals reached; (iii) in certain embodiments, the macromolecule-containing fluid is optionally chased with a non-macromolecule containing solution such as normal saline to ensure complete delivery of solution into the kidney; (iv) balloon deflation after the cessation of injection; (v) a repeat contrast injection and fluoroscopy may be optionally performed; (vi) the catheter and guidewire may be removed from the ureter, and the procedure repeated again for the un-injected kidney; (vii) removal of the catheter from the ureters, bladder, and urethra outside of the patient along with the cystoscope; and/or (viii) optional steps of monitoring creatinine, blood urea nitrogen, and glomerular filtration rate after injection to monitor damage from hydrodynamic injection.

In certain preferred embodiments of such methods, injection parameters include a flow rate is at least 1 mL/second, or at least 2 mL/second, or at least 3 mL/second. Suitably, injection parameters include a volume injected being up to 10 mL, 20 mL or 30, or 50 mL total, such that volume escapes from the renal pelvis into the parenchymal tissue.

In certain preferred embodiments of such methods, the pressure achieved during kidney injection is at minimum of 50 mmHg is achieve, or at least 75 mmHg is achieved, or at least 100 mmHg is achieved.

In certain preferred embodiments of such methods, suitably one or more additional therapeutic agents are included in injection solution for example to decrease inflammation and/or potential infection from the injection procedure. The one or more additional therapeutic agents suitably may comprise one or more small molecules. The one or more additional therapeutic agents suitably may include one or more of antibiotics, and anti-inflammatory drugs including cyclophosphamide, cyclosporin, tacrolimus, sirolumus, mycophenolate mofetil, dexamethasone and/or prednisone.

In certain preferred embodiments of such methods, the cell types include collecting duct cells, proximal tubule cells, distal tubule cells, interstitial cells, podocytes, glomerular cells, renal epithelial cells, and endothelial cells.

In certain preferred embodiments of such methods, DNA molecules are injected that contain cell-type specific promoters to target expression for specific cell types.

In certain preferred embodiments of such methods, proximal tubular epithelial cells are targeted for expression with gamma-glutaryl transpeptidase promoter, Sg1t2 promoter, or NPT2a promoter. Suitably, endothelial cells are targeted for expression with intercellular adhesion molecule-2 (ICAM-2) promoter, fms-like tyrosine kinase-1 (Flt-1) promoter, vascular endothelial cadherin promoter, or von Willenbrand Factor (vWF) promoter. Suitably, podocytes are targeted for expression with podocin promoter. Suitably, NKCC2 promoter targets cells of the thick ascending limb of Henle, AQP2 promoter targets cells of the collecting duct, and kidney-specific cadherin promoter targets renal epithelial cells.

In a further aspect, methods are provided for targeting expression into multiple cell-types within the kidney during renal pelvis hydrodynamic injection, comprising injecting DNA molecules that promoters active in two or more of collecting duct cells, proximal tubule cells, distal tubule cells, interstitial cells, podocytes, and glomerular cells.

In such methods, preferred promoters used to target two or more cell types in the kidney include cytomegalovirus promoter, EF1-alpha promoter, SV40 promoter, ubiquitin B promoter, GAPDH promoter, beta-actin promoter, or PGK-1 promoter.

In such methods, the nucleic acids suitably comprise circular DNA, linear DNA, plasmid DNA, minicircle DNA, mRNA, siRNA, antisense RNA, ribozymes, or proteins.

In a further aspect, methods are provided for treating a subject, comprising: administering with a catheter an effective amount of a nucleic acid and/or protein solution at high-fluid pressure through the subject's biliary tree, liver, kidney or pancreas, wherein the catheter is configured to administer the nucleic acids and/or protein in the forward direction only and not obliquely or perpendicular.

In such methods, suitably the catheter is configured to administer the nucleic acids or protein the forward direction only and not obliquely or perpendicular with respect to the subject's biliary tract walls, pancreatic ductal walls, or ureter walls.

In a further aspect, methods are provided for treating a subject, comprising: administering with a catheter an effective amount of a nucleic acid and/or protein solution at high-fluid pressure through the subject's biliary tree, liver, kidney or pancreas, wherein the catheter tip is at least 1 cm forward along the catheter length from the catheter balloon midpoint.

In such methods, preferably the catheter tip is at least 4 cm forward along the catheter length from the catheter balloon midpoint. Preferably, the catheter comprises at least two ports and associated lumens. Preferably, a first catheter port is used for delivery of a radiocontrast agent and a second catheter port is used for delivery of the nucleic acids and/or proteins.

Preferably, the same catheter port is be used to deliver both radiocontrast agent and subsequent nucleic acids and/or proteins. Preferably, the catheter comprises a port for guidewire placement. Preferably, the port for guidewire placement is used for administering the nucleic acids after guidewire removal. Preferably, the multiple catheter ports have differing diameters. Preferably, the port used for used for administering the nucleic acid and/or protein solution has preferably the largest cross-section among the multiple catheter ports.

In certain preferred embodiments of the present methods and systems, s contrast composition is administered at a slower flow rate than the nucleic acid and/or solution is administered.

In additional preferred embodiments of the present methods and systems, administered nucleic acid suitably comprises one or more of DNA, mRNA, siRNA, miRNA, lncRNA, tRNA, circular RNA, or antisense oligonucleotides.

In preferred certain embodiments of the present methods and systems, administered nucleic acid is an expression cassette that encodes for a protein and comprises circular DNA, a linear DNA, a single-stranded DNA, a double-stranded DNA, linear mRNA, or circular mRNA.

In certain preferred embodiments of the present methods and systems, one or more DNA or RNA molecules may be combined into the same nucleic acid solution and injected at the same time.

In additional certain preferred embodiments of the present methods and systems, increasing the DNA or RNA amount injected leads to a higher transfection percentage into cells of the liver, pancreas, or kidney.

In certain preferred embodiments of the present methods and systems, protein is administered and comprises a transcription factor, antibody fragment, site-specific nuclease enzyme including but not limited to an RNA-guided nuclease, a mega nuclease, a homing nuclease, a TALE nuclease, or a zinc finger nuclease, or other endogenous intracellular human protein.

In certain preferred embodiments of the present methods and systems, protein is administered in an amount of at least 100 micrograms of protein per kilogram of the subject's total liver weight.

In certain preferred embodiments of the present methods and systems, the nucleic acids and/or proteins are constituted in a specified fluid solution.

In certain preferred embodiments of the present methods and systems, the administered fluid solution has a viscosity within +/−10% of normal saline solution to avoid stress on the walls of the common hepatic duct, pancreatic duct, or ureter.

In certain preferred embodiments of the present methods and systems, the administered fluid solution is hyperosmolar to serum or bile to facilitate gene delivery.

In certain preferred embodiments of the present methods and systems, the administered nucleic acid and/or protein solution is endotoxin-free in order to avoid immune activation and transgene removal.

In certain preferred embodiments of the present methods and systems, the administered fluid solution constituting nucleic acids and/or proteins is normal saline solution, Dextrose 5% in Water, lactate ringer's solution, and phosphate buffered solution.

In certain preferred embodiments of the present methods and systems, the administered nucleic acid and/or protein solution also alternatively comprises a radiocontrast agent to monitor the distribution of the nucleic acid solution. Suitably, the administered solution is monitored in real time with fluoroscopic imaging. Preferably, the administered solution is monitored in real time by fluoroscopy for presence of contrast in targeted liver lobes, pancreatic tissue, and kidney tissue. Preferably, the nucleic acid solution contains 1-33% radiocontrast solution based on total weight of the nucleic acid solution to decrease viscosity.

In certain preferred embodiments of the present methods and systems, injected composition(s) are monitored with a pressure catheter inserted through one of the catheter ports. Suitably, a pressure catheter is inserted in one of the two ports or channels, and one of the ports is used for both radiocontrast injection and hydrodynamic nucleic acid and/or protein solution injection.

In certain preferred embodiments of the present methods and systems, intra-biliary, intra-ductal, or intra-pelvic pressure achieved during hydrodynamic injection is dependent on flow rate used.

In other certain preferred embodiments of the present methods and systems, intra-biliary, intra-ductal, or intra-pelvic pressure achieved during hydrodynamic injection does not substantially depend on volume injected.

In certain preferred embodiments of the present methods and systems, hydrodynamic injection yields a rapid increase in pressure yields a plateau level, which drops (preferably, substantially immediately) with the cessation of injection.

In additional certain preferred embodiments of the present methods and systems, a plateau level of pressure is achieved during the successful administration of the nucleic acid.

In certain aspects, the pressure plateau is about 80 mmHg or greater. In other aspects, the pressure plateau is about 150 mmHg or greater. In yet other aspects, the pressure plateau is about 200 mmHg or greater.

In certain preferred embodiments of the present methods and systems, a failed injection shows a loss of plateau waveform during the injection. In certain embodiments, if the expected pressure curve is not achieved due to a failure of balloon seal or other causes, then the injection can be repeated again during the same procedure.

In certain preferred embodiments of the present methods and systems, pressure curves generated by varying flow rates of the administered nucleic acid and/or protein solution generate different peaking pressure plateaus.

In additional preferred embodiments of the present methods and systems, a catheter balloon seal during the administration that effectively prevents or substantially inhibits undesired retrograde flow is confirmed using pressure tracing.

In certain preferred embodiments of the present methods and systems, the catheter balloon is deflated substantially immediately after completing the hydrodynamic injection of the nucleic acids and/or proteins from the catheter. Preferably, balloon deflation post-injection reduces fluid pressure within the subject's bile duct system, liver, kidney or pancreas. Preferably, deflation of the balloon post-injection reveals a rapid pressure drop in the system confirming effective seal.

In additional preferred embodiments of the present methods and systems, the administering comprises multiple hydrodynamic injections of a nucleic acid and/or protein solution during a single endoscopy, ureteroscopy, or percutaneous procedure to increase gene transfection into liver tissue, pancreatic tissue, or kidney tissue. In such embodiments, preferably, the catheter is repositioned between one or more of the multiple injections. In such embodiments, preferably, the multiple injections are completed within the same endoscopy, ureteroscopy, or percutaneous procedure.

In certain preferred embodiments of the present methods and systems, a second endoscopy, ureteroscopy, or percutaneous procedure and nucleic acid and/or protein injection is performed into the same patient through the biliary tract, pancreatic duct, or ureter on a different day to augment or boost gene expression. In such embodiments, preferably, successive hydrodynamic injections may be performed in the subject at intervals of days to weeks to months to years with no significant tissue damage observed.

In additional certain preferred embodiments of the present methods and systems, liver enzymes, pancreatic enzymes, or kidney biochemical markers and/or vital signs of the subject are monitored prior to each injection.

In further preferred embodiments of the present methods and systems, a repeat cholangiogram, pancreatic ductogram, or ureterogram is performed between injections.

In certain preferred embodiments of the present methods and systems, the hydrodynamic force is delivered into a biliary system through a power injector, consisting of the power injection filled with the injection solution and programmed for designated flow rates and duration of each flow rate prior to injection. In such embodiments, preferably, the power injection is capable of injecting fluids between 1-50 mL/sec in a volume up to 200 mL and at a pressure between 500-2000 psi.

In further preferred embodiments of the present methods and systems, at least two flowrates are used during the procedure with a filling time of DNA solution into the biliary system, pancreatic ductal system, or uretero-kidney system first, thereby priming these biliary systems with DNA, followed by a high flow rate to enact pressure throughout the system.

In certain preferred embodiments of the present methods and systems, the flow rate can be varied during the injection with different short period high flow rates to minimize bile duct, pancreatic duct, or ureter wall and catheter wall stress, with intermediate lower flow rates for longer periods more period.

In additional preferred embodiments of the present methods and systems, after balloon catheter placement is confirmed with radiocontrast, tubing lines and biliary system are flushed to assure removal of contrast before power injector is activated to avoid hepatotoxicity, pancreatotoxicity, or nephrotoxicity.

In further preferred embodiments of the present methods and systems, the power injector circuit is connected to the endoscopic or ureteroscope catheter circuit under sterile conditions.

In certain preferred embodiments of the present methods and systems, an initial solution without nucleic acids and/or proteins is infused at a slow flow rate into the biliary tree, pancreatic duct, or ureters first in order to remove bile, pancreatic enzymes, or urine from the system prior to nucleic acid or protein injection. In such embodiments, preferably, non-nucleic acid and/or protein solution is infused at a flow rate of 1 mL/sec or less, and at a volume greater than 20 mL into the liver, or 10 mL into the pancreas, or 10 mL into the kidney.

In further preferred embodiments of the present methods and systems, the biliary system is primed with a nucleic acid and/or protein solution prior to hydrodynamic injection at a volume equal to native biliary volume, or pancreatic ductal volume, or uretero-kidney pelvis volume.

In additional preferred embodiments of the present methods and systems, the circuit of tubing from the power injector to the catheter port connection to the catheter tip is primed with nucleic acid and/or protein solution prior to hydrodynamic injection.

In further preferred embodiments of the present methods and systems, the power injector and/or circuit is filled with additional non-nucleic acid solution less dense than nucleic acid and/or protein solution at a volume equal or greater to the dead space remaining in the circuit, in order to push all nucleic acid or protein solution into the liver, pancreas, or kidney.

In additional preferred embodiments of the present methods and systems, a double-barreled power injector is optimally used for the injection the nucleic acid and/or protein solution into the catheter. In such embodiments, preferably, one barrel is filled with saline, lactate ringer's solution, or dextrose 5% in water and the second barrel is filled with nucleic acid and/or protein solution. In such embodiments, preferably, the nucleic acid and/or protein solution is first injected, and then the second barrel fires to chase the nucleic acid and/or protein solution through the tubing to make sure it completely enters the liver, pancreas, or kidney. In such embodiments, preferably, the non-nucleic acid solution chase occurs at the same flow rate as the nucleic acid and/or protein solution in order to maintain equivalent pressure. In such embodiments, preferably, the non-nucleic acid solution is first fired through the circuit at low flow rates in order to clear bile from the biliary system, followed by injection of the nucleic acid and/or protein solution, followed by subsequent injection of non-nucleic acid solution. In such embodiments, preferably the non-nucleic acid solution chase is at most equivalent in volume to the nucleic acid and/or protein solution injected and is at minimum the equivalent in volume to the volume of the tubing and catheter circuit outside of the bile system, pancreatic duct system, or ureter-kidney pelvis system.

In further preferred embodiments of the present methods and systems, fluid pressure from the administering is sufficient to yield fluid-filled vesicles and/or dilute cytoplasm inside hepatocytes, pancreatic cells, or kidney cells.

In additional preferred embodiments of the present methods and systems, the administering results in hydrodynamic force and tissue changes including fluid-filled vesicles and/or dilute cytoplasm in both proximal and distal portions of that individual liver lobes.

In yet further preferred embodiments of the present methods and systems, other tissues outside of the targeted organ of the liver, pancreas, or kidney do not receive the hydrodynamic force and are not transfected with nucleic acid and/or protein solution.

In a preferred aspect, a composition comprise a nucleic acid expression cassette is administered within a subject's biliary tree in a retrograde manner that can readily enable elevating pressure of the administered composition. In one aspect, a nucleic acid expression cassette is administered into or within a subject's common bile duct. In one aspect, a nucleic acid expression cassette is administered into or within a subject's hepatic duct. In another aspect, a nucleic acid expression cassette is administered into or within a subject's pancreatic duct.

In a further aspect, a nucleic acid expression cassette can be administrations under conditions based on one or more of 1) measured volume of a subject's biliary tree or volume/characteristics of other administration site; and 2) measured target organ characteristics.

In a further aspect, methods of treating or preventing liver or pancreas disease by genetic therapy are provided and comprises: delivering a vector comprising a nucleic acid sequence that ameliorates a liver or pancreas disease to a subject having or prone of getting a liver or pancreas disease; creating a monodirectional fluid flow; injecting the vector under required pressure and quantity into the sealed ductal or vessel space so that the vector is transferred to the cytoplasm of cells of the liver or pancreas; and expressing the nucleic acid sequence and treating or preventing a liver or pancreas disease (or disease arising from a disorder of liver, pancreas or kidney cellular defect/deficiency) in the subject.

As referred to herein, the term substantially closed space refers to an in vivo space where the normal directional fluid flow has been blocked, such that retrograde injection against this fluid flow will create an elevated pressure can be induced upon delivery of a composition (e.g. fluid composition) that contain a nucleic acid expression cassette. In the present methods, the pressure will be elevated above the initial introduction or infusion pressure of the fluid composition, e.g. at least about a 5, 10, 15, 20, 30, 40, 50, 60, 70, 80, 90, 100, 120, 140, 160, 180, or 200 percent increase, or even higher such as 5, 8, 10, 15, 20, 25, 30, 40, 50 or 60 times increase, in pressure relative to the pressure that the fluid composition is initially introduced to the space such as by injection. It is experimentally verified that administered fluid (e.g. fluid composition comprising a nucleic acid expression cassette) will leak from the closed space during an administration protocol, such as during injection of the fluid composition to the target site, or following such injection.

In certain preferred aspects of the present methods, a device that can prevent bidirectional flow of fluid may be utilized with the administration of a nucleic acid expression cassette. For a device can be utilized that include a balloon that can establish or facilitate maintaining monodirectional flow of fluid during administration of a nucleic acid expression cassette.

In particular aspects, an endoscopic retrograde cholangiopancreatography (ERCP) that comprises balloon may be utilized with the administration of a nucleic acid expression cassette. The ERCP also may contain the nucleic acid expression cassette for administration. In use, endoscope of the ERCP may be positioned through the mouth, the stomach, duodenum, and the duct of the pancreas or the liver of a subject; and the balloon of the ERCP may be opened to thereby create in vivo retrograde, monodirectional fluid flow which can facilitate establishing and maintaining an elevated pressure of the nucleic acid expression cassette during the course of an administration protocol.

Suitable nucleic acid sequences used in the present disclosure may encode a protein or a peptide or a second nucleic acid selected from the group consisting of a shRNA, mRNA, and a combination thereof. The protein or peptide may be an antibody. In some embodiments, the nucleic acid sequence encodes a protein that treats cirrhosis, hemophilia, cystic fibrosis, urea cycle enzyme alterations, or a functional part thereof. In some embodiments, the nucleic acid sequence encodes a protein that treats a pancreas disease or a functional part thereof. Suitable vectors include a virus, a plasmid, or both, as examples. An example of a virus used in the present disclosure is AAV. In some embodiments, a plasmid may comprise an SB transposon comprising the nucleic acid sequence described above. In some embodiments, the methods of the present disclosure may include an additional step of removing the remaining vector through the endoscope.

In some embodiments, a vector comprising any of the expression cassettes disclosed herein is provided.

The present methods and systems are particularly useful to treat or prevent liver-related diseases and disorders including for example, hemophilia A, hemophilia B, alpha-lantitrypsin deficiency, familial hypercholesterolemia, progressive familial intrahepatic cholestasis, hereditary hemochromatosis, Wilson's disease, Crigler-Najjar Syndrome, methymalonic academia, phenylketonuria, and/or ornithine transcarbamylase deficiency.

In particular aspect, the present methods and systems are used to treat hemophilia, including hemophilia B, for example nucleic acid expressing Factor IX is administered.

The present methods and systems are particularly useful to treat or prevent pancreas-related diseases and disorders including for example, Type I DM, single gene disorders causing pancreatitis: CFTR, CLDN2, CPA1, PRSS1 and SPINK1.

In an aspect, the disclosure provides a method of introducing DNA into hepatocytes via hydrodynamic delivery, including the steps of: inserting a catheter into a common hepatic duct of a liver by endoscopic retrograde cholangiopancreatography (ERCP); inflating a balloon to seal the common hepatic duct; and injecting the DNA in a solution at a flow rate of at least about 2 mL/sec; wherein about 30% of the hepatocytes in the liver exhibit transgene expression from transfected DNA.

In embodiments, the DNA is a plasmid DNA.

In embodiments, the DNA is a vector comprising a promoter, 5′ UTR, a protein-coding gene, a 3′UTR, and a polyadenylation sequence; optionally wherein the DNA comprises a transposon.

In embodiments, the solution comprises at least 3 mg of the plasmid DNA.

In embodiments, the promoter is a liver-specific promoter.

In embodiments, the liver-specific promoter is selected from the group consisting of a LP1 promoter, an ApoE/AIAT promoter, an alpha-1 antitrypsin promoter, athyroxine binding globulin promoter, an albumin promoter, a HBV core promoter, a transthyretin promoter, and a hemopexin promoter.

In embodiments, the transposon comprises a piggyBac transposon or a hyperactive piggyBac transposon for enhanced integration.

In embodiments, the 5′ UTR contains an intron to enhance gene expression.

In embodiments, the protein code of the gene is codon optimized for the host organism to enhance protein expression.

In embodiments, the 3′ UTR is selected to enhance mRNA transcript stability and protein expression.

In embodiments, the 3′ UTR is the human albumin 3′ UTR.

In embodiments, transfected hepatocytes are observed in every hepatic lobule of the liver.

In embodiments, each liver lobe has comparable percentage of transfected hepatocytes.

In embodiments, proximal and distal tissue sites to the common hepatic duct within a liver lobe have a comparable percentage of transfected hepatocytes.

In embodiments, the DNA is dissolved in solution comprises normal saline solution, Dextrose 5% in Water, lactate ringer's solution, and phosphate buffered solution.

In embodiments, the transfected hepatocytes yield detectable protein expression from the gene on immunostaining of the liver tissue.

In embodiments, about 30%, about 35%, about 40%, or about 45% of the hepatocytes in the liver are transfected with the plasmid DNA.

In an aspect, the disclosure provides a method of placing a catheter into a common hepatic duct or gallbladder for biliary hydrodynamic injection, including the steps of: advancing an endoscope/echoendoscope into the small intestine or stomach; inserting a needle through the small intestine or stomach wall and into a bile duct or a gallbladder; optionally injecting fluid into the bile duct or the gallbladder in order to increase its diameter; passing a guidewire through the needle into the bile duct or the gallbladder; advancing the catheter over the guidewire into the common hepatic duct and administering a nucleic acid and/or a protein via the catheter.

In embodiments, sonographic guidance is used to visualize a location selected from the group consisting of a common bile duct, a common hepatic duct, a right hepatic duct, a left hepatic duct, a small intrahepatic ducts, and a gallbladder.

In embodiments, the method further comprising injecting a contrast agent to opacify the biliary tree.

In embodiments, the contrast agent is carbon dioxide or an isosmolar non-ionic contrast agent.

In embodiments, the catheter is localized into the right or the left common hepatic duct.

In embodiments, the bile duct accessed initially is upstream of the common hepatic duct in the liver.

In embodiments, an upstream branch of the common hepatic duct is first accessed, and a catheter is advanced antegrade to bile flow to mediate positioning in the common hepatic duct or kept within a specific bile duct branch.

In embodiments, the catheter is configured such that an open exit of an injection port where the nucleic acid and/or protein solution exits is downstream from an occlusion balloon.

In embodiments, a second balloon is utilized to occlude the catheter insertion site of the biliary tree.

In embodiments, the second balloon is adjustable along the length of the catheter such that it can be positioned or inflated at the target location of needle entry into the biliary tree.

In embodiments, the second balloon is adjustable along the length of the catheter such that it can be inflated and positioned at the target location of needle entry into the biliary tree.

In embodiments, the second balloon is adjustable along the length of the catheter such that it can be inflated and positioned at the target location.

In embodiments, an upstream branch of the common hepatic duct is first accessed by the catheter, wherein the catheter has an injection opening closer to a second proximal catheter balloon, such that fluid flows toward the common hepatic duct in the antegrade direction and is prevented from flowing in a retrograde direction by a distal first catheter balloon thereby restricting fluid flow into the intrahepatic ductal system.

In embodiments, a stent is positioned in an opening created by the needle to allow for repeated endoscopic access to the bile duct system over time for additional hydrodynamic injections.

In embodiments, the method further includes the steps of: using ultrasound, computed tomography or another imaging modality for percutaneous needle placement directly from the skin into the bile ducts of the liver or the gallbladder; injecting a contrast agent into the bile ducts of the liver or the gallbladder to opacify the biliary system; advancing a guidewire though the needle into the common hepatic duct from the initial point of entry; and advancing the catheter over the wire into the common hepatic duct and administering the nucleic acid and/or protein.

In embodiments, an upstream branch of the common hepatic duct is first accessed by a percutaneous route, and the catheter is then advanced antegrade to bile flow to mediate positioning of the catheter in the common hepatic duct, or to keep the catheter within a specific bile duct branch.

In embodiments, the catheter is configured such that the open exit of the injection port where the nucleic acid and/or protein solution exits is positioned downstream from an occlusion balloon positioned facing the intrahepatic biliary tree to mediate transfection to a specific biliary area.

In embodiments, the catheter comprises a second balloon such that it occludes the catheter insertion site of the biliary tree or gallbladder.

In embodiments, the catheter comprises a second balloon that is adjustable along the length of the catheter such that it could be inflated at a target location.

In embodiments, the guidewire is kept in place during the hydrodynamic injection, optionally wherein the guidewire is advanced into either a right or a left common hepatic duct.

In embodiments, one or more pharmacologic agents is injected into a pancreatic duct prior to or after the endoscopic retrograde cholangiopancreatography (ERCP) procedure to decrease the frequency and severity of post-procedure pancreatitis.

In embodiments, the one or more pharmacologic agents comprise one or more agents that inhibit pancreatic digestive enzymes.

In embodiments, the one or more pharmacologic agents are selected from the group consisting of gabexate mesilate, nafamostat mesylate, ulinastatin, Camostat mesylate, Aprotinin, Pefabloc, Trasylol, Urinary Trypsin Inhibitor, and enzyme suppressive agents, optionally somatostatin.

In embodiments, the one or more pharmacologic agents comprise one or more agents that suppress the immune system and include corticosteroids, tacrolimus, or sirolimus.

In embodiments, the subject's cells are transfected with the administered nucleic acid or protein.

In embodiments, the method further comprises selecting an amount of nucleic acid or protein to be administered to the subject based on the subject liver weight.

In embodiments, the nucleic acid comprises DNA administered in an amount of at least 1 mg of DNA per kilogram of the subject's total liver tissue weight.

In embodiments, the nucleic acid comprises RNA administered in amount of at least 1 mg of RNA per kilogram of the subject's total liver tissue weight.

In embodiments, the nucleic acid is administered as a fluid composition in a total volume amount of 30 mL or greater per kilogram of the subject's total liver tissue weight.

In embodiments, the nucleic acid is administered as a fluid composition in a total volume amount of 100 mL or greater per kilogram of the subject's total liver tissue weight.

In embodiments, the hydrodynamic injection results in the loss of volume injected through escape into the vascular system, such that the biliary system is not closed and not restricted by the volume injected.

In embodiments, the volume injected correlates with the subject's liver weight.

In an aspect, the disclosure provides a method of determining liver weight of individual patients for nucleic acid, protein, and/or volume dosing purposes, comprising: calculating a subject's body weight or calculating a subject's liver weight from computed tomography scan or magnetic resonance imaging; determining an injection flow rate, and conducting a biliary hydrodynamic procedure based on the flow rate.

In embodiments, the flow rate is independent of the subject's liver weight.

In embodiments, the nucleic acid is administered as a fluid composition and the flow rate is equal to or greater than 2 mL/sec and does not result in bile duct rupture.

In embodiments, the nucleic acid is administered as a fluid composition and the flow rate is equal to or greater than 5 mL/sec and does not result in bile duct rupture.

In embodiments, the nucleic acid is administered as a fluid composition and the flow rate is equal to or greater than 10 mL/sec and does not result in bile duct rupture.

In embodiments, a non-nucleic acid solution is administered at a flow rate of 1 mL/sec or less, and at a volume greater than 20 mL prior to nucleic acid injection in order to clear biliary substances from the system.

In embodiments, a first non-nucleic acid composition is administered prior to the nucleic acids or proteins.

In embodiments, the first non-nucleic acid composition is administered in an amount approximately equal to the subject's native biliary volume.

In embodiments, the non-nucleic acid solution comprises normal saline solution, Dextrose 5% in Water, lactate ringer's solution, and phosphate buffered solution.

In embodiments, liver enzymes of the subject, including aspartate aminotransferase, alanine aminotransferase, lactate dehydrogenase, gamma-glutamyl transferase, and alkaline phosphatase of the subject are monitored during and/or subsequent to the administering.

In embodiments, the flow rate is adjusted according to the degree of liver damage designated for the injection.

In embodiments, the flow rate is >5 mL/second to yield serum or plasma ALT or AST level of the subject of at least 100 U/L within 5 minutes to 5 days following administering of the nucleic acid solution.

In embodiments, the flow rate is >5 mL/second to yield serum or plasma ALT or AST level of the subject of at least 200 U/L within 5 minutes to 5 days following administering of the nucleic acid solution.

In embodiments, flow rate of the administered nucleic acid solution is equal to or less than 5 mL/sec to avoid induction of liver enzyme elevation from the injection.

In embodiments, one or more immunosuppressant or anti-inflammatory agents, including cyclophosphamide, cyclosporin, tacrolimus, sirolumus, mycophenolate mofetil, dexamethasone and/or prednisone are administered to the subject prior to, during, or after biliary hydrodynamic injection to reduce inflammation from the injection or immune responses against transgene.

In an aspect, the present disclosure provides a method of targeting nucleic acid and/or protein transfection of specific hepatocytes within the liver tissue by varying the flow rate of biliary hydrodynamic injection.

In embodiments, a higher flow rate equal to or above 4 mL/sec targets hepatocytes preferentially at the lobular borders (zone 1), at portal triads, and near large vessels.

In embodiments, a lower flow rate equal to or lower than 4 mL/sec targets hepatocytes preferentially around the central vein of the lobular (zone 3) and throughout lobules.

In embodiments, at least two different flow rates are employed during biliary hydrodynamic injection, above and below 4 mL/sec, in order to maximize the gene delivery to an increased proportion of hepatocytes within the entire hepatic lobule and liver.

In embodiments, the nucleic acid is transfected into cholangiocytes by injecting the nucleic acid and/or the protein solution at an elevated pressure through the biliary system, thereby allowing the nucleic acids and/or the proteins to directly enter the cholangiocytes bordering the bile ducts.

In embodiments, the nucleic acid is transfected into endothelial cells of the liver by injecting the nucleic acid and/or the protein solution at an elevated pressure through the biliary system to elevate pressure in portal triads, thereby allowing the nucleic acids and/or the protein to enter the endothelial cells.

In embodiments, the nucleic acid is transfected into fibroblasts of the liver by injecting the nucleic acid and/or the protein solution at elevated pressure through the biliary system to elevate pressure in portal triads, thereby allowing the nucleic acids and/or the protein to enter the fibroblasts.

In embodiments, the nucleic acid is transfected into neurons of the liver by injecting the nucleic acid and/or the protein solution at elevated pressure through the biliary system to elevate pressure in portal triads, thereby allowing the nucleic acids and/or the protein to enter the neurons.

In embodiments, the nucleic acid is transfected into smooth muscle cells of the liver by injecting the nucleic acid and/or the protein solution at elevated pressure through the biliary system to elevate pressure in portal triads, thereby allowing the nucleic acids and/or the protein to enter the smooth muscle cells.

In embodiments, the nucleic acid is transfected into hepatocytes by injecting the nucleic acid and/or the protein solution at elevated pressure through the biliary system, thereby allowing the nucleic acid and/or the proteins to enter directly into hepatocytes bordering the canaliculi and bile ducts.

In embodiments, the nucleic acid comprises a cell-type specific promoter to target expression of a transgene to specific cell types.

In embodiments, cholangiocytes are targeted for expression with a cytokeratin-19 promoter or a cytokeratin-18 promoter.

In embodiments, hepatocytes are targeted for expression with an alpha-1 antitrypsin promoter, a thyroxine binding globulin promoter, an albumin promoter, a HBV core promoter, or a hemopexin promoter.

In embodiments, endothelial cells are targeted for expression with an intercellular adhesion molecule-2 (ICAM-2) promoter, a fins-like tyrosine kinase-1 (Flt-1) promoter, a vascular endothelial cadherin promoter, or a von Willenbrand Factor (vWF) promoter.

In embodiments, fibroblasts are targeted for expression with a COL1A1 promoter, a COL1A2 promoter, a FGF10 promoter, a Fsp1 promoter, a GFAP promoter, a NG2 promoter, or a PDGFR promoter

In embodiments, smooth muscle cells of the liver are targeted for expression with a muscle creatine kinase promoter.

In embodiments, neurons of the liver are targeted for expression with a synapsin I promoter, a calcium/calmodulin-dependent protein kinase II promoter, a tubulin alpha I promoter, a neuron-specific enolase promoter and a platelet-derived growth factor beta chain promoter.

In embodiments, the nucleic acid comprises two or more promoters active in two or more of hepatocytes, cholangiocytes, endothelial cells, or fibroblasts.

In embodiments, the two or more promoters used to target two or more cell types in the liver include a cytomegalovirus promoter, a EFlalpha promoter, a SV40 promoter, a ubiquitin B promoter, a GAPDH promoter, a beta-actin promoter, or a PGK-1 promoter.

In an aspect, the disclosure provides a method of hydrodynamic gene delivery into pancreatic cells, including the steps of: placing a catheter through a major duodenal papilla into a main pancreatic duct, distal to a portion of the pancreatic duct that fuses with a common bile duct, or into a minor duodenal papilla in an accessory or a dorsal pancreatic duct, optionally advancing the catheter farther into the main pancreatic duct; optionally removing fluid residing in the pancreatic duct to remove digestive enzymes from the ductal lumen; injecting a contrast agent into the pancreatic duct to confirm correct placement of the catheter; inflating a balloon in the catheter near an entrance to the pancreatic duct past the common bile duct to prevent retrograde flow of a fluid; injecting at a flow rate of at least 2 mL/sec and a volume at least 20 mL of a solution comprising at least 1 mg DNA, wherein the flow rate, volume, and DNA dose are sufficient to mediate gene expression in all pancreatic lobes and multiple pancreatic cell types.

In embodiments, the solution corresponds to normal saline, phosphate buffer solution, or dextrose 5% water.

In embodiments, the flow rate and the volume are determined based on the weight of the pancreas.

In embodiments, the DNA composition is primed into a circuit tubing between a power injector and a distal end of the catheter tip, or the DNA composition is injected with a double-barrel power injector, such that a non-DNA composition chases the DNA composition through the circuit tubing such that there is no DNA composition remaining.

In embodiments, a level of an amylase and/or a lipase levels are monitored to assess a degree of pancreatic injury during injection.

In embodiments, one or more pharmacological agents are added to the nucleic acid solution to prevent or ameliorate the development of pancreatitis post-injection.

In embodiments, the one or more pharmacologic agents are selected from the group consisting of a cyclophosphamide, a cyclosporin, a tacrolimus, a sirolumus, a mycophenolate, a mofetil, a dexamethasone and a prednisone; or the one or more pharmacologic agents are selected from the group consisting of gabexate mesilate, nafamostat mesylate, ulinastatin, Camostat mesylate, Aprotinin, Pefabloc, Trasylol, Urinary Trypsin Inhibitor, and enzyme suppressive agents, optionally including somatostatin; or the pharmacologic agents inhibit pancreatic digestive enzymes; or the one or more pharmacologic agents are administered at a flow rate (<1 mL/sec) after the hydrodynamic injection is completed and before repeat fluoroscopy.

In embodiments, the volume injected into the pancreas is at minimum 20 mL, or can exceed 30 or 40 mL in volume, such that volume escapes from the pancreatic ducts into the parenchymal tissue.

In embodiments, the flow rate for the procedure exceeds 2 mL/sec and in other embodiments 3 mL/sec, or 4 mL/sec.

In embodiments, the injection procedure is monitored by intraductal pressure, in order to ensure pancreatic function.

In embodiments, the optimal ductal pressure for pancreatic gene delivery is greater than 50 mmHg, greater than 75 mmHg, greater than 100 mmHg, greater than 150 mmHg, or greater than 200 mmHg.

In embodiments, the pancreatic tissue targeted is a tumor, such that the catheter is placed within close proximity to the site of the tumor.

In embodiments, the nucleic acid may be circular DNA, such as plasmid DNA or minicircle DNA, or linear DNA; or wherein the solution contains a macromolecule selected from mRNA, a small interfering RNA, an antisense RNA, a ribozymes, and/or a protein.

In embodiments, the amylase and lipase levels are trended for degree of pancreatic injury.

In embodiments, the catheter is advanced farther into the pancreatic duct to deliver gene specifically into the distal portions of the pancreas.

In embodiments, the solution corresponds to normal saline, phosphate buffer solution, or dextrose 5% water.

In embodiments, one or more additional pharmacological agents are added to the nucleic acid and/or protein solution for delivery into cells of the pancreas.

In embodiments, one or more pharmacologic agents may serve to prevent or ameliorate the development of pancreatitis post-injection.

In embodiments, these pharmacologic agents suppress the immune system and include cyclophosphamide, cyclosporin, tacrolimus, sirolumus, mycophenolate mofetil, dexamethasone and/or prednisone.

In embodiments, the pharmacologic agents inhibit pancreatic digestive enzymes.

In embodiments, pharmacologic agents are selected from gabexate mesilate, nafamostat mesylate, ulinastatin, Camostat mesylate, Aprotinin, Pefabloc, Trasylol, and Urinary Trypsin Inhibitor, or enzyme suppressive agents, including somatostatin.

In embodiments, a solution with pharmacologic agents inhibiting pancreatitis is administered at a flow rate (<1 mL/sec) after the hydrodynamic injection is completed and before repeat fluoroscopy.

In embodiments, the pancreatic tissue targeted is a tumor, such that the catheter is placed within close proximity to the site of the tumor.

In embodiments, the nucleic acid may be circular DNA, such as plasmid DNA or minicircle DNA, or linear DNA, such as close-ended DNA.

In embodiments, the macromolecule may be one or more of mRNA, small interfering RNA, antisense RNA, ribozymes, or proteins.

In embodiments, the pancreas has a disease pathology that the nucleic acid and/or injection will attempt to treat, including cancer, cystic fibrosis, type 1 diabetes, type 2 diabetes, autoimmune pancreatitis, and rare monogenic pancreatic disorders.

In embodiments, the nucleic acid comprises a cell-type specific promoter to target expression of a transgene to specific cell types.

In embodiments, pancreatic acinar cells are targeted for expression with a chymotrypsin-like elastase-1 promoter, a Ptf1a promoter, or a Amy2a promoter.

In embodiments, pancreatic ductal epithelial cells are targeted for expression with a Sox9 promoter, a Hnf1b promoter, a Krt19 promoter, and/or a Muc promoter.

In embodiments, pancreatic islet cells are targeted for expression with an Insulin promoter, a glucagon promoter, a somatostatin promoter, a ghrelin promoter, or a neurogenin-3 promoter.

In embodiments, endothelial cells of the pancreas are targeted for expression with an intercellular adhesion molecule-2 (ICAM-2) promoter, a fins-like tyrosine kinase-1 (Flt-1) promoter, a vascular endothelial cadherin promoter, or a von Willenbrand Factor (vWF) promoter.

In embodiments, the nucleic acid comprises cell-type specific promoters to target gene expression for specific cell types.

In embodiments, neurons of the pancreas are targeted for expression with a synapsin I promoter, a calcium/calmodulin-dependent protein kinase II promoter, a tubulin alpha I promoter, a neuron-specific enolase promoter and a platelet-derived growth factor beta chain promoter.

In embodiments, the nucleic acid comprises two or more promoters active in two or more of a pancreatic acinar cell, a ductal epithelial cell, or an islet cell.

In embodiments, pancreatic islet cells are targeted for expression with an Insulin promoter, a glucagon promoter, a somatostatin promoter, a ghrelin promoter, or a neurogenin-3 promoter.

In embodiments, endothelial cells of the pancreas are targeted for expression with intercellular adhesion molecule-2 (ICAM-2) promoter, fins-like tyrosine kinase-1 (Flt-1) promoter, vascular endothelial cadherin promoter, or von Willenbrand Factor (vWF) promoter.

In embodiments, neurons of the pancreas are targeted for expression with synapsin I promoter, calcium/calmodulin-dependent protein kinase II promoter, tubulin alpha I promoter, neuron-specific enolase promoter and platelet-derived growth factor beta chain promoter.

In an aspect, the disclosure provides a method of retrograde ureteral hydrodynamic injection into the kidney, wherein one or more additional therapeutic agents are included in the injection solution to decrease inflammation and/or potential infection from the injection procedure.

In embodiments, the one or more additional therapeutic agents comprise one or more small molecules.

In embodiments, the one or more additional therapeutic agents include one or more antibiotics or anti-inflammatory drugs, optionally wherein the anti-inflammatory drugs are selected from the group consisting of cyclophosphamide, cyclosporin, tacrolimus, sirolumus, mycophenolate mofetil, dexamethasone and prednisone.

In embodiments, the cell types include collecting duct cells, proximal tubule cells, distal tubule cells, interstitial cells, podocytes, glomerular cells, renal epithelial cells, and endothelial cells.

In embodiments the method includes injecting nucleic acid molecules comprising one or more cell-type specific promoters to target expression for specific cell types.

In embodiments, proximal tubular epithelial cells are targeted for expression with a gamma-glutaryl transpeptidase promoter, a Sglt2 promoter, or a NPT2a promoter.

In embodiments, endothelial cells are targeted for expression with an intercellular adhesion molecule-2 (ICAM-2) promoter, a fins-like tyrosine kinase-I (Flt-1) promoter, a vascular endothelial cadherin promoter, or a von Willenbrand Factor (vWF) promoter.

In embodiments, podocytes are targeted for expression with a podocin promoter.

In embodiments, a NKCC2 promoter targets cells of the thick ascending limb of Henle, an AQP2 promoter targets cells of the collecting duct, and a kidney-specific cadherin promoter targets renal epithelial cells.

In embodiments, the nucleic acid molecule includes two or more promoters active in two or more tissues selected from the group consisting of collecting duct cells, proximal tubule cells, distal tubule cells, interstitial cells, podocytes, and glomerular cells.

In embodiments, the two or more promoters used to target two or more cell types in the kidney include a cytomegalovirus promoter, a EFI-alpha promoter, a SV40 promoter, a ubiquitin B promoter, a GAPDH promoter, a beta-actin promoter, or a PGK-1 promoter.

In embodiments, the nucleic acid comprises circular DNA, linear DNA, plasmid DNA, minicircle DNA, mRNA, siRNA, antisense RNA, ribozymes, or proteins.

In embodiments, the catheter is configured to administer the nucleic acids and/or protein in the forward direction only and not obliquely or perpendicular.

In embodiments, the catheter is configured to administer the nucleic acid or the protein in the forward direction only and not obliquely or perpendicular with respect to the subject's biliary tract walls, pancreatic ductal walls, or ureter walls thereby avoiding injury to any wall.

In embodiments, the contrast composition is administered at a slower flow rate than the nucleic acid and/or solution is administered.

In embodiments, the nucleic acid comprises one or more of DNA, mRNA, siRNA, miRNA, lncRNA, tRNA, circular RNA, or antisense oligonucleotides.

In embodiments, the nucleic acid is an expression cassette that encodes for a protein and comprises circular DNA, a linear DNA, a single-stranded DNA, a double-stranded DNA, linear mRNA, or circular mRNA.

In embodiments, one or more DNA or RNA molecules may be combined into the same nucleic acid solution and injected at the same time.

In embodiments, the injection is monitored with a pressure catheter inserted through one of the catheter ports.

In embodiments, the pressure catheter is inserted in one of the two ports or channels, and one of the ports is used for both radiocontrast injection and hydrodynamic nucleic acid and/or protein solution injection.

In embodiments, a failed injection is detected by loss of the plateau waveform during the injection.

In embodiments, if the expected pressure curve is not achieved due to a failure of a balloon seal or other causes, then the injection is repeated again during the same procedure.

In embodiments, pressure curves generated by varying flow rates of the administered nucleic acid and/or protein solution generate different peaking pressure plateaus.

In embodiments, at least two flow rates will be used during the procedure with a filling time of DNA solution into the biliary system, pancreatic ductal system, or uretero-kidney system first, thereby priming these biliary systems with DNA, followed by a high flow rate to enact pressure throughout the system.

In embodiments, the flow rate can be varied during the injection with different short period high flow rates to minimize bile duct, pancreatic duct, or ureter wall and catheter wall stress, with intermediate lower flow rates for longer periods more period.

In embodiments, a double-barreled power injector is used for injecting the nucleic acid and/or the protein solution into the catheter.

In embodiments, one barrel is filled with saline, lactate ringer's solution, or dextrose 5% in water and the second barrel is filled with nucleic acid and/or protein solution.

In embodiments, the nucleic acid and/or protein solution is first injected, and then the second barrel fires to chase the nucleic acid and/or protein solution through the tubing to make sure it completely enters the liver, pancreas, or kidney.

In embodiments, the non-nucleic acid solution chase occurs at the same flow rate as the nucleic acid and/or protein solution in order to maintain equivalent pressure.

In embodiments, the non-nucleic acid solution is first fired through the circuit at low flow rates in order to clear bile from the biliary system, followed by injection of the nucleic acid and/or protein solution, followed by subsequent injection of non-nucleic acid solution.

In embodiments, the non-nucleic acid solution chase is at most equivalent in volume to the nucleic acid and/or protein solution injected and is at minimum the equivalent in volume to the volume of the tubing and catheter circuit outside of the bile system, pancreatic duct system, or ureter-kidney pelvis system.

In embodiments, fluid pressure from the administering is sufficient to yield fluid-filled vesicles and/or dilute cytoplasm inside hepatocytes, pancreatic cells, or kidney cells.

In embodiments, the administering results in hydrodynamic force and tissue changes including fluid-filled vesicles and/or dilute cytoplasm in both proximal and distal portions of that individual liver lobes.

In embodiments, other tissues outside of the targeted organ of the liver, pancreas, or kidney do not receive the hydrodynamic force and are not transfected with nucleic acid and/or protein solution.

In an aspect, the disclosure provides a method of treating a subject, including the steps of: administering to the subject's biliary tree, liver, kidney, or pancreas, a first fluid composition that does not contain a nucleic acid and/or a protein and then an effective amount of the nucleic acid and/or the protein solution.

In embodiments, the first fluid injection serves to clear bile, urine, or pancreatic exocrine secretions from the biliary system, ureters and renal pelvis, or pancreatic ductal system, before the injection of the nucleic acid and/or protein solution.

### Definitions

The term “activity” refers to the ability of a gene to perform its function such as Indoleamine 2,3-dioxygenase (an oxidoreductase) catalyzing the degradation of the essential amino acid tryptophan (trp) to N-formyl-kynurenine.

The term “antibody,” as used in this disclosure, refers to an immunoglobulin or a fragment or a derivative thereof, and encompasses any polypeptide comprising an antigen-binding site, regardless of whether it is produced in vitro or in vivo. The term includes, but is not limited to, polyclonal, monoclonal, monospecific, polyspecific, non-specific, humanized, single-chain, chimeric, synthetic, recombinant, hybrid, mutated, and grafted antibodies. Unless otherwise modified by the term “intact,” as in “intact antibodies,” for the purposes of this disclosure, the term “antibody” also includes antibody fragments such as Fab, F(ab′)2, Fv, scFv, Fd, dAb, and other antibody fragments that retain antigen-binding function, i.e., the ability to bind, for example, PD-LI, specifically. Typically, such fragments would comprise an antigen-binding domain.

The terms “antigen-binding domain,” “antigen-binding fragment,” and “binding fragment” refer to a part of an antibody molecule that comprises amino acids responsible for the specific binding between the antibody and the antigen. In instances, where an antigen is large, the antigen-binding domain may only bind to a part of the antigen. A portion of the antigen molecule that is responsible for specific interactions with the antigen-binding domain is referred to as “epitope” or “antigenic determinant.” An antigen-binding domain typically comprises an antibody light chain variable region (VL) and an antibody heavy chain variable region (VH), however, it does not necessarily have to comprise both. For example, a so-called Fd antibody fragment consists only of a VH domain, but still retains some antigen-binding function of the intact antibody.

Binding fragments of an antibody are produced by recombinant DNA techniques, or by enzymatic or chemical cleavage of intact antibodies. Binding fragments include Fab, Fab′, F(ab′)2, Fv, and single-chain antibodies. An antibody other than a “bispecific” or “bifunctional” antibody is understood to have each of its binding sites identical. Digestion of antibodies with the enzyme, papain, results in two identical antigen-binding fragments, known also as “Fab” fragments, and a “Fe” fragment, having no antigen-binding activity but having the ability to crystallize. Digestion of antibodies with the enzyme, pepsin, results in a F(ab′)2 fragment in which the two arms of the antibody molecule remain linked and comprise two-antigen binding sites. The F(ab′)2 fragment has the ability to crosslink antigen. “Fv” when used herein refers to the minimum fragment of an antibody that retains both antigen-recognition and antigen-binding sites. “Fab” when used herein refers to a fragment of an antibody that comprises the constant domain of the light chain and the CHI domain of the heavy chain.

By “agent” is meant any small molecule chemical compound, antibody, nucleic acid molecule, or polypeptide, or fragments thereof.

By “ameliorate” is meant decrease, suppress, attenuate, diminish, arrest, or stabilize the development or progression of a disease.

By “alteration” is meant a change (increase or decrease) in the expression levels or activity of agene or polypeptide as detected by standard art known methods such as those described herein. As used herein, an alteration includes a 10% change in expression levels, preferably a 25% change, more preferably a 40% change, and most preferably a 50% or greater change in expression levels.”

By “analog” is meant a molecule that is not identical, but has analogous functional or structural features. For example, a polypeptide analog retains the biological activity of a corresponding naturally-occurring polypeptide, while having certain biochemical modifications that enhance the analog's function relative to a naturally occurring polypeptide. Such biochemical modifications could increase the analog's protease resistance, membrane permeability, or half-life, without altering, for example, ligand binding. An analog may include an unnatural amino acid.

By “disease” is meant any condition or disorder that damages or interferes with the normal function of a cell, tissue, or organ. Examples of diseases include cancer.

By “effective amount” is meant the amount of a required to ameliorate the symptoms of a disease relative to an untreated patient. The effective amount of active compound(s) used to practice the present disclosure for therapeutic treatment of a disease varies depending upon the manner of administration, the age, body weight, and general health of the subject. Ultimately, the attending physician or veterinarian will decide the appropriate amount and dosage regimen. Such amount is referred to as an “effective” amount.

By “ERCP” is meant Endoscopic retrograde cholangiopancreatography (ERCP) is a technique that combines the use of endoscopy and fluoroscopy to diagnose and treat certain problems of the biliary or pancreatic ductal systems. Through the endoscope, the physician can see the inside of the stomach and duodenum and inject a contrast medium into the ducts in the biliary tree and pancreas so they can be seen on radiographs. ERCP is used primarily to diagnose and treat conditions of the bile ducts and main pancreatic duct, including gallstones, inflammatory strictures (scars), leaks (from trauma and surgery), and cancer. ERCP can be performed for diagnostic and therapeutic reasons, although the development of safer and relatively non-invasive investigations such as magnetic resonance cholangiopancreatography (MRCP) and endoscopic ultrasound has meant that ERCP is now rarely performed without therapeutic intent.

The term “express” refers to the ability of a gene to express the gene product including for example its corresponding mRNA or protein sequence (s).

As used herein, the term “expression cassette” or “nucleic acid expression cassette” refers to a DNA sequence that encodes and is capable of producing one or more desired expression products (RNA or protein). Production of such a desired expression product may require the presence of various expression control sequences operatively linked to the DNA sequence encoding that product. Such control sequences include a promoter, as well as other non-coding nucleotide sequences. An expression cassette may include none, some or all of these expression control sequences. If some or all of these expression control sequences are absent from the expression cassette, they are supplied by a vector into which the expression cassette is inserted.

As used herein, a “subject” means a human. The terms, “patient”, “individual” and “subject” are used interchangeably herein. A subject can be one who has been previously diagnosed with or identified as suffering from or having a condition in need of treatment (e.g., brain tumors) or one or more complications related to the condition, and optionally, have already undergone treatment for the condition or the one or more complications related to the condition. Alternatively, a subject can also be one who has not been previously diagnosed as having a condition or one or more complications related to the condition. For example, a subject can be one who exhibits one or more risk factors for a condition or one or more complications related to the condition or a subject who does not exhibit risk factors. A “subject in need” of treatment for a particular condition, e.g. a neurodegenerative condition, can be a subject suspected of having that condition, diagnosed as having that condition, already treated or being treated for that condition, not treated for that condition, or at risk of developing that condition.

The term “biliary tree” includes the common bile duct and hepatic duct. The pancreatic duct also may be included with the common bile duct and/or hepatic duct in certain aspects. The ureters and the kidney may also be included with the common bile duct and/or common hepatic duct in certain aspects.

The term “polynucleotide” as used herein means a sequence of 20 or more nucleotides. A polynucleotide may RNA, DNA or a hybrid RNA or DNA molecule; and may be single stranded or double stranded. In certain embodiments, a polynucleotide is a single or double-stranded DNA molecule.

The term “promoter” as used herein is defined as a DNA sequence recognized by the synthetic machinery of the cell, or introduced synthetic machinery, required to initiate the specific transcription of a polynucleotide sequence. A “constitutive” promoter is a nucleotide sequence which, when operably linked with a polynucleotide which encodes or specifies a gene product, causes the gene product to be produced in a cell under most or all physiological conditions of the cell. An “inducible” promoter is a nucleotide sequence which, when operably linked with a polynucleotide which encodes or specifies a gene product, causes the gene product to be produced in a cell substantially only when an inducer which corresponds to the promoter is present in the cell. A “tissue-specific” promoter is a nucleotide sequence which, when operably linked with a polynucleotide encodes or specified by a gene, causes the gene product to be produced in a cell substantially only if the cell is a cell of the tissue type corresponding to the promoter.

By “fragment” is meant a portion of a polypeptide or nucleic acid molecule. This portion contains, preferably, at least 10%, 20%, 30%, 40%, 50%, 60%, 70%, 80%, or 90% of the entire length of the reference nucleic acid molecule or polypeptide. A fragment may contain 10, 20, 30, 40, 50, 60, 70, 80, 90, or 100, 200, 300, 400, 500, 600, 700, 800, 900, or 1000 nucleotides or amino acids.

“Hybridization” means hydrogen bonding, which may be Watson-Crick, Hoogsteen or reversed Hoogsteen hydrogen bonding, between complementary nucleobases. For example, adenine and thymine are complementary nucleobases that pair through the formation of hydrogen bonds.

“Immunoassay” is an assay that uses an antibody to specifically bind an antigen (e.g., a marker). The immunoassay is characterized by the use of specific binding properties of a particular antibody to isolate, target, and/or quantify the antigen.

The term, “obtaining” as in “obtaining an agent” includes synthesizing, purchasing, or otherwise acquiring the agent.

The term “mAb” refers to monoclonal antibody. Antibodies of the disclosure comprise without limitation whole native antibodies, bispecific antibodies; chimeric antibodies; Fab, Fab′, single chain V region fragments (scFv), fusion polypeptides, and unconventional antibodies.

The terms “polypeptide,” “peptide” and “protein” are used interchangeably herein to refer to a polymer of amino acid residues. The terms apply to amino acid polymers in which one or more amino acid residue is an analog or mimetic of a corresponding naturally occurring amino acid, as well as to naturally occurring amino acid polymers. Polypeptides can be modified, e.g., by the addition of carbohydrate residues to form glycoproteins. The terms “polypeptide,” “peptide” and “protein” include glycoproteins, as well as non-glycoproteins.

By “reduces” is meant a negative alteration of at least 10%, 25%, 50%, 75%, or 100%.

A “reference” refers to a standard or control conditions such as a sample (human cells) or a subject that is a free, or substantially free, of an agent such as plasmid or transposon of the present disclosure.

A “reference sequence” is a defined sequence used as a basis for sequence comparison. A reference sequence may be a subset of or the entirety of a specified sequence; for example, a segment of a full-length cDNA or gene sequence, or the complete cDNA or gene sequence. For polypeptides, the length of the reference polypeptide sequence will generally be at least about 16 amino acids, preferably at least about 20 amino acids, more preferably at least about 25 amino acids, and even more preferably about 35 amino acids, about 50 amino acids, or about 100 amino acids. For nucleic acids, the length of the reference nucleic acid sequence will generally be at least about 50 nucleotides, preferably at least about 60 nucleotides, more preferably at least about 75 nucleotides, and even more preferably about 100 nucleotides or about 300 nucleotides or any integer thereabout or there between.

By “specifically binds” is meant a compound or antibody that recognizes and binds a polypeptide of the disclosure, but which does not substantially recognize and bind other molecules in a sample, for example, a biological sample, which naturally includes a polypeptide of the disclosure.

As used herein, the term “subject” is intended to refer to any individual or patient to which the method described herein is performed. Generally, the subject is human, although as will be appreciated by those in the art, the subject may be an animal. Thus other animals, including mammals such as rodents (including mice, rats, hamsters and guinea pigs), cats, dogs, rabbits, farm animals including cows, horses, goats, sheep, pigs, etc., and primates (including monkeys, chimpanzees, orangutans and gorillas) are included within the definition of subject.

By “substantially identical” is meant a polypeptide or nucleic acid molecule exhibiting at least 50% identity to a reference amino acid sequence (for example, any one of the amino acid sequences described herein) or nucleic acid sequence (for example, any one of the nucleic acid sequences described herein). Preferably, such a sequence is at least 60%, more preferably 80% or 85%, and more preferably 90%, 95% or even 99% identical at the amino acid level or nucleic acid to the sequence used for comparison.

Sequence identity is typically measured using sequence analysis software (for example, Sequence Analysis Software Package of the Genetics Computer Group, University of Wisconsin Biotechnology Center, 1710 University Avenue, Madison, Wis. 53705, BLAST, BESTFIT, GAP, or PILEUP/PRETTYBOX programs). Such software matches identical or similar sequences by assigning degrees of homology to various substitutions, deletions, and/or other modifications. Conservative substitutions typically include substitutions within the following groups: glycine, alanine; valine, isoleucine, leucine; aspartic acid, glutamic acid, asparagine, glutamine; serine, threonine; lysine, arginine; and phenylalanine, tyrosine. In an exemplary approach to determining the degree of identity, a BLAST program may be used, with a probability score between e-³ and e-¹⁰⁰ indicating a closely related sequence.

Ranges provided herein are understood to be shorthand for all of the values within the range. For example, a range of 1 to 50 is understood to include any number, combination of numbers, or sub-range from the group consisting 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50.

As used herein, the terms “treat,” treating,” “treatment,” and the like refer to reducing or ameliorating a disorder and/or symptoms associated therewith. It will be appreciated that, although not precluded, treating a disorder or condition does not require that the disorder, condition or symptoms associated therewith be completely eliminated.

A “transgene” is used herein to conveniently refer to a polynucleotide or a nucleic acid that is intended or has been introduced into a cell or organism. Transgenes include any nucleic acid, such as a gene that encodes a polypeptide or protein. Suitable transgenes, for example, for use in gene therapy are well known to those of skill in the art. For example, the vectors described herein can deliver transgenes and uses that include, but are not limited to, those described in U.S. Pat. Nos. 6,547,099; 6,506,559; and 4,766,072; Published U.S. Application No. 20020006664; 20030153519; 20030139363; and published PCT applications of WO 01/68836 and WO 03/010180, and e.g. miRNAs and other transgenes of WO2017/152149; each of which are hereby incorporated herein by reference in their entirety.

Unless specifically stated or obvious from context, as used herein, the term “or” is understood to be inclusive. Unless specifically stated or obvious from context, as used herein, the terms “a”, “an”, and “the” are understood to be singular or plural.

Unless specifically stated or obvious from context, as used herein, the term “about” is understood as within a range of normal tolerance in the art, for example within 2 standard deviations of the mean. About can be understood as within 10%, 9%, 8%, 7%, 6%, 5%, 4%, 3%, 2%, 1%, 0.5%, 0.1%, 0.05%, or 0.01% of the stated value. Unless otherwise clear from context, all numerical values provided herein are modified by the term about.

The recitation of a listing of chemical groups in any definition of a variable herein includes definitions of that variable as any single group or combination of listed groups. The recitation of an embodiment for a variable or aspect herein includes that embodiment as any single embodiment or in combination with any other embodiments or portions thereof.

Any compositions or methods provided herein can be combined with one or more of any of the other compositions and methods provided herein.

As used herein, the terms “prevent,” “preventing,” “prevention,” “prophylactic treatment” and the like refer to reducing the probability of developing a disorder or condition in a subject, who does not have, but is at risk of or susceptible to developing a disorder or condition.

Such treatment (surgery and/or chemotherapy) will be suitably administered to subjects, particularly humans, suffering from, having, susceptible to, or at risk for pancreatic cancer or disease, disorder, or symptom thereof. Determination of those subjects “at risk” can be made by any objective or subjective determination by a diagnostic test or opinion of a subject or health care provider (e.g., genetic test, enzyme or protein marker, a marker (as defined herein), family history, and the like).

Other aspects of the disclosure are disclosed infra.

### BRIEF DESCRIPTION OF THE DRAWINGS

The patent or application file contains at least one drawing executed in color. Copies of this patent or patent application publication with color drawings will be provided by the Office upon request and payment of the necessary fee.

FIG. 1 illustrates an exemplary catheter placement in which a catheter balloon and distal catheter end are positioned in an extrahepatic common hepatic duct proximate to a subject's liver wall.

FIG. 2 illustrates an exemplary catheter placement in which a catheter balloon and distal catheter end are positioned in an intrahepatic common hepatic duct within a subject's liver.

FIG. 3 illustrates an exemplary catheter placement in which a distal catheter end is positioned in an intrahepatic duct within a subject's liver while a catheter balloon is positioned in an extrahepatic duct proximate to a subject's liver wall.

FIG. 4 illustrates an exemplary catheter placement in which a catheter balloon and distal catheter end are positioned within a right hepatic duct within a subject's liver.

FIG. 5 illustrates an exemplary catheter placement in which a catheter balloon and distal catheter end are positioned within a left hepatic duct within a subject's liver.

FIG. 6 illustrates an exemplary catheter placement in which a catheter balloon and distal catheter end are positioned within a pancreatic duct.

FIG. 7 illustrates an exemplary catheter useful in the present methods and systems having a handle allowing for different lumens to be accessed including air/balloon, injection, and guidewire lumens.

FIGS. 8A and 8B illustrate exemplary catheter cross-sections with injection port/lumen and an injection port/lumen, respectively. The diagram in 8A shows a catheter configuration of common commercial catheters with injection port injecting laterally into duct wall. 8B shows the disclosed catheter design that has all ports/lumens forward injecting in order to avoid wall stress that could rupture the ducts.

FIG. 9 shows a fluoroscopic image of rupture of the proximal common hepatic duct during a hydrodynamic injection.

FIGS. 10A-10B show images of an exemplary injection protocol. FIG. 10A shows initial localization of catheter and balloon, while FIG. 10B shows an exemplary progression of contrast agent into the liver during hydrodynamic injection.

FIG. 11 depicts a graph showing a pressure curve of intrabiliary pressure during an exemplary hydrodynamic injection protocol and subsequent pressure drop at the end of injection as described in Example 6.

FIG. 12 depicts a graph of a pressure curve of intrabiliary pressure during an exemplary injection protocol showing that pressure monitoring may detect balloon slippage and loss of ductal seal as described in Example 7.

FIGS. 13A-13E are photographs showing that pigs tolerated repeated hydrodynamic injections in the biliary system as gross examination of the livers immediately post-injection series did not show any obvious abnormalities as described in Example 8.

FIG. 14 shows images of hepatocyte cytoplasm of samples of Example 9 which show that histology of pig liver post-repeated hydrodynamic biliary injection evidences hydrodynamic effects.

FIG. 15 shows images of hepatocyte cytoplasm of samples of Example 9 which show that hydrodynamic biliary injection induces acute tissue structural changes in pigs that is similar to mouse hydrodynamic tail vein injection.

FIG. 16 shows an image of rupture of the proximal common hepatic duct following injections of 100% contrast solution above 30 mL volume at 2 mL/second as disclosed in Example 10.

FIG. 17 show images demonstrating that injection of a 25% contrast solution did not mediate rupture of hepatic ducts as disclosed in Example 10.

FIG. 18 shows a fluorescent imaging demonstrating very low transfection rates when 3 mg of DNA of a plasmid with an EF1alpha promoter was administered in 30 mL volume and injected at 2 mL/sec.

FIG. 19 shows an hFIX immunostained image depicting high transfection efficiencies of between 35-50% after use of liver-specific promoter as disclosed in Example 11.

FIGS. 20A-20B show two images and a bar graph, respectively. FIG. 20A shows images of hFIX vascular hydrodynamic injected mouse (top panel) and hFIX biliary hydrodynamic injected pig (bottom panel). FIG. 20B is a bar graph showing that increased DNA concentration results in increased transfection efficiency in pigs and that either dose in pigs is higher than the transfection efficiency achieved in mice.

FIGS. 21A-21C show a series of gels testing for the presence of plasmid DNA (pDNA) by polymerase chain reaction (PCR). FIG. 21A shows results of pDNA testing in pig plasma and bile.

FIG. 21B shows results of pDNA testing in pig stool. FIG. 21C shows results of pDNA testing in a series of tissues sampled from Pig #3one-week post-injection.

FIG. 22 shows hematologic data after hydrodynamic injection for four pigs injected via the biliary route at 30 mL at 2 mL/sec.

FIG. 23 shows biochemical data after hydrodynamic injection for four pigs injected via the biliary route at 30 mL at 2 mL/sec.

FIG. 24 shows a series of dot plots showing subject vital signs before and after hydrodynamic injection via the biliary route.

FIG. 25 shows a series of dot plots showing subject vital signs before and after a subsequent round of hydrodynamic injection three weeks later.

FIGS. 26A-26C present a series of bar graphs that quantify hFIX immunostained positive area within a hepatic lobule post-injection. FIG. 26A shows the % area of hFIX-positive immunostaining within individual lobules (n=5) in pig #3 (one-week post-injection) and pig #1, #2 and #4 (3 weeks post-injection). FIG. 26B shows that among pig #3 and #4, distal and proximal portions of an individual hepatic lobe do not show consistent differences in % area of hFIX-positive hepatocytes (n=5-6 lobules per lobe). FIG. 26C shows that higher doses of plasmid DNA led to higher hFIX % area transfected.

FIG. 27 depicts a series of H&E-stained liver tissue samples that show large vesicle formation within hepatocytes, dilated hepatic sinusoids, and dilute cytoplasm inside hepatocytes after biliary injection at 10 mL/sec with diluted cytoplasm.

FIGS. 28A-28E show design and validation of the hFIX plasmid vectors in a mouse model by way of two vector maps, a dot plot, a stained tissue, and a bar graph. FIG. 28A is a diagram of pT-LP1-hFIX. FIG. 28B is a diagram of pCMV-hyperPB. FIG. 28C shows that delivery of 8 μg hFIX transposon and 1 μg hyperPB transposase by hydrodynamic tail vein injection in mice led to 11,268±1,077 ng/mL hFIX at one-week post-injection. FIG. 28D shows that frequent hFIX-positive hepatocytes by cytoplasmic immunostaining in mouse liver. FIG. 28E shows that individual lobules were quantified in three injected mice, and stained area quantified (n=6 lobules per mouse).

FIGS. 29A-29G depict four images and three graphs showing ERCP-mediated hydrodynamic injection is well tolerated by pigs. FIG. 29A shows that cannulation of ampulla of Vater during endoscopy in pig #2 is depicted. FIG. 29B shows that branches of the biliary tree are displayed prior to injection in pig #1. FIGS. 29C and 29D show that contrast injection with a balloon inflated in the common bile duct post-hydrodynamic injection reveals intact biliary system as evidenced by no contrast extravasation (Pig #1, FIG. 29C, Pig #3, FIG. 29D). FIG. 29E shows that for all four pigs, the values of the heart rate the mean arterial pressure, the respiratory rate, and pulse oximetry were compared pre- and post-procedure, demonstrating no significant difference. An unpaired, parametric, two-tailed t-test was used for analysis; significance (P<0.05). FIG. 29F shows that liver function biomarkers monitored post-hydrodynamic injection show minimal toxicity in pigs. ALT and AST remained within normal range in the week post-injection. Similarly, total bilirubin, direct bilirubin, albumin, and GGT did not show any alterations. FIG. 29G shows that hematologic parameters monitored after hydrodynamic injection show minimal effects. White blood cell (WBC) count post-injection showed no acute changes. Platelet (PLT) count demonstrated a mild increase post-injection. Red blood cell (RBC) count and hemoglobin showed a non-significant changes post-injection. Day 0=injection day, representing pre-procedure specimen.

FIGS. 30A-30G show pig liver is anatomically and histologically normal after hydrodynamic injection with no gene delivery into non-liver tissues. FIG. 30A shows the visceral liver surface of pig #3 one-week post-injection with all five lobes observed along with the gall bladder and portal triad, showing no gross abnormalities. FIG. 30B shows that the diaphragmatic liver surface of pig #3 similarly shows no abnormalities. FIG. 30C shows that the intrahepatic biliary branches of pig #3 were dissected with no ruptures or lesions observed. FIG. 30D shows that hydrodynamically injected pigs exhibited normal liver histology one-week post-injection. H&E stains are presented for pig #3 and a non-injected pig. PCR for hFIX DNA demonstrated the expected band size (550 bp). Positive control (CTL) is the hFIX plasmid, and negative control is non-injected pig liver. FIG. 30E shows that pDNA biodistribution into porcine body fluids post-hydrodynamic injection was studied. Plasma samples were taken pre-procedure, 15 minutes post-procedure, and at day 1 post-procedure for all four pigs. Bile tested by PCR at one-week post-injection during necropsy was negative. FIG. 30F shows that the stool samples were collected from pig #2 and #4 pre-procedure, and at days 4 and 14 post-injection were tested for hFIX DNA by PCR. FIG. 30G shows that transfection of pDNA post-hydrodynamic injection into non-liver tissues was not demonstrated. A panel of 23 different tissues were sampled during necropsy in pig #3 one-week post-injection was tested by PCR.

FIGS. 31A-31D shows biliary hydrodynamic injection mediates human FIX DNA delivery and protein expression in all liver lobes. FIG. 31A shows the tissue sampling scheme of pig liver across different lobes: right lateral lobe (RLL), right medial lobe (RML), left medial lobe (LML), left lateral lobe (LLL), and quadrate lobe. Proximal and distal samples to the site of catheter injection in the CHD were taken among all lobes. Pig #3 liver is depicted in FIG. 31A, harvested one-week post-injection for subsequent tissue analysis. FIG. 31B shows that PCR on genomic DNA revealed the expected band (550 bp) for hFIX DNA in all liver lobes tested. FIG. 31C shows that RT-PCR was performed on RNA extracted at one-week post-injection demonstrated the expected band size. RNA extracted from HepG2 cells transfected with pT-LP1-hFIX plasmid serves as one positive control. For both PCR's, positive control (CTL) was hFIX plasmid, and negative control is non-injected pig liver tissue. FIG. 31D shows that Western blot on liver tissue demonstrated correct size of hFIX (70 kDa) with a low-level cross reactivity for porcine FIX at the same molecular weight. Quantification of hFIX expression by western blot band intensity and normalized with beta-actin is provided. (CTL=control, non-injected pig liver).

FIGS. 32A-32C show immunohistochemistry shows efficient human Factor IX expression in pigs after hydrodynamic delivery. FIG. 32A shows that IHC for hFIX in human liver tissue demonstrated homogenous, cytoplasmic expression in hepatocytes, with no apparent intensity difference among hepatocytes in a lobule (central vein area versus cords). Control tissue from a non-injected pig liver does not demonstrate hFIX staining as expected, with only light background staining for porcine FIX appreciated. Immunostaining in hFIX-injected pig #3 revealed abundant hFIX-hepatocytes, with similar cytoplasmic staining to human hepatocytes, although more variable expression intensity amongst positive cells. (bar=50 μm) FIG. 32B shows that tissue section of hFIX-injected pig at low power reveals hFIX expression in every lobule (bar=500 μm). FIG. 32C shows that an individual lobule demonstrates the most intense expression around the central vein radiating toward the fibrous lobule borders (bar=100 μm). Positive hFIX-hepatocytes are seen in all functional zones, although most abundant in zone 3 (bar=50 μm). hFIX-expression is most intense and uniform around the central vein (bar=20 μm), although numerous positive cells can also be seen near the portal triad (bar=50 μm). IHC images for the hFIX injected pig comes from the distal biopsy site of the right medial lobe of pig #3.

FIGS. 33A-33C show that hFIX shows uniform distribution within the pig liver and successful expression in every pig after hydrodynamic injection. FIG. 33A shows that the percentage area of hFIX-positive immunostaining within individual lobules (n=5-6) in pig #3 (one-week post-injection) and pig #1, #2 and #4 (3 weeks post-injection) is provided. FIG. 33B shows that amongst pig #3 and #4, distal and proximal portions of an individual hepatic lobe do not show consistent differences in percentage area of hFIX-positive hepatocytes (n=5-6). FIG. 33C shows that higher doses of pDNA led to higher hFIX percentage area transfected. Each pig represents the 4 lobe averages determined in FIG. 33A now pooled together with quadrate lobe removed due to small size (˜5% of liver). Data measurements are presented as mean±standard error of mean (SEM). Statistics represent unpaired, parametric, two-tailed t-tests; significance (p<0.05). **** p<0.0001; ** p<0.01; * p<0.05; n.s.=not significant.

FIGS. 34A-34C show that pig biliary hydrodynamic injection yields superior transfection efficiency to mouse hydrodynamic tail vein injection. FIG. 34A shows that mouse HTVI was primarily located around central veins in zone 2 (bar=50 μm). Pig hydrodynamic biliary injection led to hFIX-positive expression almost uniformly circling central veins in zone 3 (bar=200 μm), and then radiating outward along the chords reaching zone 1 and 2 (pig #3 LML proximal section depicted). FIG. 34B shows that a model of the mechanism of biliary versus vascular hydrodynamic delivery. FIG. 34C showed that for individual lobules, the percentage of hFIX-positive pig hepatocytes was significantly more than hFIX-positive mouse hepatocytes at matched DNA per liver weight doses (Mouse represents 3 mice, 18 lobules; Pig represents average of 8 lobes from 2 pigs, with quadrate lobe excluded). Data measurements are presented as mean±standard error of mean (SEM). Statistics represent unpaired, parametric, two-tailed t-tests; significance (p<0.05). **** p<0.0001; ** p<0.01.

FIG. 35 shows pCMV-hyperPB plasmid facilitates efficient transposition of a reporter transposon into the genome of HEK293 cells in vitro with stable expression after repeated passages of HEK293 cells reflecting integration of the GFP gene into the genome.

FIGS. 36A-36B show vital signs were monitored and ultrasound was performed during the hydrodynamic procedure displaying minimal disturbance pre- and post-procedure. FIG. 36A shows that the gallbladder measured pre- and post-procedure did not demonstrate changes in size, confirming balloon seal during hydrodynamic injection prevented fluid entry into this space. FIG. 36B shows that Vital signs taken during the procedure did not show any acute changes in blood pressure, heart rate, pulse oximetry, or respiratory tidal volumes from pre- to post-procedure.

FIG. 37 shows that hFIX DNA delivered by biliary hydrodynamic injection is detected long-term in pig liver by PCR testing at a period of 3-weeks post-injection.

FIGS. 38A-38B shows that human FIX antibody mediates low-level cross-reactivity to porcine FIX. FIG. 38A shows that Western blot of human plasma and pig plasma was performed, demonstrating that the hFIX antibody stains human plasma intensely at the correct molecular weight (70 kDa), while demonstrating minimal binding to porcine FIX. FIG. 38B shows that the band intensity on the western blot was quantified, using normalization of the input protein levels in each well (1:0.66 human plasma to pig plasma protein).

FIGS. 39A-39C show representative hFIX immunostaining of pig and mouse liver sections at low magnification power. FIG. 39A shows that an example of a liver section stained for hFIX at low magnification power is presented from pig #3, LML proximal section, demonstrating that immunostaining can be observed in every single lobule, with intensity highest in the center of the lobule (bar=1 mm). FIG. 39B shows that a magnified image from the same liver biopsy section is presented, showing intense staining bordering the central vein and radiating to the lobule borders (bar=200 m). FIG. 39C shows that a representative image of mouse liver after HTVI of hFIX is presented, demonstrating positive hFIX-hepatocytes in every lobule (bar=200 m).

FIGS. 40A-40B show fluoroscopy monitors the success of biliary hydrodynamic injection. FIG. 40A shows that the anatomy of the biliary tract and the successful seal of the balloon to prevent retrograde flow of contrast was confirmed. With placement of catheter inside the CHD and balloon inflated, bifurcation of the CHD into the right and left branches is observed. FIG. 40B shows hydrodynamic delivery to all lobes was confirmed by measuring real-time fluoroscopy of the injection. Example fluoroscopic images of one hydrodynamic injection are provided, showing a time course of images during injection (30 mL in 2 mL/sec). At the completion of injection (15 seconds), all liver lobes contain detectable contrast.

FIGS. 41A-41E show vital signs monitored during biliary hydrodynamic injection demonstrate no significant changes. All pigs had vital signs monitored under anesthesia during ERCP procedure. Values pre- and post-procedure are provided, representing possible physiologic perturbations from hydrodynamic injection. Heart rate (FIG. 41A), respiratory rate (FIG. 41B), pulse oximetry (FIG. 41C), end tidal CO2 (FIG. 41D), and mean arterial pressure (FIG. 41E) all demonstrated no significant (n.s.) changes from pre- to post-procedure (unpaired two-tailed, parametric t-test used, P<0.05).

FIGS. 42A-42D shows monitoring of intrabiliary pressure during hydrodynamic injection. Baseline pressure within the biliary system is minimal before injection in all conditions (FIGS. 42A-42D). Shortly before injection, the balloon is inflated creating a seal which appears to have minimal effect on the measured pressure. Upon initiation of injection (solid black arrow, parameters provided above graphs), pressure increases to a short peak, before equilibration during flow at a slightly lower pressure. Cessation of injection (dashed black arrows) yields a sharp decrease in pressure. The deflation of balloon (solid grey arrows) drops pressure further, suggesting a measure of baseline hydrostatic pressure remains in the system after the injection is completed. FIG. 42A shows pressure readings for the injection of 30 mL at 2 mL/sec demonstrated a plateau pressure of 80 mmHg during injection, that promptly dropped the moment the injection ended. FIG. 42B shows that a small level of pressure was released when the balloon was deflated, representing pressure generated by balloon restriction of biliary flow, although the exact value was variable between the different experiments (4.23 mmHg to 18.92 mmHg). A peak pressure point at the initial power injector was also noted in two of the conditions (114.76 mmHg in FIG. 42C and 181.36 mmHg in FIG. 42D) before slightly falling into a plateau phase.

FIGS. 43A-43B) show imaging and biochemical analysis support the safety profile ofbiliary hydrodynamic injection. FIG. 43A shows that an abdominal CT with contrast was performed on Day 1 post-procedure. The axial, sagittal and coronal images did not demonstrate any evidence of intra- or extrahepatic biliary dilation, hepatic infarction/necrosis, abnormal gallbladder dilation, or gallbladder inflammation. The systemic and portal venous systems were patent. FIG. 43B shows that transient, acute elevation in AST and total bilirubin in the three injected was noted, which were not observed in five other pigs with resolution of any biochemical abnormalities by day 1 post-injection. Pig #1 was color-coded as green dots, Pig #2 was color-coded as blue dots, Pig #3, which received multiply injections with the largest volume and highest injection speed, was color-coded as red dots.

FIGS. 44A-44B show histology of pig liver post-repeated hydrodynamic biliary injection shows evidence of hydrodynamic effects. FIG. 44A shows H&E stains from low flowrate pig injections (flow rates: <5 mL/sec; Pig #1 depicted) or high flow rate pig injections (flowrate: 10 mL/sec; Pig #3 depicted) are illustrated, along with H&E from a normal, non-injected pig liver for comparison. Histology taken from livers 15 minutes post-injection showed significant dilation of hepatic sinusoids (black arrows) and formation of intracellular vesicles, but otherwise no areas of focal necrosis. FIG. 44B shows histology of pig liver on Day 1 and Day 14 post-procedure showed normal liver histology. H&E staining on pigs euthanized on Day 1 and Day 14 post-procedure are illustrated with no obvious sinusoid spaces dilation observed. Scattered fluid-filled vesicles were observed more rarely on Day 1 post-procedure. No fluid-filled vesicles were noted on Day 14. Scale bar: 200 μm in 4× images; 50 μm in 20× images.

FIGS. 45A-45B show hydrodynamic biliary injection induces acute tissue structural changes in pigs that is similar to mouse hydrodynamic tail vein injection. FIG. 45A shows a mouse injected with 10% body fluid volume over 4-7 seconds through the tail vein and sacrificed 15 minutes later for comparison to the biliary hydrodynamic injection in pigs. Small fluid-filled cytoplasm vesicles (black arrows), scattered hepatocytes with dilute cytoplasm, and occasional hepatocytes with engulfed red blood cells are observed in the mouse liver. Scale bar: left, 50 μm; right, 20 μm. FIG. 44B shows results for Pig #3, which was a high flow rate injection pig, had largely similar changes, with larger fluid-filled cytoplasmic vesicles and more frequent hepatocytes with dilute cytoplasm. Scale bar: left, 50 μm; right, 2.0 μm.

FIG. 46 shows representative real-time vital signs during hydrodynamic injection, including temperature, electrocardiogram and heart rate. Continuous measurements were taken throughout one of the biliary hydrodynamic injection procedures in pigs. There was no change in temperature, heart rate or electrocardiogram at parameters of injection of 4 mL/sec over 10 seconds.

FIGS. 47A-47E show that gross examination of livers of pigs immediately post-injection series did not show any obvious abnormalities. The visceral surface (FIG. 47A) and diaphragmatic surface (FIG. 47B) of pig #1 are depicted, showing lobes with no obvious lesions. The common hepatic duct (CHD) of pig #1 where the catheter was placed for injection was further probed (FIG. 47C) demonstrating no wall lesions or tears. The visceral surface of pig #2 (FIG. 47D) and pig #3 (FIG. 47E) are also shown after tissue harvest post-injection, also demonstrating no obvious gross abnormalities.

FIG. 48 shows PCR for plasmid DNA in serum samples illustrates the permeability of the biliary system. To evaluate the escape of fluid from the biliary system during injection, plasmid DNA (pCLucf) was diluted into the injection solution. PCR primers were designed to target the GFP sequence in the pCLucf plasmid. PCR was performed on the serum samples obtained pre-injection, 15 min post-injection and on day 1 post-injection. The DNA molecule was detected in the 15 min post-injection sample, which was no longer detectable on day 1 post-injection. The PCR band size over the GFP gene: ˜300 bp. Negative control used molecular water as template DNA. Bright band in the ladder represents 500 bp, and each ladder band below is at intervals of 100 bp.

FIG. 49 shows that vesicle formation was observed in the proximal and distal portions of liver lobes at high flow rates. H&E stain from high flow rate injection pig #3 (10 mL/sec) shows vesicle formation across the five liver lobes and sampling proximal and distal to the common hepatic duct injection site. LLL, left lateral lobe; LML, left medial lobe; RML, right medial lobe; RLL, right lateral lobe. Scale bar: 100 m.

FIGS. 50A-50B show that bile ducts did not show signs of injury or rupture from biliary hydrodynamic injection. Liver histology of the pig injected at the highest flow rates tested (10 mL/sec) was assessed in a liver collected 15 minutes after injection. FIG. 50A shows that the morphology of large, medium and small bile duct exhibited no gross differences in a pig injected at high flow rates compared to an un-injected, normal pig liver control. FIG. 50B shows that the epithelium lining of the intrahepatic and extrahepatic bile duct was also intact in the same animal, while the peribiliary glands maintained integrity. Scale bar: 50 m.

FIGS. 51A-51E show different cell types in the liver can be targeted for gene expression with biliary hydrodynamic injection. FIG. 51A shows that reporter Firefly Luciferase staining was prominently observed in bile ducts after injection. The intensity of the stain was significantly more than the surrounding hepatocytes, suggesting more DNA delivery to these tissues. FIG. 51B shows that GFP staining was observed intensely in endothelial cells lining arterioles in the portal triads of the liver. FIG. 51C shows that Luciferase staining was also observed lightly staining interstitial fibrous tissue in the portal triads. FIG. 51D shows that Firefly luciferase staining was observed in hepatocytes scattered throughout the lobule. FIG. 51E shows that smooth muscle associated with arteries in portal triads also demonstrated efficient expression of GFP after injection. FIG. 51F shows that neurons in portal triads also demonstrated efficient expression of GFP after injection.

FIGS. 52A-52F show different cell types in the pancreas that can be targeted with ductal hydrodynamic injection. FIG. 52A shows that pancreatic ductal epithelial cells prominently stain with Firefly luciferase after injection. FIG. 52B shows that pancreatic acinar cells can be abundantly found and express GFP. FIG. 52C shows that pancreatic islet cells prominently stain with firefly luciferase after injection. FIG. 52D shows that cells of the hematopoietic lineage also demonstrated intense staining with GFP. FIG. 52E shows that endothelial cells in the pancreas were positive for Firefly luciferase staining. FIG. 52F shows that neurons stained intensely with GFP.

FIGS. 53A-53E show that different regions of the liver can be targeted when the flow rate of hydrodynamic injection through the biliary system is altered. In the example, 7 mg of plasmid encoding for CMV promoter-Firefly Luciferase and SV40 promoter-GFP (˜7 kb plasmid size) was injected into a 50 kg pig at 4 mL/sec over 40 mL volume total via hydrodynamic injection. FIG. 53A shows staining for GFP revealed prominent staining around the portal triads. FIG. 53B shows Firefly luciferase staining was present along lobular borders. By comparison, FIG. 53C shows that the central vein at this flow rate had comparatively few cells. FIG. 53D shows staining patterns at 2 mL/sec localized transfection primarily around the central vein. Of note, FIG. 53E shows expression was particularly intense near large vessels, which yielded radiation of transfection outward, irrespective of lobules.

FIGS. 54A-54D show that promoters can lead to exclusion of expression in different cell types in the liver and pancreas. FIG. 54A shows intense GFP staining of endothelial cells (solid arrow) was observed with the SV40 promoter, while bile ducts (dashed arrow) were not stained for GFP. FIG. 54B shows that, by comparison, Firefly luciferase could be observed in both cell types. FIG. 54C shows that the acinar cells only stained strongest for GFP staining under SV40 promoter, while FIG. 54D shows that the CMV promoter did not drive strong firefly luciferase expression/staining in the same cell types.

FIG. 55 shows a diagram of accessing the common bile duct by endoscopic ultrasound for the subsequent procedure biliary hydrodynamic injection.

FIG. 56 shows a diagram of accessing the common hepatic duct by percutaneous needle access to the gallbladder for subsequent procedure of biliary hydrodynamic injection.

FIG. 57 shows a diagram of accessing the common hepatic duct by endoscopic needle through the stomach wall to the intrahepatic ducts followed by retrograde hydrodynamic injection with fluid injection proximal to the balloon.

FIG. 58 shows a diagram of a multiple-balloon administration system and accessing the common hepatic duct by endoscopic needle through the stomach wall to the intrahepatic ducts followed by retrograde hydrodynamic injection.

FIG. 59 depicts a diagram showing how hydrodynamic injection in the biliary system could proceed though percutaneous ultrasound access of intrahepatic biliary ducts. A needle is advanced into these intrahepatic bile ducts through the skin, whereafter a catheter is advanced through the needle. A balloon would inflate in this catheter, and the injection would proceed through an opening proximal to the balloon, such that fluid motion would proceed in a retrograde fashion as depicted.

FIGS. 60A-60C show a diagram of catheter placement with balloon inflation into the liver, pancreas and kidney, respectively, for optimal injection and seal. Examples of the guidewire staying in place during injection are provided for the liver (FIG. 60A), the pancreas (FIG. 60B), and the kidney (FIG. 60C).

FIGS. 61A-61B show a diagram of catheter placement into the ureter and renal pelvis for optimal injection and seal. FIG. 61A depicts localization of the balloon within the ureter prior to fanning out of the renal pelvis. FIG. 61B shows that the balloon can also be inflated within the renal pelvis itself in an alternate exemplary embodiment.

FIG. 62 shows a diagram of cannulation of the ureter orifice in the bladder with a catheter.

FIG. 63 shows a model diagram of the hydrodynamic fluid force into the kidney in order to mediate efficient delivery of DNA into the tissue.

FIGS. 64A-64D show the results of ERCP experiments performed on two pigs for pancreatic gene delivery. FIG. 64A shows a pig pancreas carefully dissected away from other internal organs.

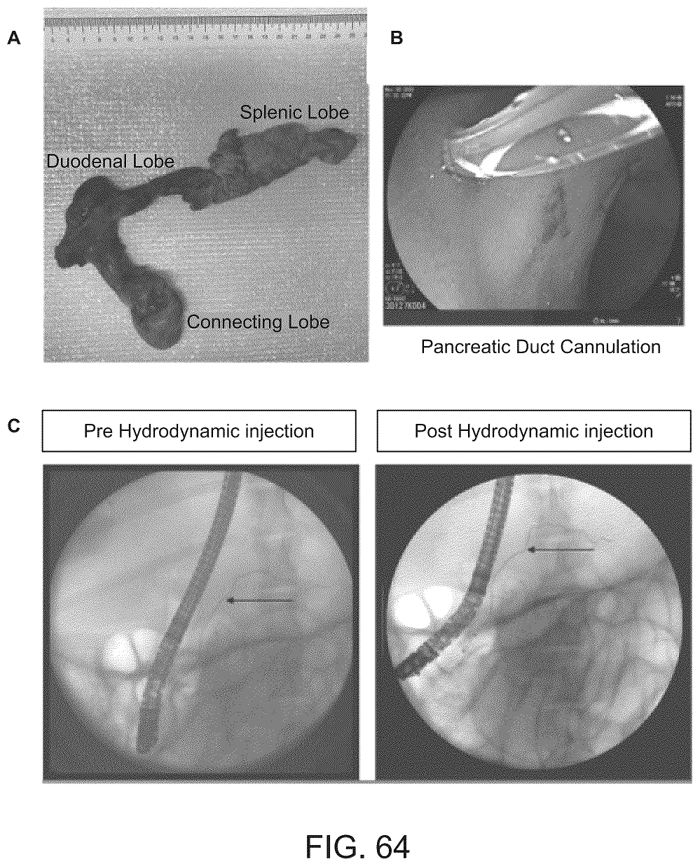

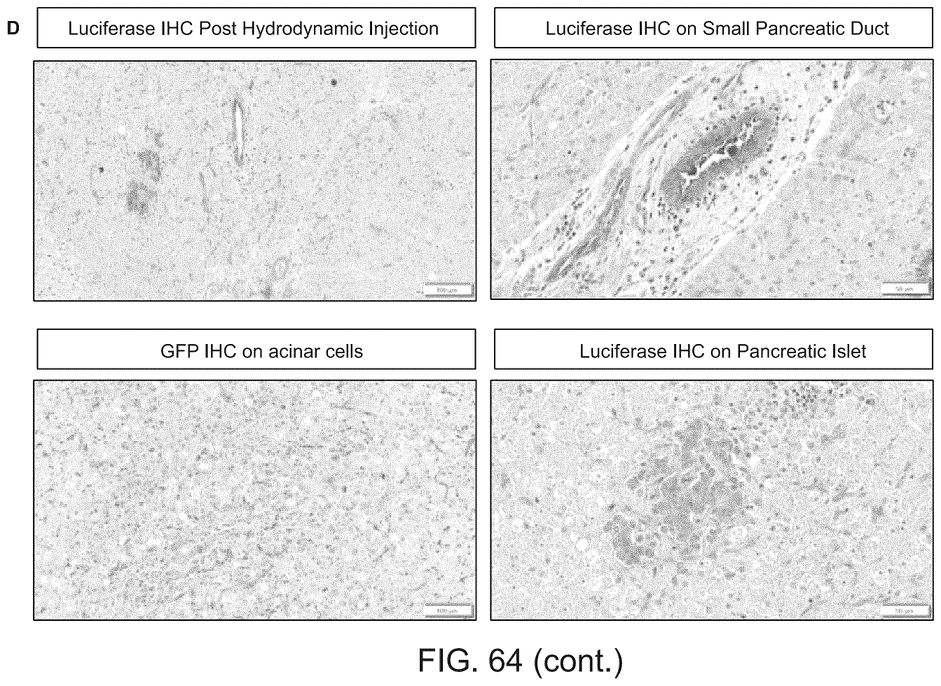

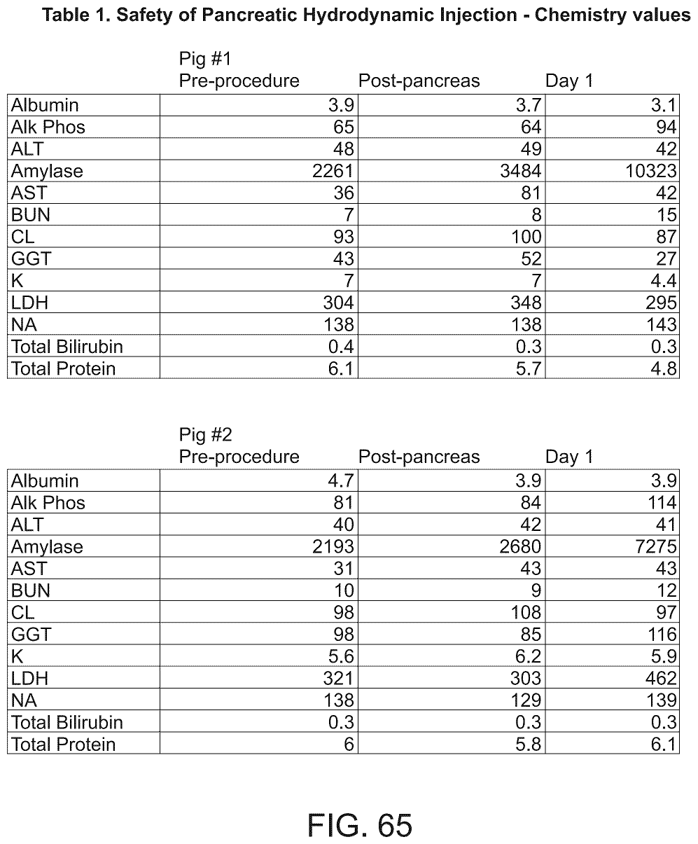

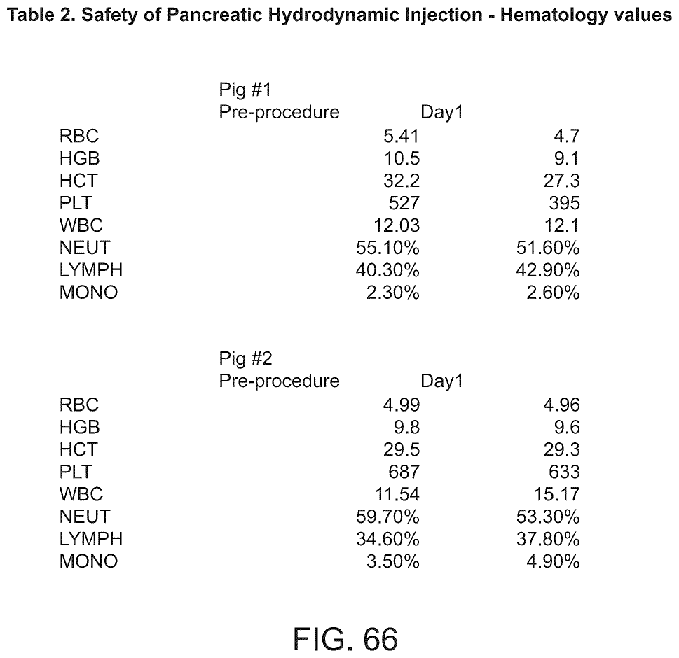

The pig pancreas has three different lobes including the duodenal lobe, the connecting lobe, and the splenic lobe. All lobes have pancreatic ducts that merge in the duodenal lobe, also merging with the bile ducts. FIG. 64B shows cannulation of the pancreatic duct orifice with a catheter. FIG. 64C shows that contrast injection before hydrodynamic injection was able to visualize the pancreatic duct (left panel; arrow points to the branch toward the splenic lobe), while a repeat contrast injection post hydrodynamic injection demonstrates that the ductal structure remains preserved (right panel; arrow points to the ductal branch toward the splenic lobe). FIG. 64D shows that different cell types throughout the pancreas demonstrated gene delivery. Among the most prominent cell types, all pancreatic ductal epithelium cells demonstrated intense luciferase staining that was consistent from large ducts to small ducts to branching ductules. GFP staining was noted prominently in pancreatic acinar cells. Luciferase staining was strong in pancreatic islet cells, appearing to be uniform among all types of islet cells (alpha, beta, and delta).

FIG. 65 shows data regarding the safety of pancreatic ductal hydrodynamic injection in two pigs as assessed by chemistry values. Effects of pancreatic ductal hydrodynamic injection on chemistry values. Blood chemistries were collected prior to injection, 10 minutes post pancreatic injection, and at day 1 post-injection. Only amylase was consistently elevated in both pigs after pancreatic injection.

FIG. 66 shows data regarding the safety of pancreatic hydrodynamic injection in two pigs as assessed by hematology values. Effects of pancreatic ductal hydrodynamic injection on hematology values. Blood was collected prior to injection, 10 minutes post pancreatic injection, and at day 1 post-injection, and various hematological parameters investigated. No significant changes were noted.

FIGS. 67A-67F illustrate the ability to target protein expression specifically to endothelial cells after biliary hydrodynamic injection. Pigs were injected with a plasmid, pICAM2-GFP, that harbors an endothelial cell-specific promoter driving expression of green fluorescent protein (GFP). The data shows immunohistochemical staining for GFP protein in pig liver tissue. FIG. 67A depicts endothelial cells bordering a blood vessel specifically staining positive for GFP expression. FIG. 67B illustrates an isolated endothelial cell positive for GFP amidst negatively stained hepatocytes. FIGS. 67C, 67D, and 67E depict portal triads within the pig liver, demonstrating that only the endothelial cells (solid arrow) are positive for protein expression. For comparison, bile ducts (dashed arrow) are negative for any GFP expression, emphasizing the specificity of the cell-specific targeting. FIG. 67F depicts hepatocytes around a central vein in a lobule, which are all negative reflective of a lack of off-target cell expression observed.

### DETAILED DESCRIPTION

The present disclosure is based, at least in part, on the discovery of improved protocols for using hydrodynamic injection for gene therapy in large animals using naked DNA. The techniques herein provide methods and systems for transfecting cells of a subject in vivo, comprising administering at elevated pressure an effective amount of a nucleic acid expression cassette to the subject's biliary tree, liver or pancreas. The techniques herein provide methods and systems of delivering a polypeptide or a vector comprising a nucleic acid sequence that ameliorates or prevents a disease or disorder of the kidney, the liver, or the pancreas. Exemplary diseases or disorders may include, but are not limited to, hemophilia A, hemophilia B, alpha-1 antitrypsin deficiency, familial hypercholesterolemia, progressive familial intrahepatic cholestasis, hereditary hemochromatosis, Wilson's disease, Crigler-Najjar Syndrome, methymalonic academia, phenylketonuria, and/or ornithine transcarbamylase deficiency.

We now provide methods and systems that include steps of delivering a polypeptide or a vector comprising a nucleic acid sequence that ameliorates a kidney, liver, or pancreas disease, or other disease or disorder such as hemophilia to a subject having or prone of getting a liver, kidney, or pancreas disease or other disease or disorder such as hemophilia.

Thus, in one aspect, methods are provided for transfecting cells of a subject in vivo, comprising administering at elevated pressure an effective amount of a nucleic acid expression cassette to the subject's biliary tree, liver or pancreas.

In one embodiment, methods are provided to treat or prevent kidney, liver, or pancreas disease by genetic therapy. In particular embodiments, methods are provided to treat or prevent hemophilia A, hemophilia B, alpha-1 antitrypsin deficiency, familial hypercholesterolemia, progressive familial intrahepatic cholestasis, hereditary hemochromatosis, Wilson's disease, Crigler-Najjar Syndrome, methymalonic academia, phenylketonuria, and/or ornithine transcarbamylase deficiency, and other diseases and disorders as disclosed herein by genetic therapy.

More particularly, in one aspect, the disclosure provides methods and systems for treating a subject comprising: administering with a catheter an effective amount of a nucleic acid and/or protein solution at high-fluid pressure through the subject's biliary tree into the subject's liver, wherein the catheter delivery portion is advanced through the biliary tract upstream of the cystic duct and into the common hepatic duct. In one aspect, preferably the catheter tip is positioned at an extrahepatic location during delivery of the nucleic acids. In a further aspect a catheter tip is positioned within an intrahepatic position within the liver parenchyma during delivery of the nucleic acids. In a yet further aspect, a catheter tip is placed within or proximate to the liver parenchyma. In a yet further aspect, the catheter is positioned whereby the catheter balloon is inflated proximate to or downstream of the liver hilum within the common hepatic duct. These aspects serve to abrogate stress on the biliary wall to eliminate the chance of bile duct rupture during the hydrodynamic injection.

In another preferred aspect, the disclosure provides methods and systems for treating a subject comprising: administering to a subject with a catheter an effective amount of a nucleic acid and/or protein solution at high-fluid pressure to subject's biliary tree, liver, kidney or pancreas, wherein the catheter balloon is deflated substantially immediately after completing administering a dose of the nucleic acids from the catheter. Preferably, the balloon deflation reduces fluid pressure within the subject's bile duct system, liver, kidney or pancreas.

In a yet additional preferred aspect, the disclosure provides methods and systems for treating a subject comprising: administering to the subject's nucleic acid and/or protein solution biliary tree, liver, kidney or pancreas, 1) a first fluid composition that does not contain nucleic acid or protein and thereafter 2) an effective amount of a nucleic acid or protein solution. Suitably, the first fluid injection serves to clear bile from the biliary system before the injection of the nucleic acid or protein solution.

In a still further preferred aspect, the disclosure provides methods and systems for treating a subject comprising: administering with a catheter an effective amount of a nucleic acid and/or protein solution at high-fluid pressure through the subject's biliary tree, liver, kidney or pancreas, wherein the catheter is configured to administer the nucleic acids or protein in the forward direction only and not obliquely or perpendicular. Suitably, the catheter is configured to administer the nucleic acids or protein in the forward direction only and not obliquely or perpendicular with respect to the subject's biliary tract walls.

In an additional aspect, the disclosure provides methods and systems for treating a subject comprising: administering with a catheter an effective amount of a nucleic acid and/or protein solution at high-fluid pressure through the subject's biliary tree, liver, kidney or pancreas, wherein the catheter tip is at least 1 cm, or at least 4 cm forward along the catheter length from the catheter balloon midpoint.

In a still further aspect, the disclosure provides methods and systems for treating a subject comprising: administering a radiocontrast agent through the subject's biliary tree, liver, kidney or pancreas to verify catheter placement after balloon inflation, and thereafter administering an effective amount of a nucleic acid and/or protein solution at high-fluid pressure through the subject's biliary tree, liver, kidney or pancreas. Suitably, the catheter position is selected and/or verified with visualization of the administered radiocontrast agent, and the radiocontrast agent suitably is administered through the subject's biliary tree into the subject's liver.

In preferred aspects of the above methods and systems, the nucleic acid and/or protein solution may be administered through the subject's biliary tree into the subject's liver.

In certain aspects, the nucleic acid or protein may be administered using endoscopic retrograde cholangiopancreatography (ERCP).

In certain aspects, the catheter may be selectively advanced into the right hepatic bile duct of the subject whereby the nucleic acid is administered within right liver lobes.

In certain aspects, the catheter may be selectively advanced into the left hepatic bile duct whereby the nucleic acids is administered within left liver lobes.

In certain aspects, a contrast composition may be administered at a slower flow rate than the nucleic acid solution is administered. A variety of nucleic acid may be administered to a subject, including one or more of DNA, mRNA, siRNA, miRNA, lncRNA, tRNA, circular RNA, or antisense oligonucleotides.

In particular aspects, an amount of nucleic acid or protein to be administered to the subject may be selected based on the subject liver weight. In particular, DNA may be administered in an amount of at least 1 mg of DNA per kilogram of the subject's total liver tissue weight. RNA also may be administered in amount of at least 1 mg of RNA per kilogram of the subject's total liver tissue weight. One or more proteins suitably may be administered in an amount of at least 100 μg of protein per kilogram of the subject's total liver weight.

In further systems, the nucleic acid may be administered as a fluid composition in a total volume amount of 30 mL or greater per kilogram of the subject's total liver tissue weight, or great amounts such as total volume amount of 40, 50, 60, 70, 80, 90, 100 mL or greater per kilogram of the subject's total liver tissue weight.

In particular system, one or more DNA or RNA molecules may be combined into the same nucleic acid solution and injected at the same time.

In certain aspects, flow rate of an administered composition may be independent of the subject's liver weight. In further aspects, the volume of an administered composition may be correlated with the subject's liver weight.

In certain aspects, the nucleic acid may be administered as a fluid composition at an injection flow rate of greater than 2 mL/sec and does not result in bile duct rupture, or higher flow rates such as 5 mL/s or 10 mL/s or greater and does not result in bile duct rupture.

In certain systems, hydrodynamic injection of an administered composition produces a rapid increase in pressure that plateaus, which immediately drops with the cessation of injection.

Exemplary plateau pressures may include for example about 80, 100, 110, 120, 130, 140, 150, 160, 170, 180, 190 or 200 mmHg or greater. The pressure plateau also can serve as a type of diagnostic of a successful administration. For instance, if an administration (injection) is not successful such as by not producing a substantially closed system for the administration, in particular where an expected pressure curve is not achieved due to a failure of balloon seal or other causes.

In related aspects, by using pressure tracing, a catheter balloon seal during the administering can be assessed to be effective to prevent or substantially inhibit undesired retrograde flow.

For certain applications, it may be preferable to administer a composition that does not contain a therapeutic agent (nucleic acid or protein). Suitably, a first non-nucleic acid composition is administered prior to administering nucleic acids or proteins. The first non-nucleic acid composition can be administered at a variety of amounts and flow rates. In a preferred aspect, the non-nucleic acid composition is administered in an amount approximately equal to the subject's native biliary volume. In a further aspect, the non-nucleic acid composition may be administered at a volume greater than 20 mL prior to nucleic acid/protein injection in order to clear biliary substances. One suitably flow of the non-nucleic acid composition is 1 mL/sec or less. One preferred non-nucleic acid composition comprises one or more of saline solution, Dextrose 50% in Water, lactate ringer's solution, and phosphate buffered solution.

Preferably, a nucleic acid or protein therapeutic is delivered into a substantially closed system, or a sealed or substantially sealed system, preferably where injection (e.g. of a therapeutic fluid composition) proceeds in a retrograde direction against normal fluid flow. For example, a vessel or organ of a subject, optionally together with a medical device or tool can be utilized to provide a sealed duct or vessel system where retrograde flow creates an elevated pressure of administered composition comprising the nucleic acid expression cassette.

In certain aspects, a nucleic acid molecule can be used in nucleic acid expression cassettes in conjunction with their natural promoter, as well as with another promoter. For instance, a liver-specific promoter may be used if desired, to increase liver-specificity and/or avoid leakage of expression in other tissues if the target of administration is the subject's liver cells. The liver-specific promoter may or may not be a hepatocyte-specific promoter. Regulatory sequences also may be used in the nucleic acid expression cassettes.

According to a particular embodiment, only one regulatory element may be included in the expression cassette. According to an alternative particular embodiment, more than one regulatory element is included in the nucleic acid expression cassette, i.e. they are combined modularly to enhance their regulatory (and/or enhancing) effect. According to a further particular embodiment, two or more copies of the same regulatory element may be used in the nucleic acid expression cassette. For instance, 2, 3, 4, or 5 or more copies of a regulatory element may be provided as tandem repeats. According to another further particular embodiment, the more than one regulatory element included in the nucleic acid expression cassette comprises at least two different regulatory elements. In certain embodiments, it is envisaged that the length of the total regulatory element(s) in the nucleic acid expression cassette does not exceed 1000 nucleotides.

The transgene may be homologous or heterologous to the promoter (and/or to the animal, in particular mammal, in which it is introduced, in cases where the nucleic acid expression cassette is used for gene therapy). In addition, the transgene may be a full-length cDNA or genomic DNA sequence, or any fragment, subunit or mutant thereof that has at least some biological activity. In particular, the transgene may be a minigene, i.e. a gene sequence lacking part, most or all of its intronic sequences. The transgene thus optionally may contain intron sequences. Optionally, the transgene may be a hybrid nucleic acid sequence, i.e., one constructed from homologous and/or heterologous cDNA and/or genomic DNA fragments. The transgene may also optionally be a mutant of one or more naturally occurring cDNA and/or genomic sequences.

In a particular aspect, a nucleic acid expression cassette does not contain a transgene, but the regulatory element(s) operably linked to the promoter are used to drive expression of an endogenous gene (that thus is equivalent to the transgene in terms of enhanced and/or tissue-specific expression). The nucleic acid expression cassette may be integrated in the genome of the cell or stay episomal. Other sequences may be incorporated in the nucleic acid expression cassette as well, typically to further increase or stabilize the expression of the transgene product (e.g. introns and/or polyadenylation sequences). Any intron can be utilized in the expression cassettes described herein. The term “intron” encompasses any portion of a whole intron that is large enough to be recognized and spliced by the nuclear splicing apparatus. Typically, short, functional, intron sequences are preferred in order to keep the size of the expression cassette as small as possible which facilitates the construction and manipulation of the expression cassette. In some embodiments, the intron is obtained from a gene that encodes the protein that is encoded by the coding sequence within the expression cassette. The intron can be located 5′ to the coding sequence, 3′ to the coding sequence, or within the coding sequence. An advantage of locating the intron 5′ to the coding sequence is to minimize the chance of the intron interfering with the function of the polyadenylation signal.

Significantly, the techniques herein provide systems and methods for avoiding rupture of the common hepatic duct, thereby allowing for increased levels of volumes and flow rate to be achieved. This includes methods of optimal catheter placement to avoid rupture of the common hepatic duct wall and methods to alter the catheter to promote for flow of injection for the same purpose. In further aspects, new tolerated levels of volume and flow rate that could be injected during the biliary procedure are disclosed herein.

Moreover, the techniques herein have discovered, unexpectedly, that flow rate of an administered composition may be an important determinant of intra-biliary pressure during biliary hydrodynamic injection, with implications for flow rate and pressure to be principally maintained from neonates adolescent to adult patients for biliary hydrodynamic injection. The present methods and systems also may avoid DNA solution wastage during an administration, which includes preloading of DNA solution in the catheter circuit and/or liver, as well as methods to chase the DNA solution with a second non-DNA solution including in certain embodiments mediated through a double-barreled application device such as a power injector.

The present methods and systems also may differentiate specific flow rates that can mediate liver damage versus conditions that will not mediate liver damage, as judged by serum liver enzyme levels and tissue histology. Different flow rates can also be used to target hepatocytes specifically within different areas of the liver.

As disclosed herein, biliary hydrodynamic gene therapy represents a novel non-viral method of delivering protein and/or nucleic acids directly inside hepatocytes of the liver. As discussed further below, the techniques herein also provide methods and systems for delivering proteins and/or nucleic acids to cells of the pancreas and kidney. It is contemplated within the scope of the disclosure that a wide variety of nucleic acid can be delivered in accordance with the present methods and systems including DNA and other forms of nucleic acids including mRNA, miRNA, siRNA, long non-coding RNAs, or ribozymes. In addition, recombinant proteins could be delivered inside hepatocytes by the present methods and systems.

### DNA Solution

A suitable procedure may begin with the preparation of a nucleic acid solution for injection. Injecting combinations of nucleic acids is already readily contemplated. In preferred embodiments, the nucleic acid injected is DNA solution. The DNA solution will be injected into the biliary system and then exit into the bloodstream circulation. In order to avoid physiological disruption, the ideal solution should maintain close to normal physiologic molarity. Examples of optimal solutions include 0.9% normal saline solution, lactate ringer's solution, 5% dextrose in water solution, or phosphate buffered solution. In alternative embodiments, a hyperosmolar solution could be utilized, but caution should be made to limit the total volume injected into the patient and into their circulatory system to avoid disruptions to physiology. Hyperosmolar solutions function by increasing the size of pores formed in the hepatocyte membrane. Important in the preparation of the DNA solution are considerations to prepare it in sterile conditions. This would be optimally prepared in a biosafety cabinet to prevent contamination, with thorough mixing of DNA and injected solution amount. In other preferred embodiments, the DNA solution will be prepared off site at a central manufacturing facility and pre-packaged into a cartridge that can fit into the power injector for use.

### DNA Amount

One key consideration with the procedure is deciding the optimal amount of DNA to be injected. In certain aspects, it was found that under constructs under the control of an optimized liver specific promoter demonstrated up to 35% transfection efficiency when injected at 3 milligrams DNA and 30 mL volume in 2 mL/sec second flow rate with pre-loading of DNA solution into the catheter. Surprisingly, when the mass of DNA was increased to 5.5 milligrams DNA the transfection efficiency increased over 50%. This result was unexpected given that there is limited correlation between increasing DNA doses in mouse hydrodynamic tail vein injection and the eventual transfection efficiency inside the liver. Without being bound by theory, it is believed that this aspect may be useful in optimizing the DNA dose of therapeutic nucleic acids for use to treat specific liver disorders, where each liver disorder requires a different correction percentage of hepatocytes in order to mediate treatment. In preferred embodiments, the DNA mass injected is at minimum 3 mg and in other embodiments at 5 mg and in still other embodiments greater than 10 milligrams of DNA injected per kilogram of liver weight. Different sizes of livers may require different DNA doses depending upon the target liver weight. All DNA was prepared with endotoxin-free qualities in order to avoid immune activation and/or death of transfected cells.

### Volume of DNA Solution

A next aspect of the biliary hydrodynamic procedure is deciding the volume of DNA solution to be prepared. Volume is an important parameter to consider because of both the physiological stress exerted on the relevant organ of the subject and, of equal importance, the mechanical stress exerted on the tubing in the catheter circuit (e.g., the endoscopic catheter in the tubing of the power injector). Larger volume use means a longer injection time, which increases both the mechanical and physiological stress. Additionally, a larger volume will eventually be expelled into the circulatory system of the patient, and thus lower volumes are preferred in order to diminish the impact of extra volume on heart and kidney function. The present disclosure demonstrated that a minimal preferred volume may be 30 mL, which corresponds to the approximate volume of the biliary system in an adult human liver. Thus, injection of 30 mL leads to expulsion of the entire biliary volume increased pressure in that system. The results herein found that much higher volumes can be tolerated for injection without significant stress on the biliary system or hepatic function or on physiologic perturbations on heart rate or echocardiogram. A volume of 140 mL volume injected 1 mL/sec or as well tolerated by the pig with no significant abnormalities in liver enzymes observed. Of note, this volume is close to the maximum volume of the barrel in the particular power injector tested (150 mL), so larger injected volumes are possible. Depending on the source of power injector, one could imagine injecting higher volumes. Optimal DNA solutions are significantly less than this between 30 mL to 60 mL in adult humans, corresponding to up to two biliary volumes. Of note, DNA solution volume may be increased in order to account for DNA solution that might remain within the catheter and power injector circuit. This would effectively reduce the fraction of DNA left inside the catheter tubing after injection concludes. Alternatively, dead space issues can be mitigated as further discussed below.

### Endoscopic Retrograde Cholangiopancreatography Procedure

The patient should be prepped for the gene therapy procedure under routine endoscopic retrograde cholangiopancreatography (ERCP) procedure guidelines. This includes NPO (nothing by mouth) overnight in order to clear the stomach and intestinal system of any food in order to allow maximal visualization for endoscopy. Before the procedure, the patient will be prepped under anesthesia according to routine surgical practices. In the beginning of the procedure, the endoscope will be advanced through the oropharynx, down into the esophagus, then into the stomach, into the small intestine, and eventually sitting in the small intestine en face with the biliary orifice. A catheter will be advanced out of the endoscope, and an attempt to cannulate the ampulla of Vater will occur. After successful cannulation, the catheter will be advanced the common bile duct, past the cystic duct, and into the common hepatic duct. The balloon located near the distal tip of the catheter will then be inflated at this point. The balloon will close to, against, or within the hepatic hilum. The provider may even feel the resistance of balloon inflation and catheter movement that would help verify the correct catheter placement. This positioning will be verified on fluoroscopy measurement, wherein the balloon has radiographic opacity to measure its position. At this point in the gene therapy procedure, radiocontrast solution will be administered through one of the ports in the catheter, usually in small doses of 5 to 10 mL's with subsequent fluoroscopic imaging to verify that the contrast solution is within the biliary tract. Preferably, both the left and right branches of the biliary tract are verified to be present and contain contrast. There is the potential for the endoscopist to be worried about advancing the catheter toward one branch or the other, but successful balloon inflation will prevent the catheter from advancing too far into the liver parenchyma. In certain embodiments, the catheter may be advanced further into the liver in order to access the right or left hepatic bile duct, respectively, for target injection of the right and left liver lobes, respectively.

### Endoscopic Ultrasound Needle Procedure

In an alternative to the ERCP procedure stated above, alternative methods to placing the catheter in the common hepatic duct can be performed. The purpose of these alternative methods is to access the common hepatic duct through a different route in order to eliminate the chance of post-ERCP pancreatitis. During ERCP, the catheter temporarily occludes the pancreatic ductal system, leading to the potential for pancreatitis in a small fraction of patients. If the catheter could be introduced into the common hepatic duct by a different means, then no pancreatic duct occlusion would occur creating a safe procedure for the patient. Furthermore, these alternative routes would be particularly beneficial in instances where many repeat procedures may occur over time in a given patient.

In one alternative embodiment of the disclosure, catheter insertion would occur by an endoscopic procedure where in ultrasound would be used to place a needle into the wall of the stomach or small intestine, thereby directly contacting a bile duct. After the needle is passed into the wall, a guidewire can be inserted through, and then a catheter eventually exchanged over the guidewire that the catheter itself can fit through the opening. In some instances, the bile duct accessed directly to the common hepatic duct or common bile duct itself, allowing for the rest of the procedure to occur in a very similar manner to ERCP. In other instances, the gallbladder can be directly accessed, such that the catheter will then be advanced from the gallbladder into the common hepatic duct. In other instances, a bile duct within the liver parenchyma will be accessed, such that the catheter will be advanced in an antegrade fashion toward the common hepatic duct.

For these methods, after the bile duct is placed into the common hepatic duct, the procedure can largely occur according to the same specifications that are described elsewhere in the patent, including the exact placement of the catheter within the common hepatic duct. one modification is if the upstream branches of the bile duct systems are first accessed by ultrasound, the catheter itself must have an opening toward the proximal side of the balloon such that injection will proceed tore the proximal direction of the catheter. in order to avoid solution leaking out of the hole that was inserted into the intestinal wall, a second balloon would be included within the catheter in order to block this opening.

### Percutaneous Procedure

In a second alternative to the ERCP procedure that would effectively eliminate the potential for pancreatitis from the gene delivery procedure, a percutaneous procedure could be performed to deliver the catheter into the common hepatic duct, whereafter all steps of the gene delivery procedure would be largely similar to what is stated elsewhere in the patent application. In this alternative, ultrasound or another imaging modality would be used to locate the gallbladder or bile ducts within the liver underneath the surface of the skin. Upon identifying these structures, a needle will be placed through the skin and into the gallbladder or bile duct.

The guidewire could be placed through this needle, and then eventually exchanged for a catheter which could be inserted into this space. In the example of first contacting the gallbladder, the catheter could be advanced from the gallbladder through the cystic duct and into the common hepatic duct. After this, all steps of the injection would occur similar to elsewhere in the pattern. In the version where an upstream bile duct is identified within the liver, the catheter would be advanced through the skin into this upstream bile duct, and then the catheter advanced in the antegrade fashion with natural bile flow and into the common hepatic duct. As mentioned in the previous section, the catheter would be modified in this version of the procedure to accommodate an injection opening below the balloon and proximal to the user, such that all injections would progress in the proximal direction of the catheter, but still anatomically retrograde to the rest of the liver. Like the endoscopic procedures, the percutaneous procedure would preferably have a balloon inflated around the needle insertion site through the skin into the organ either on the outside of the skin or more preferably a balloon inflated at the contact point within the bile duct or gallbladder. This would serve to eliminate the potential for leakage of the fluid during hydrodynamic injection through these entry points.

### Catheter Placement

The present methods and systems also include catheter placement. Prior approaches have led to rupture of the common hepatic duct (CHD) at elevated flow rates and/or volumes injected.⁴¹

In a particular aspect, delivery (e.g. via injection) of an administered composition is made within the liver parenchyma aims to reinforce the fluid wall stress and thus prevent or otherwise minimize the occurrence of any rupture of the biliary tree. In some embodiments, the catheter is be placed within the intrahepatic common hepatic duct, such that the balloon would be inflated in the intrahepatic common hepatic duct. In other embodiments, extrahepatic balloon placement leads to catheter tip placement within the liver parenchyma. In certain preferred aspects, the catheter is extended at least 1, 2, or 3 centimeters past the inflation balloon in order to assure its placement inside the liver parenchyma; e.g., in this arrangement, the catheter is place in the extrahepatic common hepatic duct. As stated above, the liver parenchyma should reinforce the biliary ducts and prevent any rupture of the biliary ducts. In another aspect, the catheter tip would be placed immediately upstream of the inflation balloon to minimize the plasmid DNA being injected from entering the left or right hepatic ducts preferentially. If the inflation balloon is placed within the left or right hepatic ducts, having the opening for the fluid injection may maximize the amount of liver parenchyma exposed to plasmid.

In a further aspect, a catheter system is provided where one or more catheter channels are forward facing to maximize forward fluid flow and further preferably to avoid lateral ejection or delivery of fluid, which would otherwise put significant wall stress on the bile duct. In these preferred systems, the forward facing catheter channel(s) deliver administered composition in a forward direction to thereby reduce stress on the duct wall.

Clearance of Bile from the Biliary System

In a preferred embodiment of the disclosure, fluid not containing nucleic acid solution is be injected through the catheter in order to remove any remaining contrast solution in the bile ducts. Similarly, fluid would also be injected at this point in order to clear bile itself from the biliary system, which otherwise might bind to the DNA an inhibit delivery or yield toxicity when introduced inside the cytoplasm of hepatocytes, or contain nucleases that could degrade DNA during injection, or contain antibodies or cytokines to cause inflammation or toxicity within hepatocytes. The total volume of fluid injected to clear the biliary system would optimally be equal to the total volume of the biliary system in an average adult human liver (30 mL, with levels adjusted depending on the size of the patient) plus additional volume from the dead space of the catheter circuit, which could equal to 5 to 10 mL's depending on the brand of catheter and the tubing available. These fluid injections could either take place by hand syringe at low manual flow rates of approximately 1 mL per second or less or could alternatively be delivered by the power injector itself as well. In some embodiments, the fluid not containing DNA solution will be delivered from the second barrel of a double-barreled power injector.

Confirmation of Power Injector Function and Balloon Seal after Placement

While contrast injection can confirm the proper balloon placement during fluoroscopy, in preferred embodiments a step would be performed to confirm that the balloon will successfully seal the common hepatic duct during hydrodynamic injection. During this step, a short burst of non-DNA solution may be injected such as using the power injector to mediate hydrodynamic force in the bile duct. At the same time, a pressure catheter inserted into the common hepatic duct may monitor the pressure achieved during this test injection. A substantially complete balloon seal should generate a plateau peak of pressure during the injection, while any leak should lead to a rapid drop in pressure and an irregular pressure curve. This step also importantly confirms the intended power injector settings, and their ability to mediate successful injection. Different power injectors from different brands could be utilized for the biliary hydrodynamic injection procedure, and the ability to confirm that a given flow rate mediates the intended peak pressure is important for the integrity of the later DNA delivery. If pressure is not achieved at this step, the flow rate may be increased if necessary to achieve the intended pressure goal for the procedure. In preferred embodiments of the disclosure, the non-DNA solution achieved would be no more than 10 mL in volume per kilogram of liver weight.

### Pre-loading of DNA Solution

After clearance of contrast solution from the biliary system, the procedure can proceed to the loading of a nucleic acid solution. In preferred embodiments, a DNA (or other nucleic acid) solution is primed into the endoscopic catheter and the tubing of the power injector, in order to ensure that the initial solution injected by hydrodynamic force contains DNA. In other embodiments, DNA solution may be pre-injected into the liver and biliary system itself, although caution should be made to ensure that the hydrodynamic injection proceeds quickly after this to avoid any degradation or non-specific binding of DNA to the surface of cholangiocytes and hepatocytes lining the bile ducts. In yet other alternative embodiments of the procedure, the tubing itself could still be primed with normal saline solution (or other physiologic, non-DNA solution) to mediate some of the aforementioned clearance function, before DNA in the power injector cartridge is pushed into the system for pre-loading prior to injection.

After the catheter system and the injection tubing are primed, DNA solution can be loaded into the power injector. For loading of the power injector with DNA itself, the DNA containing solution may be loaded into an empty cartridge using a suction system in the power injector device. In other embodiments, a pre-filled cartridge with DNA solution will be supplied prior to the procedure that can fit into the power injector for ease of use and to maintain sterile conditions.

### Programming of the Power Injector

The power injector can next be programmed for delivery of the DNA solution. Power injectors are commonly used in medical practice to inject radiocontrast into blood vessels in order to ascertain potential blockages or sources of bleeds. Power injector act quickly at high flow rates and volumes in order to counter the normal flow rates and pressure of the cardiovascular system. The term power injector, however, is a generic term describing a range of different devices delivering fluid, and should be regarded as any general system that can apply a force on a volume of liquid at high pressure in order to achieve a desired flow rate.

In present methods and systems, the power injector could be programmed at many different potential settings. In preferred embodiments, the power injector is a flow rate greater than or equal to 2 mL/sec else. The prior literature established 2 mL/sec as the maximum tolerated dose, but the results herein found, unexpectedly, that higher flow rates could be achieved, without any additional toxicity or novel histological findings. The results herein found that a flow rate up to or equal 5 mL/sec could be injected into the pig without an elevation in liver enzymes after injection. Higher flow rates up to 10 mL/sec were also described, causing only mild elevations in liver enzyme levels.

### Catheter Design and Setup

For at least certain methods and systems, the selection of an endoscopic catheter can notably impact outcome of a gene delivery/therapy procedure. Specifically, the diameter of the channel through which the hydrodynamic solution is pushed through can have a significant influence on the tolerability of hydrodynamic injection in the circuit. In preferred aspects, with biliary catheters having a smaller channel size, the same programmed flow rate would yield a higher pressure inside the catheter, causing the higher injector to automatically lower the flow rate to prevent rupture or cracking of the tubing circuit. When this pressure threshold was increased, the catheter was found too snap or crack at these same flow rates. In preferred embodiments, a catheter is utilized with higher tensile strength in the walls in order to handle these higher flow rates and pressure, preferably where the catheter remains flexible in order to pass through the endoscope and into the biliary system.

Alternatively, the diameter of a catheter channel may be as large as possible in order to reduce the pressure on the walls of the catheter at a given flow rate. This would ensure high flow rates are achieved that can still be delivered into the bile ducts. This aspect was demonstrated during in testing with subjects of pigs, where a larger catheter channel size was employed in order to achieve a higher flow rate, thereby not otherwise causing a pressure alarm in the catheter system.

In preferred embodiments, a pressure catheter is inserted through one of the ports of a catheter (e.g. endoscopic catheter) to extend into the bile duct system. Pressure catheters are already available today for many intravascular procedures. This step would occur prior to DNA (or other nucleic acid) injection, and ideally before injection of the aforementioned non-DNA solutions. The other port (or ports) in the catheter in this circumstance would serve as the port of DNA solution and clearance or chaser solution injection. In some embodiments, a short burst of fluid at the intended flow rate for the procedure could be done with non-DNA containing solution in order to check the functionality of the power injector prior to use of DNA injection in the patient. The pressure catheter is optimally inserted to a distance ideally it at least 0.5 to 1 cm past the distal tip of the endoscopic catheter to assure localization inside hepatic bile ducts to reflect pressure within that circuit. The pressure catheter should have a dynamic range between 0-400 mmHg for detection of potential pressure waves employed during the procedure in preferred embodiments. In preferred embodiments, the pressure catheter is connected to a computer, mobile device, or tablet that can record pressure waves in real time in order to monitor the correct function of the injection. For injections at expected performance, the constant flow rate should generate a flat plateau peak of pressure that abruptly drops when the injection is stopped. Evidence that this flat plateau peak is not maintained would suggest disruption of the balloon seal or a malfunction in the power injector. In other embodiments, the pressure catheter would be connected to a computer interpreting the live pressure readings, which could be connected to a power injector itself to alert feedback on the need to alter the flow rate being injected. In some embodiments of the disclosure, the power injector would come already within the biliary catheter, such that it would not be inserted through one of the channels since it is already located within the biliary catheter.

In certain embodiments of the catheter and methods of the procedure, the guidewire that is used to facilitate the localization of the catheter can also be kept in during the injection itself. In most of the embodiments previously described, the guidewire is taken out as the catheter is exchanged over it. However, without the guidewire in pace, the exact localization of the catheter can be hard to assess. Furthermore, if specific branches of the biliary system are targeted, this can be hard to discern based on visualization of the catheter alone. Thus, in some embodiments, the guidewire can be left in during the injection, and the DNA solution is injected through the injection port or lumen. Keeping the guidewire in is also useful in tracking if the catheter moves during the injection, and what is the depth of catheter insertion.

The guidewire can also be applied to pancreas and kidney injection. For the pancreas, the guidewire can be kept in during the injection and used to help gauge the depth of the insertion during catheter injection and balloon inflation, helping to facilitate specific targeting of the tail end of the pancreas. Similarly, the guidewire in renal injection can be used to identify the terminus of the ureter and pelvis, assuming that the balloon is situated in the correct place and not free floating within the renal pelvis but situated more securely in the ureter. The catheter tip can also be judged against in the guidewire in the renal pelvis, such that it can be observed to be squarely within the renal pelvis area.

### Flow Rate and Volume Settings for the Injection

There are two principle variables to hydrodynamic injection. These variables include volume and flow rate for the injection. The significance of these variables for biliary hydrodynamic injection was not identified in the previous literature published and it is not obvious which variable is necessary to maximize, and how these variables relate to the pressure achieved. The results herein discovered that a key variable to delivering pressure is flow rate.

Experiments that escalated flow rate while keeping volume constant resulted in significant increases in pressure. Unexpectedly, experiments that greatly increased volume while keeping flow rate constant did not result in significant elevations of pressure. Over all experiments, the pressure within the biliary tree correlated well with the flow rate programmed into the machine. Thus, in the preferred embodiment of the disclosure, the flow rate is the dominant parameter identified to govern pressure in order to optimize gene delivery through membrane pores in hepatocytes.

In consideration for the choice of flow rate to be programmed into the power injector, the disclosure also describes metrics that can govern the procedure in order to choose the appropriate flow rate. These will be chosen depending on the potential goal of the gene therapy, including duration. Aside from gene delivery, the main outcomes of the hydrodynamic procedure are the level of tissue damage that accompanies gene therapy. This tissue damage revolves around diluted cytoplasm caused by fluid rushing into the cell through membrane pores, and small vesicles appearing inside the cytoplasm of cells caused by fluid pinching off portions of the cell membrane. Sub-optimally, hydrodynamic injection can cause the death of a small proportion of hepatocytes from the injection. Indeed, mouse hydrodynamic tail vein injection yields a small but sizable portion of mouse hepatocytes death from the procedure, while the majority of hepatocytes recover and normal histology is rapidly re-established. While mouse hydrodynamic injection procedure has been rigorously optimized in numerous studies, the pig biliary hydrodynamic procedure has only been described in one study and there were no optimization studies around injection and resultant tissue injury.

Tissue injury is an important consideration for hydrodynamic injection. In one aspect, tissue injury must be tolerated and safe for the patient, particularly if the patient has liver disease or lower than normal synthetic liver function. A second consideration is that any hepatocyte death or damage can lead to inflammation and an immune response, which can lead to stimulation of an adaptive immune response that will remove cells expressing the transgene (i.e. possibly through direct recognition of the transgene as a foreign protein). This obviously is not a desired outcome for human gene transfer from this procedure. On the other hand, the magnitude of liver injury in mouse models is correlated with the gene transfection efficiency. Thus, liver enzymes can be useful biomarker to ascertain if effective pressures are being achieved through hydrodynamic injection. Clearly, both of these outcomes could be desirable within different genetherapy contexts and thus are important aspects of the disclosure. Toward the goal of injecting fluid into patients without causing any liver damage, a flow rate up to 5 mL/second would lead to no elevation ALT or AST well and only achieve dilute cytoplasm on histology, but no large intracellular vesicle formation. Toward optimizing the pressure and flow rate, a flow rate between 5-10 mL/second or greater can be tolerated by the pig and leads to extensive formation of large intracellular vesicles within hepatocytes on histology, as well as a relatively mild elevation of AST into the 200's U/L immediately post-procedure. Given that differences in catheter diameter may slightly influence the tissue damage from a programmed flow rate in the procedure, the current disclosure describes a process for screening these settings in a pig model in order to control for ALT and AST elevation in human patients.

In certain embodiments, only one flow rate will be applied for the injection, with a constant flow rate for the DNA solutions during the entire injection. In other embodiments of the disclosure, a higher flow rate is utilized for small portions of the injection and/or varied throughout the injection. In one example, a 50 mL DNA solution could be injected initially at a 5 mL per second flow rate for 4 seconds, before the power injector switches to a 10 mL per second for 3 seconds before switching to a 5 mL/sec for the remaining 2 seconds to complete the entire volume injected. In this manner, the DNA solution could be injected rapidly into the biliary system and then a brief period of high flow could enhance gene delivery without lasting too long in order to avoid significant tissue injury and toxicity. Furthermore, the results herein revealed that the rupture of the catheter typically occurred in the latter half of the injection, suggesting that a longer time at higher flow rates leads to more force on catheter walls. This could be ameliorated by making the higher flow rates last in shorter intervals. There are potentially a multitude of different combinations of flow rates to be explored, and this disclosure description does not restrict one to any one design.

The results herein also found that flow rate can be important in determining which hepatocytes are preferentially transfected during the procedure. The results herein demonstrated that modifying the flow rate by increasing it to 4 mL/sec or greater preferentially transfected regions around the portal triad as well as large vessels within the liver parenchyma. This finding is completely unexpected. Without being bound by theory, it is believed that this effect may be related to the increased biliary pressure during injection causing fluid to prematurely leave these spaces before extended deep into canaliculi. Interestingly, this transfection pattern was the opposite from other experiments with a lower transfection rate at 2 mL/sec wherein transfection was optimally seen around the central veins at the center of hepatic lobules. For these experiments, the areas around the portal triads had some positive staining but comparatively much less than seen at higher flow rates. This finding is particularly important in light of the fact that certain diseases including urea cycle disorders are preferentially treated in the zone one of hepatic lobules and thus would be better targeted using higher flow rates. By contrast, treating acute liver toxin injury normally occurs around zone 3, which metabolizes acetaminophen, for example. In this instance, the hydrodynamic procedure could be modified with a lower flow rate in order to target these hepatocytes. In other preferred embodiments, one may desire to optimize transfection of hepatocytes across as many total liver cells as possible. In these embodiments, one may want desire to vary the flow rate doing the procedure such that a flow rate and low flowrate are used during the procedure eventually yielding an even distribution of transfected hepatocytes. This strategy could be built into more complex flow rate patterns that also take into account the potential liver toxicity from the injection.

Minimizing DNA Loss from the Procedure

After the injection of DNA solution by the power injector at a programmed flow rate(s) of choice, there is the inherent problem of some of the DNA solution remaining in the catheter and power circuit tubing. Depending on the system and the power injectors and catheters employed, this could equal between 5-10 mL of volume. At lower total volumes of injected DNA solution, this could amount to a significant loss of total DNA approaching 10 to 20% have the DNA scheduled to be injected. The current disclosure describes several solutions for this issue. In one embodiment of the disclosure, the volume of DNA solution to be injected is increased, such that the proportional loss of total DNA solution will be less. The downside of this approach is that the volume and flow rate the patient is subjected to will be altered and thus may not be optimal for the pressure and/or level of transfection efficiency or tissue injury desired. In a second embodiment of the disclosure, the problem can be solved by mixing the DNA solution with a second solution that has a lower density, such that the solution would float on its surface. During injection through a single barrel power injector, the DNA solution will be pushed through the circuit and fully into the liver well the less dense liquid would also be ejected but remain within the tubing circuit and not enter into the patient. The volume of this second solution would equal the dead space volume of the catheter and power injector circuit. The downside of this approach is the use of a second exogeneous fluid mixed with the DNA solution, which could have negative impacts on the DNA solution, including binding some of the DNA molecules.

In a preferred aspect, a double-barreled power injector would be utilized to overcome the issue of dead space remaining post injection. In this scheme, one barrel of the power injector would contain a saline or dextrose or lactated ringer solution or other physiological solution that does not contain DNA. This solution could be initially used for the aforementioned priming and/or clearance/washing of the of the catheter and biliary system. In the context of solving the problem of residual DNA solution in the circuit, this second barrel would be connected a proximal location in the circuit to the primary barrel containing DNA, such that this second barrel could be activated to push the residual DNA solution in the circuit into the patient's liver.

In preferred embodiments, the flow rate of the second barrel with saline solution would be equivalent or greater than the final flow rate of the DNA solution chamber, in order to keep similar pressure for gene delivery. In other embodiments, the minimal volume injected from the second barrel would be equal to the volume of the tubing circuit and catheter. In other preferred embodiments, the maximum volume injected by the saline solution would equivalent to the catheter and tubing circuit volume, plus the calculated volume of the biliary system inside the patient's liver. In this manner, the disclosure effectively chases the DNA solution injected with a second non-DNA containing solution to assure maximal delivery of DNA into the patient's hepatocytes. In certain embodiments of the disclosure, the second solution is the same composition of the DNA solution, except without containing the DNA.

### Post-Injection Monitoring

The disclosure also describes several steps post injection to ensure successful injection and safety. In one embodiment of the disclosure, the intrabiliary pressure tracings during the injection would be reviewed. Pressure tracings are an important element for verifying that the procedure worked, that the balloon seal was secure without losing pressure because of backflow, and that the intended pressure was achieved with power injector achieving the desired performance. Furthermore, there is the possibility that different patients will have slightly different liver physiology and anatomy, such that a given flow rate in one individual will not obtain the desired pressure. In certain embodiments, failure of the power injector to achieve the desired pressure would lead to the physician repeating the procedure the same day. Thus, live pressure tracings in the disclosure would serve as quality assurance for this process and educate the endoscopist if a repeat injection is necessary.

The second step post-injection is that the balloon should be deflated and opened to allow fluid to be drained from the biliary system. Testing demonstrated that there is no need for the balloon to be opened for prolonged periods, unlike what was published previously in the prior art, given that the pressures with the balloon open alone are too low to mediate transfection. Thus, the balloon can be closed almost immediately post-injection.

After releasing fluid from the balloon, the balloon can be re-inflated and a small amount of radiocontrast injected into the biliary system in order to confirm that no rupture of any bile ducts have occurred. In certain embodiments, this step may be optional given that successful initial placement almost eliminates the chances of any bile duct rupture from occurring. Subsequent to checking the integrity of the biliary structures, the catheter can be removed from the bile ducts and withdrawn back into the endoscope and into the duodenum. After this, in some embodiments of the disclosure, the patients will be subjected to a blood draw within 15 minutes hydrodynamic injection to monitor for tissue injury from the procedure. This would ideally be performed in a rapid point of care assay in order to inform the endoscopist if a repeat procedure is needed to be performed on the patient that same day.

### Ability for the Biliary Hydrodynamic Injection Procedure to be Repeated

Beyond the steps of describing a single biliary hydrodynamic procedure, there are also important embodiments of the disclosure concerning the ability to repeat the injection in a single procedure day, either to make up for potential mistakes from the injection, or as a method to increase transfection efficiency. Another important embodiment of the disclosure is the potential for biliary hydrodynamic injection to be repeated on separate procedure days in order to re-dose gene therapy. It is not obvious from the prior literature if the biliary system and liver could manage the trauma of multiple injections either on the same day or on separate days. Moreover, there was a published concern of dilation of the biliary duct after hydrodynamic injection, that could be a cause of concern for permanent liver damage or reduced efficacy during redosing. Toward this goal of answering these questions, the current disclosure demonstrates the feasibility of repeated injections for the first time. In the first goal of determining repeat injections on the same day, the preferred embodiment of the disclosure shows that repeat injections are well tolerated with minimal effects on vital signs. There are no clear limitations for the nature of the volume and/or flow rate injected during these repeat injections. In some embodiments, a user may wait at least 1 minute or at least 5 minutes would lapse between injections in order to ensure vital signs return to baseline and that the patient's cardiovascular system could recalibrate to handle the excess volume injected.

In another aspect, the biliary hydrodynamic injections can be safely repeated within three weeks of each other. Data is presented here demonstrating that normalization of hematologic and liver chemistry profiles occurs after the first injection allowing the patient to resemble a previously non-injected human. The second injection can proceed without any disturbances or increase levels of liver damage, indicating that the prior injection does not sensitize the patient to a greater increase liver enzymes with repeated injections on separate days. This was not obvious before the prior work tested this in pigs. In the preferred embodiment of the disclosure, injections could be repeated with a minimum of two weeks after the first injection with no upper limit on the time course to repeat a second injection long term. In other embodiments, it is feasible to contemplate further injections over months to years. For example, depending on the disease being treated, it is possible that injections may be repeated years into the future to redose a monogenetic liver disease with a declining gene expression level. Preferred embodiments of the disclosure would necessitate monitoring liver enzymes and vital signs before any second injections to validate that they are within the normal range before proceeding with this second injection.

### Alternative Methods of Biliary Hydrodynamic Injection Monitoring

In certain embodiments, an additional method beyond pressure monitoring may be used for live monitoring of biliary hydrodynamic injection. This method will consist of mixing radiocontrast solution with the DNA solution being injected. Contrast solution will be diluted into the DNA solution, such that it's total percentage will be between 1 to 33%. During biliary hydrodynamic injection, fluoroscopy will demonstrate contrast injected into every liver lobe, growing in intensity during the injection. Successful injection will demonstrate contrast in all parts in the liver.

### Pancreatic Ductal Hydrodynamic Injection

Methods of gene delivery into the pancreatic tissue hydrodynamic gene delivery through the ductal system are also presented. The method consists of using endoscopic retrograde cholangiopancreatography (ERCP) in order to insert a catheter into the pancreatic duct. The pancreatic duct effectively permeates the entire pancreatic organ from the head of the pancreas into the distal tip of the tail of the pancreas. The ductal system then fans out into numerous small ductal branches the contact acinar cells, which secrete digestive enzymes into the pancreatic cavity. The unidirectional flow of fluid through the pancreatic ductal system makes it an appealing target for retrograde hydrodynamic gene therapy, since there is not an effective release valve of increased fluid pressure, unlike venous and arterial spaces. The central challenge for ductal hydrodynamic gene delivery rests in the potential for tissue injury causing pancreatitis. For that reason, the exact details of the gene delivery procedure seek to treat or ameliorate that possibility. In the preferred embodiment of the gene delivery method, a catheter will cannulate the ampulla of Vater and be advanced into the pancreatic duct. In many embodiments, a guidewire will complete this first step, followed by exchanging the catheter over the guidewire. After this step, the pancreatic juices will be suctioned and removed from the pancreas, such that later injection of fluid won't push these digestive enzymes into the tissue parenchyma. After this step, the catheter localization can be confirmed with contrast injection and fluoroscopic imaging, minding that afterwards, the contrast is readily suctioned to avoid irritation to the pancreatic tissue. The injection procedure then commences with DNA solution priming from a power injector all the way into the distal tip of the catheter. Given the relatively small volumes of the DNA solution injected in the pancreas versus the large dead volume in the catheter circuit (˜5 mL), it is preferred for the DNA priming to occur in order to avoid DNA wasted from the procedure along with wasted non-DNA solution going into the pancreas that simply causes tissue injury and is not contribute to gene expression.

Concerning the volume to be injected into the pancreas, the pancreatic ductal system has a relatively small volume of 2-3 mL's in human subjects and no larger than 5 mL's in total volume. The volume to be injected can be quite variable ranging from 5 mL's to 10 mL's to 20 mL's or at most 50 mL's for most applications. The fact that such a large volume could be injected inside the pancreatic ductal system was entirely unexpected. This likely reflects the escape of fluid into the surrounding tissues and vasculature. Preferred embodiments of the gene delivery method, the flow rate injected into the pancreas will be correlate directly with the pressure achieved in the duct, which in turn will correlate with gene delivery efficiency. In preferred embodiments of the method, the animal flow rate will be 1 mL/sec. In other embodiments, the flow rate will be 2 mL/sec, 3 mL/sec, 4 mL/sec, or 5 mL/sec.

In another method, the minor papilla would be cannulated (hence avoiding the ampulla of Vater and the biliary system) and a guidewire would be inserted through the accessory duct and lodged in the main pancreatic duct in the body or tail. The balloon catheter would be inserted over the guidewire and after a small amount of contrast injection, the balloon would be placed at the fusion of the dorsal and ventral pancreatic ducts. Then hydrodynamic injection would be performed. The advantage of this strategy is that: 1) there is no inadvertent biliary cannulation 2) access to the pancreatic duct in the body and tail would still be possible in patients with pancreasdivisium 3) hydrodynamic injection would selectively target the body and tail of the pancreas which would: a) target the region of the pancreas with the highest concentration of beta islet cells and b) spare the head of the pancreas from receiving hydrodynamic injection which may decrease the incidence and/or severity of post-procedure pancreatitis.

For the nucleic acid solution itself, several modifications will be made as compared to other hydrodynamic gene therapy procedures. For injecting into the pancreas, additional small molecules will be added to the DNA solution in order to abrogate the development of pancreatitis. These small molecules will be selected from a variety of drugs each having documented evidence of suppressing one of the mechanisms of pancreatitis permission. The purpose of adding these drugs into the hydrodynamic solution will be to facilitate their direct delivery into all aspects of the organ that received the DNA solution. This includes intracellular delivery into acinar cells of the digestive enzymes that otherwise facilitate degradation of the pancreatic tissue. The selection of small molecule drugs or pharmacologic agents for this purpose are selected from inhibitors of pancreatic enzymes including gabexate mesilate, nafamostat mesylate, ulinastatin, Camostat mesylate, Aprotinin, Pefabloc, Trasylol, and Urinary Trypsin Inhibitor, or enzyme suppressive agents, including somatostatin. Inhibitors of inflammation that could be co-injected include corticosteroids, tacrolimus, sirolimus. In some embodiments of the method, these drugs may not be included with the DNA solution, but rather injected into the pancreas before or after the hydrodynamic injection. In these methods, the drugs will be dissolved in a physiological solution like normal saline, lactate ringer's, or dextrose 5% water.

The drugs would be injected in a small volume similar to the total volume of the pancreatic duct (2-5 mL) at a slow flow rate (less than or equal to 1 mL/sec). The DNA solution will be prepared with or without the listed pharmacologic agents in a similar solution of normal saline, lactate ringer's, or dextrose 5% water. After hydrodynamic injection, the balloon will be promptly deflated, and any residual solution suctioned from the pancreatic ductal system. A repeat contrast injection and fluoroscopic procedure may be performed post-hydrodynamic injection to verify patency of the pancreatic duct. Similar to pre-procedure imaging, contrast should be promptly suctioned out to avoid toxicity to the pancreatic tissue. The catheter should be promptly removed from the pancreatic duct subsequently to avoid further irritation.

### Renal Hydrodynamic Injection

Methods of gene delivery into the kidney or renal tissue hydrodynamic gene delivery through the ureter system are presented. To deliver hydrodynamic fluid force into the kidney the readers are chosen as a conduit for this purpose. Ureters are ideal since they represent a vessel system with unidirectional flow, such that retrograde flow in the other direction would lead to exclusion of fluid tissue into the surrounding kidney parenchyma. The exam act mechanism how to leverage this unique anatomy has not been fully explored or enabled in the prior art. Previous study demonstrated that a renal pelvic hydrodynamic injection could mediate gene delivery into different kidney cells (Scientific Reports volume 7, Article number: 44904 (2017)). However, the relative efficiency was very low in and the renal pelvis itself was accessed directly by a surgical method placing a needle into the renal pelvis, which would be too invasive to translate into human patients. Moreover, fluid flow in the antegrade direction toward the bladder was not abrogated during their hydrodynamic injection procedure (J Vis Exp. 2018 Jan. 8; (131):56324), resulting in loss of pressure during their hydrodynamic injection. Methods here are presented fora procedure to accomplish efficient gene delivery procedure through a cystoscopic and ureteroscopic procedure. Briefly, a cystoscopic procedure would comment, inserting the scope through the urethra and into the bladder. A camera at the end of the scope would help visualize the two ureteral orifices. A guidewire inserted through the cytoscope can facilitate cannulation of one of the two ureteral orifices. The guidewire can then be exchanged for a catheter, or kept in and advanced all of the into the kidney pelvis, to help position the catheter at the correction location for injection described below.

A catheter inserted through the scope and either directly or with the help of the aforementioned guidewire placed into one of the two ureteral offices. The catheter could be advanced up the ureter optionally along the guidewire, and the position of the catheter monitored via contrast injection. The catheter would be advanced until it is located just in proximity to the end of the ureter and opening into the renal pelvis. The renal pelvis itself is fan-shaped or triangle-shape, which presents unique challenges in trying to create an effective seal with a balloon catheter. To solve this issue, the balloon would optimally be inflated in the distal ureter, which still has a relatively cylindrical shape like other vessels targeted such as the common hepatic duct and the pancreatic duct. It is essential to inject contrast before the procedure in order to confirm the seal of the balloon. Any room along the balloon on either side could have one of two outcomes: (1) the balloon could be pressed backwards, such that the seal is made tighter and naturally located in the correct location. (2) the fluid turbulence could push the balloon forward, thereby closing a jostling of the catheter during the hydrodynamic injection. Because of this possibility, it is better for the catheter balloon to be inflated deeper into the ureter versus the renal pelvis. Moreover, this point also highlights the utility of real-time pressure monitoring during the procedure, in order to verify the effectiveness of the balloon seal during renal hydrodynamic injection.

The guidewire and catheter tip could also facilitate proper catheter localization, since the guidewire extends to the end of the renal pelvis, and the catheter itself also has a tip that extends 1 to 4 cm past the balloon tip and well into the renal pelvis. In certain embodiments of the method, the catheter itself will hit against the renal pelvis wall, setting the appropriate distance for balloon inflation, which will otherwise still be localized in the ureter. Once the catheter is positioned and contrast injected, the contrast should be promptly removed before injection to avoid nephrotoxicity, which otherwise is a common toxicity from radio-contrast studies in patients. In addition, any urine in the renal pelvis should be optionally removed to decrease the potential for toxicity.

A pressure catheter can be optionally extended at this point from the catheter and into the renal pelvis space in order to monitor the injection. The catheter would then be primed with DNA solution from the power injector to the tip of the catheter. Injection would commence from the power injector. Volumes in preferable embodiments would be range from 10 mL, 15 mL, 20 mL, 25 mL, or 30 mL. Flow rates for the procedure are optimally 1 mL/sec, 2 mL/sec, 3 mL/sec, 4 mL/sec, or 5 mL/sec. The balloon should be promptly deflated after the injection stops, and residual DNA solution in the renal pelvis should be suctioned to remove it. Nephrotoxicity from the hydrodynamic injection should be monitored serial measurement of creatinine, blood urea nitrogen, and glomerular filtration. Each individual kidney should be done on a separate day in order to assure that baseline filtration functions are maintained. Biochemical markers should normalize before a procedure on the second kidney is attempted preferably on a separate day.

Ability to Target Specific Cell Types from Hydrodynamic Injection

The methods of gene delivery into the liver, kidney, and pancreas are further defined by methods of targeting specific cell types in the organs. The hydrodynamic gene delivery technique through the biliary and pancreatic ducts and ureters is unique since it ultimately targets multiple different cell types in the parenchyma of the tissue. Surprisingly, this includes different cell types that are not directly connected to the ductal lining that the gene transfer occurs along or is delivered through. An example of this is efficient transfer from biliary ducts into nerve tissue in the liver, wherein each component is not directly connected to each other. Another example for the liver is efficient expression in the endothelium of arteries in liver, which is again not directly connected into the bile duct system. A prominent example in the pancreas would be efficient gene transfer into islet cells, which are not connected to the pancreatic ductal system. This tissue distribution was very surprising and unexpected, especially the communication of the DNA across different cellular barriers without the help of vessel systems. While rodent hydrodynamic gene delivery findings are largely not translatable into large animal models, it is interesting to note that vascular hydrodynamic injection of mouse liver typically only delivers the gene into hepatocytes and not into other cell types. Studies about vascular hydrodynamic gene delivery into pancreatic cells also only appear to deliver into limited cell types, and lack a wide distribution. While the ability to target different cell types is a major advantage of this gene delivery strategy, it necessitates as a method of the disclosure that the DNA vector injected into the organ has a method of cell-specific targeting. This includes methods of using cell-specific promoters in order to restrict expression to the cell types. An example provided in this patent application revolves around the use of hepatocyte-specific promoters, which only restrict the expression inside of hepatocytes. In other experiments with ubiquitous, non-specific promoter, expression can be seen in all different types of cell types in the liver. Even when using ubiquitous promoters, differences can still be seen across cell types; an example of this is in the pancreas, where the SV40 promoter gives much stronger neuron expression compared to the CMV promoter. Herein, in the method disclosure, for each particular disease, examples of cell specific promoters are provided, that would allow for specific targeting of these cell types for a given disease.

### List of Cell Types and Cell-Specific Promoter for Targeting in Hydrodynamic Injection

Cholangiocytes: cytokeratin-19 promoter and cytokeratin-18 promoter.
Hepatocytes: alpha-1 antitrypsin promoter, thyroxine binding globulin promoter, albumin promoter, HBV core promoter, or hemopexin promoter.
Endothelial cells: intercellular adhesion molecule-2 (ICAM-2) promoter, fms-like tyrosine kinase-1 (Flt-1) promoter, vascular endothelial cadherin promoter, or von Willenbrand Factor (vWF) promoter.
Fibroblasts: COL1A1 promoter, COL1A2 promoter, FGF10 promoter, Fsp1 promoter, GFAPpromoter, NG2 promoter, or PDGFR promoter.
Smooth muscle cells: muscle creatine kinase promoter.
Neurons: synapsin I promoter, calcium/calmodulin-dependent protein kinase II promoter, tubulin alpha I promoter, neuron-specific enolase promoter and platelet-derived growth factor beta chain promoter.
pancreatic acinar cells: chymotrypsin-like elastase-1 promoter, Ptf1a promoter, or Amy2a promoter.
pancreatic ductal epithelial cells: Sox9 promoter, Hnf1b promoter, Krt19 promoter, and Mucipromoter.
pancreatic islet cells: Insulin promoter, glucagon promoter, somatostatin promoter, ghrelin promoter, or neurogenin-3 promoter.
proximal tubular epithelial cells: gamma-glutaryl transpeptidase promoter, Sglt2 promoter, or NPT2a promoter.
Podocytes: podocin promoter.
cells of the thick ascending limb of Henle: NKCC2 promoter cells of the collecting duct: AQP2 promoter.
renal epithelial cells: kidney-specific cadherin promoter.
Multiple cell types: cytomegalovirus promoter, EFlalpha promoter, SV40 promoter, ubiquitin B,GAPDH, beta-actin, or PGK-1 promoter.
   Using Drugs to Modulate Inflammation from Injection

In certain embodiments of the hydrodynamic gene delivery method, additional medications may be given before, during, or after the procedure to inhibit potential inflammation and cell injury. hydrodynamic delivery has the potential to introduce extracellular substances into the cytoplasm of cells, which could trigger stress pathways and cause apoptosis or necrosis in a fraction of cells, and/or otherwise stimulate immune cells directly. Indeed, this is observed inhydrodynamic tail vein injection in mice. In order to avoid this result of inflammation post-hydrodynamic gene injection, certain embodiments of the disclosure include giving immunosuppressive medications before, during, or after hydrodynamic gene delivery. These immunosuppressive medications include cyclophosphamide, cyclosporin, tacrolimus, sirolumus, mycophenolate mofetil, or a variety of different corticosteroids including dexamethasone and prednisone. In certain embodiments, the immunosuppressive medications can be administered by the catheter directly into the tissues themselves, before or after hydrodynamic injection. In other embodiments, the immunosuppressive medications will be dissolved into the nucleic acid and/or protein solution and injected with the hydrodynamic injection. Still in other embodiments, these medications may be given via routes approved by the FDA, such as oral, subcutaneous, or intravenous. In this case, the regimens may be started before the procedure, or after the hydrodynamic procedure concludes.

### Additional Description

In one aspect, it was assessed: (1) optimal parameters for intra-biliary-delivered hydrodynamic gene delivery; (2) demonstrate feasibility of liver cell transduction; and (3) assess whether successful transduction results in stable expression of the delivered plasmid proteins.

This disclosure establishes a minimally invasive method of non-viral gene delivery to the liver.

The injection formulations disclosed herein typically include an effective amount of a nucleic acid expression cassette in a pharmaceutically acceptable carrier suitable for hydrodynamic injection.

The formulations can include a physiologically-acceptable canier (such as physiological saline or phosphate buffer) selected in accordance with the route of administration and standard pharmaceutical practice.

Pharmaceutical compositions including a nucleic acid expression cassette are prepared according to standard techniques and include a pharmaceutically acceptable carrier, In some embodiments, normal saline is employed as the pharmaceutically acceptable carrier. Other suitable carriers include, e.g., water, buffered water, 0.9 saline, 0.3% glycine, and the like, including glycoproteins for enhanced stability, such as albumin, lipoprotein, globulin, etc. The resulting pharmaceutical preparations can be sterilized by conventional, well known sterilization techniques. The aqueous solutions can then be packaged for use or filtered under aseptic conditions and lyophilized, the lyophilized preparation being combined with a sterile aqueous solution prior to administration. The compositions cab contain pharmaceutically acceptable auxiliary substances as required to approximate physiological conditions, such as pH adjusting and buffering agents, tonicity adjusting agents and the like, for example, sodium acetate, sodiumlactate, sodium chloride, potassium chloride, calcium chloride, etc.

The compositions can be administered and taken up into the cells of a subject with or without the aid of a delivery vehicle. For example, nucleic acids may also be delivered by other carriers, including liposomes, polylneric micro- and nanoparticles and polycations such as asialoglycoprotein/polylysine, which can enhance transfection efficiency. In some embodiments, the composition is inc0113orated into or encapsulated by a nanoparticle, rnicroparticle, micelle, synthetic lipoprotein particle, or carbon nanotube. Preferred carriers include targeted liposomes (Liu, et al. Curr. Med. Chem., 10: 1307-1315 (2003)) such as immunoliposomes, which can incorporate acylated mAbs into the lipid bilayer, Polycations such as asialoglycoprotein/polylysine may be used, where the conjugate includes a molecule which recognizes the target tissue (e.g., asialoorosomucoid for liver) and a DNA binding compound to bind to the DNA to be transfected. Polylysine is an example of a DNA binding molecule which binds DNA without damaging it. This conjugate is then complexed with plasmid DNA for transfer.

Typically the compositions disclosed herein are administered to a subject in a therapeutically effective amount. As used herein the term “effective amount” or “therapeutically effective amount” means a dosage sufficient to treat, inhibit, or alleviate one or more symptoms of the disorder being treated or to otherwise provide a desired pharmacologic and/or physiologic effect. The precise dosage will vary according to a variety of factors such as subject-dependent variables (e.g., age, immune system health, etc.), the disease, and the treatment being effected.

Typically, the formulations include an amount of a nucleic acid expression cassette effective to modify the genome of one or more targets cells in a subject following hydrodynamic administration of the formulation to the subject. In preferred embodiments, the amount is effective to modify the genome of enough of the target cells to treat, reduce, or prevent one or symptoms a disease being treated, or to produce an alteration in a physiological or biochemical manifestation thereof.

Preferred dosage amounts also can be determined empirically as well as upon consideration of for example the therapeutic context and desired result age, and general health of the recipient.

The examples which follow also demonstrate effective dosages. Additionally, Khorsandi, et al, Cancer Gene Therapy, 15:225-230 (2008) reported administering pigs dosages of between 10 mg and 20 mg of plasmid, and humans dosages of 1 mg to 45 mg of plasmid by selective hydrodynamic injection (regional circulation), and all dosages were found to be safe and tolerated by the subjects. Therefore, generally, the dosages can range from about 0.001 mg to about 1,000 mg, more preferable about 0.01 mg to about 100 mg of nucleic acid expression cassette genome editing composition, depending on the subject to be treated, the route of administration, the targets cells.

Hydrodynamic injection, also referred to as high pressure injection, is a method of administering nucleic acids in vivo. Hydrodynamic injection is amenable to delivery of “naked” nucleic acids, and therefore does not require viral carriers that can require laborious procedures for preparation and purification, and carry with them concerns about the possibility for recombination with endogenous virus to produce a deleteriously infectious form. Hydrodynamic injection also does not appear to cause the immune response and other side effects that render the repeated administration of viral vectors problematic. Being different from carrier-based strategy and the earlier work employing hypertonic solution and elevated hydrostatic pressure to facilitate intracellular DNA transfer, hydrodynamic gene delivery relies on hydrodynamic pressure generated by a rapid injection of a large volume of fluid to deliver genetic materials into parenchyma cells. See also Suda and Liu, et al, Molecular Therapy, 15(12):2063-2069 (2007), Al-Dosari, et al, Adv. Genet. 54: 65-82 (2005), Kobayashi, et al, Adv Drug Deliv. Rev. 57: 713-731 (2005), and Herweijer and Wolff, Gene Ther. 14: 99-107 (2007).

Injection volume and injection speed can also be important consideration in the efficacy of hydrodynamic delivery. Faster injection speeds are generally preferred.

The solution can be delivered by any means suitable for delivering the desired volume at the desired rate. For example, the solution can be administered using an injection device such as a catheter, syringe needle, cannula, stylet, balloon catheter, multiple balloon catheter, single lumen catheter, and multilumen catheter. Single and multi-port injectors may be used, as well as single or multi-balloon catheters and single and multilumen injection devices. A catheter can be inserted at a distant site and threaded through the lumen of a vein so that it resides in or near a target tissue. The injection can also be performed using a needle that traverses the skin and enters the lumen of a vessel.

Administration can be aided by the incorporation of pump or other system to facilitate delivery of the desired volume at the desired pressure. In a particular embodiment, administration includes use of a computer-assisted system enabling real-time control of the injection based on the hydrodynamic pressure at the injection site of the tissue. Precise control of injection can avoid tissue damage caused by too heavy an injection, or low gene delivery efficiency due to insufficient volume or injection speed.

For instance, in certain preferred system, upon injection concluding, the intrabiliary pressure may remain at 1, 2, 3, 4, 5, 6, 7, 8, 9, 10, 12, 14, 15, 16, 18 or 20 mmHg or more higher than the pre-injection intrabiliary pressure, at least until the extraction balloon is deflated.

Gene delivery can also be optimized and toxicity (tissue damage) minimized by varying the volume of the solution and the speed of injection; varying the osmotic pressure by the addition of mannitol to the injection solution; increasing fluid and DNA extravasation, e.g., by vessel dilation using papaverine, hyaluronidase, or VEGF protein pre-injection, and the like.

In some embodiments one or more vessels or ducts are occluded to reduce or prevent flow of the solution in one or more directions, for example, back flow. Methods of occluding ducts or vessels can be accomplished by varying methods. For example, the injection apparatus itself can reduce back flow. In some embodiments one or more cuffs, tourniquets or combination thereof is used to reduce or prevent solution flow in one or more directions. The cuff or tourniquets can be applied directly to the vessel, or to the tissue surrounding the vessel. In some embodiments, one or more balloon catheters is used to reduce or prevent solution flow in one or more directions. For at least certain systems, use of a balloon catheter to create a substantially closed system may be preferred.

The occlusion(s) can be carried out using non-invasive procedures, minimally invasive procedures, or invasive procedures. For example, in some embodiment, the ducts vessels are occluded by an open surgical procedure. In other embodiments, the ducts or vessels are occluded using a minimally invasive procedure such as percutaneous surgery. Varying approaches can be carried out through the skin or through a body cavity or anatomical opening and may incorporate the use of catheters, arthroscopic devices, laparoscopic devices, and the like, and remote-control manipulation of instruments with indirect observation of the surgical field through an endoscope or large scale display panel, etc.

The disclosed hydrodynamic delivery methods are preferably via the biliary tree of a subject and do not involve delivery through the blood vessels such as an artery or a vein.

The target cells, and therefore the particular method of hydrodynamic injection, are typically selected based on disease to be treated. In some embodiments, the target cells are liver cells, kidney cells, or pancreatic cells.

In preferred embodiments, the target cells are parenchymal cells. Parenchymal cells are the distinguishing cells of a gland or organ contained in and supported by the connective tissue framework.

The parenchymal cells typically perform a function that is unique to the particular organ.

The term “parenchymal” often excludes cells that are common to many organs and tissues such as fibroblasts and endothelial cells within blood vessels.

In a liver organ, the parenchymal cells include hepatocytes, Kupffer cells and the epithelial cells that line the biliary tract and bile ductules. The major constituent of the liver parenchyma are polyhedral hepatocytes (also known as hepatic cells) that presents at least one side to an hepatic sinusoid and opposed sides to a bile canaliculus. Liver cells that are not parenchymal cells include cells within the blood vessels such as the endothelial cells or fibroblast cells.

Applications of the disclosed compositions and methods include gene therapy, e.g., to treat a disease, or as an antiviral, antipathogenic, or anticancer therapeutic. The disclosed methods can be used to treat any disease or condition wherein genome modification of target cells is effective to treat the disease or condition, and wherein the target cells can be transfected with the disclosed compositions by hydrodynamic injection.

Preferably the cells leading to the disease pathology can be transfected by hydrodynamic injection. Preferred target cells include those discussed above, including liver cells, kidney cells and pancreatic cells.

### Catheter Delivery Systems Such as to Deliver to Biliary Tree Liver, Pancreatic Duct/Kidney

In preferred systems, such as to access and deliver therapeutic agents to the biliary tree/liver, pancreatic duct/pancreas, renal collecting system/kidney, a catheter delivery system suitably may be detectable, for example fluoroscopically visible. In an administration protocol, a catheter suitably may be placed in position for administration with or without a guidewire. If used, guidewire of varying configurations may be suitably used, include those having a inner diameter of from about 0.01 to 0.04 inches for example diameters of 0.018 inch, 0.025 inch, 0.035 inch or other diameters. The guidewire suitably can be radio-opaque if desired. Preferred catheters may have one or a plurality of ports of equal or varying diameters, for example 2, 3, 4 or more ports. The largest diameter port would be for the hydrodynamic delivery. Preferred catheters suitably can be 6 Fr-12 Fr in size as well as other sizes.

If two or more ports are used, then one port can be used to inflate the occlusion balloon or activate a mechanism to occlude the lumen and prevent/decrease the risk of catheter migration during injection and the other port will allow for the guidewire initially to run through it but then the guidewire can be removed and contrast/hydrodynamic delivery of a solution can be performed through that same port.

If three ports are used, a preferred configuration can include a first port for the occlusion balloon etc, a second distinct port for the guidewire/contrast/hydrodynamic delivery, and a third distinct port for the pressure catheter.

If four ports are used, a preferred configuration can include one for the occlusion balloon etc, one for the guidewire/contrast, one for contrast/hydrodynamic delivery, one for the pressure catheter.

In certain preferred aspects, the catheter may have the pressure catheter embedded in its tip so that only 2-3 additional ports are required.

If two additional ports are utilized, one can be used for the occlusion balloon etc, and a second can be used for the guidewire/contrast/hydrodynamic delivery. If three additional ports are utilized, one can be used for the occlusion balloon etc, a second can be used for the guidewire/contrast, and a third can be used for the contrast/hydrodynamic delivery.

In additional preferred systems, in addition to the catheter, a mechanism can be utilized of injecting the solution at a variety of pressures, volumes, duration, flow rates that may be fixed or titrated to the feedback from the pressure sensor.

### Biliary Hydrodynamic Injection

For particularly preferred biliary hydrodynamic injection of therapeutic materials (e.g. nucleic acid expression cassette), the systems will be suitable for both male and female patients. In a preferred protocol, an endoscope or echoendoscope may be used, and a catheter is placed via the transoral route/mouth into the bile duct either through the major papilla or via direct puncture through the duodenal and advanced to the upstream biliary tree in a retrograde fashion. A balloon or any mechanism can be used to occlude the lumen is inflated/activated and the catheter is withdrawn (or kept in position) to occlude one of the common hepatic duct, left hepatic duct, right hepatic duct. The purpose of the balloon/other mechanism is to not only occlude the lumen and prevent antegrade flow of the solution injected but also to anchor the catheter in position.

The catheter suitably can be placed into the above position under direct visualization (cholangioscopy) and/or under fluoroscopic visualization. If fluoroscopy is used, the biliary tree could opacify and this would aid in positioning the catheter tip in the area of interest. A guidewire suitably can be used to aid in the advancement of the catheter to the optimal position. An optimal position could be the common hepatic duct to allow for the solution injected to enter the entire biliary system of the liver. An optimal position also could be one of the hepatic ducts (left hepatic duct, right hepatic duct) such that only a portion of the hepatic parenchyma/hepatocytes is subject to hydrodynamic injection. Bile and or contrast agent if used can be aspirated to confirm the position in the biliary tree and to remove as much bile as possible to minimize retrograde reflux of bile into the hepatic parenchyma/hepatocytes. Suitably, the biliary tree is lavaged with a solution such as saline (but could be other solutions) to remove as much bile as possible to minimize retrograde efflux of bile into the hepatic parenchyma/hepatocytes. Suitably, the biliary tree is primed with the solution of interest.

Suitably, the balloon could be inflated at this point (prior to the hydrodynamic injection) or at any point prior to the catheter tip being in position. Hydrodynamic injection is then suitably performed preferred such that the solution of interest enters the various cells of hepatic parenchyma/hepatocytes. The injection suitably may be performed under fluoroscopic guidance. The injection may or may not be performed with a power injector. The volume of solution injected, the speed of injection, the duration of injection, and the pressure of injection could be detected in real time by a pressure sensor incorporated into the catheter or running through the working channel of the catheter. The pressure sensor maybe connected to a system which is able to regulate the injection parameters such that a variety of pressure waveforms could be generated (eg. initial high pressure followed by stable moderate pressure, or a stable pressure throughout OR multiple bursts of high pressure on a background of moderate pressure (sawtooth pattern)) prior to returning the baseline when the injection stops and/or the balloon/occluding mechanism is deflated. Suitably, contrast in reinjected to opacify the biliary tree to assess for leak. This can be performed by re-inflating the balloon or not using the balloon at all. The catheter is then suitably removed.

The method recognizes that the system is not an entirely “closed” system in the absolute sense as a significantly greater volume can be injected into the biliary tree than what the biliary tree could otherwise hold. The solution therefore likely/almost certainly does enter the branches of the hepatic sinusoids, portal vein and hepatic vein and this could result in the system spread of the solution.

The method may or may not be performed with prophylactic and/or post procedure antibiotics injected intravenously or lavaged into the biliary tree.

Suitably, the flow rate of the injection could be up to or greater than 2 mL/sec, 3 ml/sec, 5 ml/s, 10 ml/sec, or other value.

Suitably, the pressure in the biliary during hydrodynamic gene delivery could be up to or greater than 40 mmHg, 50 mmHg, 100 mmHg, 150 mmHg, or more. In at least some aspects, an upper limit may be 200 mmHg, although in certain systems higher pressures may be employed.

The delivery injected suitably may have various concentration of the substance of interest (gene/DNA etc) and the solution could have various viscosity.

Hydrodynamic injection could occur using various volumes of solution, for example 20 mL, 30 mL, 40 mL, 60 mL, 80 mL, 100 mL, 120 mL, 150 mL, 180 mL, 200 mL or other amount.

The balloon suitably can be kept inflated for just the duration of injection or for an extended period of time after the hydrodynamic injection/power injection is completed.

The injection suitably can be repeated at the same site or at other sites in the biliary tree during the same procedure. For example, the other hepatic duct or at the same site to optimize transfection.

The entire process of hydrodynamic injection can be repeated one or more times to the same site or new sites in the biliary tree.

### Pancreatic Hydrodynamic Injection:

In preferred systems, such as to access and deliver therapeutic agents to the biliary tree/liver, pancreatic duct/pancreas, renal collecting system/kidney, suitably patients may be male or female mammals such as humans. In preferred aspects, an endoscope or echoendoscope or other device may be used and a catheter suitably placed via the transoral route/mouth into the pancreatic duct either through the major papilla or via direct puncture through the duodenal/gastric wall and advanced to the upstream biliary tree in a retrograde or antegrade fashion. A balloon or other mechanism suitably can be used to occlude the lumen is inflated/activated and the catheter is withdrawn (or kept in position) to occlude one of the main pancreatic duct. The purpose of the balloon/other mechanism is to not only occlude the lumen and prevent antegrade flow of the solution injected but also to anchor the catheter in position.

Suitably, a catheter can be placed into the above position under direct visualization (pancreatoscopy) and/or under fluoroscopic visualization.

If fluoroscopy is used, the pancreatic tree could opacify and this would aid in positioning the catheter tip in the area of interest.

A guidewire suitably can be used to aid in the advancement of the catheter to the optimal position.

In certain aspects, an optimal position could be the main pancreatic duct to allow for the solution injected to enter the entire pancreatic parenchyma.

In additional aspects, an optimal position could be upstream from the entry of the accessory pancreatic duct such that the is no or minimal leakage of solution/pressure through the accessory pancreatic duct and minor papilla.

Suitably, if pancreatic ductal fluid and or contrast agent if used is aspirated to confirm the position in the pancreatic tree and to remove as much pancreatic fluid as possible to minimize retrograde reflux of pancreatic fluid into the pancreatic parenchyma,

Suitably, the pancreatic parenchmya includes acinar cells and islet cells. In certain aspects, acinar cells are treated.

In preferred applications, the pancreatic tree is lavaged with a solution such as saline (but could be other solutions) to remove pancreatic fluid to minimize retrograde efflux of pancreatic fluid into the pancreatic parenchyma.

Again, in preferred protocols, the pancreatic tree is primed with the solution of interest. The balloon suitably could be inflated at this point (prior to the hydrodynamic injection) or at any point prior to the catheter tip being in position.

Hydrodynamic injection is then suitably performed such that the solution of interest enters the various cells of the pancreatic parenchyma. The injection may be performed under fluoroscopic guidance. The injection may or may not be performed with a power injector.

The volume of solution injected, the speed of injection, the duration of injection, and the pressure of injection could be detected in real time by a pressure sensor incorporated into the catheter or running through the working channel of the catheter. The pressure sensor suitably may be connected to a system which is able to regulate the injection parameters such that a variety of pressure waveforms could be generated (e.g. initial high pressure followed by stable moderate pressure, or a stable pressure throughout OR multiple bursts of high pressure on a background of moderate pressure (e.g. sawtooth pattern)) prior to returning the baseline when the injection stops and/or the balloon/occluding mechanism is deflated. Contrast suitably may be reinjected to opacify the pancreatic tree to assess for leak. This can be performed by re-inflating the balloon or not using the balloon at all. The catheter is then suitably removed.

The method recognizes that the system may not be entirely “closed” system in the absolute sense as a significantly greater volume can be injected into the pancreatic tree than what the pancreatic tree could otherwise hold. The solution therefore likely/almost certainly does enter the branches of the superior mesenteric vein, splenic vein, superior and inferior pancreatoduodenal veins and this could result in the system spread of the solution.

The method may or may not be performed with prophylactic and/or post procedure antibiotics injected intravenously or lavaged into the pancreatic tree.

The flow rate of the injection suitably can vary for example up to or greater than 2 mL/sec, 3 ml/sec, 5 ml/s, 10 ml/sec or other. The pressure in the pancreatic duct during hydrodynamic gene delivery also may vary and suitably may be up to or greater than 40 mmHg, 50 mmHg or other. In certain systems, an upper limit may be 200 mmHg, although higher pressures may be useful in certain systems.

The solution injected suitably can have various concentration of the substance of interest (gene/DNA etc) and the solution could have various viscosity.

Hydrodynamic injection suitably can use various volumes of solution, for example up to or greater than 20 mL, 30 mL, 40 mL, 60 mL, 80 mL, 100 mL, 120 mL, 150 mL, 180 mL, 200 mL or other amount.

In certain aspects, the balloon can be kept inflated for just the duration of injection or for an extended period of time after the hydrodynamic injection/power injection is completed.

In certain aspects, the injection can be repeated at the same site or at other sites in the pancreatic duct during the same procedure to optimize transfection.

In additional aspects, the entire process of hydrodynamic injection can be repeated one or more times to the same site or new sites in the pancreatic ductal system.

Renal Hydrodynamic Injection: In preferred systems of renal hydrodynamic injection, suitably patients may be male or female mammals such as humans.

In a preferred protocol, preferably under sterile technique, a catheter may be placed via the urethra into the ureter (either the left or right) and advanced to the renal pelvis. A balloon or other mechanism suitably can be used to occlude the lumen is inflated/activated and the catheter is withdrawn (or kept in position) to occlude one of the major calyx, the renal hilus, the renal pelvis, or the proximal aspect of the ureter. The purpose of the balloon/other mechanism is to not only occlude the lumen and prevent antegrade flow of the solution injected but also to ancho the catheter in position.

Suitably, the catheter can be placed into the above position under direct visualization (ureteroscopy) and/or under fluoroscopic visualization.

Suitably, if fluoroscopy is used, the renal collecting system could opacify and this would aid in positioning the catheter tip in the area of interest.

A guidewire suitably can be used to aid in the advancement of the catheter to the optimal position.

An optimal position could be the renal pelvis, renal hilum, or proximal ureter to allow for the solution injected to enter the entire collecting system of that kidney.

An optimal position also could be one of the major calyx such that only a portion of the renal parenchyma is subject to hydrodynamic injection.

If urine and or contrast agent if used is aspirated to confirm the position in the renal collecting system and to remove urine to minimize retrograde efflux of urine into the renal parenchyma Suitably, the collecting system is lavaged with a solution such as saline (but could be other solutions) to remove as much urine as possible to minimize retrograde efflux of urine into the renal parenchyma.

The renal collecting system preferably is primed with the solution of interest. The balloon if used could be inflated at this point (prior to the hydrodynamic injection) or at any point prior to the catheter tip being in position.

Hydrodynamic injection is then suitably performed such that the solution of interest enters the various cells of renal parenchyma. The injection may be suitably performed under fluoroscopic guidance. The injection may or may not be performed with a power injector.

The volume of solution injected, the speed of injection, the duration of injection, and the pressure of injection could be detected in real time by a pressure sensor incorporated into the catheter or running through the working channel of the catheter.

The pressure sensor if used suitably may be connected to a system which is able to regulate the injection parameters such that a variety of pressure waveforms could be generated (e.g. initial high pressure followed by stable moderate pressure, or a stable pressure throughout or multiple bursts of high pressure on a background of moderate pressure (e.g. sawtooth pattern))prior to returning the baseline when the injection stops and/or the balloon/occluding mechanism is deflated.

In certain systems, contrast may be reinjected to opacify the collecting system to assess for leak. This can be performed by re-inflating the balloon or not using the balloon at all. The catheter is then suitably removed.

The method recognizes that the system is not an entirely “closed” system in the absolute sense as a significantly greater volume can be injected into the collecting system than what the collecting system could otherwise hold. The solution therefore likely/almost certainly does enter the branches of the renal artery and vein and this could result in the system spread of the solution

The method may or may not be performed with prophylactic and/or post procedure antibiotics injected intravenously or lavaged into the collecting system

The flow rate of the injection suitably may be up to or greater than 2 mL/sec, 3 ml/sec, 5 ml/s, 10 ml/sec or other amount.

The pressure in the collection system during hydrodynamic gene delivery suitably may be up to or greater than 40 mmHg, 50 mmHg, 100 mmHg, 150 mmHg, or other amount. In certain systems an upper pressure limit may be about 200 mmHg, although in certain systems higher pressure may be suitably employed.

The solution injected suitably may have various concentration of the substance of interest (gene/DNA etc) and the solution could have various viscosity.

Hydrodynamic injection could occur using various volumes of solution, for example up to or greater than 20 mL, 30 mL, 40 mL, 60 mL, 80 mL, 100 mL, 120 mL, 150 mL, 180 mL, 200 mL or other amount.

The balloon if used suitably can be kept inflated for just the duration of injection or for an extended period of time after the hydrodynamic injection/power injection is completed.

The injection suitably can be repeated at the same site or at other sites in the renal collecting system during the same procedure. For example, at another major calyx, at the other kidney, or at the same site to optimize transfection.

The entire process of hydrodynamic injection suitably can be repeated one or more times to the same site or new sites in the collecting system.

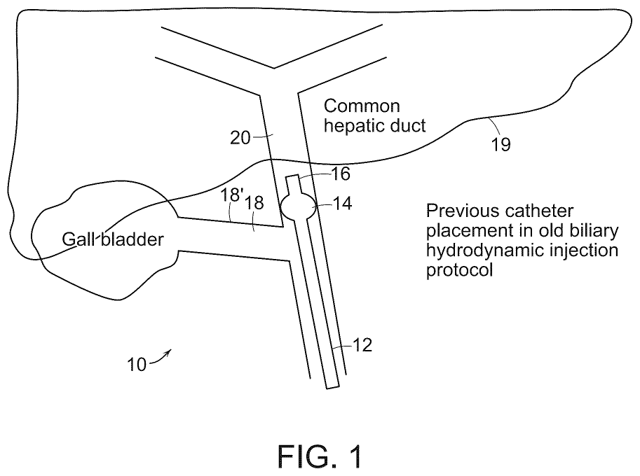

Referring now to the drawings, FIG. 1 shows a delivery arrangement 10 where catheter 12 with balloon 14 and distal delivery end 16 is positioned within hepatic duct 20 aside duct 18. In this system, catheter balloon 14 and delivery end 16 which injects a nucleic acid composition 20 is beyond duct 18 and top wall 18′ thereof but prior to liver wall 19.

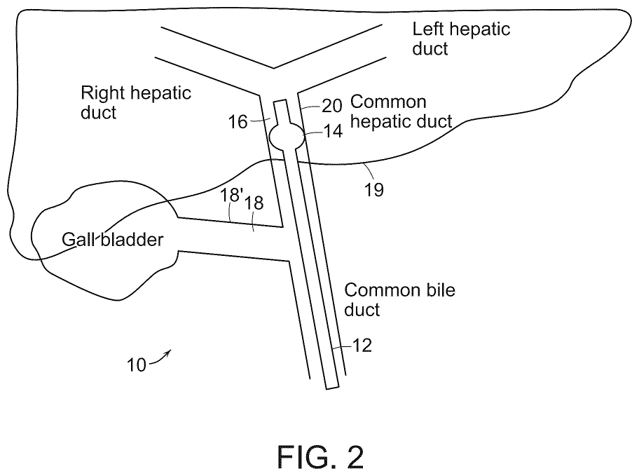

FIG. 2 shows a preferred delivery arrangement 10 where catheter 12 with balloon 14 and distal delivery end 16 is positioned within hepatic duct 20 beyond duct 18, where both balloon 14 and catheter distal delivery end 16 are positioned within the liver beyond liver wall 19. As discussed, by such positioning of the delivery end 16 as well as balloon 14 within the subject's liver, potential rupture of duct 20 can be minimized during delivery of nucleic acid under elevated pressure through catheter end 16.

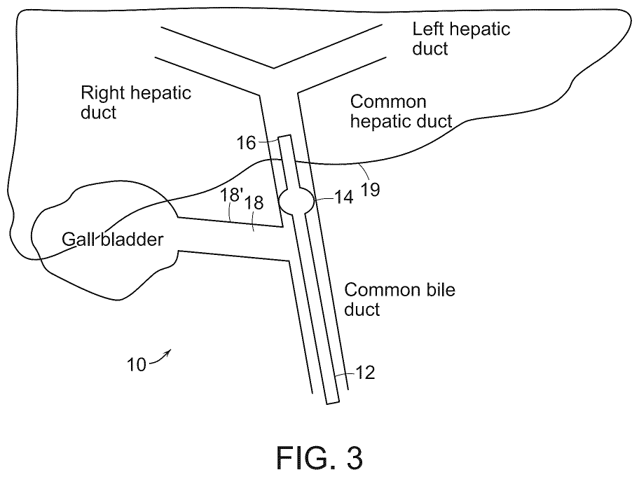

FIG. 3 shows a preferred delivery arrangement 10 where catheter 12 with balloon 14 and distal delivery end 16 is positioned within hepatic duct 20 beyond duct 18, where distal delivery end 16 is positioned within the liver beyond liver wall 19. As discussed, by such positioning of the delivery end 16 within the subject's liver, potential rupture of duct 20 can be minimized during delivery of nucleic acid under elevated pressure through catheter end 16. In this arrangement, balloon 14 is positioned beyond duct wall 18′ but before liver wall 19.

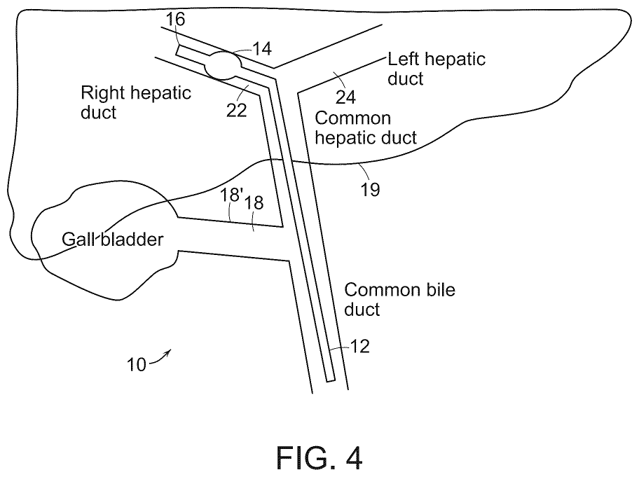

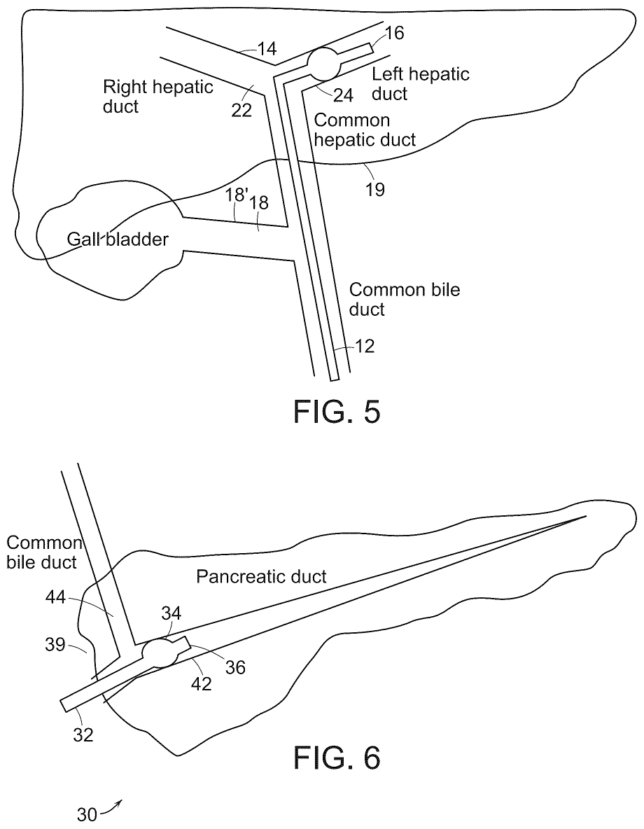

FIGS. 4 and 5 show additional preferred delivery arrangements where catheter 12 with balloon 14 and distal delivery end 16 is positioned within hepatic duct 20 beyond duct 18, where both balloon 14 and catheter distal delivery end 16 are positioned within the liver beyond liver wall 19. In FIG. 4, balloon 14 and distal delivery end 16 is positioned within right hepatic duct 22 and in FIG. 5 balloon 14 and distal delivery end 16 is positioned within left hepatic duct 24. Again, by such positioning of the delivery end 16 as well as balloon 14 within the subject's liver, potential rupture of duct 22 (for the arrangement of FIG. 4) or duct 24 (for the arrangement of FIG. 5) can be minimized during delivery of nucleic acid under elevated pressure through catheter end 16.

FIG. 6 shows a preferred pancreatic delivery arrangement 30 where catheter 32 with balloon 34 and distal delivery end 36 is positioned within pancreatic duct 42, where both balloon 34 and catheter distal delivery end 36 are positioned within the subject's pancreas beyond pancreas wall 39. By such positioning of the catheter delivery end 36 as well as balloon 34 within the subject's liver, potential rupture of pancreatic duct 42 can be minimized during delivery of nucleic acid under elevated pressure through catheter end 36.

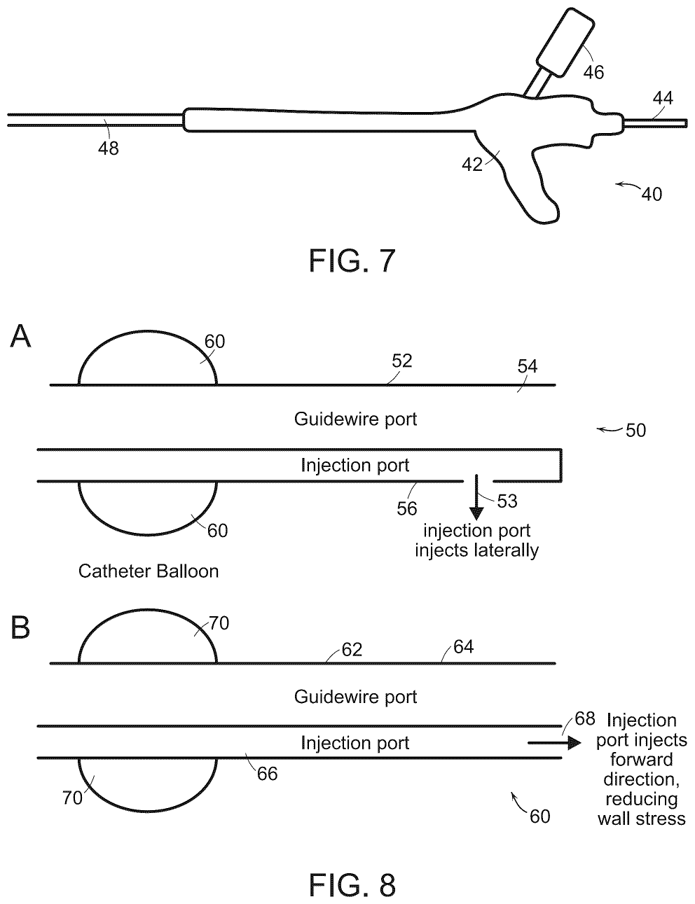

FIG. 7 shows a suitable delivery device 40 for administration of a nucleic acid composition in accordance with the present methods and systems.

FIG. 8A shows an injection catheter system 50 that can be utilized in the present methods and system. Catheter 52 include multiple lumens of guidewire port 54, injection port 56 for flow of a nucleic acid composition, injection or delivery opening 58 and balloon 60. As depicted in FIG. 8A, positioning of opening 58 provides for lateral injection of an administered composition.

FIG. 8B shows a preferred injection catheter system 60 that can be utilized in the present methods and system. Catheter 62 include multiple lumens of guidewire port 64, injection port 66 for flow of a nucleic acid composition, injection or delivery opening 68 and balloon 70. As depicted in FIG. 8B, positioning of opening 68 provides for forward injection of an administered composition.

Thus, in this preferred system, a nucleic acid composition is not delivered laterally as shown in FIG. 8A and instead the delivery direction of the FIG. 8B system is forward along the duct length and substantially perpendicular to that of the FIG. 8A system. As discussed, the forward delivery of the FIG. 8B system can provide notable advantages, including reduced force against the duct wall and thus the possibility of duct wall rupture may be minimized.

The following non-limiting examples are illustrative.

### Example 1

This Example shows inter alia limited tensile strength of current endoscopic catheters for hydrodynamic pressure.

### Catheter and Power Injector Testing In Vitro

As a first step toward defining injection parameters, a series of experiments were conducted using the endoscopic catheter and power injection in vitro, connecting the catheter directly to the power injector tubing. Contrast and injection ports were explored, which have different widths and thus may tolerate different flow rates. Testing was also conducted at the 999 and 1200 psi setting on the power injector with no apparent differences noted during in vitro testing. Indeed, the maximal flow route in the wider injection port was 10 mL/sec. At 15 mL/seconds, the circuit was burst with breaking of the catheter wall. When the injection port was tested, up to 5 mL/sec was achieved, although the volume ultimately delivered was less than the programmed rate and took a longer time. The medical power injector has a build-in system for the pressure tolerated within the circuit (1200 psi is maximum), so this threshold was likely reached causing a decreased flow rate. Thus, the tensile strength of the endoscopic catheter cannot withstand the full pressure of potential power injector options and represents a limitation for the procedure.

### Example 2

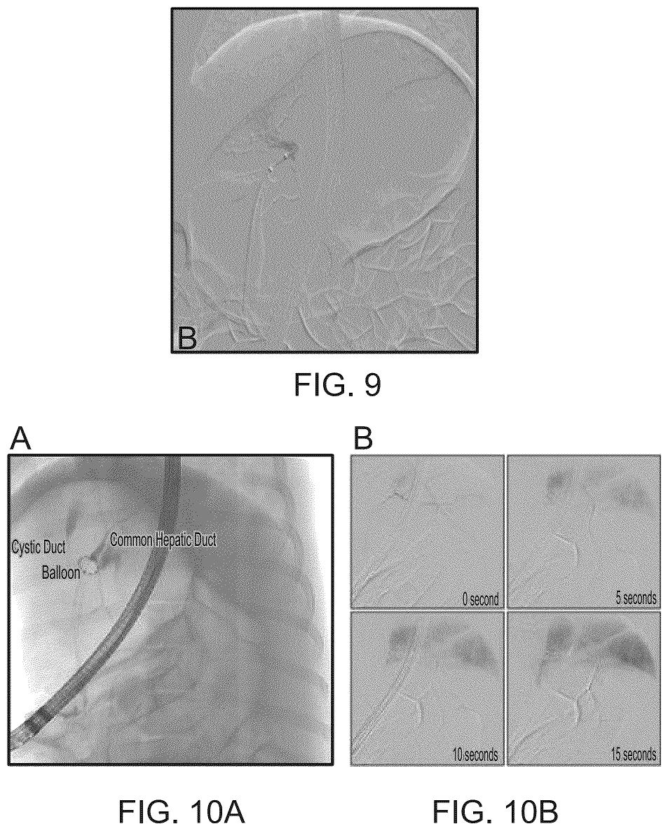

This Example shows inter alia optimization of balloon location and modification of the catheter tip. Previous data indicates that the common bile duct ruptures at parameters above 30 mL and/or 2 mL/sec. As an example, previous data showed that at an injection of 40 mL at 2 mL/sec caused rupture of the proximal common hepatic duct, which is represented by contrast extravasation immediately distal to the tip of the balloon catheter just below the hepatic hilum. A fluoroscopic image of this rupture is shown in FIG. 9.

In current systems, by optimizing the balloon location at the liver hepatic hilum and changing the end of the catheter tip from a lateral injection to a forward injection tip (i.e. cutting off the end), this enabled efficiency contrast injection with no bursting of the CHD at any flow rate or volume injected. Images of the initial localization of the catheter and the balloon are seen in FIG. 10A, while FIG. 10B shows the typical progression of contrast into the liver during injection.

### Example 3

This Example shows inter alia test subjects are able to tolerate repeated injections within a single procedure.

Different biliary hydrodynamic injection parameters were tested that could be tolerated by pigs within a single procedure day (Table 1, see below). The central question was if multiple hydrodynamic injections could be repeated within the same pig during one operation. This strategy has not traditionally be explored in rodent models, while other pig studies have isolated specific lobes in succession, but not repeated hydrodynamic injection within the same lobe.³⁴ If feasible, the repeated injection could in theory function as multiple transfections, thus boosting total gene delivery. Toward this goal, more hydrodynamic injections were repeated after several minutes post-injection in the pig. The next injection into Pig #1 increased the flow rate to 4 mL/sec at the same volume, which was tolerated with no issues. A higher volume (50 mL) and flow (5 mL/sec) were next employed, although these parameters triggered a power injector alarm leading to decreasing in flow rate.

As another example, a second pig, Pig #2, was next injected. Believing that the pressure parameter on the power injector could be increased in order to remove the error message, an injection was repeated at 5 mL/sec flow rate and 1200 psi. During injection into the pig, however, the circuit tubing burst near the end of the injection near the exit from the power injector, indicating limitations to the catheter materials. Interestingly, the pig itself did not suffer any issues during the procedure. Because of this, the circuit pressure was reduced back to 999 psi, which appears to be a strength the catheter materials can tolerate. With this limitation, a higher volume with a lower flow rate was tested (50 mL and 3 mL/sec) to ensure no error message would be observed. A higher volume (37 mL) at 4 mL/sec was also tested. Both were well tolerated by the pigs with no issues at the power injector, suggesting that maximal limits were not reached.

TABLE 1

| | | | | | |
|---|---|---|---|---|---|
| Injection | Volume | Flow rate | Pressure | | |
| Attempt | (mL) | (mL/sec) | (psi) | Port | Notes |

Pig #1

| | | | | | |
|---|---|---|---|---|---|
| 1 | 30 | 2 | 999 | Injection (small) | Well-tolerated |
| 2 | 30 | 4 | 999 | Injection (small) | Well-tolerated |
| 3 | 50 | 5 | 999 | Injection (small) | Alarm during injection, flow rate |
| | | | | | decreased to balance pressure |

Pig #2

| | | | | | |
|---|---|---|---|---|---|
| 1 | 45 | 5 | 1200 | Injection (small) | Circuit burst where line connected to |
| | | | | | the power injector and to the port. |
| | | | | | No evidence of liver parenchymal damage |
| 2 | 50 | 3 | 999 | Injection (small) | Well-tolerated |
| 3 | 37 | 4 | 999 | Injection (small) | Well-tolerated |

Pig #3

| | | | | | |
|---|---|---|---|---|---|
| 1 | 30 | 2 | 999 | Injection (small) | Balloon slipped, lower pressure reading |
| 2 | 30 | 2 | 999 | Injection (small) | Well-tolerated |
| 3 | 60 | 3 | 999 | Injection (small) | Well-tolerated, Alarm by the end of |
| | | | | | the injection, flow rate decreased |
| 4 | 140 | 1 | 999 | Injection (small) | Well-tolerated |
| 5 | 80 | 4 | 999 | Injection (small) | Well-tolerated |
| 6 | 47 | 10 | 999 | Guide port (big) | Well-tolerated |

### Example 4

This Example shows inter alia there is no clear volume limit for biliary hydrodynamic injection, beyond considerations of cardiovascular volume load.

Considerations of maximum volume injected during biliary hydrodynamic injection were explored, and how that would influence the relative intrabiliary pressure achieved. Seeing that increased volume at high flow rates appears to stress the system, there was a question if a significantly larger, supraphysiologic volume at a low flow rate would similarly stress the injection system or the pig vital signs. Toward testing this question, 140 mL of volume was injected, near the volume limit of the power injector, into the system at 1 mL/sec. The pig tolerated the injection with large volume with no significant changes in vital signs and the circuit had no issues. This indicates the biliary system has significantly higher and undefined upper limits of volume during the injection.

Pig #3

| Injection | Volume | Flow rate | Pressure | | |
|---|---|---|---|---|---|
| Attempt | (mL) | (mL/sec) | (psi) | Port | Notes |
| 1 | 30 | 2 | 999 | Injection (small) | Balloon slipped, lower pressure reading |
| 2 | 30 | 2 | 999 | Injection (small) | Well-tolerated |
| 3 | 60 | 3 | 999 | Injection (small) | Well-tolerated, Alarm by the end of the |
| | | | | | injection, now rate decreased |
| 4 | 140 | 1 | 999 | Injection (small) | Well-tolerated |
| 5 | 80 | 4 | 999 | Injection (small) | Well-tolerated |
| 6 | 47 | 10 | 999 | Guide port (big) | Well-tolerated |

### Example 5

This Example shows inter alia flow rate can be constrained by the diameter of the catheter injection port and pigs can tolerate flow rate during biliary hydrodynamic injection at least up to 10 mL/sec.

In testing biliary hydrodynamic injection in pigs, two different sized ports were used in the catheter for injection. Through multiple testing in pigs, it was discovered that the smaller diameter injection port appeared to have an upper limit of 4-5 mL/sec flow rate for the exemplary power injector circuit described herein. It was then tested if the pig could tolerate a higher flow rate condition through the wider diameter guide port, which the present studies outside the pig in vitro demonstrated tolerated 10 mL/sec. Using a volume of 47 mL and a flow rate of 10 mL/sec, it was found that the pig tolerated this injection well with no acute changes in vital signs. There were no error messages on the power injector during the injection. This indicates that the diameter of the injectional channel for the DNA solution has a strong influence on the flow rate the power injector could achieve, and that pigs can at least physiologically tolerate flow rates into their biliary system up to 10 mL/sec.

Pig #3

| Injection | volume | Flow rate | | | |
|---|---|---|---|---|---|
| Attempt | (mL) | (mL/sec) | Pressure | Port | Notes |
| 1 | 30 | 2 | 999 | Injection (small) | Balloon slipped, lower pressure reading |
| 2 | 30 | 2 | 999 | Injection (small) | Well-tolerated |
| 3 | 60 | 3 | 999 | Injection (small) | Well-tolerated, Alarm by the end of |
| | | | | | the injection, flow rate decreased |
| 4 | 140 | 1 | 999 | Injection (small) | Well-tolerated |
| 5 | 80 | 4 | 999 | Injection (small) | Well-tolerated |
| 6 | 47 | 10 | 999 | Guide port (big) | Well-tolerated |

### Example 6

This Example shows inter alia pressure monitoring during biliary hydrodynamic injection and preferred pressures.

The pressure achieved during biliary hydrodynamic injection was evaluated, since pressure has shown to be instrumental to the efficacy of hydrodynamic delivery.²⁵ A pressure probe connected to an external laptop was obtained to record pressure in real-time before, during, and after injection. After endoscopic catheter placement and opening the balloon, the pressure probe was inserted through the guide port and into the catheter at a distance 1 cm past the tip.

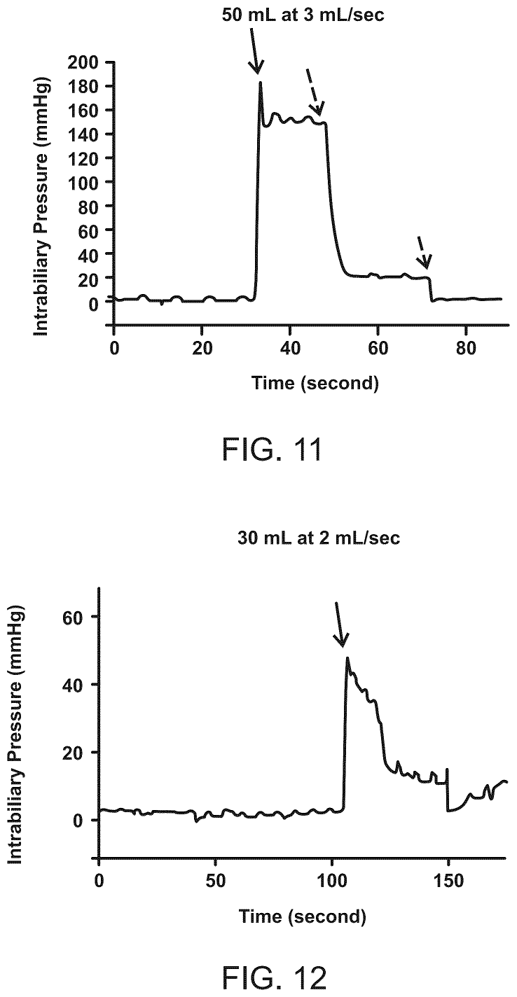

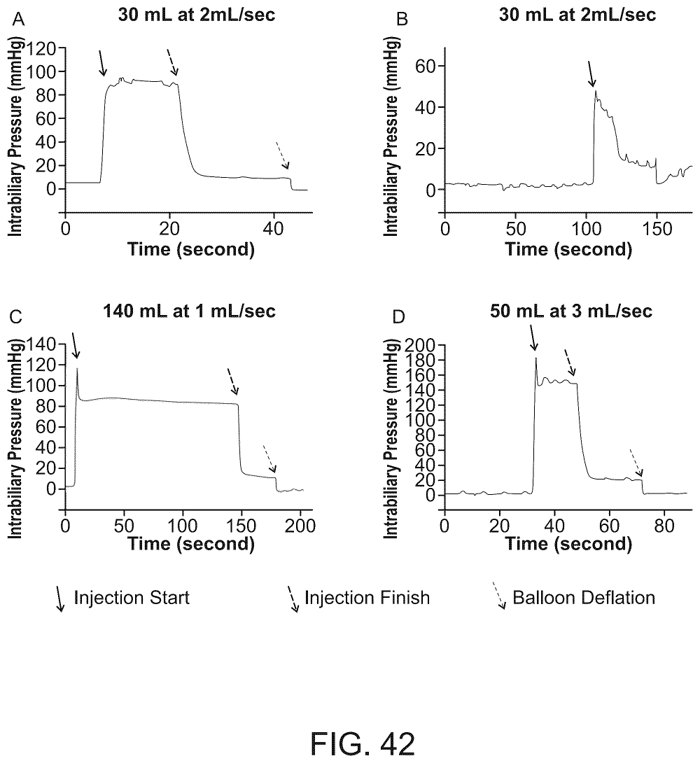

Pressure readings for the injection of 30 mL at 2 mL/sec demonstrated a plateau pressure of 80 mmHg during injection, that promptly dropped the moment the injection ended (see FIG. 11). This indicates the flow rate principally dictates pressure. As shown in FIG. 42B, a small level of pressure was released when the balloon was deflated, representing pressure generated by balloon restriction of biliary flow, although the exact value was variable between the different experiments (4.23 mmHg to 18.92 mmHg), and seemingly correlated with volume or pressure. A peak pressure point at the initial power injector was also noted in two of the conditions (114.76 mmHg and 181.36 mmHg in table below) before falling into a plateau phase. This represents the pressure in the biliary system immediately before capacity is reached and fluid reaches a steady-state of exit into the vascular system.

The flow rate to pressure relationship appears to be non-linear, since a 1 mL/sec injection and 2 mL/sec injection both similar pressure, 82.12 mmHg and 89.12 mmHg, respectively, during injection, while the 3 mL/sec injection yielded 148.58 mmHg. Thus, flow rate is related to the pressure achieved, but it is always not acutely linear. A complete listing of the pressure achieved in different experiments is provided in the table below.

SUPPLEMENTAL TABLE 1

| | | | Peak Pressure | Steady-State | Pressure before |
|---|---|---|---|---|---|
| | | | during | Pressure during | Balloon |
| | Volume | Flow rate | Injection | Injection | Deflation |
| Trial Name | (mL) | (mL/sec) | (mmHg) | (mmHg) | (mmHg) |
| Pig #2 Trial #1 | 50 | 3 | 181.36 | 148.58 | 18.92 |
| Pig #3 Trial #1 | 30 | 2 | 46.08 | 36.42 | 10.71 |
| Pig #3 Trial #2 | 30 | 2 | 89.12 | 85.08 | 4.23 |
| Pig #3 Triai #3 | 140 | 1 | 114.76 | 82.49 | 9.98 |

### Example 7

This Example shows inter alia pressure monitoring during biliary hydrodynamic injection can monitor injection integrity.

During biliary hydrodynamic injection, the pressure curve was able to detect the balloon accidentally slipping backward, thereby releasing fluid into the gall bladder (FIG. 12 below), which was also confirmed during fluoroscopy during the injection. Thus, pressure monitoring is useful to routinely confirm successful injection.

### Example 8

This Example shows inter alia liver injury can be predicted by flow rate during biliary hydrodynamic injection.

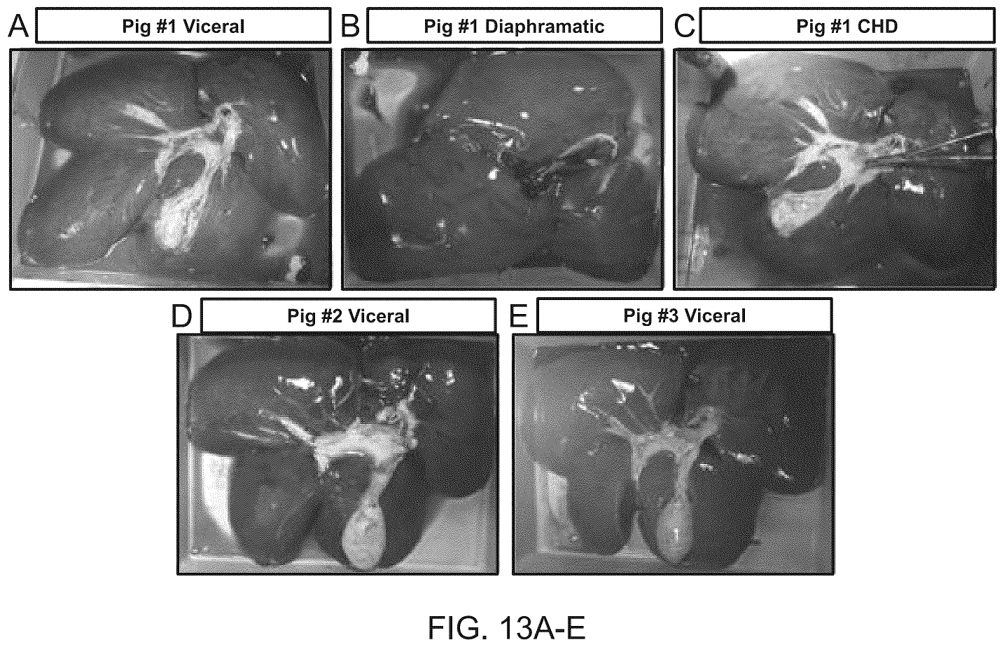

Acute pathogenic changes occurring in pigs immediately post-procedure were examined, particularly in the context of repeated hydrodynamic injections. All pigs had blood draws performed before and after the procedure, and pigs were sacrificed within 15 minutes of the last injection. Organs were examined during immediate necropsy post-procedure. All three pigs showed grossly normal anatomy upon examination without significant swelling, bruising, or rupture (see FIG. 13). The CHD was probed with an instrument in Pig #1, confirming intact status with no rupture (see FIG. 13C). The dorsal and ventral surfaces were intact in the pig (see FIGS. 13A, 13B).

TABLE 1

| | | | | | |
|---|---|---|---|---|---|
| Injection | Volume | Flow rate | Pressure | | |
| Attempt | (mL) | (mL/sec) | (psi) | Port | Notes |

Pig #1

| | | | | | |
|---|---|---|---|---|---|
| 1 | 30 | 2 | 999 | Injection (small) | Well tolerated |
| 2 | 30 | 4 | 999 | Injection (small) | Well-tolerated |
| 3 | 50 | 5 | 999 | Injection (small) | Alarm during injection, flow rate decreased |
| | | | | | to balance pressure |

Pig #2

| | | | | | |
|---|---|---|---|---|---|
| 1 | 45 | 5 | 1200 | Injection (small) | Circuit burst where line connected to the |
| | | | | | power injector and to the port. No evidence |
| | | | | | of liver parenchymal damage |
| 2 | 50 | 3 | 999 | Injection (small) | Well-tolerated |
| 3 | 37 | 4 | 999 | Injection (small) | Well-tolerated |

Pig #3

| | | | | | |
|---|---|---|---|---|---|
| 1 | 30 | 2 | 999 | Injection (small) | Balloon slipped, lower pressure reading |
| 2 | 30 | 2 | 999 | Injection (small) | Well-tolerated |
| 3 | 60 | 3 | 999 | Injection (small) | Well-tolerated, Alarm by the end of the |
| | | | | | injection, flow rate decreased |
| 4 | 140 | 1 | 999 | Injection (small) | Well-tolerated |
| 5 | 80 | 4 | 999 | Injection (small) | Well-tolerated |
| 6 | 47 | 10 | 999 | Guide post (big) | Well tolerated |

Looking at laboratory values, a series of liver function tests were analyzed pre- and post-injection (see Table 2 below). Alanine aminotransferase (ALT) was not significantly changed before and after injection. By contrast, aspartate aminotransferase (AST) showed a notable increase from 19 U/L to 137 U/L in pig #2 and 59 U/L to 252 U/L in pig #3. Pig #1 did not exhibit increases in AST or ALT. Total and direct bilirubin trended upwards in pig #1 and #2, although it remained within normal limits. Injection parameters are provided for reference in Table 1 below.

TABLE 2

| | Pig #1 | | Pig #2 | | Pig #3 | | |
|---|---|---|---|---|---|---|---|
| | pre | post | pre | post | pre | post | Reference |
| AST (units/L) | 44 | 48 | 19 | 137 | 59 | 252 | 32-84 |
| ALT (units/L) | 57 | 56 | 51 | 49 | 88 | 90 | 31-58 |
| Albumin (g/dL) | 3.3 | 2.7 | 3.5 | 3.3 | 3.5 | 3.2 | 1.9-2.4 |
| Total bilirubin | 0.3 | 0.5 | 0.2 | 0.6 | 0.3 | 0.3 | 0-10 |
| (mg/dL) | | | | | | | |
| Direct bilirubin | 0.2 | 0.5 | 0.2 | 0.5 | 0.2 | 0.3 | 0-0.3 |
| (mg/dL) | | | | | | | |
| Creatinine | 1.9 | 1.6 | 1.9 | 1.8 | 1.7 | 1.6 | 1.0-2.7 |
| (mg/dL) | | | | | | | |

AST, aspartate aminotransferase;
ALT, alanine aminotransferase
References: Peter G. G. Jackson and Peter D. Cockcroft, Clinical Examination of Farm Animals, 2002, 303-305.

### Example 9

This Example shows inter alia increased flow rates can mediate histological changes associated with efficient hydrodynamic delivery.

The histology of organs immediately post-injection was examined. Biliary hydrodynamic injection demonstrates characteristic pathological pattern after injection, characterized by the formation of large, fluid-filled vesicles in the cytoplasm of hepatocytes, along with dilute cytoplasm.

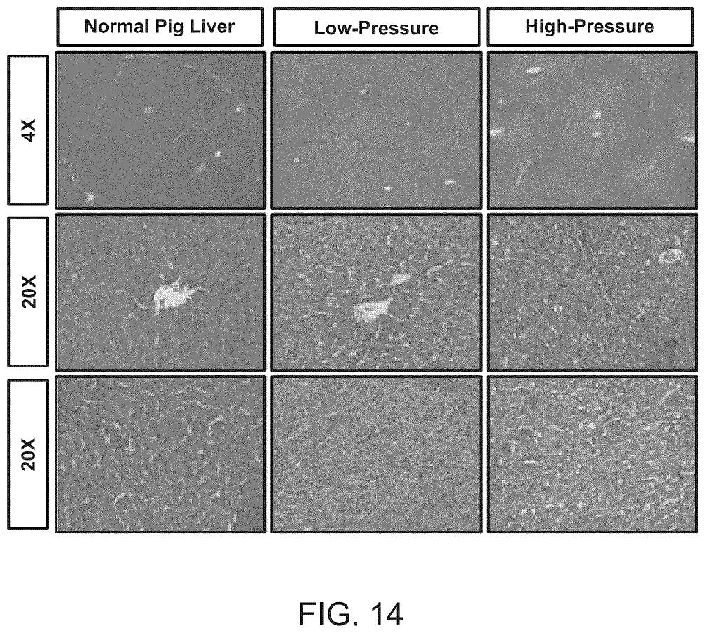

Pig #1 and #2, as the low-pressure injection group, demonstrated acute dilation of sinusoid spaces within hepatic lobules compared to control, non-injected pig liver (see figure below), consistent fluid volume exiting biliary canaliculi, and entering sinusoidal spaces. Central veins appeared to be the same size between injected and non-injected animals. Hepatocyte cytoplasm also appeared to be more dilute than control pig liver the low-pressure injection pigs (see FIG. 14), consistent with intracellular entry of fluid from hydrodynamic injection.³¹ Pig #3, which received a 10 mL/sec injection immediately before sacrifice as a high-pressure injection pig, had significantly more dilation of the sinusoid spaces within lobules than the low-pressure injection group (see figure below). Moreover, Pig #3 exhibited numerous large, intracellular fluid-filled vesicles scattered throughout the hepatocyte cytoplasm, which were not observed in Pig #1 and Pig #2 (see FIG. 14).

### Comparison to Murine Hydrodynamic Tail Vein Injection

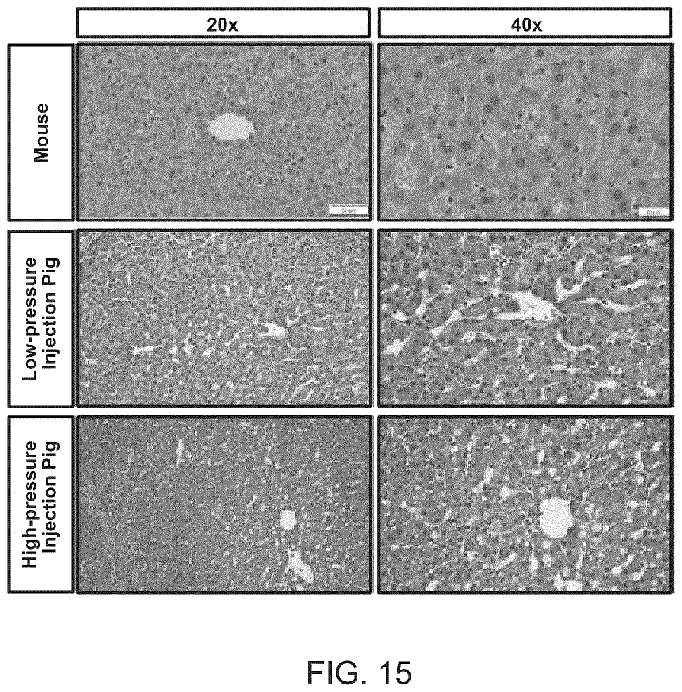

Given that hydrodynamic tail vein injection in mice results in efficient gene delivery and expression, the histopathological effects in the mouse liver shortly after hydrodynamic injection were compared with the histopathological effects on pig liver after biliary hydrodynamic injection. Scattered hepatocytes with dilute cytoplasm in mouse liver were observed, along with occasional hepatocytes containing red blood cells, the latter reflective of the vascular route of the procedure (see FIG. 15). Numerous fluid-filled vesicles were seen within murine hepatocytes, although generally smaller than the vesicles seen in pig #3. The combination of histological changes most resembles the high-pressure injections in pig #3, suggesting these high pressure/flow rates generated could closely mimic the efficient hydrodynamic gene delivery conditions in mice.

### Example 10

This Example shows inter alia decreased solution viscosity during biliary hydrodynamic injection can lead to better tolerance for injection.

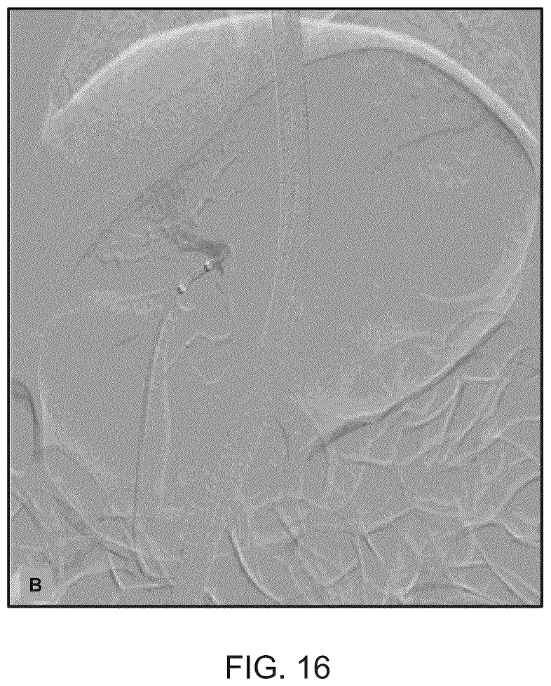

In a previous study, under fluoroscopy, different injection volumes (10, 20, 30, 40 mL) of one-third strength iohexol radiocontrast medium were injected at different flow rates (1, 2, 3 mL/sec) with the maximal pressure set to 999 psi. The balloon remained inflated for 30 seconds after completion of each injection. Injection parameters were sequentially tested at 10-minute intervals in ascending order until the rupture of the bile duct as evidenced by extravasation of contrast medium. These previous parameters resulted in rupture of the proximal common hepatic duct at injections above 30 mL volume and 2 mL/second (see FIG. 16).

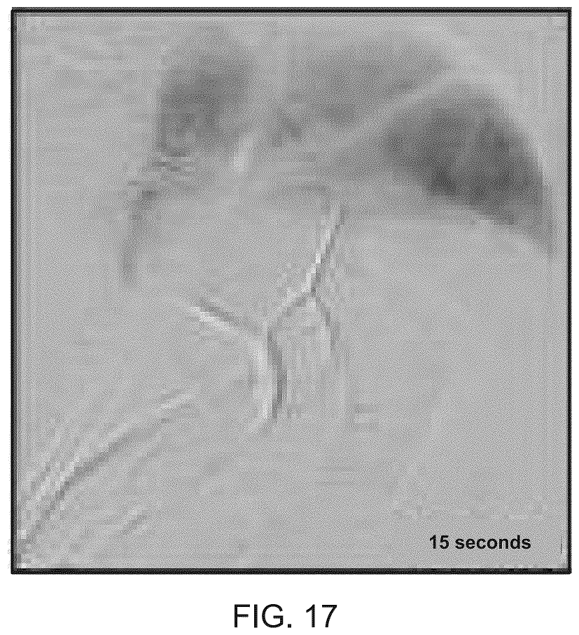

For the current disclosure, a 25% contrast solution was employed, which together with other changes, did not mediate any rupture of the common hepatic duct, even at volumes exceeding the previous test. See FIG. 17. This evidence points to viscosity having a negative impact on bile duct integrity and points to optimal injection solutions with viscosity approaching normal physiologic solutions. This experiment also outlines a concentration of contrast solution that could be mixed in with DNA solution to perform hydrodynamic injection monitoring.

### Example 11

This Example shows inter alia changing the promoter in the DNA to a liver-specific promoter for biliary hydrodynamic injection can lead to higher DNA transfection efficiency.

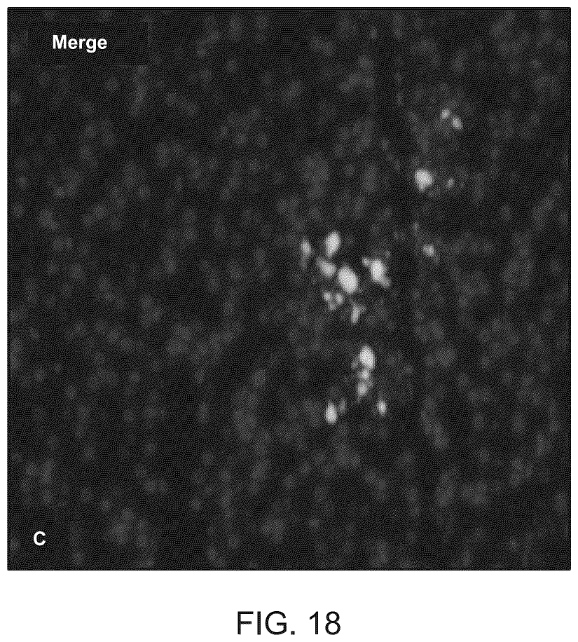

The previous investigation into biliary hydrodynamic injection achieved very low transfection rates when 3 mg of DNA was administered in 30 mL volume injected at 2 mL/sec. The results from prior investigations are shown FIG. 18. The promoter employed in these studies was the elongation-factor 1 alpha (EF1alpha) promoter.

Based on these previous investigations with very low transfection efficiency, a modification was made for the current disclosure to improve the inefficient delivery and expression of DNA into the pig's liver. The estimated transfection efficiency of the prior investigation was around 0.1-1%.

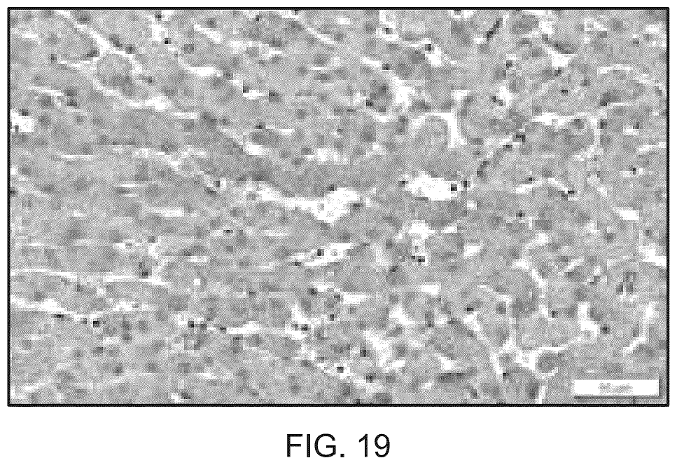

For the current disclosure, a modification was made to use a liver-specific promoter in the plasmid DNA to expression the gene of interest. An exemplar liver-specific promoter, LP1 was used, which is a composite promoter consisting of human APO-HCR enhancer and hAAT promoter. This change in DNA vector allowed for significant improvement in transfection efficiency using human Factor IX as a reporter. Transfection efficiencies between 35-50% of liver hepatocytes staining positive for human Factor IX on tissue section were achieved in pigs after biliary hydrodynamic injection. An example of a hFIX immunostained image is shown in FIG. 19.

### Example 12

This Example shows inter alia increased transfection efficiency of biliary hydrodynamic injection with higher DNA doses.

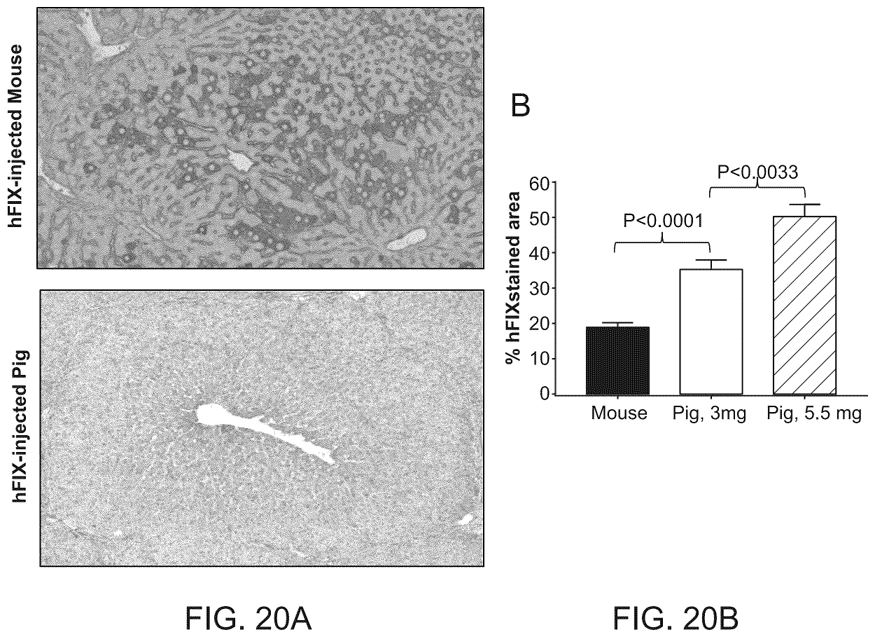

Pig biliary hydrodynamic injection yields superior transfection efficiency to mouse hydrodynamic tail vein injection. As shown in FIG. 20A, mouse hydrodynamic tail vein injection is primarily located around central veins in zone 2. FIG. 20B shows that pig hydrodynamic biliary injection leads to human factor IX (hFIX)-positive expression almost uniformly circling central veins in zone 3 and then radiates outward along the chords reaching zone 1 and 2 (pig no. 3 left medial lobe proximal section depicted). Mouse lobules are smaller than pig lobules, respectively.

Previous investigations into hydrodynamic tail vein injection in mice revealed that the maximal transfection efficiency approached 20% of mouse hepatocytes. Increasing DNA dose for mouse hydrodynamic tail vein injection seemingly only increased DNA delivery to the same hepatocytes and did not result in an increase in transfection efficiency.

Unexpectedly, the current biliary hydrodynamic gene therapy described herein showed a significant increase in transfection efficiency in pigs versus mice, and also showed a pronounced dose dependent response for transfection efficiency in pigs. As shown in FIG. 20B, higher DNA doses (e.g., 3 mg vs. 5.5 mg) in pig led to more hepatocytes (e.g., about 35% vs. about 50% hFIX-stained area) staying positive for human Factor IX. This finding helps define transfection response for this strategy, which will be useful and to find doses for different diseases.

### Example 13

This Example shows inter alia biliary hydrodynamic injection leads to a lack of off-target tissue transfection and persistence in other body fluids.

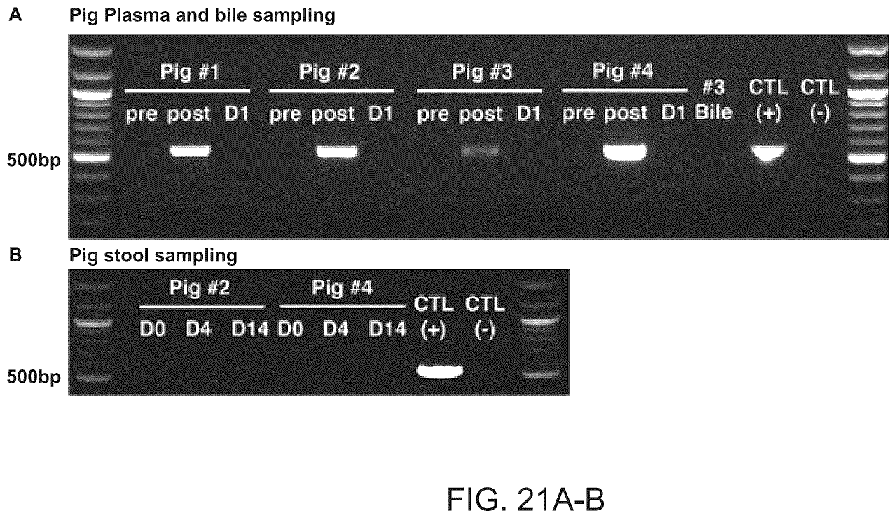

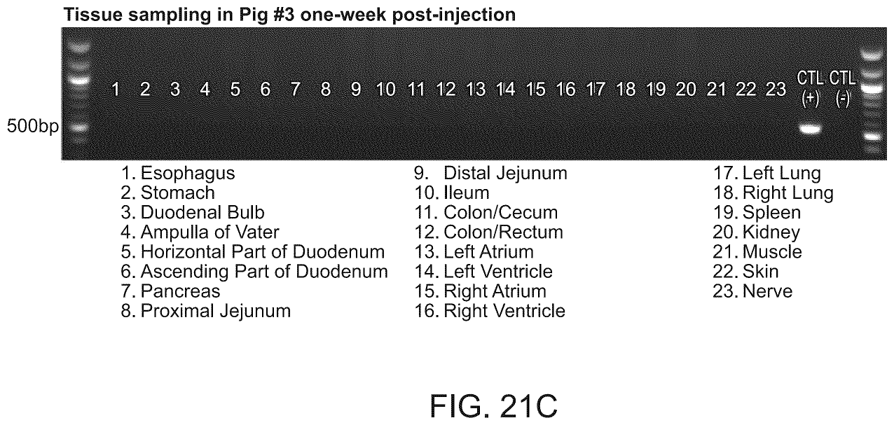

Off-target distribution of the gene therapy delivered by biliary hydrodynamic injection was examined, testing for the presence of pDNA beyond the liver in other tissues. Plasma samples before and after injection were collected to observe for the entry of pDNA into the circulation. PCR testing revealed the presence of pDNA in the plasma 15 minutes post injection (see FIG. 21A), explained by escape of fluid from biliary caniculi into venous circulation. By day 1 post injection, pDNA was absent consistent with degradation by serum DNases at this timepoint^{47,48} Given that pDNA in the bile ducts eventually drains into the small intestine, the ability of any pDNA to undergo transformation into the host gut bacteria was assessed. Stool DNA collected from two pigs was negative for hFIX DNA at all time points, showing pDNA transmission did not occur to host intestinal microbiome (see FIG. 21B). With the presence of pDNA in systemic circulation, the ability of DNA transfection to occur in other organs outside of the liver was assessed. As shown in FIG. 21C, different tissues (23 biopsies in total) throughout pig #3 were investigated, including the kidney, lungs, heart, spleen, pancreas, and several locations along the GI tract (stomach, duodenum, colon). All tissues were negative for hFIX DNA by PCR indicating no pDNA delivery (see FIG. 21C). The elimination of pDNA within the biliary tract was investigated one-week post-injection, and it was confirmed that hFIX DNA was eliminated from bile by that time point (see FIG. 21A).

### Example 14

This Example shows inter alia biliary hydrodynamic injection can lack or mlmmlze inflammatory responses at low flow rates.

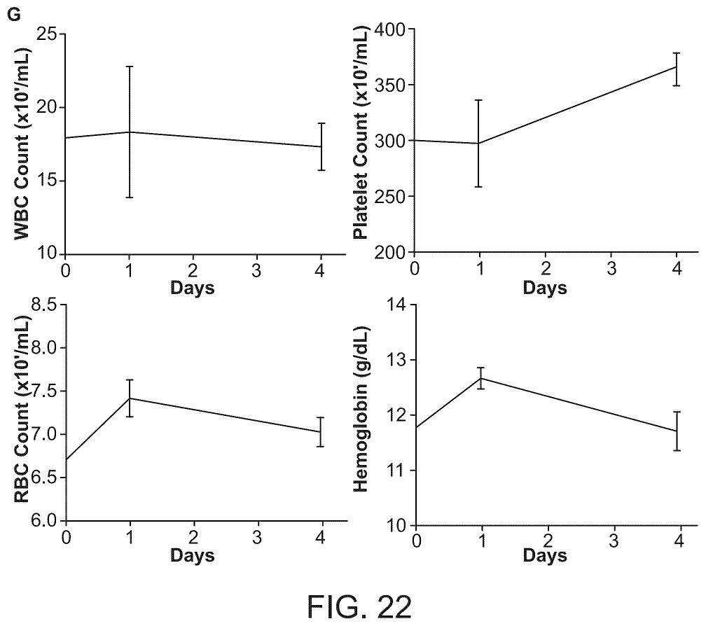

Hematologic data are presented and in FIG. 22 for four pigs injected via the biliary route at 30 mL at 2 mL/sec, demonstrating no significant changes in the days subsequent to injection. This suggested no acute inflammatory response at this flow rate of biliary hydrodynamic injection, and that it could be advantageous in this low range.

### Example 15

This Example shows inter alia biliary hydrodynamic injection can lack or minimize AST and ALT increases at low flow rates.

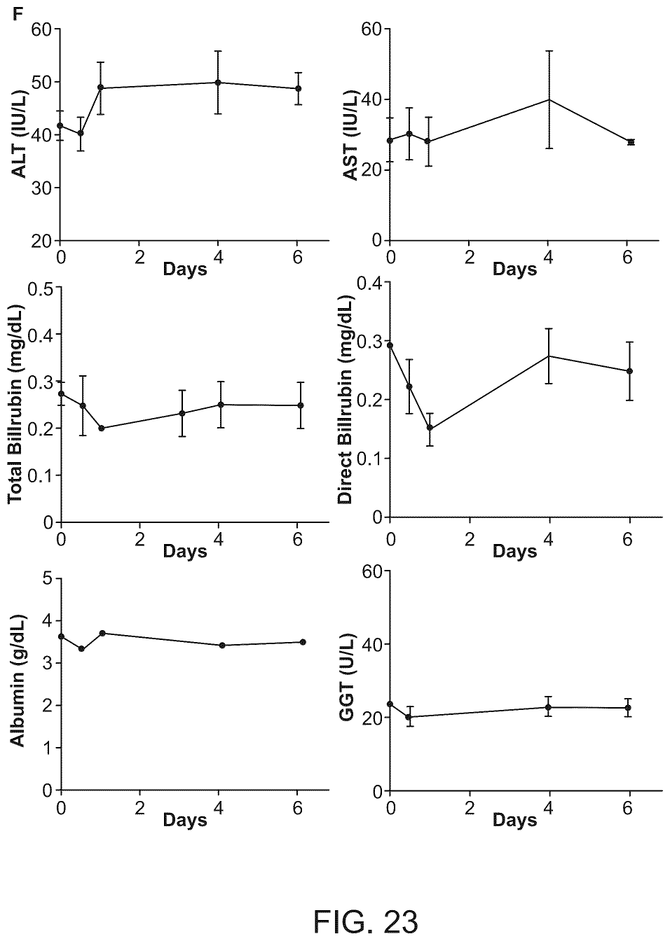

Biochemical data are presented here for four pigs injected via the biliary route at 30 mL at 2 mL/sec, demonstrating no significant increases above the normal range. See FIG. 23. This suggests no tissue injury at this flow rate.

In another pig, higher flow rates and volumes were utilized, up to 5 mL/sec at 50 mL. Importantly, this higher flow rate did not yield an increase in AST and ALT levels, which remained normal. This suggests this flow rate level range (2-5 mL/sec) may be ideal for efficient transfection efficiency without causing tissue damage and inflammation.

TABLE 1
Pig #1

| Injection | | Flow rate | Pressure | | |
|---|---|---|---|---|---|
| Attempt | Volume | (mL/sec) | (psi) | Port | Notes |
| 1 | 30 | 2 | 999 | Injection (small) | Well-tolerated |
| 2 | 30 | 4 | 999 | Injection (small) | Well-tolerated |
| 3 | 50 | 5 | 999 | Injection (small) | Alarm during injection, flow rate |
| | | | | | decreased to balance pressure |

TABLE 2

Pig #1

| | pre | post |
|---|---|---|
| AST (units/L) | 44 | 48 |
| ALT (units/L) | 57 | 56 |
| Albumin (g/dL) | 3.3 | 2.7 |
| Total bilirubin (mg/dL) | 0.3 | 0.5 |
| Direct bilirubin (mg/dL | 0.2 | 0.5 |
| Creatinine (mg/dL) | 1.9 | 1.6 |

### Example 16

This Example shows inter alia pigs can tolerate repeated biliary hydrodynamic injections on separate dates.

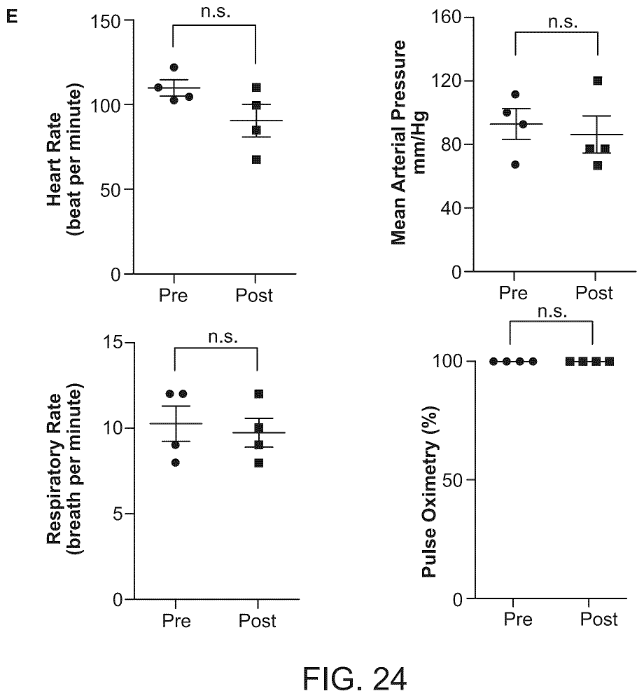

Four pigs were injected on an initial date via the biliary route, all at 30 mL volume at a rate of 2 mL/sec. Their vital signs before and after the procedure are shown in FIG. 24, demonstrating tolerance to the procedure.

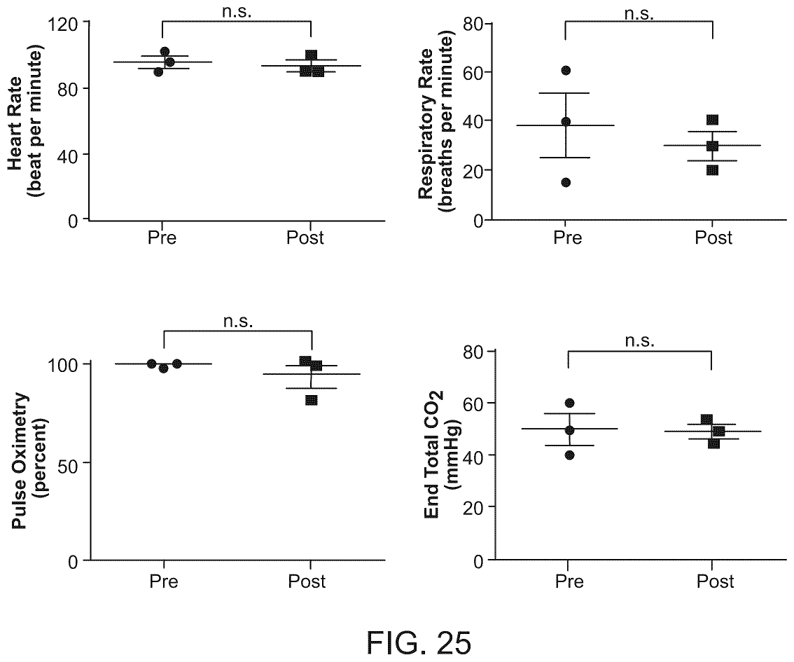

Three of the pigs were injected again 3 weeks later again by biliary hydrodynamic injection. Their vital signs had normalized before the second injection at this time point, and after repeat procedure and injections on this day, the vital signs again did not exhibit significant physiological perturbations and did not experience any permanent effects. See FIG. 25. This demonstrates for the first time that repeat biliary hydrodynamic injections are feasible and physiologically well tolerated.

### Example 17

This Example shows inter alia biliary hydrodynamic delivery with pre-loaded DNA solution of hFIX plasmid shows uniform distribution within the pig liver and successful expression in every pig injected.

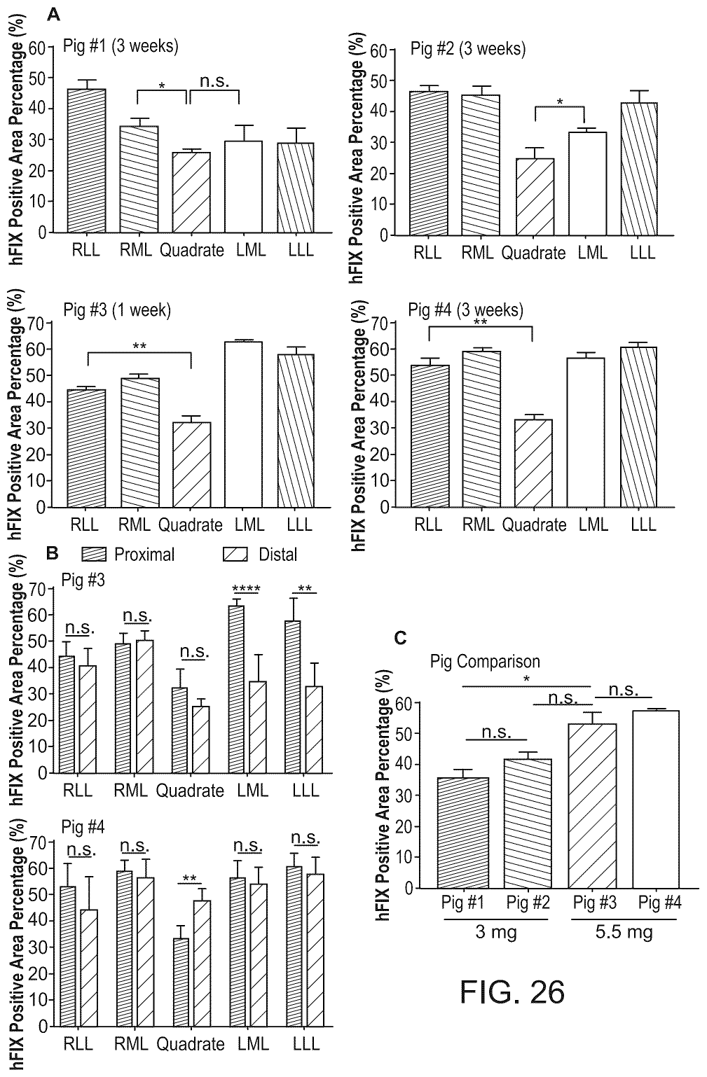

Pigs were injected with a mixture of 3-5.5 mg hFIX transposon and piggyBac transposase with catheter pre-loaded with DNA solution at 30 mL volume for 2 mL/sec. Additional DNA solution was loaded in the power injector cartridge to ameliorate deadspace volume in the circuit, so that the complete 30 mL into the pig's liver could be achieved. The hFIX immunostained area within one hepatic lobule was quantified, and then 5-6 lobules were averaged to provide lobe value. As shown in FIG. 26A, the % area of hFIX-positive immunostaining within individual lobules (n=5) in pig #3 (one-week post-injection) and pig #1, #2 and #4 (3 weeks post-injection) is provided. As shown in FIG. 26B, among pig #3 and #4, distal and proximal portions of an individual hepatic lobe do not show consistent differences in % area of hFIX-positive hepatocytes (n=5-6 lobules per lobe). As shown in FIG. 26C, Higher doses of plasmid DNA led to higher hFIX % area transfected. Each pig represents the lobe averages determined in FIG. 26A now pooled together with quadrate lobe removed due to small size (˜5% of liver). Data measurements are presented as mean±standard error of mean (SEM). Statistics represent unpaired, parametric, two-tailed t-tests; significance (p<0.05). **** p<0.0001; ** p<0.01; * p<0.05; n.s.=not significant.

### Example 18

This Example shows hydrodynamic pressure from biliary hydrodynamic injection Is effectively distributed to both proximal and distal sites to the common hepatic duct injection site.

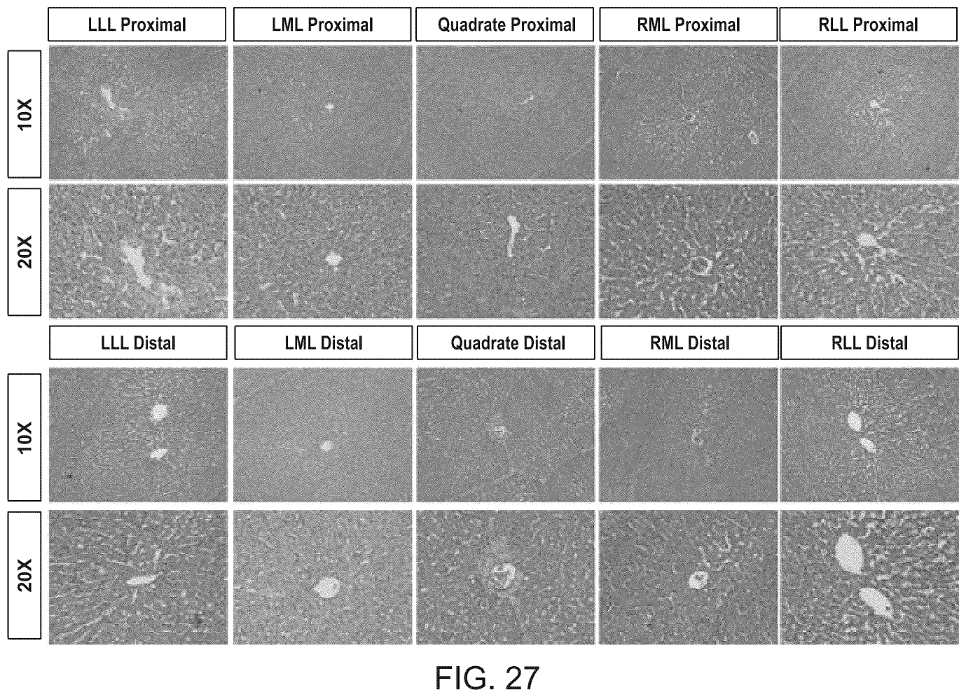

Liver tissue was sampled in a pig at proximal and distal sites within each liver lobe to the common hepatic duct where the catheter was located. Biliary hydrodynamic injection was performed in a pig at 10 mL/sec with diluted cytoplasm and intracellular vesicles observed as a result of the injection in all lobes and at all sampling locations. H&E staining was performed, revealing large vesicle formation within hepatocytes, dilated hepatic sinusoids, and dilute cytoplasm inside hepatocytes as shown in FIG. 27.

### References for Example 1-18

1. Baruteau, J., Waddington, S. N., Alexander, I. E. & Gissen, P. Gene therapy for monogenic liver diseases: clinical successes, current challenges and future prospects. J Inherit. Metab. Dis. 40, 497-517 (2017).
2. Wang, D., Tai, P. W. L. & Gao, G. Adeno-associated virus vector as a platform for genetherapy delivery. Nat Rev Drug Discov 18, 358-378 (2019).
3. Doshi, B. S. & Arruda, V. R. Gene therapy for hemophilia: what does the future hold?Ther Adv Hematol 9, 273-293 (2018).
4. Franchini, M., Frattini, F., Crestani, S., Sissa, C. & Bonfanti, C. Treatment of hemophiliaB: focus on recombinant factor IX. Biologics 7, 33-38 (2013).
5. Manno, C. S. et al. Successful transduction of liver in hemophilia by AAV-Factor IX andlimitations imposed by the host immune response. Nature Medicine 12, 342-347 (2006).
6. Nathwani, A. C. et al. Long-term safety and efficacy of factor IX gene therapy in hemophilia B. N. Engl. J Med. 371, 1994-2004 (2014).
7. Nathwani, A. C. et al. Adenovirus-associated virus vector-mediated gene transfer in hemophilia B. N Engl. J Med. 365, 2357-2365 (2011).
8. Kok, C. Y. et al. Adeno-associated virus-mediated rescue of neonatal lethality in argininosuccinate synthetase-deficient mice. Mol Ther 21, 1823-1831 (2013).
9. Manning, W. C., Zhou, S., Bland, M. P., Escobedo, J. A. & Dwarki, V. Transient immunosuppression allows transgene expression following readministration of adeno-associated viral vectors. Human Gene Therapy 9, 477-485 (1998).
10. Caicedo, R. et al. Adeno-associated virus antibody profiles in newborns, children, andadolescents. Clin. Vaccine Immunol. 18, 1586-1588 (2011).
11. George, L. A. et al. Hemophilia B Gene Therapy with a High-Specific-Activity Factor IX Variant. N. Engl. J Med. 377, 2215-2227 (2017).
12. Fang, B. et al. Gene therapy for hemophilia B: host immunosuppression prolongs the therapeutic effect of adenovirus-mediated factor IX expression. Human Gene Therapy 6,1039-1044 (1995).
13. Brunetti-Pierri, N. et al. Balloon catheter delivery of helper-dependent adenoviral vector results in sustained, therapeutic hFIX expression in rhesus macaques. Mol Ther 20, 1863-1870 (2012).
14. Powell, J. S. et al. Phase 1 trial of FVIII gene transfer for severe hemophilia A using a retroviral construct administered by peripheral intravenous infusion. Blood 102, 2038-2045 (2003).
15. Cantore, A. et al. Liver-directed lentiviral gene therapy in a dog model of hemophilia B. Science Translational Medicine 7, 277ra28-277ra28 (2015).
16. DeRosa, F. et al. Therapeutic efficacy in a hemophilia B model using a biosynthetic mRNA liver depot system. Gene Therapy (2016). doi:10.1038/gt.2016.46
17. Ramaswamy, S. et al. Systemic delivery of factor IX messenger RNA for protein replacement therapy. Proc. Natl. Acad. Sci. U.S.A. 114, E1941-E1950 (2017).
18. Sendra, L., Herrero, M. J. & Alifio, S. F. Translational Advances of Hydrofection by Hydrodynamic Injection. Genes (Basel) 9, 136 (2018).
19. Dul, M. et al. Hydrodynamic gene delivery in human skin using a hollow microneedle device. J Control Release 265, 120-131 (2017).
20. Kamimura, K., Zhang, G. & Liu, D. Image-guided, intravascular hydrodynamic gene delivery to skeletal muscle in pigs. Mol Ther 18, 93-100 (2010).
21. Woodard, L. E. et al. Hydrodynamic Renal Pelvis Injection for Non-viral Expression of Proteins in the Kidney. J Vis Exp e56324 (2018). doi:10.3791/56324
22. Sebestyen, M. G. et al. Mechanism of plasmid delivery by hydrodynamic tail vein injection. I. Hepatocyte uptake of various molecules. J Gene Med. 8, 852-873 (2006).
23i. Zhang, G., Budker, V. & Wolff, J. A. High levels of foreign gene expression in hepatocytes after tail vein injections of naked plasmid DNA. Human Gene Therapy 10,1735-1737 (1999).
24. Liu, F., Song, Y. & Liu, D. Hydrodynamics-based transfection in animals by systemic administration of plasmid DNA. Gene Therapy 6, 1258-1266 (1999).
25. Zhang, G. et al. Hydroporation as the mechanism of hydrodynamic delivery. Gene Therapy 11, 675-682 (2004).
26. Suda, T. & Liu, D. Hydrodynamic gene delivery: its principles and applications. Mol Ther 15, 2063-2069 (2007).
27. Kamimura, K. et al. Safety assessment of liver-targeted hydrodynamic gene delivery in dogs. PLoS ONE 9, e107203 (2014).
28. Andrianaivo, F., Lecocq, M., Wattiaux-De Coninck, S., Wattiaux, R. & Jadot, M. Hydrodynamics-based transfection of the liver: entrance into hepatocytes of DNA that causes expression takes place very early after injection. J Gene Med. 6, 877-883 (2004).
29. Kobayashi, N., Nishikawa, M., Hirata, K. & Takakura, Y. Hydrodynamics-based procedure involves transient hyperpermeability in the hepatic cellular membrane: implication of a nonspecific process in efficient intracellular gene delivery. J Gene Med.6, 584-592 (2004).
30. Crespo, A. et al. Hydrodynamic liver gene transfer mechanism involves transient sinusoidal blood stasis and massive hepatocyte endocytic vesicles. Gene Therapy 12, 927-935 (2005).
31. Suda, T., Gao, X., Stolz, D. B. & Liu, D. Structural impact of hydrodynamic injection on mouse liver. Gene Therapy 14, 129-137 (2007).
32. Herweijer, H. et al. Time course of gene expression after plasmid DNA gene transfer to the liver. J Gene Med. 3, 280-291 (2001).
33. Viecelli, H. M. et al. Treatment of phenylketonuria using minicircle-based naked-DNA gene transfer to murine liver. Hepatology 60, 1035-1043 (2014).
34. Kamimura, K., Suda, T., Xu, W., Zhang, G. & Liu, D. Image-guided, lobe-specific hydrodynamic gene delivery to swine liver. Mol Ther 17, 491-499 (2009).
35. Herrero, M. J. et al. DNA delivery to ‘ex vivo’ human liver segments. Gene Therapy 19, 504-512 (2012).
36. Khorsandi, S. E. et al. Minimally invasive and selective hydrodynamic gene therapy of liver segments in the pig and human. Cancer Gene Ther 15, 225-230 (2008).
37. Zhang, G. et al. Expression of naked plasmid DNA injected into the afferent and efferent vessels of rodent and dog livers. Human Gene Therapy 8, 1763-1772 (1997).
38. Hu, J. et al. A remarkable permeability of canalicular tight junctions might facilitate retrograde, non-viral gene delivery to the liver via the bile duct. Gut 54, 1473-1479 (2005).
39. Chen, C.-Y., Liu, H.-S. & Lin, X.-Z. Hydrodynamics-based gene delivery to the liver by bile duct injection of plasmid DNA—the impact of lasting biliary obstruction and injection volume. Hepatogastroenterology 52, 25-28 (2005).
40. Jiang, X., Ren, Y., Williford, J.-M., Li, Z. & Mao, H.-Q. Liver-targeted gene delivery through retrograde intrabiliary infusion. Methods Mol. Biol. 948, 275-284 (2013).
41. Kumbhari, V. et al. Successful liver-directed gene delivery by ERCP-guided hydrodynamic injection (with videos). Gastrointest. Endosc. 88, 755-763.e5 (2018).
42. Indrajit, I. K. et al. Pressure injectors for radiologists: A review and what is new. Indian J Radiol Imaging 25, 2-10 (2015).
43. Liu, F., Song, Y. & Liu, D. Hydrodynamics-based transfection in animals by systemic administration of plasmid DNA. Gene Therapy 6, 1258-1266 (1999).
44. Zhou, T., Kamimura, K., Zhang, G. & Liu, D. Intracellular gene transfer in rats by tail vein injection of plasmid DNA. AAPS J 12, 692-698 (2010).
45. Dai, C. et al. Liver gene transfection by retrograde intrabiliary infusion facilitated by temporary biliary obstruction. J Gene Med. 22, e3144 (2020).
46. Kumbhari, V. et al. Successful liver-directed gene delivery by ERCP-guided hydrodynamic injection (with videos). Gastrointest. Endosc. 88, 755-763.e5 (2018).
47. Kawabata, K., Takakura, Y. & Rashida, M. The fate of plasmid DNA after intravenous injection in mice: involvement of scavenger receptors in its hepatic uptake. Pharm. Res. 12, 825-830 (1995).
48. Liu, F., Shollenberger, L. M., Conwell, C. C., Yuan, X. & Huang, L. Mechanism of naked DNA clearance after intravenous injection. J Gene Med. 9, 613-619 (2007).

### Example 19

This Example shows inter alia endoscopic-mediated hydrodynamic gene delivery through the biliary system can mediates efficient transfection of pig liver.

Gene therapy could provide curative therapies to many inherited monogenic liver diseases. Clinical trials have largely focused on adeno-associated viruses (AAV) for liver gene delivery. These vectors, however, are limited by small packaging size, capsid immune responses, and inability to re-dose. As an alternative, non-viral, hydrodynamic injection through vascular routes can successfully deliver plasmid DNA into mouse liver, but has achieved limited success in large animal models. Toward demonstrating efficient and safe hydrodynamic injection in human-sized animals, injection through the biliary system was tested in pigs using a routine clinical procedure, endoscopic retrograde cholangiopancreatography (ERCP). Biliary hydrodynamic injection was well tolerated without significant changes in vital signs, liver enzymes, hematology, or histology. Using human factor IX (hFIX) as a model gene therapy, immunohistochemistry revealed 50.19% of the liver stained positive for hFIX after hydrodynamic injection at high plasmid DNA doses, with every hepatic lobule in all liver lobes demonstrating hFIX-expression. hFIX-positive hepatocytes were mainly distributed around the central vein, radiating outward across all three metabolic zones. Biliary hydrodynamic injection in pigs resulted in significantly higher transfection efficiency than mouse vascular hydrodynamic injection at matched DNA dose per liver weight dose (32.7-51.9% vs 18.9%, p<0.0001). hFIX was not detected in pig plasma, however, which may due to species differences in protein secretion. Overall, the present results demonstrate that biliary hydrodynamic injection can achieve higher transfection efficiency compared to AAV at magnitudes less cost in a relevant human-sized large animal. This technology may serve as a platform for gene therapy of human liver diseases.

The results herein utilized pigs, which have similar organ size to human patients and employed commercially available equipment currently in clinical care across the globe. It was found that hFIX genes could be delivered to all liver lobes at transfection efficiencies surpassing AAV technology with no toxicities. This approach merits further development for translation into the treatment of monogenic liver diseases in patients.

### Materials and Methods

### Animal Experiments

All animal experiments were conducted under approval of the animal care and use committee of Johns Hopkins Hospital and adhere to the guidelines of the NIH Guide for the Care and Use of Laboratory Animals. Mouse experiments were performed on C57BL/6 strain using mice between 20 and 30 grams of weight. Yorkshire pigs were acquired from Archer Farms, Darlington, MD. All pigs were female and were acquired for their targeted weight before procedure. Pigs were acclimated and housed in conditions as previous described (39).

### Gene Construction and Plasmid Preparation

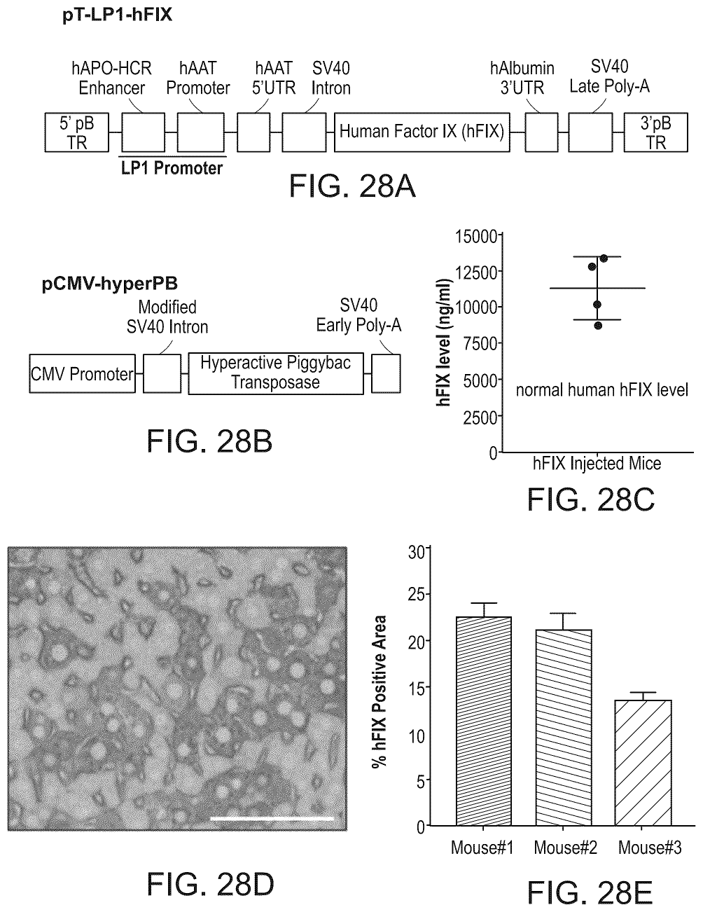

Pig-codon optimized human Factor IX using tools from IDTDNA and prepared as a gene fragment (Twist Bioscience) and cloned to form pT-LP1-hFIX (see FIG. 28A). The plasmid, pCMV-hyperPB (see FIG. 28B), was constructed by gene synthesis (BioBasic). The sequence was pig-codon optimized using tools from IDTDNA based on the published hyperactive piggyBac transposase sequence (55). The hFIX expression cassette used in AAV trials (44) was synthesized into a pUC backbone (BioBasic). There was one addition of the human albumin 3′ UTR for increased mRNA transcript stability.

Plasmids were prepared for in vitro experiments using maxiprep kits (Qiagen) and for in vivo injection using gigaprep kits (Zymo Research). Ratio of transposon to transposase for injection was 7.5 to 9.2, to try to optimize transposition efficiency (42). Plasmid DNA was diluted in normal saline solution for in vivo experiments in mouse and pigs.

### Transfection Experiments

pCMV-pB and pIRII-eGFP were previously published (56, 57). Briefly, 2.5 μg of pIRII-eGFP and pCMV-pB or pCMV-hyperPB were transfected (Lipofectamine 3000, Thermofisher) into a 6-well plate of 293T cells according to manufacturer's protocol. Green fluorescent protein (GFP) was visualized with fluorescent microscope. Transfection of pT-LP1-hFIX and pCMV-hyperPB was also carried out in HepG2 cells with Lipofectamine 3000 in a similar manner.

### Hydrodynamic Tail Vein Injection

C57BL/6 mice weighing between 20 and 25 grams were selected for HTVI. Mice were treated with a heat lamp for 5-10 minutes until vasodilation was achieved. 8 μg transposon and 1 μg transposase were diluted in 2.2 mL (8-10% body weight) of normal saline and injected into mice between 4-7 seconds. This dose was selected to give a matching liver weight-based dosing comparison for pig hydrodynamic studies. Post HTVI, >90% of inject DNA is localized to the liver (58), such that 8 μg transposon DNA into a 1.5 g mouse liver equates to 4-5.3 mg in a 1 kg pig liver.

### Endoscopy Procedure

Food was withheld from pigs the night before the procedure and all pigs weighed prior to the procedure for proper anesthetic doses. Once sedated, pre-treatment blood draws were performed from the jugular veins of all pigs, and pre-treatment stool was collected by rectal exam. Vital signs were monitored throughout the procedure by veterinary care on site. The endoscope (therapeutic video duodenoscope, ED-580XT, FUJIFILM Medical Systems U.S.A.) was advanced through the mouth, into the stomach, small intestine. A sphincterotome (CleverCut3V, Olympus Medical) preloaded with a 0.025 inch hydrophilic guidewire (VisiGlide, Olympus Medical) was then advanced into the common hepatic duct as verified by fluoroscopy using 4-5 mL of radiocontrast solution (Omnipaque, 350 mg/mL; GE Health Co) and a Philips Allura C-Arm. The sphincterotome was then exchanged over the wire for an extraction balloon catheter (Multi-SY Plus, Olympus Medical) with the balloon catheter being inflated to 11.5 mm at the CHD 1-2 cm below the hepatic hilum. A medical grade power injector (MEDRAD Mark 7 Arterion, Bayer) was filled with DNA solution and attached to the injection port of the balloon. DNA solution was used to prime the tubing and catheter to fill dead volume before injection commenced. Injection parameters were executed as listed in Table 1. The balloon was deflated 30 seconds post-injection and repeat fluoroscopy with 4-5 mL of contrast injection performed to assess intact biliary system. Transabdominal ultrasound was performed on pig #2 by board certified interventional radiologist prior to and post hydrodynamic injection. Endoscope was withdrawn post-injection. Post-treatment blood draw and post-treatment stool were then obtained as described above. Total procedure time was monitored from insertion of the endoscope through injection and removal of the endoscope. Specific details on anesthesia doses prior to and during the procedure are previously described (39).

### Blood Collection and Analysis

All pigs were anesthetized with ketamine/xylazine prior to bleeding through the jugular vein by a veterinary technician. Blood was collected into EDTA tubes for plasma proteins, and serum chemistries in SST tubes (Becton Dickinson). Stool was collected whilst the pigs were anesthetized for phlebotomy by manual rectal exam. Pigs were also weighed during this period. Blood from mice were collected by retro-orbital eye bleeding under isoflurane anesthesia and prepared similarly.

ELISA testing for hFIX on cell culture supernatant, human plasma, mouse plasma, and pig plasma was performed according to manufacturer's protocol (AssayMax™ HumanFactor IX ELISA Kit, Assay Pro); the ELISA kit lacks cross-reactivity against mouse hFIX (15).

Discarded, de-identified human plasma was used as a positive control. Serum chemistries and hematology were performed by the Johns Hopkins Phenotyping Core on Diasys Respons®910 chemistry analyzer and Procyte automated analyzer, respectively. Stool and Tissue analysis

Pigs were euthanized at time points 1 and 3 weeks post-injection and necropsy performed within 15 minutes of death. Veterinary and medical pathologists were consulted for proper technique. Biopsies were taken across multiple sites in proximal and distal within each liver lobe (FIG. 23A). The quadrate lobe was sampled since it connects to the liver hilum with a direct bile duct branch like the four other largest lobes, while the caudate process was included with right lateral lobe (RLL) for analysis, since they share the same biliary duct branch.

Preliminary studies found that RLL, right medial lobe (RML), left lateral lobe (LLL), and left medial lobe (LML) all had approximately similar masses within 5%, together accounting for 95% of the liver mass. Mice were euthanized at 1 week post-injection. For both mice and pigs, tissue was fixed in 10% formaldehyde. The Johns Hopkins Phenotyping Core did tissue embedding and sectioning, along with hematoxylin & eosin (H&E) staining.

The DNeasy Blood & Tissue kit (Qiagen) was used for DNA extraction from tissues, blood, and bile samples. Fecal DNA was purified using a Quick-DNA Fecal/Soil Microbe (ZymoResearch). RNA extraction was performed with RNeasy kit (Qiagen), and reverse transcription was performed (SuperScript IV, Thermofisher). PCR (DreamTaq, Thermofisher) was performed using internal (Sigma) directed against the synthetic hFIX sequence, Internal FIX For: GATAATAAGGTGGTCTGCTCTTGCACG, Internal FIX Rev: GTCACGTAGGAGTTGAGGACCAG

An antibody against human Factor IX (GAFIX-AP, Affinity Biologicals) was used for western blot detection, as well as staining by IHC on pig and mouse liver sections. Western blot was performed with 10% SDS-PAGE gel using 2.5 μg/mL antibody dilution, and mouse anti-goat IgG-HRP secondary (sc-2354, Santa Cruz). IHC was performed by VitroVivo Biotech (Rockville, MD) with negative control un-injected pig and positive control, human liver sections. For quantification of transfection efficiency of hydrodynamic injection, whole slide scanning was performed (Olympus) and quantification was performed in ImageJ. Briefly, individual hepatic lobules were identified, and the area of entire lobule and areas of hepatocytes with hFIX staining were outlined in ImageJ, and the % area calculated. As the lobule is the functional unit of the liver, the transfection efficiency in lobules would be representative of the entire lobe and liver.

To calculate the percentage of hFIX compared to pig FIX in liver tissue, the b-actin normalized band intensity was taken for each lobe and the control pig band intensity was subtracted to yield hFIX contribution; this result was divided by the calculated porcine FIX level, determined from the hFIX antibody cross-reactivity (band intensity/6.4%). The average of all five liver lobes is presented in the text (10.11±4.05%).

### Statistical Analysis

GraphPad Prism 7 software (GraphPad Software) was used to perform statistical analysis and generate graphs. Data are presented as mean±standard error of mean (SEM). Unpaired, parametric, two-tailed t-tests were used to test mean differences. Significance level used was P<0.05.

### Results

### Design and Validation of the Human Factor IX Transposon Vector

The results herein designed and validated the hFIX gene delivery vector. Transposons were pursued over episomal vectors to ensure stable expression of hFIX in pig hepatocytes. This would avoid potential silencing issues with plasmids (40), as well as the complexities of minicircle DNA production (41). To enhance integration, a hyperactive piggyBac (hyperPB) transposon system that has 10-fold enhanced activity compared to SB100X in mouse HTVI models was used (42). This transposon system was chosen because in a mouse model, it mediated supraphysiologic levels of hFIX at low-dose plasmid DNA (pDNA) levels (43).

For the vector, an expression cassette currently in AAV vector clinical trials for Factor IX gene therapy (6), consisting of a chimeric liver specific promoter with enhancer from the APO-HCR gene and the hAAT promoter was used (44). The expression cassette included the human albumin 3′ untranslated region (UTR), which can stabilize mRNA to enhance expression (45). The hFIX sequence was codon-optimized for expression in pigs, a strategy which demonstrated a 12-fold expression increase in mice (43). The results herein co-delivered hFIX expression transposon (FIG. 28A) and hyperPB transposase (FIG. 28B) plasmids to facilitate integration into pig genome.

The disclosed results first validated the efficacy of the synthesized hyperPB transposase, which was also pig-codon optimized, demonstrating the formation of stably transfected 293T cells and cell culture that continued GFP expression after 8 passages (FIG. 35). Next the transposon vector for hFIX expression was tested in a mouse model using HTVI (17). Delivery of 8 μg hFIX transposon and 1 μg hyperPB transposase led to 11,268±1,077 ng/mL hFIX at one-weekpost-injection (FIG. 28C), more than twice normal human levels. Immunohistochemistry (IHC) of mouse liver showed abundant hFIX-positive hepatocytes (FIG. 28D), with transfection efficiency of approximately 20% of hepatocytes (FIG. 28E).

FIG. 28A shows that the pT-LP1-FIX plasmid encodes terminal repeats (TR) of the piggyBac (pB) transposon to facilitate integration. The LP1 promoter is a composite promoter consisting of human APO-HCR enhancer and hAAT promoter. The SV40 intron located in the 5′ UTR enhances expression. The hFIX gene is codon optimized for pig expression. The 3′ UTR from the human albumin gene enhances mRNA transcript stability. The SV40 late polyadenylation (poly-A) sequence completes the expression cassette. FIG. 28B shows that a second plasmid encoding a hyperactive piggyBac transposase (hyperPB) is driven by a cytomegalovirus (CMV) promoter to mediate high level, transient expression in the liver. FIG. 28C shows that mouse HTVI of these plasmids yields high level hFIX levels in plasma (n=4) and FIG. 28D shows that frequent hFIX-positive hepatocytes by cytoplasmic immunostaining. FIG. 28E shows that individual lobules were quantified in three injected mice, and stained area quantified (n=6 lobules per mouse). Mean±SEM are depicted.

In FIG. 35, transfection of a reporter piggyBac transposon plasmid, pIRII-eGFP, encoding green fluorescent protein into HEK 293T cells was used to confirm activity of the synthesized hyperactive piggyBac transposase (hyperPB). The wildtype piggyBac plasmid, pCMV-pB, served as a control. The repeated passage of HEK 293T cells effectively dilutes out any unintegrated GFP plasmid, leaving only cells expressing GFP from integrated transposon cassettes. Cells are shown at passage 8. Bar=100 μm.

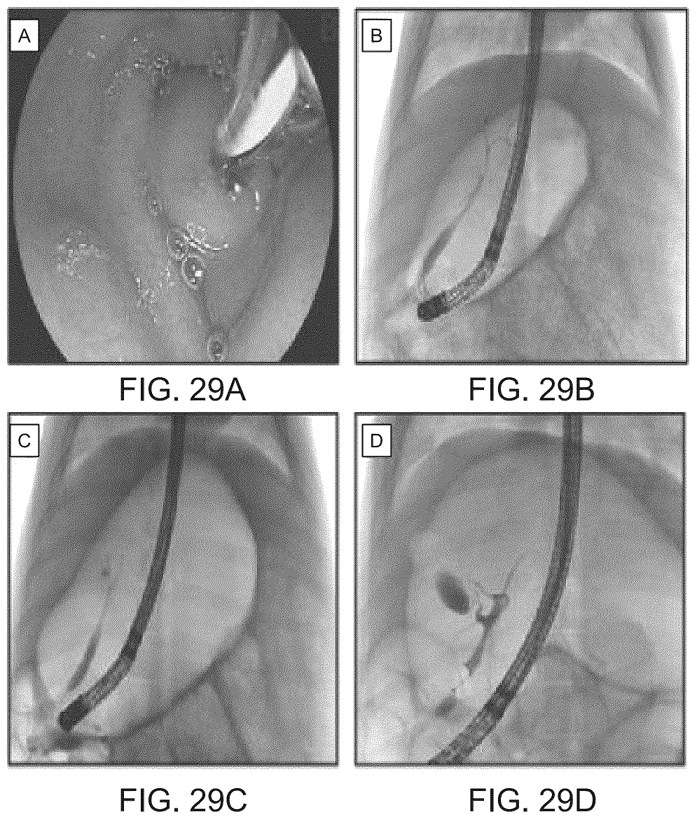

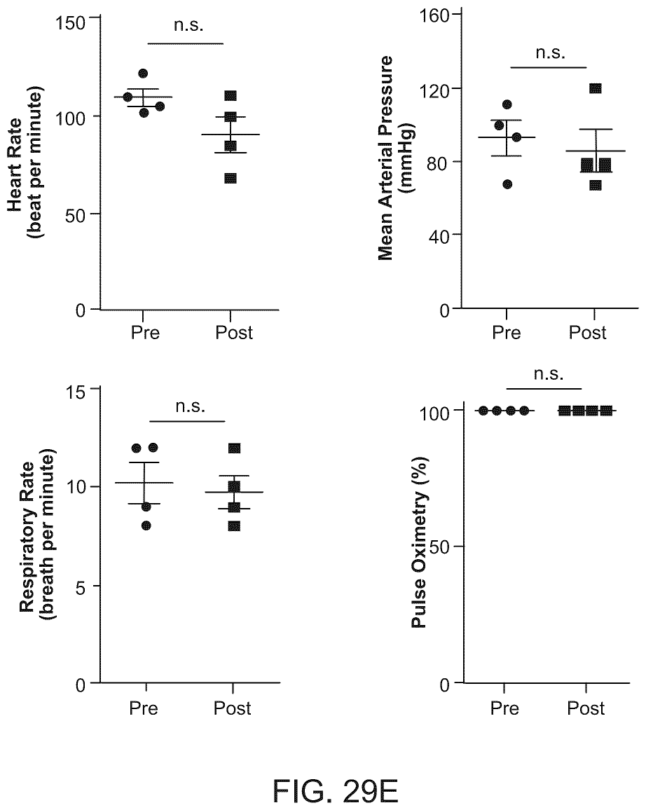

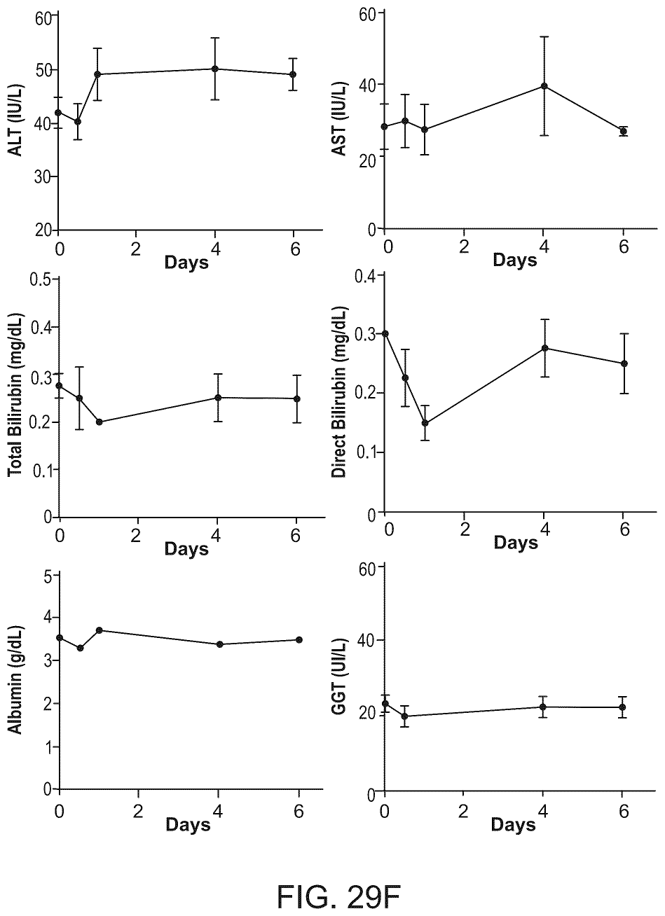

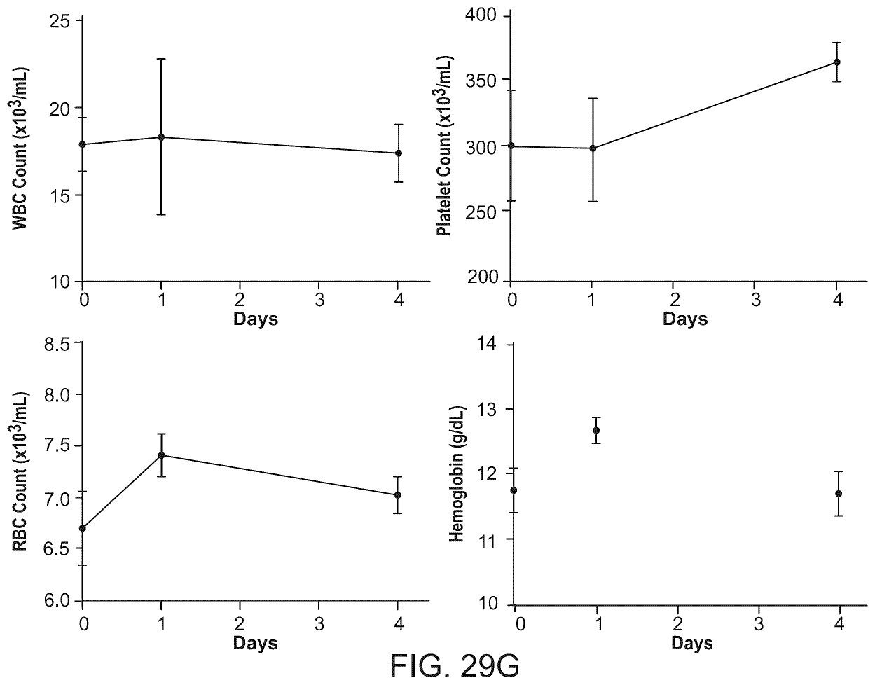

Biliary hydrodynamic injection procedure To translate hydrodynamic injection from mice into pigs, four female Yorkshire pigs weighing between 35 to 37 kilograms were obtained. Vital signs and weight were obtained prior to the procedure, and pigs were anesthetized as routine prior to endoscopy in patients. During each gene delivery procedure, the endoscope was advanced through the mouth, into the esophagus and stomach, and finally into the small intestine. A camera-mounted on the endoscope visualized the ampulla of Vater, the terminus of the biliary system (FIG. 29A). An occlusion balloon tipped catheter was advanced through the working channel of the endoscope, through the ampulla, and into the common bile duct and eventually the common hepatic duct (CHD). The location of the CHD was confirmed by injection of a small volume of contrast agent. Prior to pDNA injection, retrograde contrast injection opacified the biliary tree to ensure the catheter placement would result in access to the entire liver parenchyma and that no aberrant ductal anatomy was present (FIG. 29B, FIG. 29C). Post-hydrodynamic injection, fluoroscopy was repeated to demonstrate that the biliary system was intact without leakage of contrast solution into the liver parenchyma or abdominal cavity (FIG. 29D). Overall, this demonstrates the safety of the technique and maintenance of the integrity of the biliary tree.

FIG. 29A shows that cannulation of ampulla of Yater during endoscopy in pig #2 is depicted. FIGS. 29B-D show that Fluoroscopic imaging with contrast is utilized during ERCP to visualize the biliary tree. FIG. 29B shows that branches of the biliary tree are displayed prior to injection in pig #1.

FIGS. 29C and 29D show that Contrast injection with the balloon inflated in the common bile duct post-hydrodynamic injection reveals intact biliary system as evidenced by no contrast extravasation (Pig #1, FIG. 29C, Pig #3, FIG. 29D). FIG. 29E shows that for all four pigs, the values of the heart rate the mean arterial pressure, the respiratory rate, and pulse oximetry were compared pre- and post-procedure, demonstrating no significant difference. An unpaired, parametric, two-tailed t-test was used for analysis; significance (P<0.05). FIG. 29F shows that liver function biomarkers monitored post-hydrodynamic injection show minimal toxicity in pigs. ALT and AST remained within normal range in the week post-injection. Similarly, total bilirubin, direct bilirubin, albumin, and GGT did not show any alterations. Day O=injection day, pre-injection sample. The 15 minute post-injection sample is set at 0.5 days for representative purposes. FIG. 29G shows that hematologic parameters monitored after hydrodynamic injection show minimal effects. White blood cell (WBC) count post-injection showed no acute changes. Platelet (PLT) count demonstrated a mild increase post-injection. Red blood cell (RBC) count and hemoglobin showed a non-significant changes post-injection. Day 0=injection day, representing pre-procedure specimen.

For biliary hydrodynamic injection, the results herein used parameters of 30 mL of volume injected at a flow rate of 2 mL/sec (Table 3), similar to a previous study (39). Two doses of hFIX DNA transposon were employed, 3 mg and 5.5 mg, at similar transposon to transposase ratios, to evaluate if any dose dependence in hFIX expression was observed (Table 3). The four pigs averaged 43±11 minutes for the entire ERCP hydrodynamic procedure, emphasizing the speed of the approach despite additional time for imaging and data collection in pigs.

TABLE 3

| Pig | | Weight | Injection | Flow Rate | hFIX traspos on | hyperPB |
|---|---|---|---|---|---|---|
| Number | Sex | (kg) | Volume (ml) | (mL/sec) | plasmid (mg) | plasmid (mg) |
| 1 | Female | 35 | 30 | 2 | 3 | 0.4 |
| 2 | Female | 35 | 30 | 2 | 3 | 0.4 |
| 3 | Female | 37 | 30 | 2 | 5.5 | 0.6 |
| 4 | Female | 36 | 30 | 2 | 5.5 | 0.6 |

Yorkshire, female pigs weighing between 35.0-37.0 kg were used in the study. Each pig was injected at the same flow rate and total volume injected. Two sets of DNA doses were employed (3 mg and 5.5 mg) to evaluate transfection response; transposon: transposase ratio was kept similar between doses 7.5:1 versus 9.2:1.

### Safety of Biliary Hydrodynamic Injection

Given the unknown consequences of high pressure and large volume retrobiliary injection, the results herein evaluated the tolerability and safety of the procedure and its impact on the biliary system and liver parenchyma. Furthermore, vascular hydrodynamic injection is noted to cause rapid hemodynamic changes that could be dangerous during an injection procedure, along with mild-to-significant transient hepatotoxicity (21). Benefitting from the lower volume and flow rate employed, the results herein investigated the safety metrics of the disclosed approach.

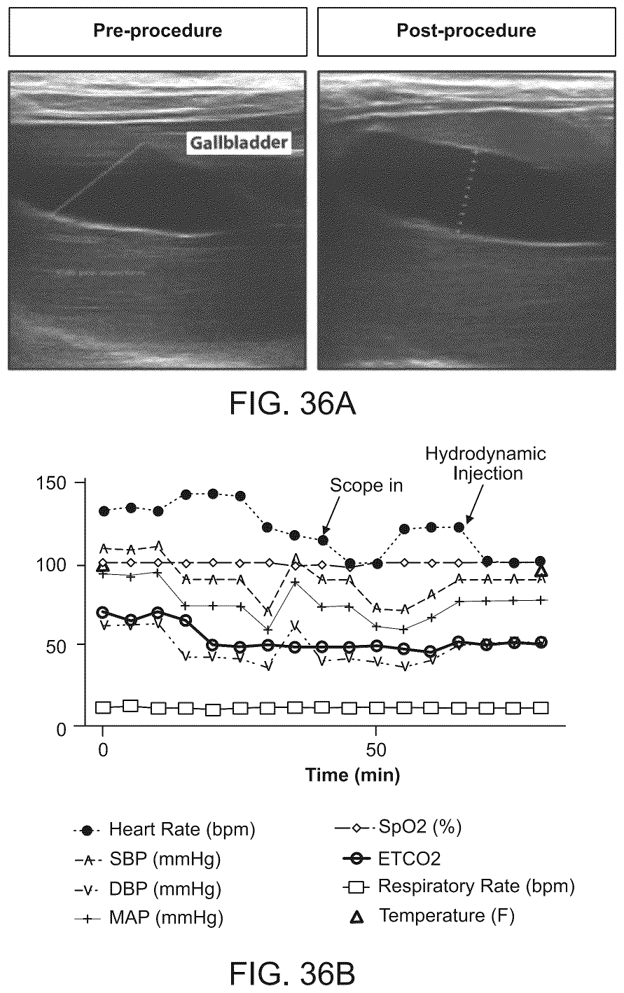

During the procedure, transabdominal ultrasound was performed to evaluate for any acute biliary or liver abnormalities. Post-procedure liver ultrasound did not reveal any abnormalities. The gallbladder measured pre- and post-procedure did not demonstrate changes in size, confirming balloon seal during hydrodynamic injection prevented fluid entry into this space (FIG. 36A).

Vital signs taken during the procedure did not show any acute changes in blood pressure, heart rate, pulse oximetry, or respiratory tidal volumes from pre- to post-procedure (FIG. 29E; FIG. 36B). Monitoring during the hydrodynamic injection itself revealed transient increase in heart rate from 105 to 128 beats per minute during the second half of the injection that returned back to baseline within ten seconds of injection cessation; cardiac rhythm remained normal throughout (Video SI). Pigs recovered from anesthesia normally and did not exhibit any acute behavioral changes. In the week post-hydrodynamic injection, liver function was monitored, and there were no significant changes in alanine aminotransferase (ALT), aspartate aminotransferase (AST), total or direct bilirubin, albumin, and gamma-glutamyl transferase (GGT) (FIG. 29F). The hematological parameters were measured to investigate any bleeding or inflammation post-injection (FIG. 29G). There was no leukocytosis in the four days post-injection, while hemoglobin, red blood cell (RBC) count, platelet values did not show significant changes in the pigs from prior to injection. Pigs displayed normal growth patterns (1 kg per week) in the weeks post-hydrodynamic injection, increasing weight from 35.33±0.33 kg to 38.83±0.33 kg in three weeks (p=0.0018).

FIG. 28A shows that ultrasound monitoring during procedure reveals no abnormalities from the hydrodynamic procedure. Pre-procedure ultrasound images revealed a normal sized gallbladder. Post-procedure, the gallbladder showed no increase in size suggesting successful balloon placement upstream of the cystic duct. No other liver abnormalities were seen. FIG. 36B shows that different vital signs were monitored during the entire endoscopic procedure, from entry of endoscope to withdraw after the biliary hydrodynamic injection, demonstrating minimal fluctuations. Representative anesthesia record was shown for Pig #3.

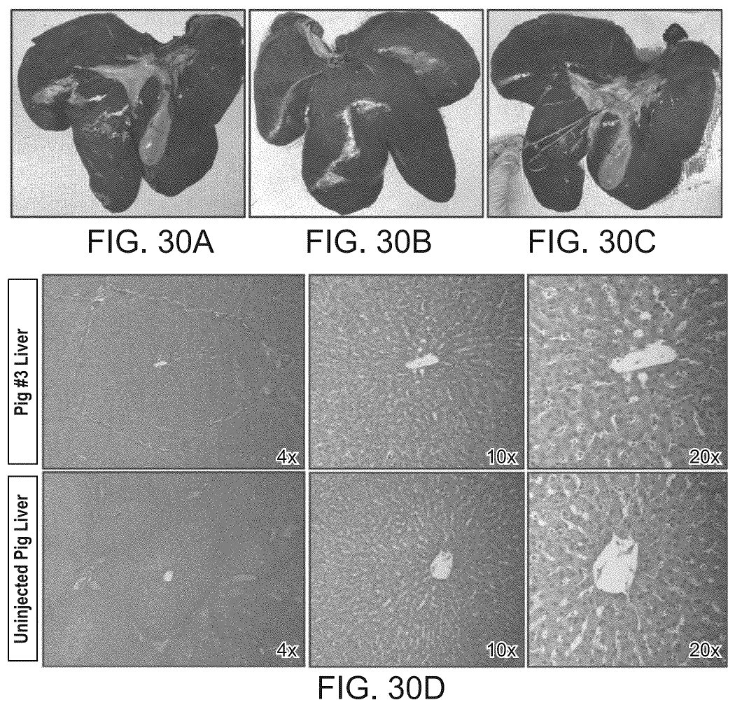

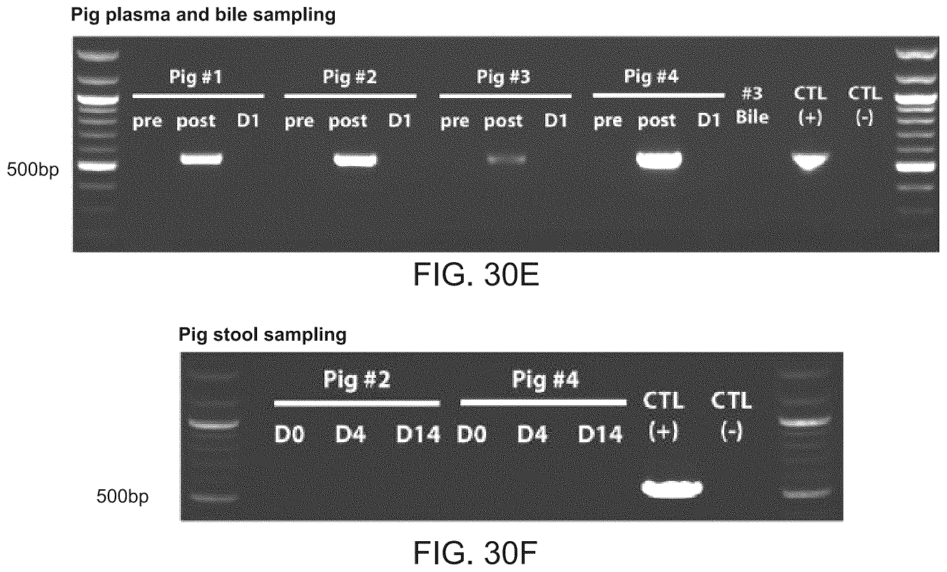

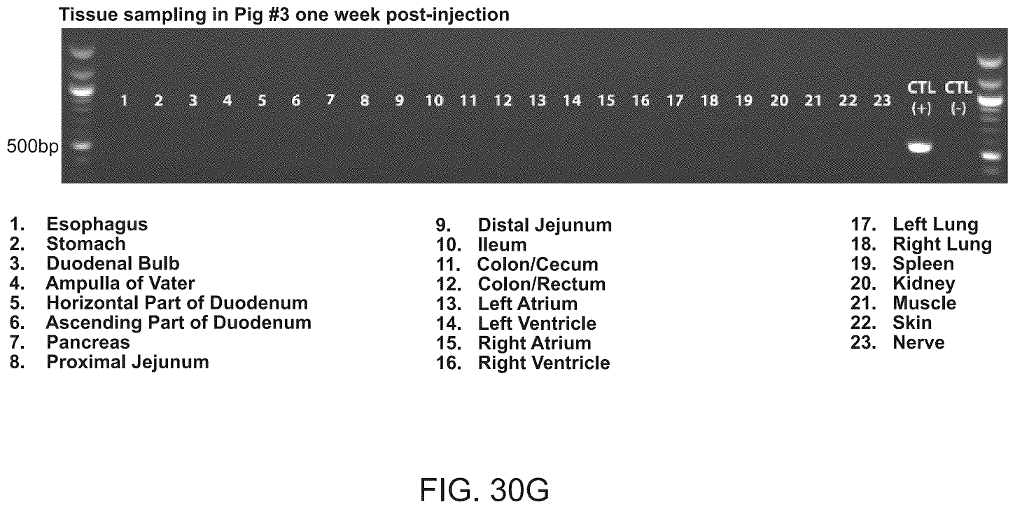

Pig #3 was selected for tissue analysis at one-week post-injection, since it received a higher pDNA dose. Moreover, at this timepoint, hFIX expression levels should be near peak before possible transgene silencing and/or immune response later against hFIX. Upon necropsy of pig #3, gross visual inspection of the visceral and diaphragmatic surfaces of the liver did not show any abnormalities (FIG. 30A, FIG. 30B). During examination of the pig's liver, the biliary tract was dissected observing that the right and left hepatic bile ducts were intact (FIG. 30C), confirming post-procedure fluoroscopic imaging (FIG. 29D). Review of liver histology showed normal findings with no immune infiltrate or necrosis, resembling an un-injected pig (FIG. 30D).

The elimination of pDNA within the biliary tract one-week post-injection was investigated, confirming that hFIX DNA was eliminated from bile by that time point (FIG. 30E). Given that pDNA in the bile ducts eventually drains into the small intestine, it was determined whether any pDNA undergoes transformation into pig intestinal bacteria. Stool DNA collected from two pigs was negative for hFIX DNA at all time points, showing pDNA transmission did not occur to host intestinal microbiome (FIG. 30F).

FIG. 30A shows that the visceral liver surface of pig #3 one-week post-injection is depicted with all five lobes observed along with the gall bladder and portal triad, showing no gross abnormalities. FIG. 30B shows that the diaphragmatic liver surface of pig #3 similarly shows no abnormalities. FIG. 30C shows that the intrahepatic biliary branches of pig #3 were dissected with no ruptures or lesions observed. FIG. 30D shows that hydrodynamically injected pigs exhibited normal liver histology one-week post-injection. H&E stains are presented for pig #3 and a non-injected pig. PCR for hFIX DNA demonstrated the expected band size (550 bp). Positive control (CTL) is the hFIX plasmid, and negative control is non-injected pig liver. FIG. 30E shows that pDNA biodistribution into porcine body fluids post-hydrodynamic injection was studied. Plasma samples were taken pre-procedure, 15 minutes post-procedure, and at day 1 post-procedure for all four pigs. Bile tested by PCR at one-week post-injection during necropsy was negative. FIG. 30F shows that the Stool samples were collected from pig #2 and #4 pre-procedure, and at days 4 and 14 post-injection were tested for hFIX DNA by PCR. FIG. 30G shows that transfection of pDNA post-hydrodynamic injection into non-liver tissues was not demonstrated. A panel of 23 different tissues were sampled during necropsy in pig #3 one-week post-injection was tested by PCR.

Off-target distribution of the gene therapy was assessed within the pig by testing for pDNA beyond the liver in other tissues. Plasma samples before and after injection were collected to observe for the entry of pDNA into the circulation. PCR testing revealed the presence of pDNA in the plasma 15 minutes post-injection (FIG. 30E), explained by escape of fluid from biliary caniculi, through hepatocyte tight junctions, and into venous circulation (33). By day 1 post-injection, pDNA was absent consistent with degradation by serum DNases at this time point (46, 47). With the presence of pDNA in systemic circulation, the ability of any DNA transfection to occur in other organs outside of the liver was assessed. Different tissue samples (23 in total) throughout pig #3 were investigated, including the kidney, lungs, heart, spleen, pancreas, and several locations along the GI tract (stomach, duodenum, colon). All tissues were negative for hFIX DNA by PCR indicating no pDNA delivery (FIG. 22G).

### Evaluation of Human FIX Gene Expression in Pigs

hFIX expression was next evaluated in the four pigs injected. All four pigs were monitored for plasma levels of hFIX, but surprisingly, no hFIX was detected in the plasma at day one and day four post-injection, when pDNA expression levels should be highest based on previous hydrodynamic injection literature in mice (17). Since protein expression in the tissue versus blood could be discordant, liver tissue from each injected pig was analyzed to interrogate hFIX expression.

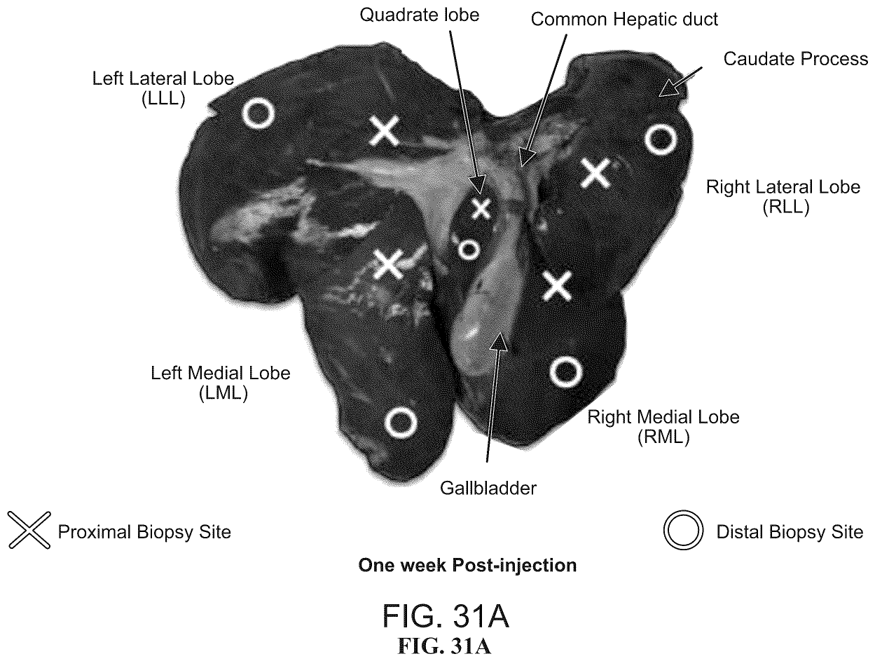

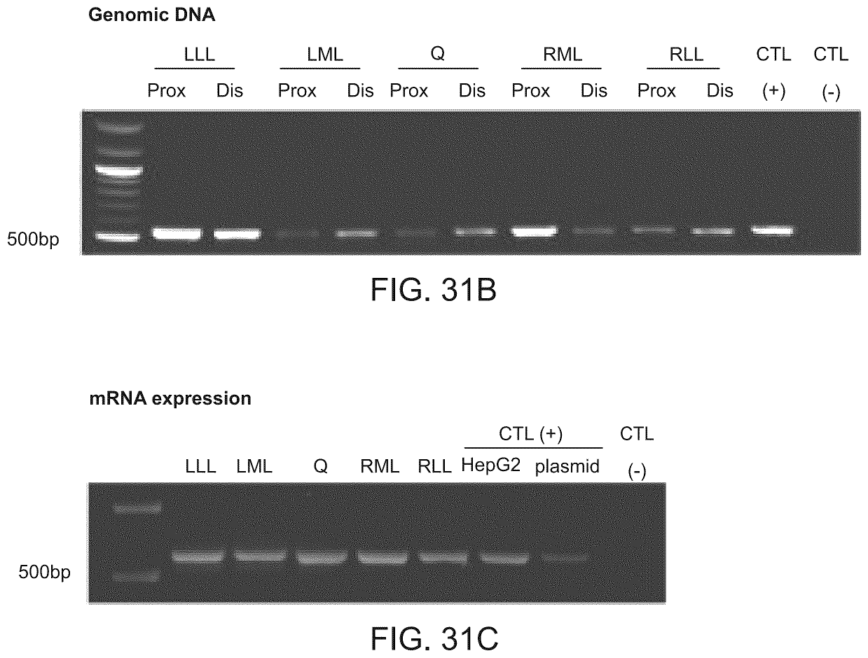

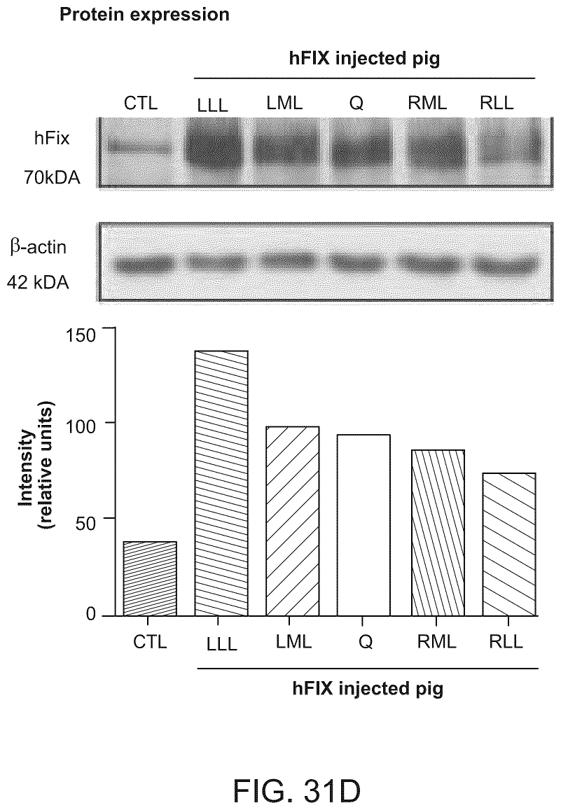

As depicted in FIG. 31A, samples were taken in proximal and distal portions of every liver lobe in relation to the hydrodynamical injection point in the CHD. The proximal versus distal areas of the liver may have received different pressures from the hydrodynamic injection; proximal portions are closer to the pressure source, although the distal portions may have smaller diameter biliary branches that would facilitate generation of increased pressure.

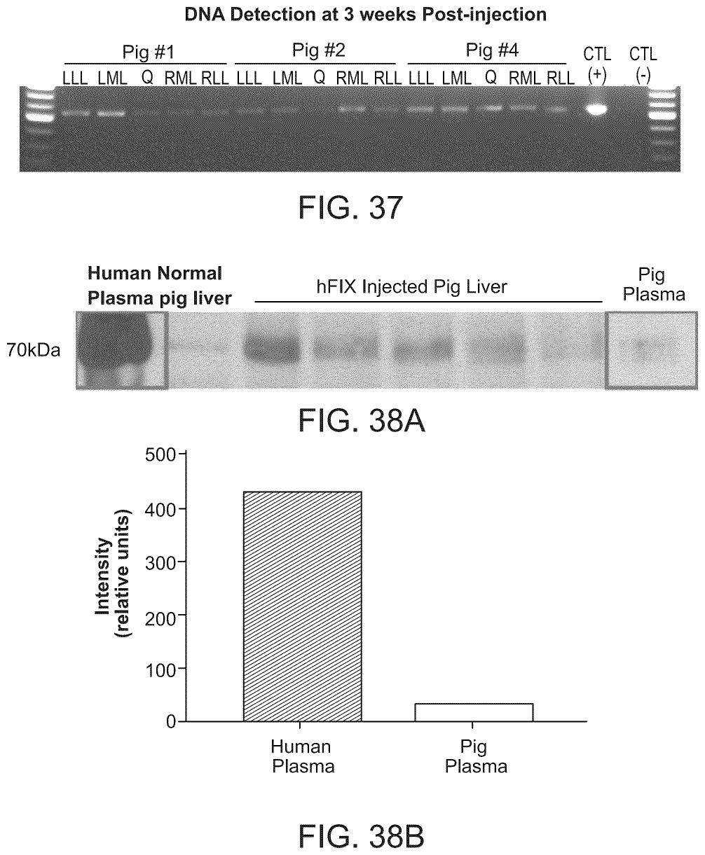

The presence of hFIX DNA was assessed by PCR among different liver samples. Since the hFIX gene sequence is codon optimized, an NIH BLAST analysis of hFIX primers did not reveal any significant matches among other organisms in the database, emphasizing high specificity. As shown in FIG. 31B, the results herein found successful delivery of the hFIX DNA into all pig liver lobes. Similarly, liver tissue from the other three pigs at 3 weeks post-hydrodynamic injection was analyzed, successful amplification of hFIX DNA was observed in every liver lobe (FIG. 37). The presence of hFIX DNA at 3 weeks post-injection provides evidence for the stable transfection inside cells.

In FIG. 37, genomic DNA was extracted from liver tissue of pig #1, #2, and #4 at three weeks post-injection. PCR was performed for hFIX DNA and revealed the expected band size (550 bp) for hFIX DNA in all liver lobes tested.

The results herein validated that the detected hFIX DNA was transcribed into mRNA. Analyzing tissue from pig #3, mRNA expression was observed in every liver lobe, indicating presence inside the nucleus (FIG. 31C). The presence of hFIX protein was next evaluated by western blot of liver tissue. Antibody specificity for hFIX versus pig factor IX, important for interpretation of the western blot and IHC, was examined. The hFIX antibody stained the correct hFIX protein size (70 kDa) in human plasma, while low level of porcine FIX cross-reactivity was observed in pig plasma, calculated to be 6.4% (FIGS. 38A-38B). Western blot of pig liver tissue demonstrated clear bands of hFIX staining in all hFIX gene injected liver lobes at varying expression levels (FIG. 31D). A small amount of immunostaining was observed in the normal, un-injected pig liver, consist with aforementioned hFIX antibody cross-reactivity. Semi-quantitative analysis of beta-actin normalized band intensity demonstrated all hFIX-injected bands well above un-injected control liver (FIG. 31D). Furthermore, the tissue hFIX protein level was calculated as 10.11±4.05% of the control pig liver's porcine factor IX level.

FIG. 38A shows that Western blot of human plasma and pig plasma was performed, demonstrating that the hFIX antibody stains human plasma intensely at the correct molecular weight (70 kDa), while demonstrating minimal binding to porcine FIX. FIG. 38B shows that the band intensity on the western blot was quantified, using normalization of the input protein levels in each well (1:0.66 human plasma to pig plasma protein).

FIG. 31A shows that tissue sampling scheme of pig liver across different lobes: right lateral lobe (RLL), right medial lobe (RML), left medial lobe (LML), left lateral lobe (LLL), and quadrate lobe. Proximal and distal samples to the site of catheter injection in the CHD were taken among all lobes. Pig #3 liver is depicted in A, harvested one-week post-injection for subsequent tissue analysis. FIG. 31B shows that PCR on genomic DNA revealed expected band (550 bp) for hFIX DNA in all liver lobes tested. FIG. 31C shows that RT-PCR was performed on RNA extracted at one-week post-injection demonstrated the expected band size. RNA extracted from HepG2 cells transfected with pT-LP1-hFIX plasmid serves as one positive control. For both PCR's, positive control (CTL) is hFIX plasmid, and negative control is non-injected pig liver tissue. FIG. 31D shows that Western blot on liver tissue demonstrated correct size of hFIX (70 kDa) with a low-level cross reactivity for porcine FIX at the same molecular weight.

Quantification of hFIX expression by western blot band intensity and normalized with beta-actinis provided. (CTL=control, non-injected pig liver).

Analysis of Transfection Efficiency of hFIX in Pig Liver

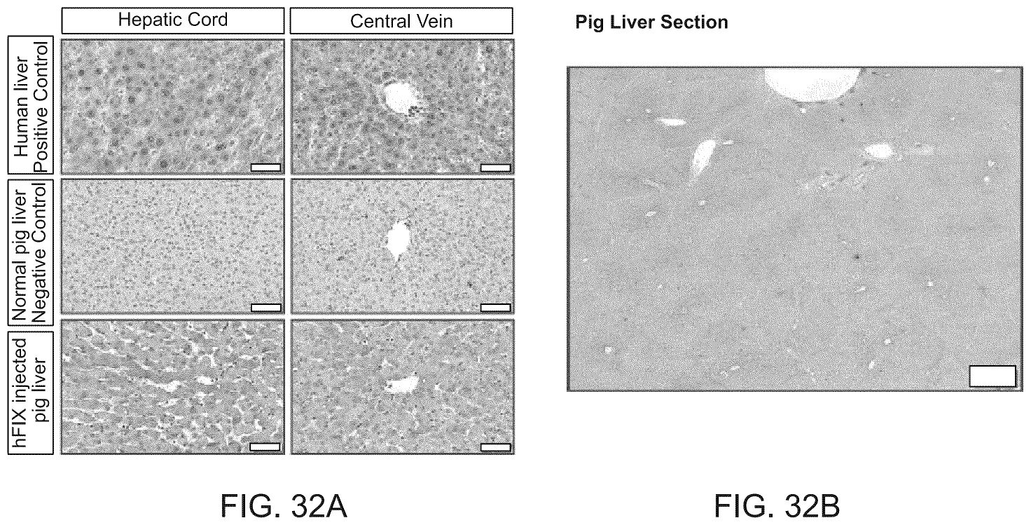

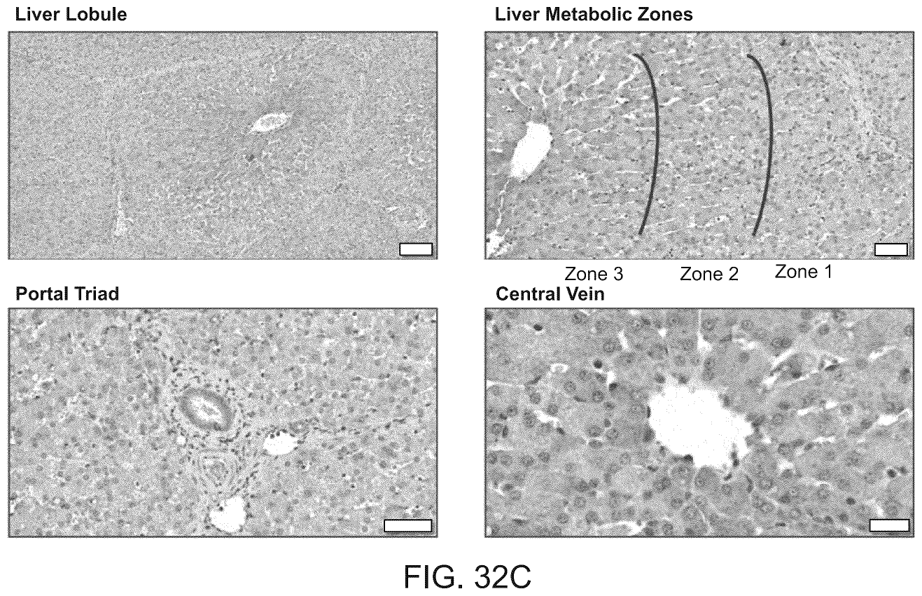

hFIX expression was evaluated by IHC in pig liver. Hepatocytes positive for hFIX were observed in the injected pig liver confirming the western blot results. The hFIX stain was cytoplasmic within hepatocytes of injected pigs without any staining in the nucleus, matching hFIX staining in a human liver control (FIG. 32A). By comparison, the un-injected control pig liver demonstrated only light background staining consistent with low-level porcine FIX cross-reactivity (FIG. 32A).

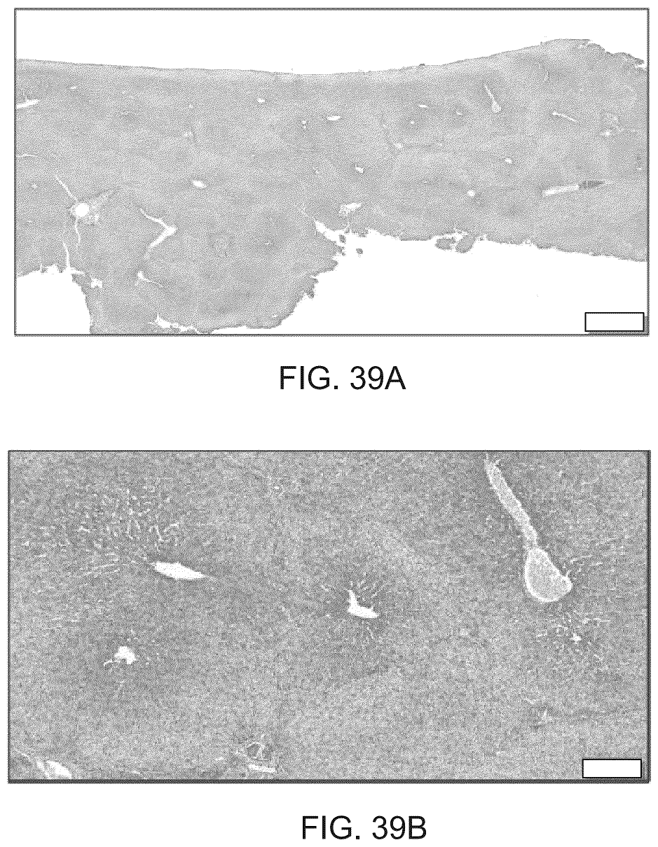

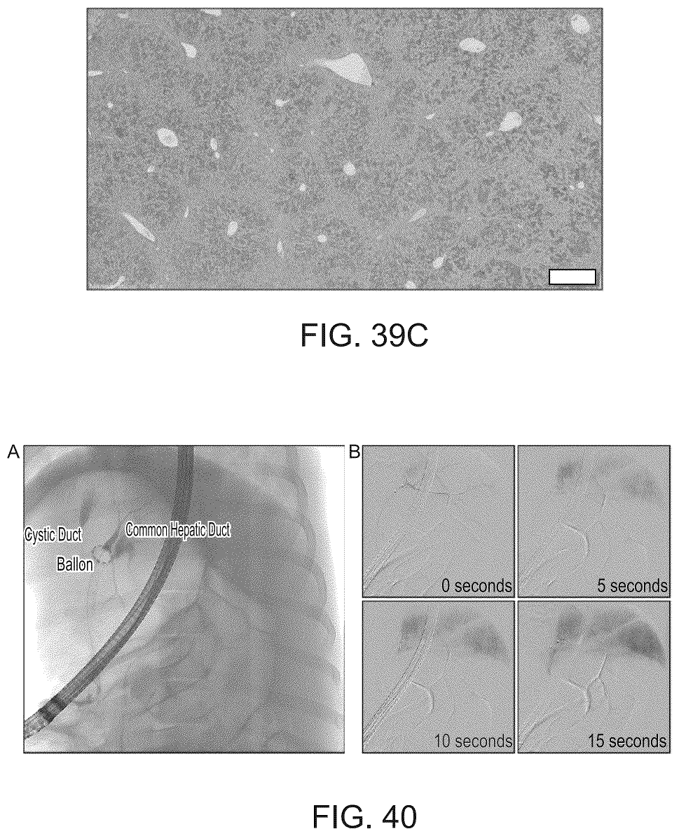

Given that the hepatocytes within the lobule have different metabolic functions depending on zone (48) and that hydrodynamic injection in mice shows pericentral vein predominance (21), the distribution of hFIX-positive hepatocytes within pig lobules was evaluated. hFIX-positive hepatocytes were always seen around the central vein of individual liver lobules (FIG. 32B, FIG. 32C). In fact, across entire tissue sections, hFIX-positive hepatocytes were observed around 100% of central veins analyzed and within every lobule (FIG. 32B, FIG. 39A). While the most intense immunostaining was observed around the central vein, hFIX-positive hepatocytes could be identified across all three metabolic zones, including in proximity to the portal triad (FIG. 32C, FIG. 39B).

FIG. 32A shows that IHC for hFIX in human liver tissue demonstrated homogenous, cytoplasmic expression in hepatocytes, with no apparent intensity difference among hepatocytes in a lobule (central vein area versus cords). Control tissue from a non-injected pig liver does not demonstrate hFIX staining as expected, with only light background staining for porcine FIX appreciated. Immunostaining in hFIX-injected pig #3 revealed abundant hFIX-hepatocytes, with similar cytoplasmic staining to human hepatocytes, although more variable expression intensity amongst positive cells. (bar=50 μm) FIG. 32B shows that tissue section of hFIX-injected pig at low power reveals hFIX expression in every lobule (bar=500 μm). FIG. 32C shows that an individual lobule demonstrates the most intense expression around the central vein radiating toward the fibrous lobule borders (bar=100 μm). Positive hFIX-hepatocytes are seen in all functional zones, although most abundant in zone 3 (bar=50 μm). hFIX-expression is most intense and uniform around the central vein (bar=20 μm), although numerous positive cells can also be seen near the portal triad (bar=50 μm). IHC images for the hFIX injected pig comes from the distal biopsy site of the right medial lobe of pig #3.

FIG. 39A shows that an example of a liver section stained for hFIX at low magnification power is presented from pig #3, LML proximal section, demonstrating that immunostaining can be observed in every single lobule, with intensity highest in the center of the lobule (bar=1 mm). FIG. 39B shows that a magnified image from the same liver biopsy section is presented, showing intense staining bordering the central vein and radiating to the lobule borders (bar=200 μm). FIG. 39C shows that a representative image of mouse liver after HTVI of hFIX is presented, demonstrating positive hFIX-hepatocytes in every lobule (bar=200 μm).

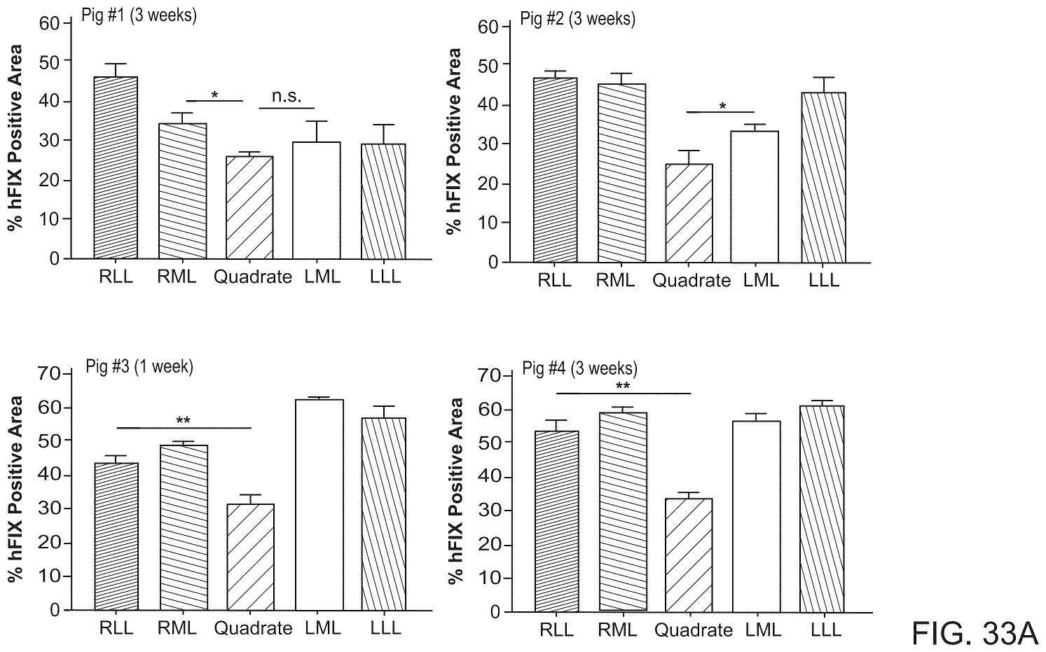

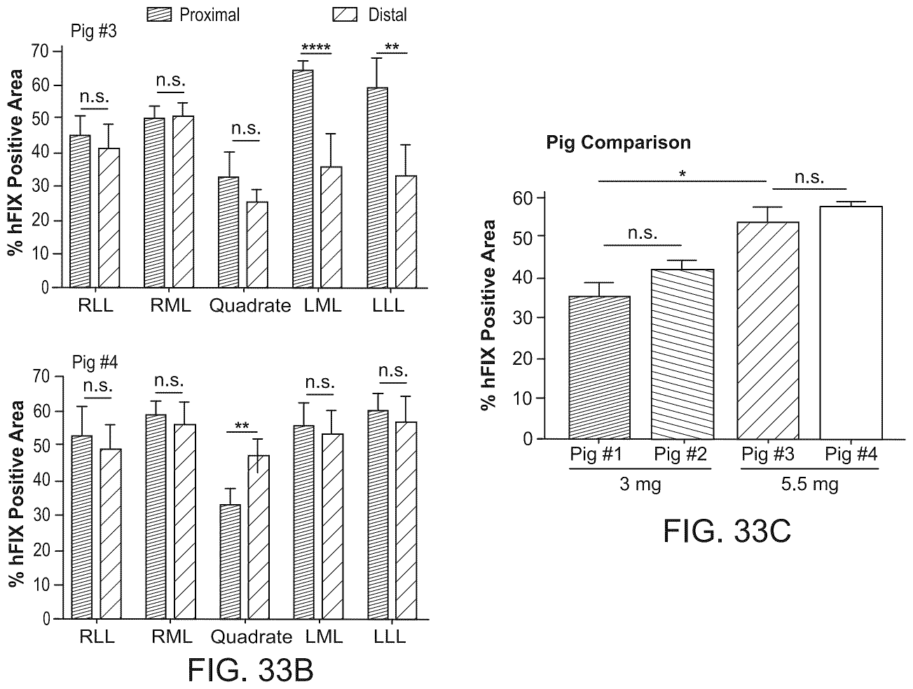

As suggested by previous DNA, mRNA and western blot results, it was hypothesized that hFIX-positive hepatocytes would be observed in every liver lobe of the pig liver. Indeed, the results herein found hFIX-positive hepatocytes in every liver lobe tested (FIG. 33A), indicating that a single CHD injection site could reach all liver lobes efficiently. Transfection efficiency was quantified by calculating the hFIX stained area within multiple lobules per section. The quadrate lobe showed significantly lower percentage area hFIX-stained in 3 out of the 4 pigs, while there was no clear right versus left liver predominance in transfection efficiency (FIG. 33A). The results herein also assessed if there was an expression gradient (distal vs. proximal) within an individual lobe as a function of CHD injection site. In general, it was found that the proximal and distal biopsy sites had similar hFIX-positive cell percentage; a few lobules showed statistical differences that were not replicated between pigs (FIG. 25B).

In testing reproducibility of hFIX transduction, hFIX-positive hepatocytes were found in all four pigs injected, in all five liver lobes of each animal, and in every lobule examined, indicating a 100% injection success rate across animals (FIG. 33A). There was a trend toward a dose dependent response to plasmid DNA, with a significant difference in the percentages of hFIX-positive cells from pig #1 (3 mg) and the two 5.5 mg dosed pigs (pig #3, #4) (FIG. 33C).

The hFIX immunostained area within one hepatic lobule was quantified, and then 5-6 lobules were averaged to yield a liver lobe value. FIG. 33A shows that the percentage area of hFIX-positive immunostaining within individual lobules (n=5-6) in pig #3 (one-week post-injection) and pig #1, #2 and #4 (3 weeks post-injection) is provided. FIG. 33B shows that amongst pig #3 and #4, distal and proximal portions of an individual hepatic lobe do not show consistent differences in percentage area of hFIX-positive hepatocytes (n=5-6). FIG. 33C shows that higher doses of pDNA led to higher hFIX percentage area transfected. Each pig represents the 4 lobe averages determined in FIG. 33A now pooled together with quadrate lobe removed due to small size (5% of liver). Data measurements are presented as mean±standard error of mean (SEM). Statistics represent unpaired, parametric, two-tailed t-tests; significance (p<0.05). **** p<0.0001; ** p<0.01; * p<0.05; n.s.=not significant.

### Comparison of Pig and Mouse Hydrodynamic Injection

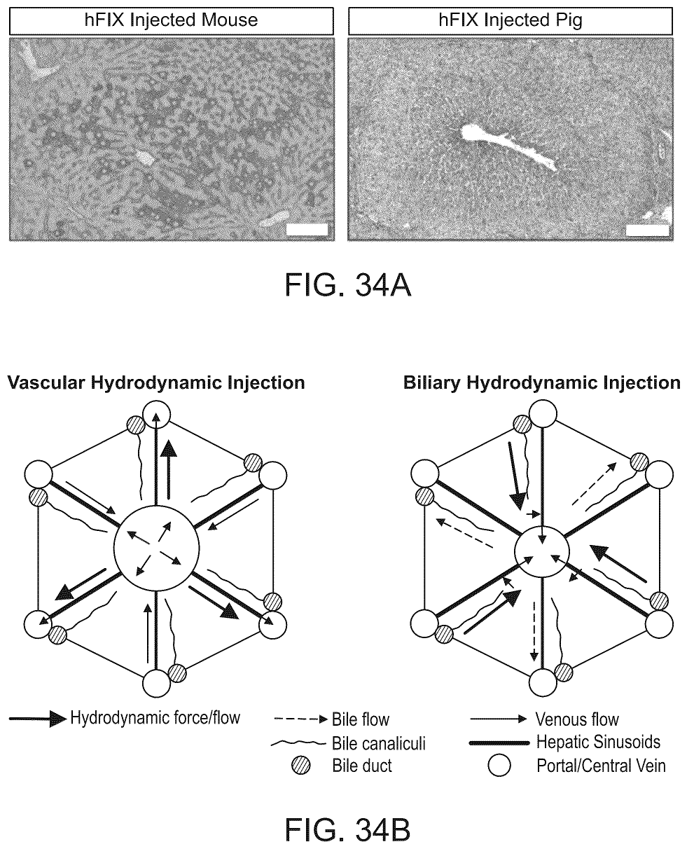

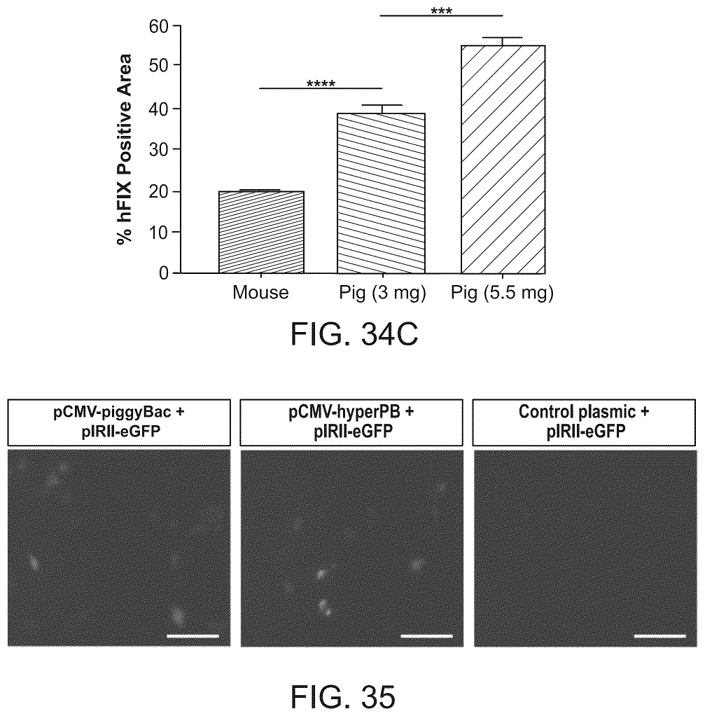

Gene delivery efficiency of the mouse HTVI and pig biliary hydrodynamic injection procedures were compared. The mouse HTVI was successful at mediating supraphysiologic levels of plasma hFIX (FIG. 20C), while the results herein did not identify plasma hFIX in the pig serum. At the tissue level, however, pig hydrodynamic injection yielded significantly more hFIX-positive pig hepatocytes than hFIX-positive mouse hepatocytes (FIG. 34A, FIG. 34C). Mouse HTVI yielded hFIX-positive hepatocytes primarily in proximity the central vein, although often not directly adjacent to the central vein, and almost never in periportal areas of the lobule (FIG. 34A). By comparison, pig hydrodynamic injection was also strongest around the central vein, with almost uniform transfection of cells adjacent to the central vein; hFIX-positive periportal hepatocytes could be readily detected as well. Both techniques were efficient at delivery to every lobule in the liver biopsy (FIG. 39A, FIG. 39C). FIG. 34A shows that mouse HTVI is primarily located around central veins in zone 2 (bar=50 μm). Pig hydrodynamic biliary injection leads to hFIX-positive expression almost uniformly circling central veins in zone 3 (bar=200 μm), and then radiating outward along the chords reaching zone 1 and 2 (pig #3 LML proximal section depicted). Mouse lobules are smaller than pig lobules, respectively. FIG. 34B shows that a model of the mechanism of biliary versus vascular hydrodynamic delivery is presented, explaining the differences between pig and mouse hydrodynamic transfection results. FIG. 34C shows that for individual lobules, the percentage of hFIX-positive pig hepatocytes is significantly more than hFIX-positive mouse hepatocytes at matched DNA per liver weight doses (Mouse represents 3 mice, 18 lobules; Pig represents average of 8 lobes from 2 pigs, with quadrate lobe excluded). Data measurements are presented as mean±standard error of mean (SEM). Statistics represent unpaired, parametric, two-tailed t-tests; significance (p<0.05). 5 * p<0.0001; ** p<0.01.

### Discussion

In this example, the ability to translate hydrodynamic gene delivery into a large animal model was confirmed. In particular, the results herein found that all subjects (pigs) were able to tolerate the nucleic acid delivery procedures with no acute changes in vital signs. Gross organ findings and histology at 1 and 3 weeks were normal. Moreover, unlike viral approaches for gene therapy where an AAV vector transduces many off target issues (2), the results herein found that pDNA inside the liver offering evidence for excellent targeting and subsequent safety of this approach. Overall, hFIX expression was found in every lobule of every lobe in every pig that underwent the procedure, with no clear transfection preference between the right and left liver lobes. All pigs continued expressing hFIX until the end of the experiment at three weeks, suggesting expression stability and immune tolerance, given that adaptive immune responses typically form by two weeks. Of note, a similar hyperPB-transposon experiment in mice showed stable hFIX expression for one year (43).

Impressively, the results herein found 50.19±3.50% of hepatocytes were hFIX-positive in the high-dose pDNA pigs. The majority of transfected cells clustered around the central vein, radiating outward along the hepatic sinusoids. hFIX-positive hepatocytes, usually with weaker expression, could also be observed in periportal areas along lobular borders. This IHC pattern follows the proposed hydrodynamic mechanism, that sees retrograde DNA solution rushing toward the biliary tract termination at the hepatocytes around the central vein (FIG. 34B). At this terminus, the increased fluid pressure onto hepatocyte membranes delivers genes into cells either directly via the canalicular surface or after extrusion through tight junctions and onto the sinusoidal membrane (33). Excess DNA solution exits into the hepatic vein and eventually NC. Similarly, vascular hydrodynamic approaches in pigs mediate gene delivery around the central vein (25, 28). However, the hydrodynamic pressure comes from the central vein, temporarily increasing it in size (28), before rushing up sinusoids toward portal vein (FIG. 34). Fluid pressure increases in the narrowing sinusoid channels, explaining mid-lobule transfection prevalence in vascular hydrodynamic injection (FIG. 34B).

The distribution of hepatocytes transfected or transduced is important for liver gene therapy, since hepatocytes perform different metabolic functions within zones of the lobule (48). For example, gene therapy for urea cycle disorders requires targeting of hepatocytes in periportal areas (zone 1), where ammonia is metabolized and the defective genes (ex: omithine transcarbamylase) are expressed (49). Thus, bias in hepatic transfection could be a negative in the treatment of these inherited diseases. AAV has demonstrated a preference for periportal or pericentral hepatocyte transduction depending on species and age of animal (49). Importantly, the biliary approach disclosed herein mediated transfection of hepatocytes across all three zones of the hepatic lobule, which suggests utility in treatment of all potential liver disorders.

No hFIX was detected in the plasma of pigs post-hydrodynamic gene delivery, even though abundant hFIX-positive hepatocytes was observed by immunostaining and hFIX expression by western blot. This indicates a discordance between protein expression in the liver and the ability of pig hepatocytes to secrete hFIX. Such a discordance has previously been noted for hAAT in pig models of vascular hydrodynamic gene delivery (25, 28, 29). In one study, two vascular hydrodynamic gene injections yielded up to 15.3% hAAT-positive pig hepatocytes in one lobe, but no hAAT protein was detected in the plasma (28). Similarly, another study found miniscule amounts of hAAT (20-50 ng/mL, 0.006% of normal) in the plasma, despite hAAT expression 10% of human liver in the injected pig liver tissue (29).

Moreover, another recent study created a transgenic pig with hFIX cDNA inserted into the pig FIX locus; 100% hepatocytes with hFIX cDNA under an endogenous promoter yielded strikingly low hFIX plasma levels (80 ng/mL) (50).

Comparing the non-viral liver transfection efficiency in pigs to AAV vector transduction efficiency in large animal studies, higher transfection efficiency was observed (32.7-51.9%) compared to ssAAV8, which yielded an average 17% transduced area in cynomolgus and rhesus macaques at 3×1012 GC/kg dose (51), and to scAAV3B yielded 36% in rhesus macaques at a dose of 1×1013 GC/kg (52). Both doses exceed the maximum AAV dose in the hFIX clinical trial (2×1012 GC/kg), which yielded 7 to 12% of normal hFIX levels, but led to T cell responses in the two high-dose patients (6). Importantly, significant hFIX protein expression (10.11±4.05% of pig FIX level) was observed in liver tissue, which could portend clinically significant hFIX plasma levels in a different animal models that secrete hFIX efficiently. Without being bound by theory, it is believed that additional efficiency gains could be made to hepatocyte transfection, which is suggested by the increased transfection area at the higher 5.5 mg DNA dose.

The biliary hydrodynamic injection showed improvements over vascular hydrodynamic approaches in pigs. In these other investigations much larger volumes of fluid (200-300 mL per lobe vs. 30 mL total liver), faster flow rates (20-100 mL/sec vs. 2 mL/sec), and larger DNA doses (15-20 mg vs 3-5 mg) were required to obtain a measurable transfection efficiency. Of note, in spite of larger volumes of fluid, faster flow rates and larger DNA doses, vascular approaches demonstrate only 5-15% transfection efficiency (25, 28, 29, 53). Moreover, individual liver lobes were injected in some studies in order to increase transfection efficiency, since a strategy injecting from the IVC after double ballooning was largely inefficient (27). Indeed, the complexity of vascular approaches for hydrodynamic injection may limit widespread clinical adoption. Vascular hydrodynamic injection in pigs generally elicit ALT/AST spikes (100-200 U/L) post-injection compared to no elevation in the current study(28).

The efficiency gains from biliary hydrodynamic injection likely result from the low total volume of the biliary system in the liver (estimated 29 mL in adult humans) (35), which enables small volumes injected to have a more effective dispersion throughout the liver and potentially more pronounced impact on biliary pressure, compared to overloading the higher volume vascular system. The results herein also injected at a higher flow rate than the previous biliary studies of non-viral gene delivery in rats and dogs, which led to higher transfection efficiency (32, 33). Improvements in vector expression cassette and integration also led to these increases as well. Overall, the endoscopic approach can reach the entire liver through one entry point, at similar transfection efficiencies among lobes and within the lobe, at minimal volume and flow rates, without observed liver toxicity, and a short total procedure time. The results herein also demonstrated that the transfection efficiency of hydrodynamic gene delivery in mice can be surpassed in a larger animal model, at comparable weight-based DNA doses.

Large animals had been previously thought to be intrinsically more resistant to hydrodynamic transfection (53). This emphasizes the importance of testing gene therapy strategies in large animal models, where different anatomy and tissue structure could lead to divergent results.

In conclusion, the results herein demonstrate that biliary hydrodynamic gene delivery into the liver through biliary tract in pigs has higher levels of transfection efficiency than AAV-mediated vectors, while possessing no toxicity. Important for clinical application, the plasmid DNA production costs are magnitudes less in the presently disclosed retrobiliary, transposon-mediated approach when compared to AAV vector production. The high transfection efficiency achieved herein greatly exceeds that which intracellular protein liver disorders like Wilson's Disease, Crigler-Najjar, and familial hypercholesteremia require for clinical cure (54). The successful use of medical devices utilized in clinical practice for this gene delivery procedure suggests the potential for rapid clinical translation using the same parameters described in this study. Future investigations will continue refining the technique for optimal DNA doses and injection settings to mediate effective protein expression for human disease.

### REFERENCES FOR EXAMPLE 19

1. J. Baruteau, S. N. Waddington, I. E. Alexander, P. Gissen, Gene therapy for monogenic liver diseases: clinical successes, current challenges and future prospects, J. Inherit. Metab. Dis. 40, 497-517 (2017).
2. D. Wang, P. W. L. Tai, G. Gao, Adeno-associated virus vector as a platform for gene therapy delivery, Nat Rev Drug Discov. 18, 358-378 (2019).
3. B. S. Doshi, V. R. Arruda, Gene therapy for hemophilia: what does the future hold? Ther Adv Hematol. 9, 273-293 (2018).
4. M. Franchini, F. Frattini, S. Crestani, C. Sissa, C. Bonfanti, Treatment of hemophilia B: focus on recombinant factor IX, Biologics. 7, 33-38 (2013).
5. C. S. Manno, G. F. Pierce, V. R. Arruda, B. Glader, M. Ragni, J. J. Rasko, J. Rasko, M. C. Ozelo, K. Hoots, P. Blatt, B. Konkle, M. Dake, R. Kaye, M. Razavi, A. Zajko, J. Zehnder, P.
6. K. Rustagi, H. Nakai, A. Chew, D. Leonard, J. F. Wright, R. R. Lessard, J. M. Sommer, M. Tigges, D. Sabatino, A. Luk, H. Jiang, F. Mingozzi, L. Couto, H. C. Ertl, K. A. High, M. A. Kay, Successful transduction of liver in hemophilia by AAV-Factor IX and limitations imposed by the host immune response, Nature Medicine. 12, 342-347 (2006).
7. A. C. Nathwani, E. G. D. Tuddenham, S. Rangarajan, C. Rosales, J. McIntosh, D. C. Linch, P. Chowdary, A. Riddell, A. J. Pie, C. Harrington, J. O'Beime, K. Smith, J. Pasi, B. Glader, P. Rustagi, C. Y. C. Ng, M. A. Kay, J. Zhou, Y. Spence, C. L. Morton, J. Allay, J. Coleman, S. Sleep, J. M. Cunningham, D. Srivastava, E. Basner-Tschakarjan, F. Mingozzi, K. A. High, J. T. Gray, U. M. Reiss, A. W. Nienhuis, A. M. Davidoff, Adenovirus-associated virus vector-mediated gene transfer in hemophilia B, N. Engl. J. Med. 365, 2357-2365 (2011).
8. A. C. Nathwani, U. M. Reiss, E. G. D. Tuddenham, C. Rosales, P. Chowdary, J. McIntosh, M. Della Peruta, E. Lheriteau, N. Patel, D. Raj, A. Riddell, J. Pie, S. Rangarajan, D. Bevan, M. Recht, Y.-M. Shen, K. G. Halka, E. Basner-Tschakarjan, F. Mingozzi, K. A. High, J. Allay, M. A. Kay, C. Y. C. Ng, J. Zhou, M. Cancio, C. L. Morton, J. T. Gray, D. Srivastava, A. W. Nienhuis, A. M. Davidoff, Long-term safety and efficacy of factor IX gene therapy in hemophilia B, N. Engl. J. Med. 371, 1994-2004 (2014).
9. L. A. George, S. K. Sullivan, A. Giermasz, J. E. J. Rasko, B. J. Samelson-Jones, J. Ducore, A. Cuker, L. M. Sullivan, S. Majumdar, J. Teitel, C. E. McGuinn, M. V. Ragni, A. Y. Luk, D. Hui, J. F. Wright, Y. Chen, Y. Liu, K. Wachtel, A. Winters, S. Tiefenbacher, V. R. Arruda, J. C. M. van der Loo, O. Zelenaia, D. Takefman, M. E. Carr, L. B. Couto, X. M. Anguela, K. A. High, Hemophilia B Gene Therapy with a High-Specific-Activity Factor IX Variant, N. Engl. J. Med. 377, 2215-2227 (2017).
10. C. Y. Kok, S. C. Cunningham, K. H. Carpenter, A. P. Dane, S. M. Siew, G. J. Logan, P. W. Kuchel, I. E. Alexander, Adeno-associated virus-mediated rescue of neonatal lethality m argininosuccinate synthetase-deficient mice, Mol Ther. 21, 1823-1831 (2013).
11. W. C. Manning, S. Zhou, M. P. Bland, J. A. Escobedo, V. Dwarki, Transient immunosuppression allows transgene expression following readministration of adeno-associated viral vectors, Human Gene Therapy. 9, 477-485 (1998).
12. R. Caicedo, H. Morizono, L. Wang, R. McCarter, J. He, D. Jones, M. L. Batshaw, J. M. Wilson, Adeno-associated virus antibody profiles in newborns, children, and adolescents, Clin. Vaccine Immunol. 18, 1586-1588 (2011).
13. N. Brunetti-Pierri, A. Liou, P. Patel, D. Palmer, N. Grove, M. Finegold, P. Piccolo, E. Donnachie, K. Rice, A. Beaudet, C. Mullins, P. Ng, Balloon catheter delivery of helper-dependent adenoviral vector results in sustained, therapeutic hFIX expression in rhesus macaques, Mol Ther. 20, 1863-1870 (2012).
14. J. S. Powell, M. V. Ragni, G. C. White, J. M. Lusher, C. Hillman-Wiseman, T. E. Moon, V. Cole, S. Ramanathan-Girish, H. Roehl, N. Sajjadi, D. J. Jolly, D. Hurst, Phase 1 trial of FVIII gene transfer for severe hemophilia A using a retroviral construct administered by peripheral intravenous infusion, Blood. 102, 2038-2045 (2003).
15. A. Cantore, M. Ranzani, C. C. Bartholomae, M. Volpin, P. D. Valle, F. Sanvito, L. S. Sergi, P. Gallina, F. Benedicenti, D. Bellinger, R. Raymer, E. Merricks, F. Bellintani, S. Martin, C. Doglioni, A. D'Angelo, T. VandenDriessche, M. K. Chuah, M. Schmidt, T. Nichols, E. Montini, L. Naldini, Liver-directed lentiviral gene therapy in a dog model of hemophilia B, Sci Transl Med. 7, 277ra28-277ra28 (2015).
16. F. DeRosa, B. Guild, S. Karve, L. Smith, K. Love, J. R. Dorkin, K. J. Kauffman, J. Zhang, B. Yahalom, D. G. Anderson, M. W. Heartlein, Therapeutic efficacy in a hemophilia B model using a biosynthetic mRNA liver depot system, Gene Therapy. doi: 10.1038/gt.2016.46. (2016).
17. S. Ramaswamy, N. Tonnu, K. Tachikawa, P. Limphong, J. B. Vega, P. P. Karmali, P. Chivukula, I. M. Verma, Systemic delivery of factor IX messenger RNA for protein replacement therapy, Proc. Natl. Acad. Sci. U.S.A. 114, E1941-E1950 (2017).
18. F. Liu, Y. Song, D. Liu, Hydrodynamics-based transfection m animals by systemic administration of plasmid DNA, Gene Therapy. 6, 1258-1266 (1999).
19. B. Bonamassa, L. Hai, D. Liu, Hydrodynamic gene delivery and its applications m pharmaceutical research, Pharm. Res. 28, 694-701 (2011).
20. R. L. Kruse, T. Shum, X. Legras, M. Barzi, F. P. Pankowicz, S. Gottschalk, K.-D. Bissig, In Situ Liver Expression of HBsAg/CD3-Bispecific Antibodies for HBV Immunotherapy, Mol Ther Methods Clin Dev. 7, 32-41 (2017).
21. L. Sendra, M. J. Herrero, E. M. Montalva, I. Noguera, F. Orbis, A. Diaz, R. Femandez-Delgado, R. Lopez-Andujar, S. F. Alifio, H. Iwase, Ed. Efficacy of interleukin 10 gene hydrofection in pig liver vascular isolated “in vivo” by surgical procedure with interest in liver transplantation, PLoS ONE. 14, e0224568 (2019).
22. G. Zhang, X. Gao, Y. K. Song, R. Vollmer, D. B. Stolz, J. Z. Gasiorowski, D. A. Dean, D. Liu, Hydroporation as the mechanism of hydrodynamic delivery, Gene Therapy. 11, 675-682 (2004).
23. C. H. Miao, A. R. Thompson, K. Loeb, X. Ye, Long-term and therapeutic-level hepatic gene expression of human factor IX after naked plasmid transfer in vivo, Mol Ther. 3, 947-957 (2001).
24. T. Yokoo, K. Kamimura, T. Suda, T. Kanefuji, M. Oda, G. Zhang, D. Liu, Y. Aoyagi, Novel electric power-driven hydrodynamic injection system for gene delivery: safety and efficacy of human factor IX delivery in rats, Gene Therapy. 20, 816-823 (2013).
25. H. Yoshino, K. Hashizume, E. Kobayashi, Naked plasmid DNA transfer to the porcine liver using rapid injection with large volume, Gene Therapy. 13, 1696-1702 (2006).
26. S. F. Alifio, M. J. Herrero, I. Noguera, F. Dasi, M. Sanchez, Pig liver gene therapy by noninvasive interventionist catheterism, Gene Therapy, 14, 334-343 (2007).
27. K. Kamimura, T. Suda, W. Xu, G. Zhang, D. Liu, Image-guided, lobe-specific hydrodynamic gene delivery to swine liver, Mol Ther. 17, 491-499 (2009).
28. J. W. Fabre, A. Grehan, M. Whitehorne, G. J. Sawyer, X. Dong, S. Salehi, L. Eckley, X. Zhang, M. Seddon, A. M. Shah, M. Davenport, M. Rela, Hydrodynamic gene delivery to the pig liver via an isolated segment of the inferior vena cava, Gene Therapy. 15, 452-462 (2008).
29. K. Kamimura, T. Suda, G. Zhang, Y. Aoyagi, D. Liu, Parameters Affecting Image-guided, Hydrodynamic Gene Delivery to Swine Liver, Mol Ther Nucleic Acids. 2, e128 (2013).
30. L. Sendra, A. Miguel, D. Perez-Enguix, M. J. Herrero, E. Montalva, M. A. Garcia-Gimeno, I. Noguera, A. Diaz, J. Perez, P. Sanz, R. Lopez-Andujar, L. Marti-Bonmati, S. F. Alifio, K. Stieger, Ed. Studying Closed Hydrodynamic Models of “In Vivo” DNA Perfusion in Pig Liver for Gene Therapy Translation to Humans, PLoS ONE. 11, e0163898 (2016). E. L. Aronovich, K. A. Hyland, B. C. Hall, J. B. Bell, E. R. Olson, M. U. Rusten, D. W. Hunter, N. M. Ellinwood, R. S. Mclvor, P. B. Hackett, Prolonged Expression of Secreted Enzymes in Dogs After Liver-Directed Delivery of Sleeping Beauty Transposons: Implications for Non-Viral Gene Therapy of Systemic Disease, Human Gene Therapy. 28, 551-564 (2017).
31. S. E. Khorsandi, P. Bachellier, J. C. Weber, M. Greget, D. Jaeck, D. Zacharoulis, C. Rountas, S. Helmy, A. Helmy, M. Al-Waracky, H. Salama, L. Jiao, J. Nicholls, A. J. Davies, N. Levicar, S. Jensen, N. Habib, Minimally invasive and selective hydrodynamic gene therapy of liver segments in the pig and human, Cancer Gene Ther. 15, 225-230 (2008).
32. G. Zhang, D. Virgo, V. Budker, N. Armstrong, S. Knechtle, J. A. Wolff, Expression of naked plasmid DNA injected into the afferent and efferent vessels of rodent and dog livers, Human Gene Therapy. 8, 1763-1772 (1997).
33. J. Hu, X. Zhang, X. Dong, L. Collins, G. J. Sawyer, J. W. Fabre, A remarkable permeability of canalicular tight junctions might facilitate retrograde, non-viral gene delivery to the liver via the bile duct, Gut. 54, 1473-1479 (2005).
34. Y. Chen, L. Bai, Y. Zhou, X. Zhang, J. Zhang, Y. Shi, Fine-scale visualizing the hierarchicalstructure of mouse biliary tree with fluorescence microscopy method, Biosci. Rep. 40, 609 (2020).
35. J. Ludwig, E. L. Ritman, N. F. LaRusso, P. F. Sheedy, G. Zumpe, Anatomy of the human biliary system studied by quantitative computer-aided three-dimensional imaging techniques, Hepatology. 27, 893-899 (1998).
36. H. Kjekshus, C. Risoe, T. Scholz, O. A. Smiseth, Regulation of hepatic vascular volume: contributions from active and passive mechanisms during catecholamine and sodium nitroprusside infusion, Circulation. 96, 4415-4423 (1997).
37. F. Jacobs, S. C. Gordts, I. Muthuramu, B. De Geest, The liver as a target organ for gene therapy: state of the art, challenges, and future perspectives, Pharmaceuticals (Basel). 5, 1372-1392 (2012).
38. M. S. Cappell, D. M. Friedel, Stricter national standards are required for credentialing of endoscopic-retrograde-cholangiopancreatography in the United States, WJG. 25, 3468-3483 (2019).
39. V. Kumbhari, L. Li, K. Piontek, M. Ishida, R. Fu, B. Khalil, C. M. Garrett, E. Liapi, A. N. Kalloo, F. M. Selaru, Successful liver-directed gene delivery by ERCP-guided hydrodynamic injection (with videos), Gastrointest. Endosc. 88, 755-763.e5 (2018).
40. J. Schilttrumpf, P. Milanov, D. Abriss, S. Roth, T. Tonn, E. Seifried, Transgene loss and changes in the promoter methylation status as determinants for expression duration in nonviral gene transfer for factor IX, Human Gene Therapy. 22, 101-106 (2011).
41. Z.-Y. Chen, C.-Y. He, A. Ehrhardt, M. A. Kay, Minicircle DNA vectors devoid of bacterial DNA result in persistent and high-level transgene expression in vivo, Mol Ther. 8, 495-500 (2003).
42. J. E. Doherty, L. E. Huye, K. Yusa, L. Zhou, N. L. Craig, M. H. Wilson, Hyperactive piggyBac gene transfer in human cells and in vivo, Human Gene Therapy. 23, 311-320 (2012).
43. M. Di Matteo, E. Samara-Kuko, N. J. Ward, S. N. Waddington, S. N. Waddingon, J. H. McVey, M. K. L. Chuah, T. VandenDriessche, Hyperactive piggyBac transposons for sustained and robust liver-targeted gene therapy, Mol Ther. 22, 1614-1624 (2014).
44. A. C. Nathwani, J. T. Gray, C. Y. C. Ng, J. Zhou, Y. Spence, S. N. Waddington, E. G. D. Tuddenham, G. Kemball-Cook, J. McIntosh, M. Boon-Spijker, K. Mertens, A. M. Davidoff, Self-complementary adeno-associated virus vectors containing a novel liver-specific human factor IX expression cassette enable highly efficient transduction of murine and nonhuman primate liver, Blood. 107, 2653-2661 (2006).
45. M. J. Pearson, S. Khazaipoul, A. Optun, I. F. Pryme, B. Stem, J. E. Hesketh, Albumin 3′untranslated region facilitates increased recombinant protein production from Chinese hamster ovary cells, Biotechnol J. 7, 1405-1411 (2012).
46. K. Kawabata, Y. Takakura, M. Rashida, The fate of plasmid DNA after intravenous injection in mice: involvement of scavenger receptors in its hepatic uptake, Pharm. Res. 12, 825-830 (1995).
47. F. Liu, L. M. Shollenberger, C. C. Conwell, X. Yuan, L. Huang, Mechanism of naked DNA clearance after intravenous injection, J. Gene Med. 9, 613-619 (2007).
48. T. Kietzmann, Metabolic zonation of the liver: The oxygen gradient revisited, Redox Biol. 11, 622-630 (2017).
49. P. Bell, L. Wang, G. Gao, M. E. Haskins, A. F. Tarantal, R. J. McCarter, Y. Zhu, H. Yu, J. M. Wilson, Inverse zonation of hepatocyte transduction with AAV vectors between mice and non-human primates, Molecular Genetics and Metabolism. 104, 395-403 (2011).
50. J. Chen, B. An, B. Yu, X. Peng, H. Yuan, Q. Yang, X. Chen, T. Yu, L. Wang, X. Zhang, H. Wang, X. Zou, D. Pang, H. Ouyang, X. Tang, CRISPR/Cas9-mediated knockin of human factor IX into swine factor IX locus effectively alleviates bleeding in hemophilia B pigs, Haematologica, haematol.2019.224063 (2020).
L. Wang, R. Caicedo, P. Bell, J. Lin, R. L. Grant, D. L. Siegel, J. M. Wilson, Impact of pre-existing immunity on gene transfer to nonhuman primate liver with adeno-associated virus 8 vectors, Human Gene Therapy. 22, 1389-1401 (2011).
51. S. Li, C. Ling, L. Zhong, M. Li, Q. Su, R. He, Q. Tang, D. L. Greiner, L. D. Shultz, M. A. Brehm, T. R. Flotte, C. Mueller, A. Srivastava, G. Gao, Efficient and Targeted Transduction of Nonhuman Primate Liver With Systemically Delivered Optimized AAV3B Vectors, Mol Ther. 23, 1867-1876 (2015).
52. L. Sendra, M. J. Herrero, S. F. Alifio, Translational Advances of Hydrofection by Hydrodynamic Injection, Genes (Basel). 9, 136 (2018).
53. S. Fagiuoli, E. Daina, L. D'Antiga, M. Colledan, G. Remuzzi, Monogenic diseases that can be cured by liver transplantation, Journal of Hepatology. 59, 595-612 (2013).
54. K. Yusa, L. Zhou, M. A. Li, A. Bradley, N. L. Craig, A hyperactive piggyBac transposase for mammalian applications, Proc. Natl. Acad. Sci. U.S.A. 108, 1531-1536 (2011).
55. M. H. Wilson, C. J. Coates, A. L. George, PiggyBac transposon-mediated gene transfer in human cells, Mol Ther. 15, 139-145 (2007).
56. Y. Nakazawa, S. Saha, D. L. Galvan, L. Huye, L. Rollins, C. M. Rooney, M. H. Wilson, Evaluation of long-term transgene expression in piggyBac-modified human T lymphocytes, Journal of immunotherapy. 36, 3-10 (2013).
57. H. Herweijer, J. A. Wolff, Gene therapy progress and prospects: hydrodynamic gene delivery, Gene Therapy. 14, 99-107 (2007).

### Example 20

This Example shows inter alia parameters of biliary hydrodynamic injection during endoscopic retrograde cholangio-pancreatography in pigs for applications in gene delivery.

### Materials and Methods

### Animal Experiments

All animal experiments were conducted under approval of the institutional animal care and use committee (IACUC) of Johns Hopkins Hospital (protocol #SW19M428) and University of Maryland School of Medicine (protocol #0720003) and adhere to the guidelines of the NIH Guide for the Care and Use of Laboratory Animals.

Yorkshire pigs (Sus scrofa domestica) were acquired, weighing 35-54 kg. Pigs (8 total) were provided by Archer Farms (Darlington, Maryland). Pigs were housed in cages either singlyor in pairs with different toys for enrichment, water ad libitum, and food provided each day. A detailed protocol of the biliary hydrodynamic injection procedure was previously described [24]. Briefly, after pigs were anesthetized and placed supine, a duodenoscope was inserted and positioned such that the biliary orifice in the duodenal bulb was enface, Under fluoroscopic guidance (Phillips Allura C-arm), the bile duct was cannulated with a triple lumen sphincterotome and hydrophilic guidewire. A cholangiogram was attained after injection of 5-10 mL of radio opaque contrast (Omnipaque, 350 mg/mL; GE Health Co). The sphincterotome was exchanged for a stone extraction balloon which was inflated to 12 mm in the common hepatic duct.

Hydrodynamic injections were performed using a power injector (MEDRAD® Mark 7 Arterion) that contains up to 150 mL and can inject up to 50 mL/sec at a maximum of 1200 pounds per square inch (psi). For each injection, 25% contrast solution diluted with 0.9% saline solution was used to allow for real-time visualization to evaluate hepatic distribution and acinarization. In one pig, 5 milligrams of plasmid DNA, pCLucf, isolated with a gigaprep kit (Zymo Research) and dissolved into 0.9% saline solution and subsequently injected. pCLucf was a gift from John Schiller (Add gene plasmid #37328). For the acute pig studies, several different injection parameters were tested as described in Table 1. For the day 1 studies, parameters of 4 mL/sec at 40 mL volume were utilized in pigs, while day 14 studies used 2 mL/sec at 30 mL volume in pigs. Between each injection, at least five minutes were allowed to lapse in time, and contrast was verified to be no longer visualized on fluoroscopy prior to repeat injection. For several experiments, a pressure catheter (FOP-M260, FISO Technologies) was advanced through the guidewire channel with the sensor positioned 1 cm beyond the distal tip of the catheter, allowing it to measure intrabiliary pressures. Pressure readings were monitored in real-time by the connection of the catheter to a computer able to illustrate pressure tracings in real time. At the completion of the study, pigs were euthanized using potassium chloride overdose (>2 mmol/kg) following by verification of cardiac arrest.

C57BL6 mice (4 mice) were a gift of Svetlana Lutsenko of Johns Hopkins, originally sourced from Jackson Labs). Mice were housed with littermates with water and food ad libitum, and cotton enrichment in the cage. For HTVI, C57BL/6 mice weighing between 20 and 25 grams were selected, and 2.2 mL normal saline (8-10% body weight) was subsequently injected into the lateral tail vein of mice within 4-7 seconds. At the completion of the study, mice were euthanized using carbon dioxide. Mice were harvested within 15 minutes post-injection for tissue analysis.

TABLE 1
Biliary hydrodynamic injection parameters used in the acute pig studies.

| | | | | | |
|---|---|---|---|---|---|
| Injection | Volume | Flow rate | Pressure | | |
| Attempt | (mL) | (mL/sec) | (psi) | Port | Notes |

Pig #1

| | | | | | |
|---|---|---|---|---|---|
| 1 | 30 | 2 | 999 | Injection | Well-tolerated by pig, no power injector |
| | | | | (small) | deviation |
| 2 | 30 | 4 | 999 | Injection | Well-tolerated by pig, no power injector |
| | | | | (small) | deviation |
| 3 | 50 | 5 | 999 | Injection | Flow rate reduced by power injector |
| | | | | (small) | due to pressure limit reached |

Pig #2

| | | | | | |
|---|---|---|---|---|---|
| 1 | 45 | 5 | 1200 | Injection | Circuit burst where line connected to the power injector and |
| | | | | (small) | to the port. No evidence of liver parenchymal damage |
| 2 | 50 | 3 | 999 | Injection | Well-tolerated by pig, no power injector |
| | | | | (small) | deviation |
| 3 | 37 | 4 | 999 | Injection | Well-tolerated by pig, no power injector |
| | | | | (small) | deviation |

Pig #3

| | | | | | |
|---|---|---|---|---|---|
| 1 | 30 | 2 | 999 | Injection | Balloon slipped, rapidly dropping pressure reading |
| | | | | (small) | |
| 2 | 30 | 2 | 999 | Injection | Well-tolerated by pig, no power injector |
| | | | | (small) | deviation |
| 3 | 60 | 3 | 999 | Injection | Flow rate reduced by power injector due |
| | | | | (small) | to pressure limit reached |
| 4 | 140 | 1 | 999 | Injection | Well-tolerated by pig, no power injector |
| | | | | (small) | deviation |
| 5 | 80 | 4 | 999 | Injection | Well-tolerated by pig, no power injector |
| | | | | (small) | deviation |
| 6 | 47 | 10 | 999 | Guide port | Well-tolerated by pig, no power injector |
| | | | | (big) | deviation |

Three pigs were subjected to repeated hydrodynamic injections during one ERCP procedure. Different volumes, flow rates, and device catheter pressures were investigated. Clinical notes were also taken during the procedure, where any variations were reported, particularly reduction in flow rates by the power injector due to pressure limits being reached.

### Tissue Analysis

A subset of animals (pigs and mice) was euthanized, underwent necropsy and were harvested for organs within 15 minutes of hydrodynamic injection. Another cohort of pigs was similarly euthanized and livers harvested on Day 1 post-injection (n=2) or on Day 14 post-injection (n=3), respectively, to monitor long-term effects of hydrodynamic injection. Gross inspection of the liver and abdomen was performed for each dissection. Pig livers were sampled at sites proximal and distal to the injection point in the CHD. During the dissection, the integrity of the CHD and right and left hepatic ducts in the pig liver were verified. Tissue from pig and mouse liver were fixed in 10% formaldehyde and underwent Hematoxylin & Eosin (H&E) staining.

### Blood Analysis

Blood samples were collected before and after the procedure by a certified veterinary technician, the post-procedure blood draw was conducted within 15 minutes after the hydrodynamic injection. Additional blood samples were also collected before the euthanasia of pigs on Day 1 and Day 14. Blood draw was performed via the internal jugular vein of the pigs for later chemistry analysis. Liver function panel and routine serum chemistries were performed on a DiaSys Respons®910 chemistry analyzer. Samples were excluded if the chemistry analyzer showed gross hemolysis, due to its significant impact on the aspartate aminotransferase (AST), bilirubin and lactate dehydrogenase (LDH) levels. For plasmid DNA detection, DNA was isolated from serum using the QIAgen DNeasy Blood & Tissue kit, and then subjected to PCR (DreamTaq, ThermoFisher).

### Statistical Analysis

GraphPad Prism 7 software (GraphPad Software) was used to perform statistical analysis and generate graphs. Unpaired, parametric, two-tailed t-tests were used to test mean differences. Significance level used was P<0.05.

### Results

### Interrogating Maximum Volumes and Flow Rates During ERCP Injection

Our previous efforts defined 30 mL and 2 mL/sec as the maximally tolerated injection parameters during ERCP-mediated hydrodynamic injection [24]. At higher volumes or flow rates, the CHD upstream to the balloon ruptured, likely due to stress on the bile duct wall. In order to achieve higher flow rates and solve this issue, the procedure was adjusted for the current studies by placing the balloon immediately inferior to the liver hilum, such that the catheter tip would lie within the liver parenchyma. The pressure on the walls of bile duct walls would thus be reinforced by the liver parenchyma surrounding it, thereby preventing rupture. Initially, the injection port was used instead of the guidewire port, since the pressure catheter required the wider diameter of the guidewire port.

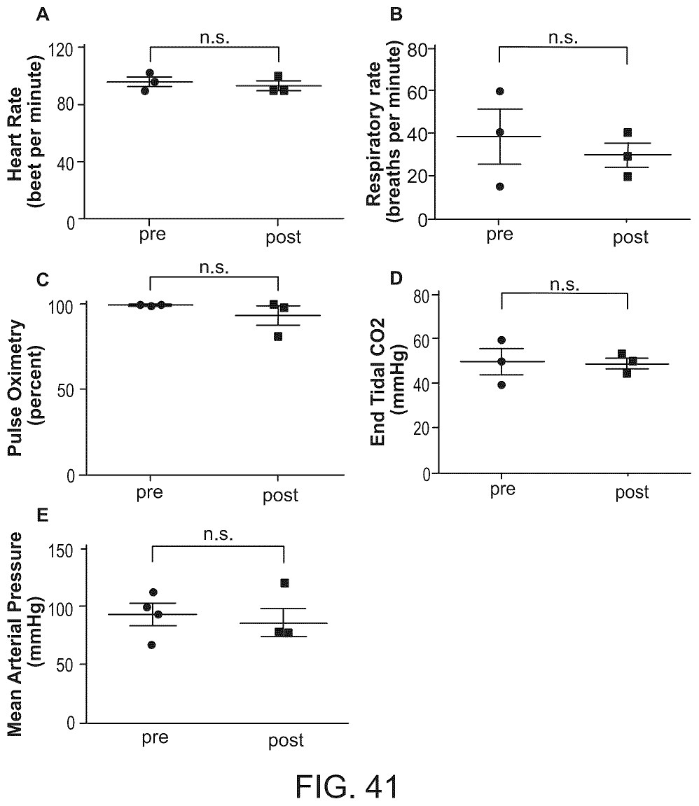

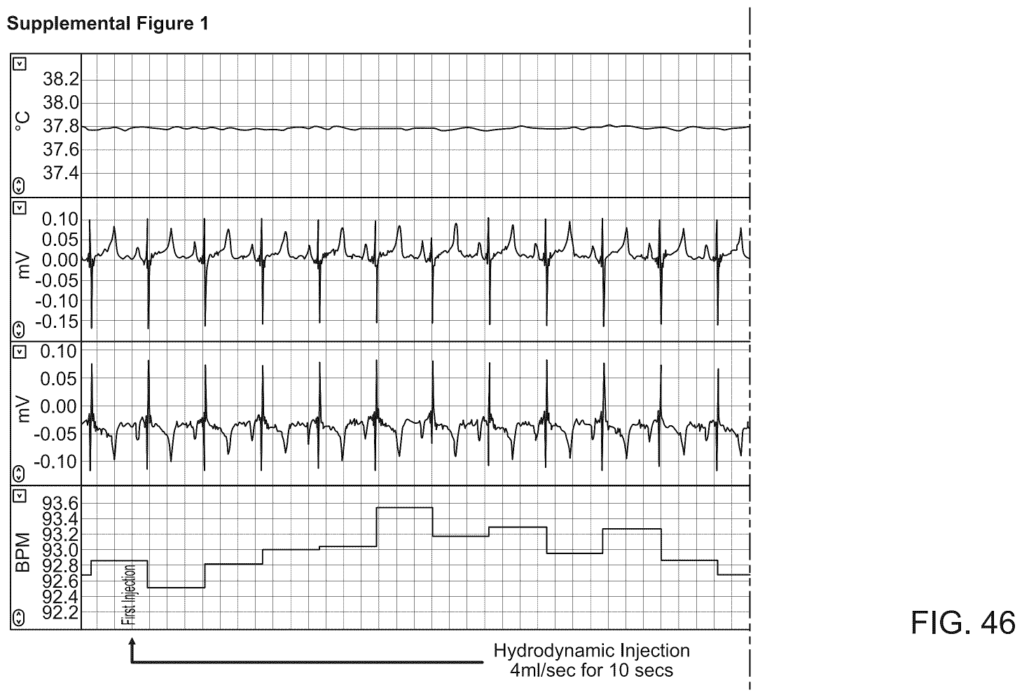

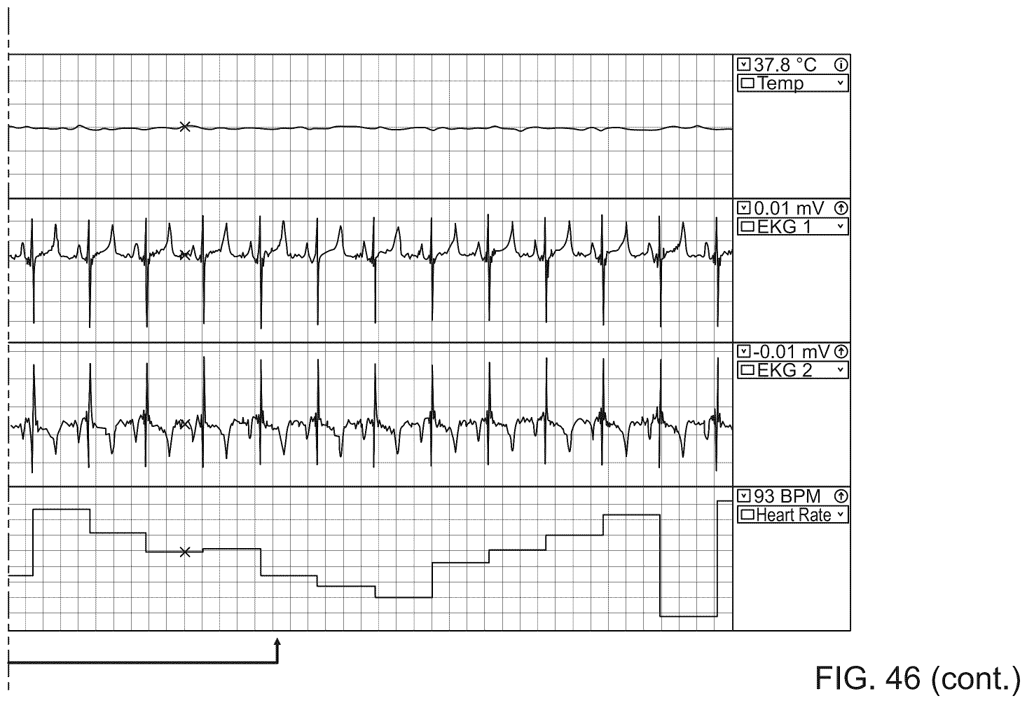

It was first confirmed that the entire biliary tree could be visualized prior to injection (FIG. 20 40A). Employing contrast solution during hydrodynamic injection, the techniques herein monitored the progress of the injection during the entirety of the hydrodynamic procedure, finding the efficient flow of contrast solution into all lobes of the liver (FIG. 40B). With the method validated, different injection parameters were then tested for their tolerability by pigs (Table 1). The results herein repeated multiple hydrodynamic injections within the same pig during one operation to conserve resources and also assess toleration to multiple injections. As judged by the vital signs during procedures (heart rate, respiratory rate, pulse oximetry, mean arterial pressure, and end-tidal CO2) monitored before and after the injection, the pigs could tolerate all the injections with no abnormalities (FIG. 41). Representative real-time vital signs during hydrodynamic injection, including temperature, electrocardiogram and heart rate, are provided in FIG. 46.

As an initial test, the published parameters (30 mL at 2 mL/sec) were repeated, and found to be well-tolerated as expected. Increasing flow rate to 4 mL/sec at the 30 mL was tolerated with no issues, but a higher volume (50 mL) and flow rate (5 mL/sec) next tested in the same pig triggered a flow rate reduction in the power injector near the end of the injection to avoid exceeding the circuit pressure limit (999 psi). Increasing the pressure limit to 1200 psi in the next pig to avoid the flow rate reduction led to the circuit tubing bursting towards the end of the injection, indicating physical limitations to the tubing and catheter materials.

Given that the smaller diameter injection port appeared to have an upper limit between 4-5 mL/sec flow rate, experiments were switched to the guidewire channel (due to its large caliber lumen) to test tolerability to increased flow rates. The results herein injected 47 mL at 10 mL/sec in pig #3, which tolerated this injection well with no acute changes in vital signs. The cholangiogram post-injected did not illustrate extravasation of contrast confirming the ductal anatomy remained intact.

Volume limits during biliary injection were also tested. A higher volume with a lower flow rate was tested (50 mL at 3 mL/sec) and triggered no flow rate reduction. A slightly higher volume (60 mL) at same flow rate did result in flow rate reduction during the last third of the injection. This indicated that the longer volume time adds additional wall stress to the catheter. However, when 80 mL volume was injected at 4 mL/sec flow rate in an attempt to overwhelm the biliary anatomy, no flow rate reduction occurred. The reasons for this discrepancy are unclear and could be related to physiological changes in biliary-sinusoid communication with recurrent injections. Seeing that increased volume at high flow rates may stress the system, it was also asked if a larger volume at a low flow rate would similarly stress the injection system or the pig's vital signs. A 140 mL of volume, near the volume limit of the power injector, at 1 mL/sec was well tolerated with no change in vital signs, and the power injector had no issues.

### Pressure Monitoring During Hydrodynamic ERCP Injection

Pressure achieved during hydrodynamic injections was also evaluated, given its importance to the efficacy of hydrodynamic delivery [8]. A pressure sensing probe was inserted through the guidewire lumen and successfully positioned 1 cm upstream of the catheter tip. Pressure readings for the injection of 30 mL at 2 mL/sec demonstrated a plateau pressure of 80 mmHg during injection, that promptly dropped the moment the injection ended (FIG. 42A). A small level of pressure was released when the balloon was deflated, representing pressure generated by balloon restriction of biliary flow, although the exact value was variable between the different experiments (4.23 mmHg to 18.92 mmHg). A peak pressure point at the initial power injector was also noted in two of the conditions (114.76 mmHg in FIG. 42C and 181.36 mmHg in FIG. 42D) before slightly falling into a plateau phase. This peak pressure point may represent the pressure in the biliary system immediately before fluid begins escaping into the vascular system, and the plateau phase may represent a steady-state pressure of fluid entry and exit into the vascular system.

The pressure curve was also able to detect the balloon accidentally slipping backward, releasing fluid into the gall bladder (FIG. 42B) as confirmed using fluoroscopy. Pressure monitoring may thus be useful to routinely confirm successful injection. The flow rate to pressure relationship appears to be non-linear, since a 1 mL/sec injection and 2 mL/sec injection both similar pressure, 82.12 mmHg and 89.12 mmHg, respectively, during injection, while the 3 mL/sec injection yielded 148.58 mmHg (FIGS. 42C, 42D). The results herein were unable to perform pressure measurements during the other pig injections, but the results suggest the potential for even higher pressures to be achieved at higher flow rates.

### Organ Damage and Tissue Analysis

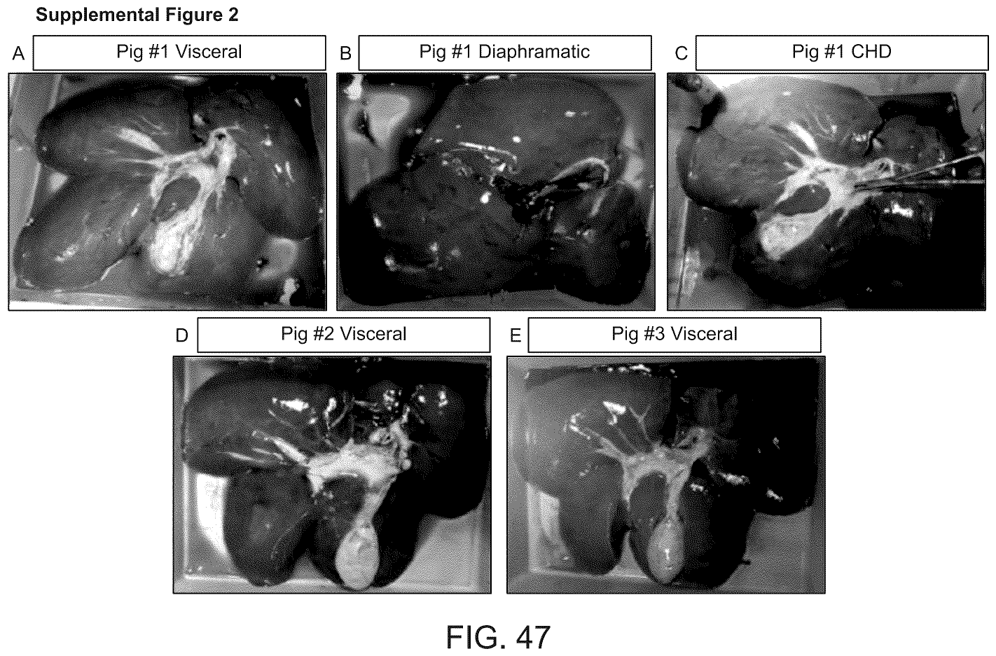

Acute pathogenic changes occurring in pigs immediately post-procedure after repeated hydrodynamic injections were next examined. Pigs were sacrificed within 15 minutes of the last injection, and all showed grossly normal anatomy upon examination without swelling, bruising, or rupture (FIG. 47). The CHD was probed with an instrument in Pig #1, confirming intact status with no lesions (FIG. 47C). The diaphragmatic and visceral surfaces were intact in the pig (FIGS. 47S, 47B). Looking at blood sampled pre- and post-injection, aspartate aminotransferase (AST) showed a notable increase from 19 U/L to 137 U/L in pig #2 and 59 U/L to 252 U/L in pig #3 (Table 2). All other measurements, including alanine aminotransferase (ALT) remained within normal limits.

TABLE 2
Serum chemistry before and after repeated biliary
hydrodynamic injections in pig liver was evaluated.

| | Pig #1 | | Pig #2 | | Pig #3 | | Normal |
|---|---|---|---|---|---|---|---|
| | pre | post | pre | post | pre | post | Reference |
| AST (units/L) | 44 | 48 | 19 | 137 | 59 | 252 | 32-84 |
| ALT (units/L) | 57 | 56 | 51 | 49 | 88 | 90 | 31-58 |
| Amylase (relative units) | 1752 | 1584 | 1853 | 1464 | 758 | 705 | |
| Albumin (g/dL) | 3.3 | 2.7 | 3.5 | 3.3 | 3.5 | 3.2 | 1.9-3.9 |
| Total bilirubin (mg/dL) | 0.3 | 0.5 | 0.2 | 0.6 | 0.3 | 0.3 | 0-10 |
| Direct bilirubin (mg/dL) | 0.2 | 0.5 | 0.2 | 0.5 | 0.2 | 0.3 | 0-0.3 |
| Creatinine (mg/dL) | 1.9 | 1.6 | 1.9 | 1.8 | 1.7 | 1.6 | 1.0-2.7 |

AST, aspartate aminotransferase;
ALT, alanine aminotransferase
References: Peter G. G. Jackson and Peter D. Cockcroft, Clinical Examination of Farm Animals, 2002, 303-305.

A panel of chemistry tests measuring liver function was performed on pre- and post-treatment samples. AST showed an acute rise in Pig #2 and Pig #3, while Pig #1 remained within normal limits. Total and direct bilirubin showed increases in Pig #1 and Pig #2 post-injection, although the increase remained within normal limits. All other values showed no significant changes.

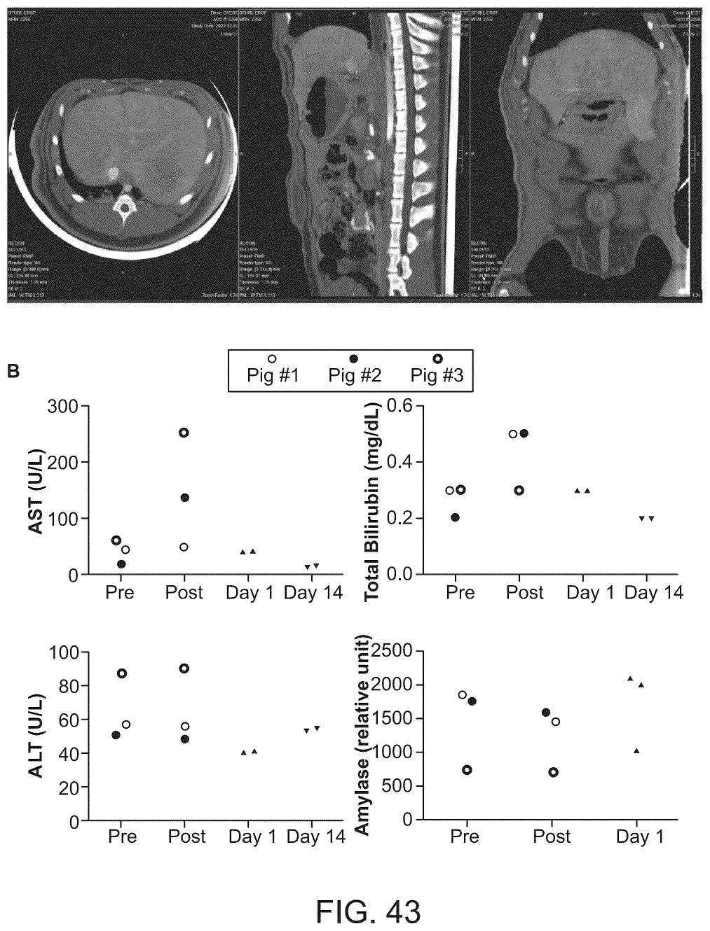

Beyond monitoring biochemical markers for injury, abdominal imaging was performed to evaluate injury from hydrodynamic injections. Abdominal CT with contrast was performed on Day 1 post-injection at parameters of 4 mL/sec and 40 mL of volume. Axial, sagittal and coronal images did not demonstrate any evidence of intra- or extrahepatic biliary dilation, and liver did not show any sign of injury with lack of infarction or necrosis (FIG. 43A).

Short and long-term toxicity from injection was also characterized acutely 15 minutes after injection, as well as on Day 1 and Day 14 post-injection (FIG. 43B). While there was a transient elevation in AST and total bilirubin at 15 minutes post-injection, these values returned to normal range on Day 1 post-injection and remained within normal range 14 days post-injection (FIG. 43B). ALT and amylase did not show any obvious change after hydrodynamic injection at any time point. To investigate where this excess fluid went during the biliary injection, plasmid DNA was dissolved into the solution and injected into one of the pigs. PCR targeted to an internal sequence on the plasmid DNA was performed on DNA isolated from serum collected at all time points. Plasmid DNA was detected in serum 15 minute post-injection illustrating passage from bile into vascular circulation and was no longer detectable in serum on day 1 post-injection (FIG. 48).

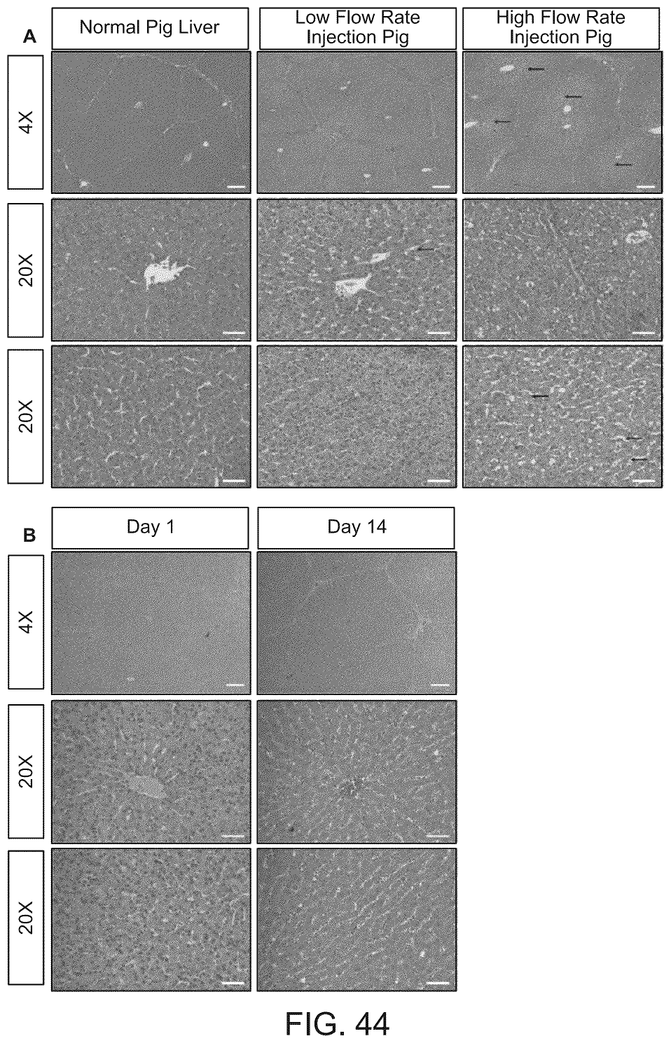

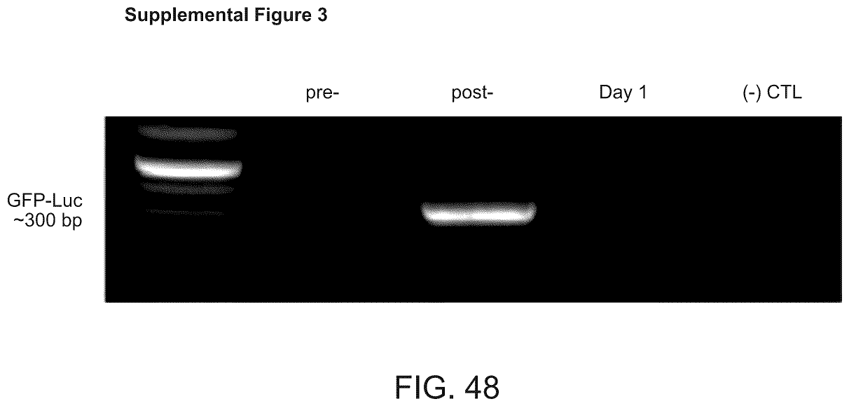

Liver histology in pig #3 acutely injected at the higher flow rate demonstrated larger dilation of sinusoid spaces within hepatic lobules compared to both pigs injected at lower flow rates (FIG. 44A), consistent with fluid rapidly exiting the biliary canaliculi and expanding sinusoidal spaces [21]. Central veins appeared to be the same size between injected and non-injected animals, while the hepatocyte cytoplasm appeared to be more dilute in the injected pigs than an un-injected pig (FIG. 5A), resulting from intracellular entry of fluid [14]. Pig #3 exhibited numerous large, intracellular fluid-filled vesicles scattered throughout the hepatocyte cytoplasm, which were not observed in pig #1 and pig #2 (FIG. 44A). These effects were observed in proximal and distal segments to injection of all five lobes of pig #3, suggesting pressure was able to be distributed evenly throughout the entire organ (FIG. 49).

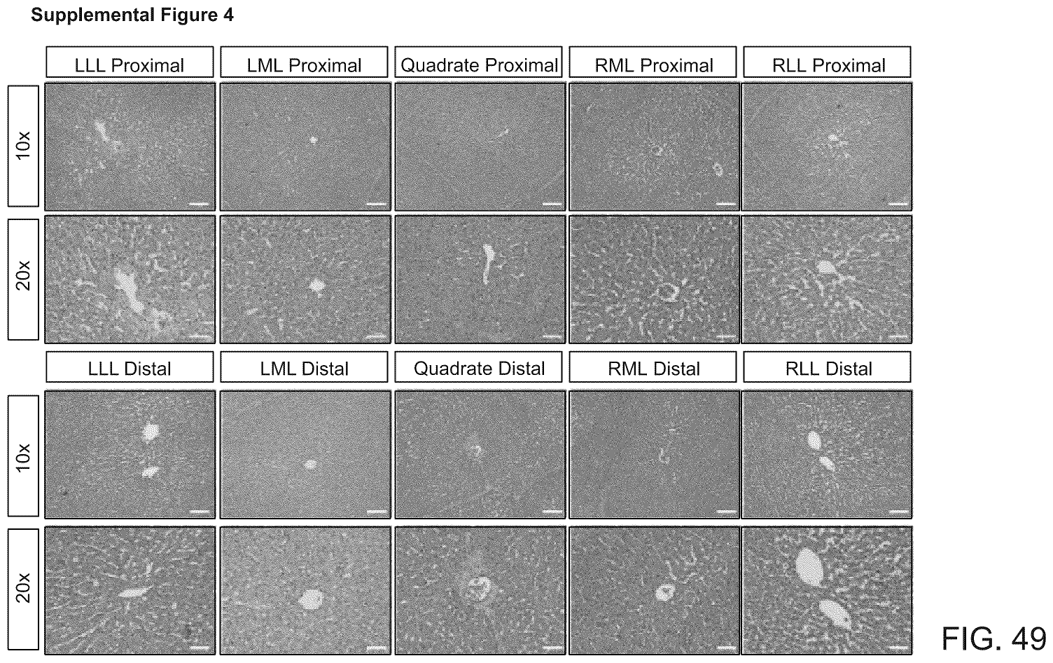

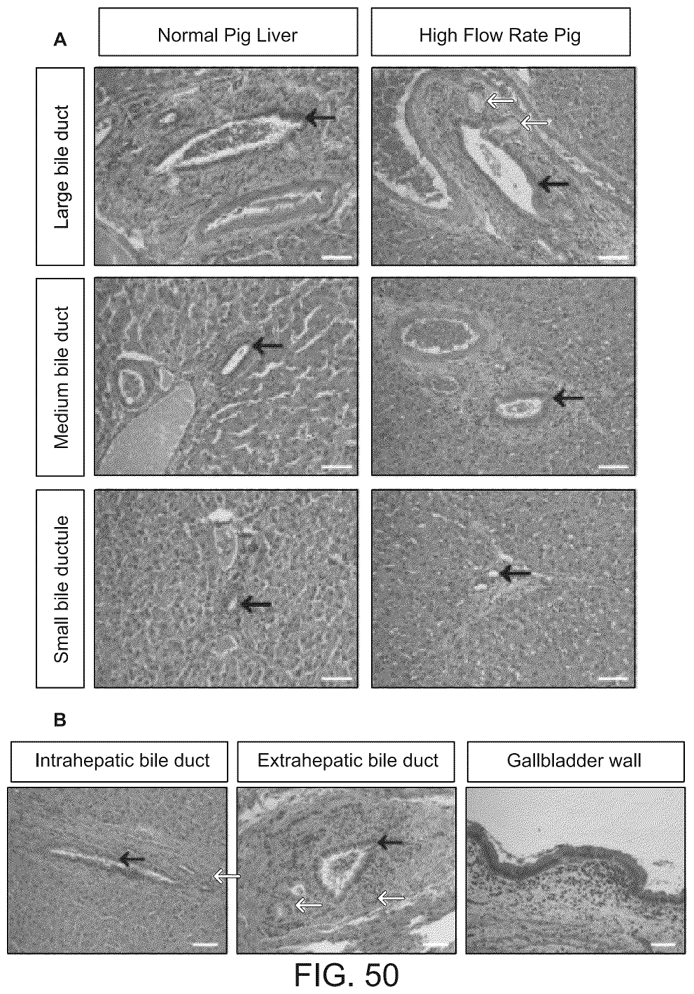

To evaluate the long-term impact of biliary hydrodynamic injection, histological analysis of pigs euthanized on Day 1 and Day 14 post-injection was performed. There was no obvious dilation of sinusoid spaces. Scattered fluid-filled vesicles were still able to be noted on Day 1 post-procedure but were much less compared to 15 minutes post-injection. No fluid-filled vesicles were noted on Day 14 post-injection (FIG. 5B). Looking at biliary injury from hydrodynamic injection, the morphology of large, medium and small bile ducts showed no apparent rupture at the highest flow rates tested, looking histologically similar to un-injected, control pig liver (FIG. 50A). Similarly, the epithelium lining of the intrahepatic and extra hepatic bile duct was intact, while peribiliary glands kept integrity (FIG. 50B).

### Comparison to Murine Hydrodynamic Tail Vein Injection

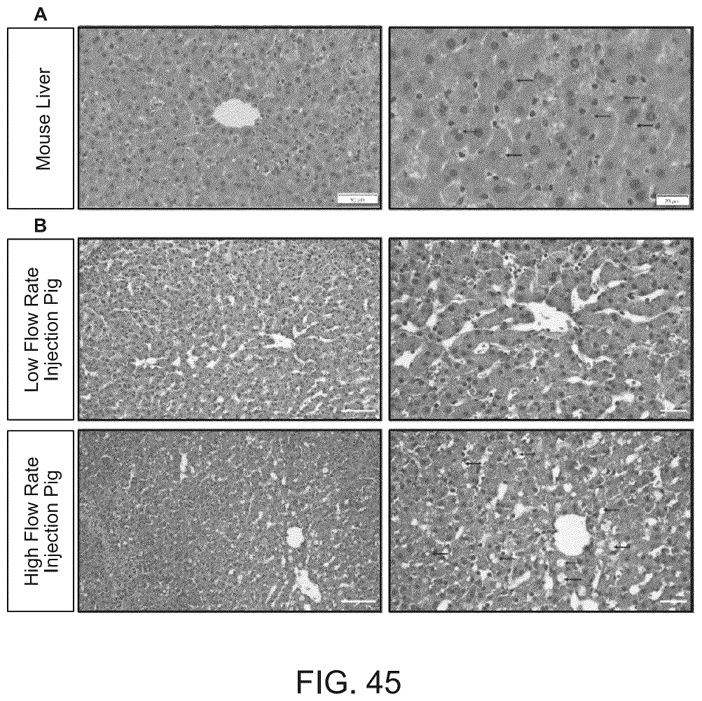

The histopathology of the mouse liver shortly after HTVI was compared with the histopathology of pig liver after biliary hydrodynamic injection. Scattered hepatocytes contained dilute cytoplasm in mouse liver, along with occasional hepatocytes containing red blood cells, the latter reflective of the vascular route of the procedure (FIG. 45). Numerous fluid-filled vesicles were seen within murine hepatocytes, although generally smaller than the vesicles seen in pig #3. The combination of histological changes most resembles the high-pressure injections in pig #3, suggesting these high pressure/flow rates generated could mimicked HTVI in mice effectively.

### Discussion

In this Example 20, a systematic characterization of hydrodynamic injection parameters during ERCP as method for liver directed gene therapy was performed. Novel injection parameters were characterized in vivo in pigs, and found that the positioning of the balloon within the intrahepatic CHD allowed higher volumes and higher flow rates during injection than in the previous testing [24]. Injected fluid escapes into the vascular system, with pressure measured correlating with increases in flow rate (up to 150 mm Hg measured). Flow rate also correlated with histological findings in pig liver, with similar fluid-filled vesicles to mouse hydrodynamic injection. Additionally, potential areas for improvement in current clinical equipment to optimize the procedure going forward were identified.

The techniques herein discovered no clear upper limit on injection volume during biliary injection. Volume only seemed to be important with regards to prolonging time the catheter walls were subjected to stress under high flow rates, which caused pressure thresholds to be reached. As evidenced by the detection of plasmid DNA in the serum, the injected fluid exits from bile canaliculi and through junctions between hepatocytes and into the space of Disse and sinusoids [21]. Interestingly, likely due to the relatively small volumes injected, contrast was not seen on fluoroscopy in the hepatic veins or inferior vena cava, although radiocontrast can be detected in peripheral circulation post-ERCP [26]. Permeability of canalicular tight junctions in the setting of DNA transfer has been observed in a rat model previously [21], and the results herein demonstrate here that is occurs in pigs as well, and thus likely would happen in human patients.

Analysis of pressure during injection demonstrated that flow rate appeared to be the key determinant, closely correlating with the injection initiation and cessation. A flow rate of 5 mL/sec appeared to be the highest achievable via the injection channel of the catheter before approaching circuit pressure limit and triggering flow rate reduction by the power injector, while the larger guidewire channel tolerated at least 10 mL/sec flow rate. Compared to vascular hydrodynamic pig studies, the plateau pressure of 148 mmHg during 3 mL/sec injection is similar to pressure achieved in other studies through vascular routes that demonstrated gene delivery (Table 3) [13,17,27-32]. These other studies often employed much higher flow rates to achieve these pressures, suggesting that compliance and greater volume (600 mL) [33] of the venous system handicap vascular approaches.

By contrast, the relatively small diameter and/or volume of the biliary system (estimated 29 mL in humans [34]) should serve to rapidly increase the pressure even at low flow rates, as compared to higher flow rates required to achieve similar pressure from vascular approaches. It is noted herein that the potential for even higher pressures being achieved exists, given that no measurements for 4 mL/sec, 5 mL/sec, and 10 mL/sec, respectively, were recorded.

TABLE 3
Comparison of intravascular pressures achieved in
previous hydrodynamic liver gene therapy studies.

| | | Spe- | Description of Procedure and Pressure |
|---|---|---|---|
| Year | PMID | cies | Achieved |
| 2005 | 15729372 | Mouse | Achieved 20-30 mmHg pressure in portal |
| | | | vein and IVC after hydrodynamic tail vein |
| | | | injection |
| 2006 | 16871229 | Pig | 150 mL injected at 3 mL/sec (achieving 44 |
| | | | mmHg portal vein pressure) and at 5 mL/sec |
| | | | (58 mmHg achieved) |
| 2008 | 18004400 | Pig | 360-400 mL injected at 100 mL/sec |
| | | | achieving 101-126 mmHg; clamped IVC |
| | | | for delivery |
| 2009 | 19156134 | Pig | 600 mL injected at 40 mL/sec, achieved |
| | | | of 75 mmHg in hepatic vein; pressure up |
| | | | to 100-125 mmHg with IVC occlusion |
| 2011 | 21091276 | Pig | 200 mL injected at 50 mL/sec in isolated |
| | | | lobe, peaking perfusion pressure 103.9 |
| | | | and 226.7 mmHg in two pigs |
| 2013 | 24129227 | Pig | 600 mL injected at 40 mL/sec, catheter |
| | | | advanced into specific liver lobes through |
| | | | hepatic vein. Proximal site achieved 100 |
| | | | mmHg, while distal site was 200 mmHg. |
| 2015 | 26398117 | Pig | 30 mL injected at 20 mL/sec into 4 week |
| | | | old pigs at weaning; portal vein pressure |
| | | | 93 mmHg achieved |
| 2017 | 28447859 | Dog | 200 mL injected at 20 mL/sec yielding |
| | | | peak intravascular pressure between |
| | | | 85-140 mmHg |

Studies exploring hydrodynamic gene delivery in mouse, pigs, and dogs are listed, along with the reported intravascular pressure achieved in them. These comparisons show that the biliary hydrodynamic injection strategy compares favorably to these approaches (150 mmHg at 3 mL/sec) with significantly less volume and flow rate utilized.

To summarize the mechanism of these findings, biliary hydrodynamic injection rapidly increases pressure in the biliary system (peak pressure) before reaching a plateau of steady-state pressure of infusion and escape into the vascular space, explaining wide toleration of volume in the procedure. Importantly, no significant changes in vital signs were noted before and after procedures, regardless of volume or flow rate. This differs from intravascular hydrodynamic injections in pig liver, wherein modulation of heart rate, blood pressure, and respiratory rate during balloon occlusion and opening [17]. Long-term, the human application should optimally use as little volume as possible while balancing transfection efficiency, in order to avoid any effect on rapid increases in intravascular volume.

Also of note was the histological findings of large, fluid-filled vesicles in the cytoplasm of pig hepatocytes, which were also observed in the mouse hepatocytes injected by hydrodynamic tail vein injection (FIG. 6), as seen in other studies [13]. Fluid-filled vesicles are speculated to be directly from pinched off cytoplasmic membrane, resembling micropinocytosis vesicles [14], and may help deliver DNA during hydrodynamic injection, as an alternative to direct transfer through cytoplasmic membrane and nuclear pores [8]. This study is the second time that generation of fluid vesicles have been achieved in large animals after hydrodynamic injection [35]. Given that the lower flow rate parameters failed to induce these vesicles, higher flow rates could mediate higher gene delivery efficiencies. Importantly, despite the large volume and flow rate employed, bile ducts themselves were observed to be intact and un-injured after hydrodynamic injection.

This results herein also exemplifies important parameters concerning liver damage induced by different injection parameters. A mild increase in AST (252 U/L) occurred in pig #3 injected at the highest flow rate, which resolved in other pigs by day 1 and day 14 post-injection. At lower flow rates in pig #1, no elevation in liver enzymes occurred. Together, these findings suggest a wide range of tolerability to differ injection parameters. Another important finding was the influence of psi settings in the power injector; 999 psi was tolerated, while 1200 psi broke the tubing. This limitation could be resolved with catheter materials optimized for this application in the future at higher tensile strength.

The long-term, two week data post-injection demonstrated the normalization of liver function, which was expected based on previous reports of hydrodynamic injection in mice, dogs, and pigs [9,10]. That said, additional studies should be conducted at even higher flow rates to confirm normalization of liver histology. Moreover, while validating the ability of pigs to tolerate repeated injection is enticing for strategies to increase transfection efficiency, there is a risk that the prior injections may have altered the liver tissue. Thus, studies should repeat high volume, flow rate parameters on injection naive pigs to ensure similar results.

In conclusion, the results herein have identified new preferred injection parameters, safety data and constraints of hydrodynamic injection via ERCP into pigs, while replicating aspects of the technique's mechanism from mice to pigs. It was also confirmed the permeability of the biliary system in pigs for the first time. Given that humans and pigs have similar liver size and anatomy and that the results herein employed clinical instruments in the procedures, it is believed that these parameters are applicable toward improving gene delivery methods in human patients. Beyond gene therapy, these findings may be applicable to development of new applications of ERCP, where large injected volumes or flow rates could be used.

### References for this Example 20

1. Sendra L, Herrero M J, Alifio S F. Translational Advances of Hydrofection by Hydrodynamic Injection. Genes (Basel). Multidisciplinary Digital Publishing Institute; 2018; 9: 136. doi:10.3390/genes9030136
2. Dul M, Stefanidou M, Porta P, Serve J, O'Mahony C, Malissen B, et al. Hydrodynamic gene delivery in human skin using a hollow microneedle device. J Control Release. 2017; 265: 120-131. doi:10.1016/j.jconrel.2017.02.028
3. Kamimura K, Zhang G, Liu D. Image-guided, intravascular hydrodynamic gene delivery to skeletal muscle in pigs. Mol Ther. 2010; 18: 93-100. doi:10.1038/mt.2009.206
4. Woodard L E, Welch R C, Williams F M, Luo W, Cheng J, Wilson M H. Hydrodynamic Renal Pelvis Injection for Non-viral Expression of Proteins in the Kidney. J Vis Exp. 2018; e56324. doi:10.3791/56324
5. Sebestyen M G, Budker V G, Budker T, Subbotin V M, Zhang G, Monahan S D, et al. Mechanism of plasmid delivery by hydrodynamic tail vein injection. I. Hepatocyte uptake of various molecules. J Gene Med. John Wiley & Sons, Ltd; 2006; 8: 852-873. doi:10.1002/jgm.921
6. Zhang G, Budker V, Wolff J A. High levels of foreign gene expression in hepatocytes after tail vein injections of naked plasmid DNA. Human Gene Therapy. 1999; 10: 1735-1737. doi:10.1089/10430349950017734
7. Liu F, Song Y, Liu D. Hydrodynamics-based transfection in animals by systemic administration of plasmid DNA. Gene Therapy. Nature Publishing Group; 1999; 6: 1258-1266. doi:10.1038/sj.gt.3300947
8. Zhang G, Gao X, Song Y K, Vollmer R, Stolz D B, Gasiorowski J Z, et al. Hydroporation as the mechanism of hydrodynamic delivery. Gene Therapy. 2004; 11: 675-682. doi:10.1038/sj.gt.3302210
9. Suda T, Liu D. Hydrodynamic gene delivery: its principles and applications. Mol Ther. 2007; 15: 2063-2069. doi:10.1038/sj.mt.6300314
10. Kamimura K, Kanefuji T, Yokoo T, Abe H, Suda T, Kobayashi Y, et al. Safety assessment of liver-targeted hydrodynamic gene delivery in dogs. Mori K, editor. PLoSONE. Public Library of Science; 2014; 9: e107203. doi:10.1371/journal.pone.0107203
11. Andrianaivo F, Lecocq M, Wattiaux-De Coninck S, Wattiaux R, Jadot M. Hydrodynamics-based transfection of the liver: entrance into hepatocytes of DNA that causes expression takes place very early after injection. J Gene Med. John Wiley & Sons, Ltd; 2004; 6: 877-883. doi:10.1002/jgm.574
12. Kobayashi N, Nishikawa M, Hirata K, Takakura Y. Hydrodynamics-based procedure involves transient hyperpermeability in the hepatic cellular membrane: implication of anonspecific process in efficient intracellular gene delivery. J Gene Med. John Wiley & Sons, Ltd; 2004; 6: 584-592. doi:10.1002/jgm.541
13. Crespo A, Peydr6 A, Dasi F, Benet M, Calvete J J, Revert F, et al. Hydrodynamic liver gene transfer mechanism involves transient sinusoidal blood stasis and massive hepatocyte endocytic vesicles. Gene Therapy. Nature Publishing Group; 2005; 12: 927-935. doi:10.1038/sj.gt.3302469
14. Suda T, Gao X, Stolz D B, Liu D. Structural impact of hydrodynamic injection on mouse liver. Gene Therapy. Nature Publishing Group; 2007; 14: 129-137. doi:10.1038/sj.gt.3302865
15. Herweijer H, Zhang G, Subbotin V M, Budker V, Williams P, Wolff J A. Time course of gene expression after plasmid DNA gene transfer to the liver. J Gene Med. John Wiley & Sons, Ltd; 2001; 3: 280-291. doi:10.1002/jgm.178
16. Viecelli H M, Harbottle R P, Wong S P, Schlegel A, Chuah M K, VandenDriessche T, et al. Treatment of phenylketonuria using minicircle-based naked-DNA gene transfer to murine liver. Hepatology. 2014; 60: 1035-1043. doi:10.1002/hep.27104
17. Kamimura K, Suda T, Xu W, Zhang G, Liu D. Image-guided, lobe-specific hydrodynamic gene delivery to swine liver. Mol Ther. 2009; 17: 491-499. doi:10.1038/mt.2008.294
18. Herrero M J, Sabater L, Guenechea G, Sendra L, Montilla Al, Abargues R, et al. DNA delivery to “ex vivo” human liver segments. Gene Therapy. Nature Publishing Group; 2012; 19: 504-512. doi:10.1038/gt.2011.144
19. Khorsandi S E, Bachellier P, Weber J C, Greget M, Jaeck D, Zacharoulis D, et al. Minimally invasive and selective hydrodynamic gene therapy of liver segments in the pig and human. Cancer Gene Ther. Nature Publishing Group; 2008; 15: 225-230. doi:10.1038/sj.cgt.7701119
20. Zhang G, Vargo D, Budker V, Armstrong N, Knechtle S, Wolff J A. Expression of naked plasmid DNA injected into the afferent and efferent vessels of rodent and dog livers. Human Gene Therapy. 1997; 8: 1763-1772. doi:10.1089/hum.1997.8.15-1763
21. Hu J, Zhang X, Dong X, Collins L, Sawyer G J, Fabre J W. A remarkable permeability of canalicular tight junctions might facilitate retrograde, non-viral gene delivery to the liver via the bile duct. Gut. BMJ Publishing Group; 2005; 54: 1473-1479. doi:10.1136/gut.2005.070904
22. Chen C-Y, Liu H-S, Lin X-Z. Hydrodynamics-based gene delivery to the liver by bile duct injection of plasmid DNA—the impact of lasting biliary obstruction and injection volume. Hepatogastroenterology. 2005; 52: 25-28.
23. Jiang X, Ren Y, Williford J-M, Li Z, Mao H-Q. Liver-targeted gene delivery through retrograde intrabiliary infusion. Methods Mol Biol. Totowa, N J: Humana Press; 2013; 948: 275-284. doi:10.1007/978-1-62703-140-0 19
24. Kumbhari V, Li L, Piontek K, Ishida M, Fu R, Khalil B, et al. Successful liver-directed gene delivery by ERCP-guided hydrodynamic injection (with videos). Gastrointest Endosc. 2018; 88: 755-763.e5. doi:10.1016/j.gie.2018.06.022
25. Indrajit I K, Sivasankar R, D'Souza J, Pant R, Negi R S, Sahu S, et al. Pressure injectors for radiologists: A review and what is new. Indian J Radiol Imaging. Medknow Publications; 2015; 25: 2-10. doi:10.4103/0971-3026.150105
26. Draganov P, Cotton P B. Iodinated contrast sensitivity in ERCP. Am J Gastroenterol.2000; 95: 1398-1401. doi:10.1111/j.1572-0241.2000.02069.x
27. Yoshino H, Hashizume K, Kobayashi E. Naked plasmid DNA transfer to the porcine liver using rapid injection with large volume. Gene Therapy. Nature Publishing Group; 2006; 13: 1696-1702. doi:10.1038/sj.gt.3302833
28. Fabre J W, Grehan A, Whitehome M, Sawyer G J, Dong X, Salehi S, et al. Hydrodynamic gene delivery to the pig liver via an isolated segment of the inferior vena cava. Gene Therapy. Nature Publishing Group; 2008; 15: 452-462. doi:10.1038/sj.gt.3303079
29. Fabre J W, Whitehome M, Grehan A, Sawyer G J, Zhang X, Davenport M, et al. Criticalphysiological and surgical considerations for hydrodynamic pressurization of individual segments of the pig liver. Human Gene Therapy. 2011; 22: 879-887. doi:10.1089/hum.2010.144
30. Kamimura K, Suda T, Zhang G, Aoyagi Y, Liu D. Parameters Affecting Image-guided,Hydrodynamic Gene Delivery to Swine Liver. Mol Ther Nucleic Acids. 2013; 2: e128. doi:10.1038/mtna.2013.52
31. Stoller F, Schlegel A, Viecelli H M, Rufenacht V, Cesarovic N, Viecelli C, et al. Hepatocyte Transfection in Small Pigs After Weaning by Hydrodynamic Intraportal Injection of Naked DNA/Minicircle Vectors. Human Gene Therapy Methods. 2015; 26: 181-192. doi:10.1089/hgtb.2014.140
32. Hyland K A, Aronovich E L, Olson E R, Bell J B, Rusten M U, Gunther R, et al. Transgene Expression in Dogs After Liver-Directed Hydrodynamic Delivery of Sleeping Beauty Transposons Using Balloon Catheters. Human Gene Therapy. 2017; 28: 541-550. doi:10.1089/hum.2017.003
33. Kjekshus H, Risoe C, Scholz T, Smiseth O A. Regulation of hepatic vascular volume: contributions from active and passive mechanisms during catecholamine and sodium nitroprusside infusion. Circulation. 1997; 96: 4415-4423. doi:10.1161/01.cir.96.12.4415
34. Ludwig J, Ritman E L, LaRusso N F, Sheedy P F, Zumpe G. Anatomy of the human biliary system studied by quantitative computer-aided three-dimensional imaging techniques. Hepatology. John Wiley & Sons, Ltd; 1998; 27: 893-899. doi:10.1002/hep.510270401
35. Alifio S F, Herrero M J, Noguera I, Dasi F, Sanchez M. Pig liver gene therapy by noninvasive interventionist catheterism. Gene Therapy. Nature Publishing Group; 2007; 14: 334-343. doi:10.1038/sj.gt.3302873

### Example 21

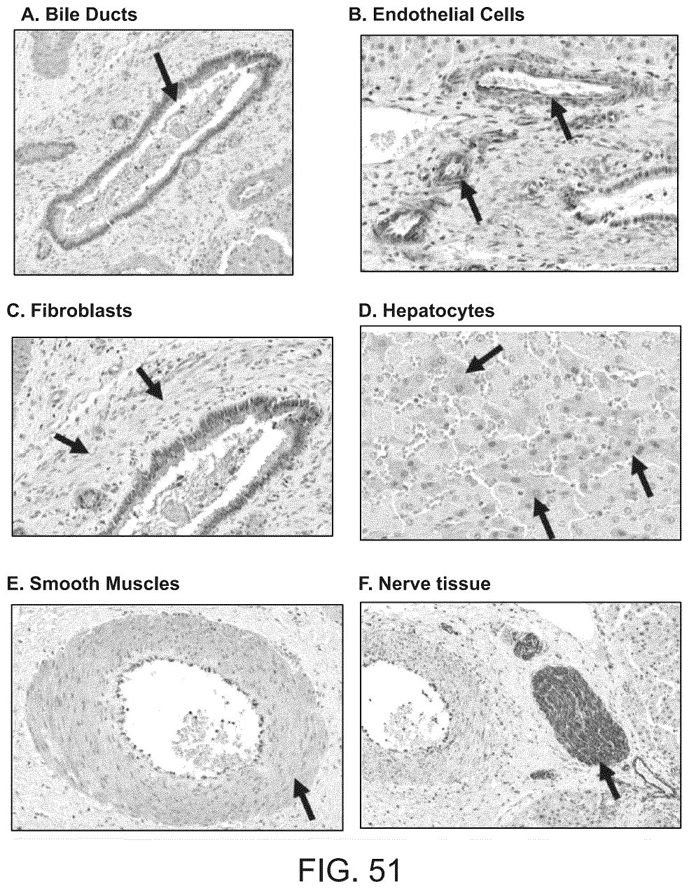

These examples demonstrate inter alia different cell types in the liver can be targeted with biliary hydrodynamic injection. Hydrodynamic injection of 7 mg of plasmid encoding for CMV-Firefly Luciferase and SV40-GFP (˜7 kb) proceeded into a 50 kg pig at 4 mL/sec over 40 mL volume total. Both viral promoters are known to express in multiple cell types. Results are shown in FIGS. 51A-D. In FIG. 51A: Firefly Luciferase staining is prominently observed in bile ducts after injection. The intensity of the stain was significantly more than the surrounding hepatocytes, suggesting more DNA delivery to these tissues. FIG. 51B: GFP staining was observed intensely in endothelial cells lining arterioles in the portal triads of the liver. This observation was very surprising, since this cell type is not specifically connected to the biliary system but suggests transfer of injected fluid across the portal triads to the other vessels. FIG. 51C: Luciferase staining is also observed lightly staining interstitial fibrous tissue in the portal triads. FIG. 51D: Firefly luciferase staining can be observed in hepatocytes scattered through thel obule. FIG. 51E: Smooth muscle associated with arteries in portal triads also demonstrated efficient expression of GFP after injection. FIG. 51F: Neurons in portal triads also demonstrated efficient expression of GFP after injection.

### Example 22

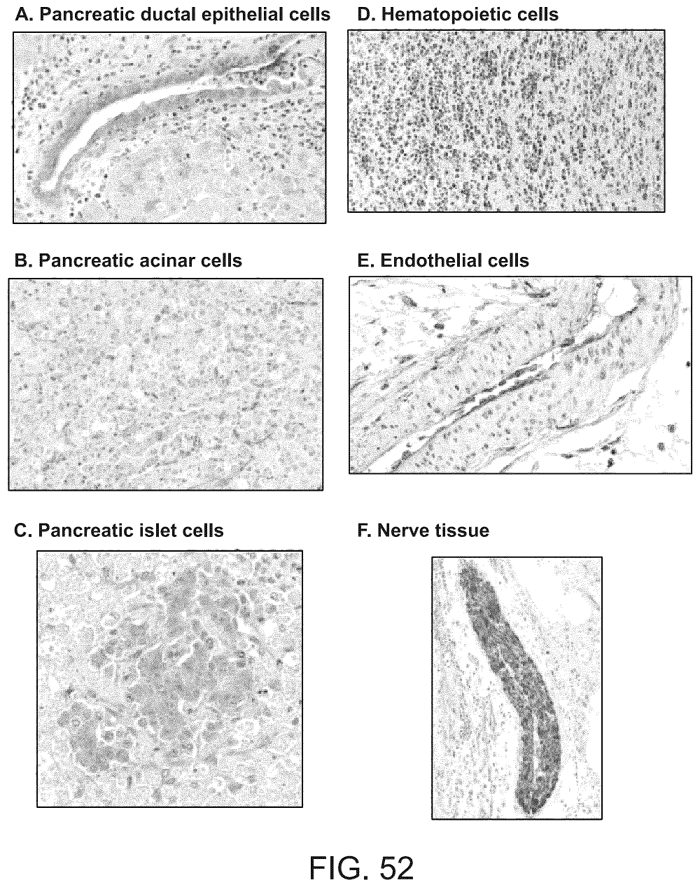

This Example demonstrates inter alia different cell types in the pancreas that can be targeted with ductal hydrodynamic injection. Hydrodynamic injection of 1 mg of plasmid encoding for CMV-Firefly Luciferase and SV40-GFP (7 kb) proceeded into a 50 kg pig at 2 mL/sec over 20 mL volume total. Results are shown in FIGS. 52A-F. FIG. 52A: Pancreatic ductal cells prominently stain with firefly luciferase after injection. FIG. 52B: Pancreatic acinar cells can be abundantly found and express GFP. FIG. 52C: Pancreatic islet cells prominently stain with firefly luciferase after injection. FIG. 52D: Cells of the hematopoietic lineage also demonstrated intense staining with GFP. FIG. 52E: Endothelial cells in the pancreas were observed to have positive Firefly luciferase staining. FIG. 52F: Neurons stained intensely with GFP.

### Example 23

This Example demonstrates inter alia that different regions of the liver can be targeted when the flow rate of hydrodynamic injection through the biliary system is altered.

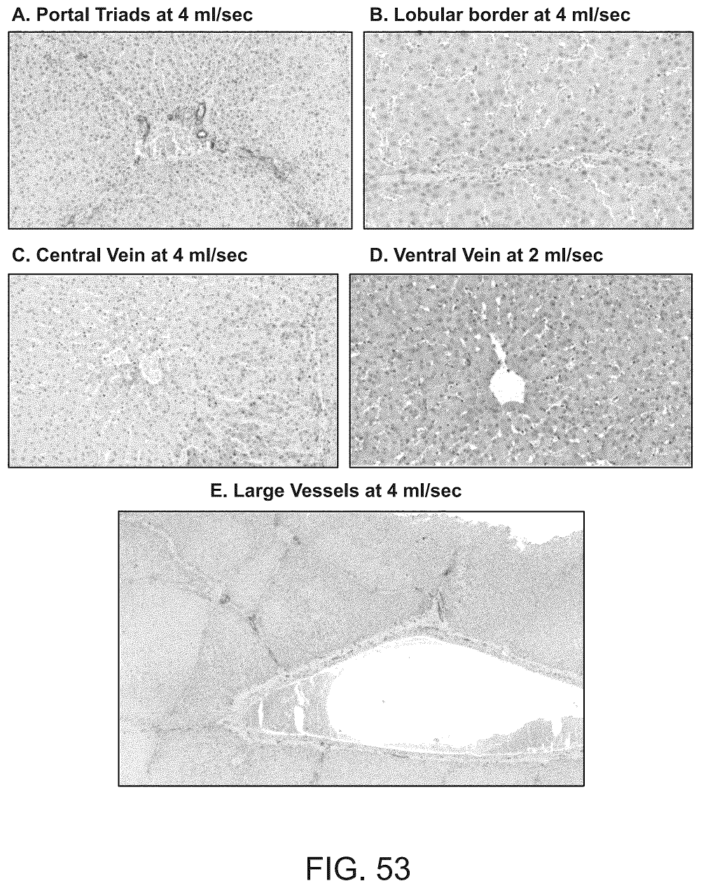

Hydrodynamic injection of 7 mg of plasmid encoding for CMV-Firefly Luciferase and SV40-GFP (7 kb) proceeded into a 50 kg pig at 4 mL/sec over 40 mL volume total. Results are shown in FIGS. 53A-E. Staining for GFP revealed prominent staining around the (FIG. 53A) portal triads. Firefly luciferase staining could be found along (FIG. 53B) lobular borders. By comparison, the (FIG. 53C) central vein at this flow rate had comparatively few cells. Prior staining patterns at 2 mL/sec localized transfection primarily around the (FIG. 53D) central vein. Of note, expression was particularly intense near (FIG. 53E) large vessels, which yielded radiation of transfection outward, irrespective of lobules.

### Example 24

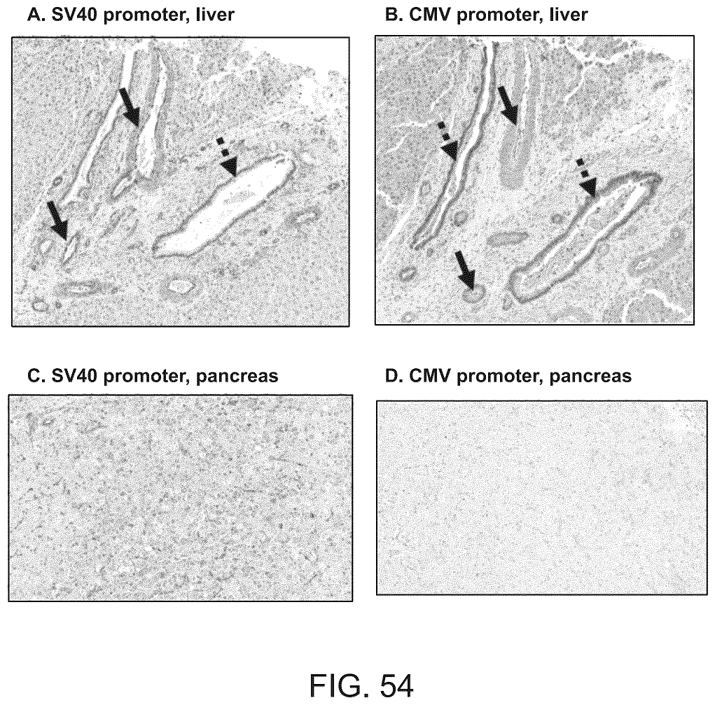

This Example shows inter alia that promoters can lead to exclusion of expression in different cell types in the liver and pancreas. Hydrodynamic injection of 7 mg of plasmid encoding for CMV-Firefly Luciferase and SV40-GFP (7 kb) proceeded into a 50 kg pig at 4 mL/sec over 40 mL volume total. Results are shown in FIGS. 54A-D. FIG. 54A: Intense GFP staining of endothelial cells (solid arrow) was observe with SV40 promoter, while bile ducts (dashed arrow) were not stained for GFP. FIG. 54B: By comparison, Firefly luciferase could be observed in both cell types. Hydrodynamic injection of 1 mg of the same plasmid proceeded into a 50 kg pig at 2 mL/sec over 20 mL volume total. FIG. 54C: The acinar cells only stained strongest for GFP staining under SV40 promoter, while (FIG. 54D) CMV promoter driving firefly luciferase did not yield strong and consistent staining in the same cell types.

### Example 25

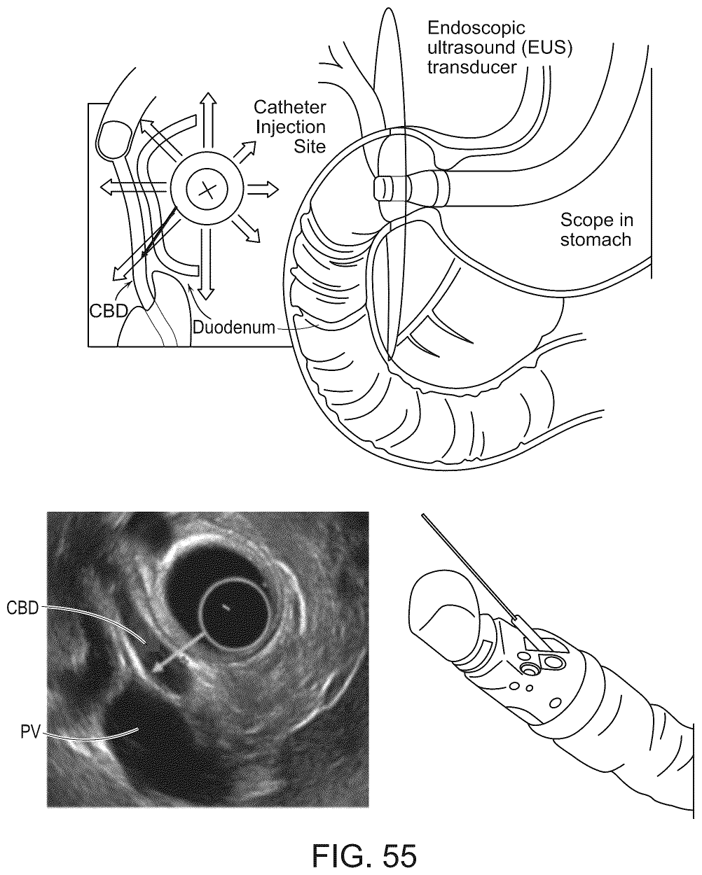

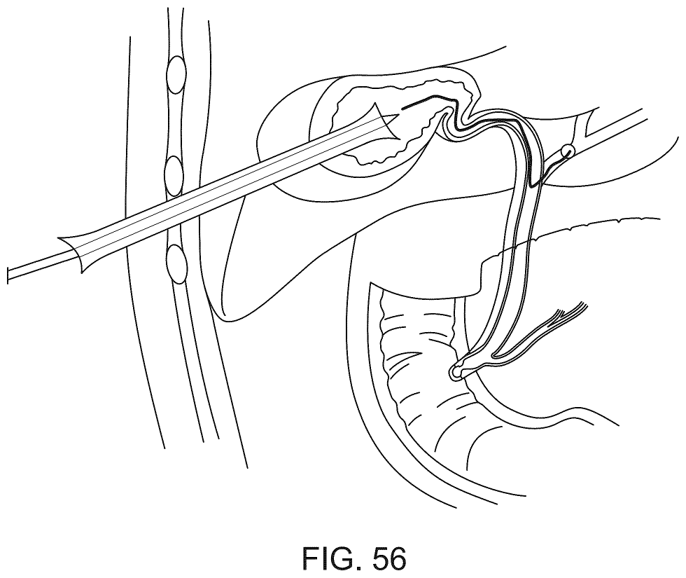

FIG. 55 shows a diagram of accessing the common bile duct by endoscopic ultrasound for biliary hydrodynamic injection. Endoscopic mediated ultrasound can be used to identify the common bile duct, which lies on the other side of the wall of the duodenum. In other embodiments, endoscopic ultrasound may be used to identify the gallbladder, right hepatic duct, left hepatic duct, or common hepatic duct. In one iteration, after localization of the target structure in the duodenum, a needle can be inserted to reach the common bile duct. Depending on size, the needle switched out, so that the hole can be expanded to create a wider diameter. A guide wire and subsequently a catheter can be inserted through this hole to gain entrance into the common bile duct. As shown in FIG. 55, from there, the catheter can be advanced into the common hepatic duct, and the injection procedure undertaken as disclosed herein such as above examples. A diagram showing this alternative method of catheter placement is depicted.

### Example 26

FIG. 56 shows a diagram of accessing the common hepatic duct by percutaneous needle access to the gallbladder for subsequent biliary hydrodynamic injection. Bile is sometimes drained through an external drain when a significant obstruction develops of the biliary system. In these instances, a percutaneous needle is placed under imaging support from the skin through the liver and into the gall bladder, followed by a catheter to drain bile. As shown in FIG. 56, using this same procedure, a catheter is placed within the gall bladder, and subsequently advanced through the cystic duct and into the common hepatic duct. Variations of this procedure would leverage percutaneous access of bile ducts directly, followed by similar subsequent localization into common hepatic duct or branch of interest. The injection procedure would subsequently be undertaken as disclosed herein such as above examples.

### Example 27

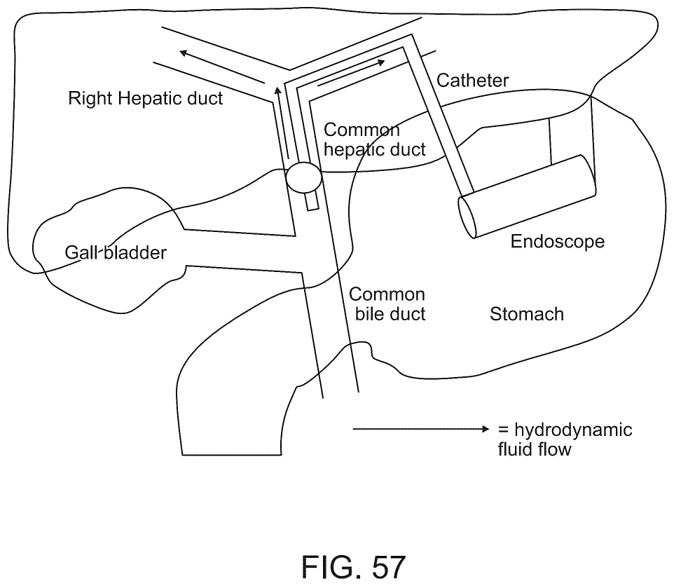

FIG. 57 shows a diagram of accessing the common hepatic duct by endoscopic needle to the intrahepatic ducts followed by retrograde hydrodynamic injection with fluid injection proximal to the balloon. The balloon could also be placed in the right or left main hepatic ducts. Access to the right hepatic duct from the left hepatic duct, and vice versa, could be performed bypassing a wire from the right to the left and then railroading the catheter over the wire towards the left hepatic duct. Using this method, if the catheter was coming from the right to the left hepatic duct, the fluid in the left system would be deployed in a retrograde manner (the balloon would be downstream/towards the hilum with the fluid being deployed upstream).

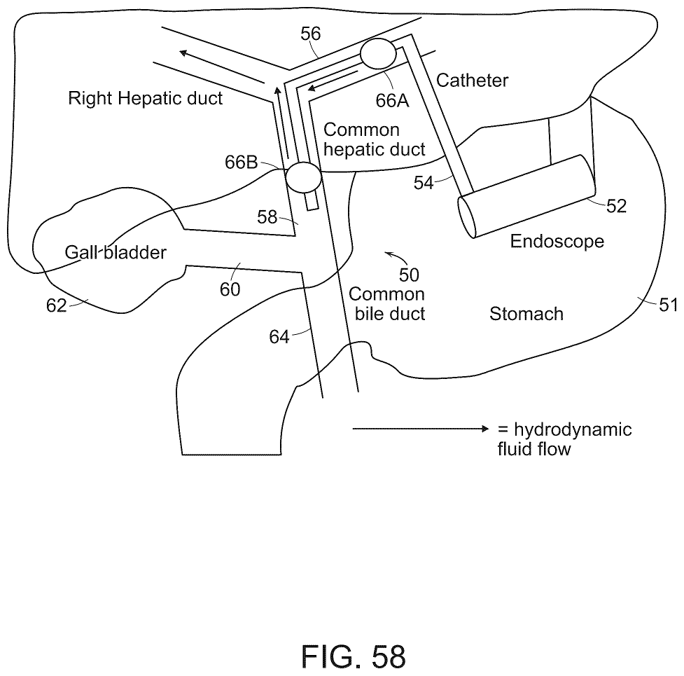

In another method, a catheter with two balloons can be used. A suitable system 50 is illustrated in FIG. 58 that shows stomach 51 with endoscope 52, catheter 54, multiple balloons 66A, 66B positioned within hepatic duct 56, 58 past duct 60 and gall bladder 62 and bile duct 64. As shown in FIG. 58, one balloon 66A could be placed at the right main hepatic duct 56 and a second balloon 66B towards the periphery of the liver parenchyma though still within the ductal system. When inflated, these balloons would create seals such that the injection of plasmid would not escape down the common bile duct in an antegrade fashion or between the liver and peritoneum in a retrograde fashion. In a similar manner, the placement of the balloons could be as follows to facilitate plasmid delivery to the entire liver parenchyma: 1) balloon placed at the periphery of the liver parenchyma of the ductal system and 2) balloon placed in the common hepatic duct. Injection of plasmid in between the balloons should facilitate transfection to almost the entire liver. The plasmid could enter in an antegrade/towards the hilum fashion of the balloon at the periphery of the liver and/or in a retrograde fashion from the more proximal/liver parenchyma end of the balloon placed more centrally.

Upstream branches of hepatic bile ducts may be accessed by endoscopic routes. In these embodiments, the catheter will be advanced in the direction of the bile duct flow (antegrade) into the desired position in the common hepatic duct. Variations of the procedure may leave the catheter within the right or left hepatic ducts for specific targeting of a portion of the liver. In these embodiments, a modified catheter must be utilized that has the injection port below the balloon toward the proximal aspects of the catheter, in order to yield fluid flow in the retrograde direction throughout the liver. This hydrodynamic fluid flow will then mediate transfection of liver cells in the desired target area.

### Example 28

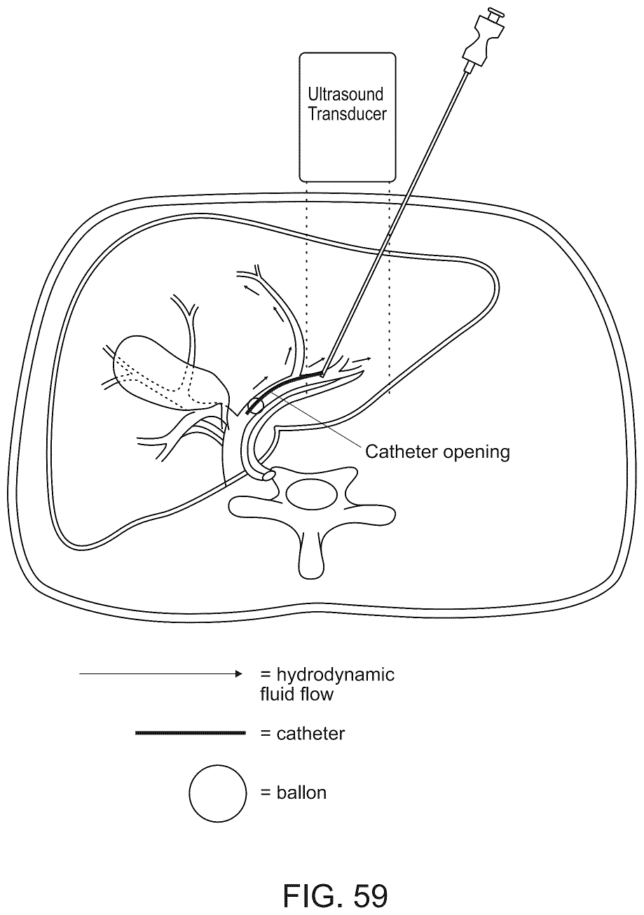

FIG. 59 shows a diagram of accessing the common hepatic duct by percutaneous needle to the intrahepatic ducts followed by retrograde hydrodynamic injection with fluid injection proximal to the balloon. Upstream branches of hepatic bile ducts may be accessed by percutaneous routes. In these embodiments, the catheter will be advanced in the direction of the bile duct flow (antegrade) into the desired position in the common hepatic duct. Variations of the procedure may leave the catheter within the right or left hepatic ducts for specific targeting of a portion of the liver. This variation is depicted in the figure, wherein the catheter is positioned in the left hepatic duct. In both of these embodiments, a modified catheter must be utilized that has the injection port below the balloon toward the proximal aspects of the catheter, in order to yield fluid flow in the retrograde direction throughout the liver. This hydrodynamic fluid flow will then mediate transfection of liver cells in the desired target area.

### Example 29

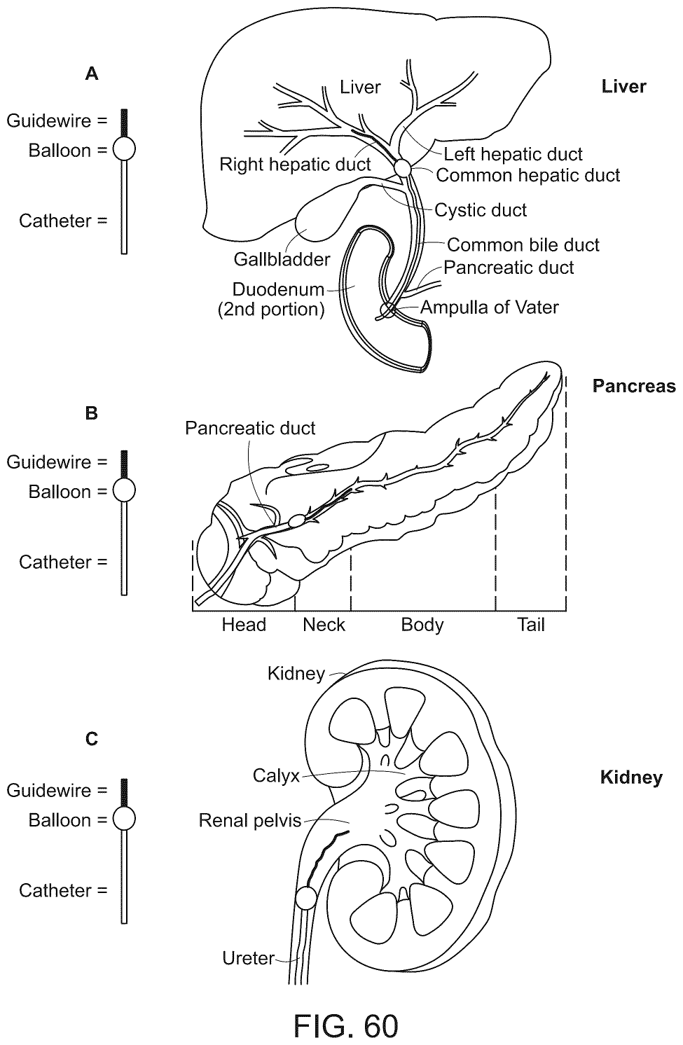

FIG. 60 depicts a diagram showing the guidewire in place during hydrodynamic injection. In order to facilitate cannulation of the ampulla of Yater and entry of the catheter into the bile duct system, a guidewire is commonly used as a part of ERCP. Moreover, the guidewire can also be used to assure placement of the catheter in the common hepatic duct and not the cystic duct. The guidewire is similar used to assure the placement of the catheter into the pancreatic ductal system. During cystoscopy procedures, a guidewire is also used to help canulate the ureteral orifice and can help guide the catheter up to the renal pelvis. In many embodiments of the disclosure, the guidewire is subsequently removed prior to hydrodynamic injection. Guidewire removal is a requirement in certain embodiments of the disclosure that inject the DNA solution through the guidewire port. However, for some procedures, the guidewire can be left in during the hydrodynamic injection procedure in order to verify place of the catheter throughout the injection and stabilize the catheter during injection. Examples of the guidewire staying in place during injection are provided for the (FIG. 60A) liver, (FIG. 60B) pancreas, and (FIG. 60C) kidney.

### Example 30

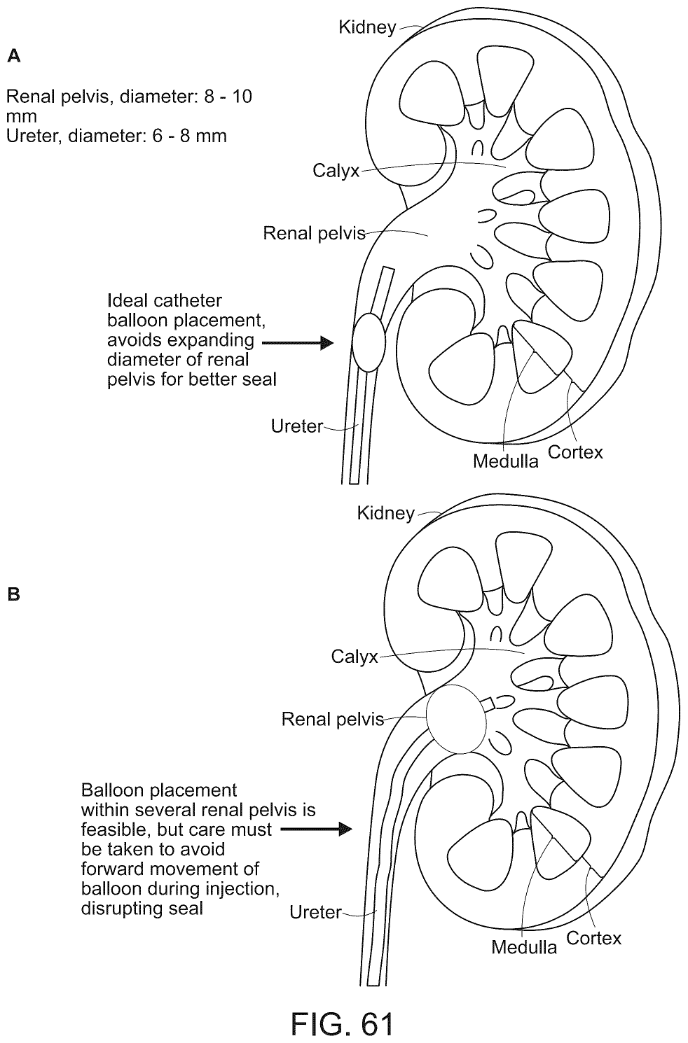

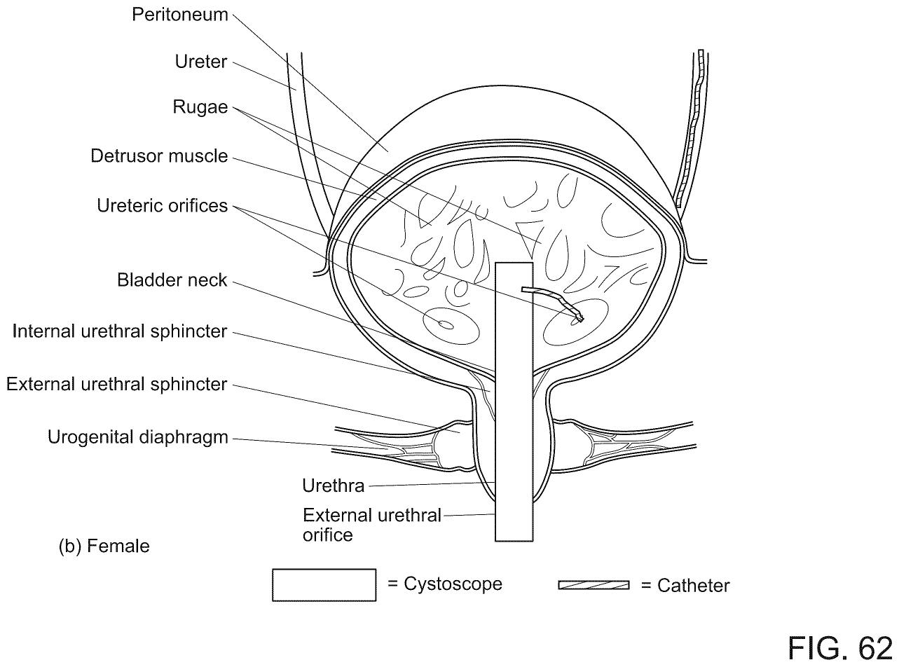

FIG. 61 shows a diagram of catheter placement into the ureter and renal pelvis for optimal injection and seal. Hydrodynamic injection into the renal pelvis requires precise catheter and balloon placement to optimize pressures achieved and prevent antegrade flow of fluid. FIG. 61A: Depicted is localization of the balloon within the ureter before fanning out of the renal pelvis. Contrast injection prior to hydrodynamic injection will confirm proper balloon placement. Catheter tip is ideally at least 1 cm past the balloon inflation extending free into the renal pelvis. FIG. 61B: As an alternative, the balloon can be inflated within the renal pelvis itself, though care must be made to ensure balloon stability so that it doesn't move forward and break seal during hydrodynamic injection.

### Example 31

FIG. 62 shows a diagram of cannulation of the ureter orifice in the bladder with a catheter. A cystoscopy procedure is used to insert a scope with camera into the bladder. A catheter can be inserted through the cystoscope and steered toward canulation of the ureteral orifice on the desired side (right or left) for kidney targeted. In other embodiments, a guidewire can be used first to canulate a specific ureteral orifice, followed driving the catheter over the guidewire.

### Example 32

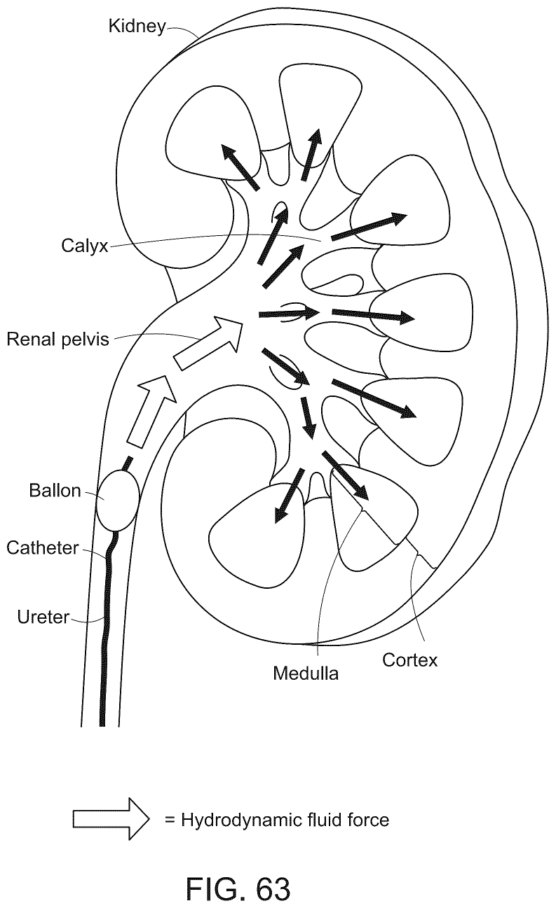

FIG. 63 shows a diagram of the hydrodynamic fluid force into the kidney in order to mediate efficient transfection. After proper balloon catheter positioning in the ureter or renal pelvis, the injected fluid will proceed in a retrograde direction, ascending up the collecting ducts and leaking into the interstitial tissue. This will deliver DNA, RNA, or proteins directly into different renal cell types.

### Example 33: Demonstration of ERCP-Mediated Gene Delivery to Pancreas

Endoscopic retrograde choangio-pancreatography (ERCP) to mediate non-viral hydrodynamic gene delivery through the pancreatic duct in pigs. Non-viral gene therapy was chosen due to its significantly lower-cost which may allow for routine use in metabolic conditions such as diabetes. It was previously demonstrated that ERCP can be used to deliver plasmid DNA efficiently into hepatocytes.² The results disclosed herein assess whether using the disclosed procedure allows for efficient transduction porcine pancreatic cells.

### Methods

### Plasmid DNA

pCLucf was a gift from John Schiller (Add gene plasmid #37328 http://n2t.net/addgene:37328; RRID: Add gene_37328).2. pCLucf encodes for firefly luciferase under a CMV promoter and GFP under an SV40 promoter. The plasmid was prepared for injection using a gigaprep kit from ZymoResearch. One milligram of pCLucf plasmid DNA was diluted into sterile normal saline prior to injection in a total of 25 mL solution.

### Pancreatic Injection Procedure

Endoscopy was performed in two pigs (45-50 kg) under anesthesia. All pigs were weighed before to determine anesthesia drug testing, and a pre-procedure blood draw (red and purple top tubes) obtained. The endoscope was advanced into the small intestine and the pancreatic orifice was visualized. Guidewire and subsequently catheter were advanced into the pancreatic duct. Balloon placement was just inside the pancreatic duct in the duodenal lobe of the pig pancreas before the branching of the pancreatic duct into a superior and inferior terminus, covering the duodenal and splenic lobes and the connecting lobes respectively. Contrast injection confirmed proper placement of the catheter as seen by visualization of both branches. The balloon was next inflated in the duodenal lobe of the pancreas, to prevent retrograde motion of any injected fluid at high pressure. 25 mL of the plasmid DNA dissolved in saline solution was loaded into the power injector prior to injection with subsequently 5 mL of the DNA solution discharged to priming the circuit from the power injector to the distal end of the catheter. Hydrodynamic injection was commenced using a power injector under settings of 20 mL of DNA solution injected at 2 ml per second. A repeat fluoroscopy was performed with the contrast post-procedure to evaluate integrity of the pancreatic duct. 15 minutes post-injection, a second blood draw was obtained (red and purple top tubes). CT scan was performed on each pig 24 hours post-injection.

### Tissue Analysis

Pigs were harvested at 24 hours post-injection. Injected pancreas was surgically dissected in isolation from duodenum and stomach, weighed, and each of the three lobes sampled for tissue analysis. Control, non-injected pig pancreas tissue was also obtained separately and a similar analysis performed.

Tissue was fixed in 10% formaldehyde, and frozen tissue fixed in OCT. PCR was performed 30 on frozen tissue from different samples to confirm presence of pCLucf DNA. Immunohistochemistry was performed by VitroVivo (Rockville, MD) using polyclonal firefly luciferase antibodies and polyclonal GFP antibodies, respectively.

### Blood Analysis

Serum chemistries and hematology were performed by the Johns Hopkins Phenotyping Core on Diasys Respons®910 chemistry analyzer and Procyte automated analyzer, respectively.

### Results

Two pigs weighing between 45-50 kg were obtained and subjected to an ERCP. The pancreatic orifice was readily visualized during the procedure (FIG. 64B), and fluoroscopy prior to injection (FIG. 64C) confirmed that both branches of the pancreatic duct were accessible reaching the entire organ. Hydrodynamic injection proceeded using a power injector, injecting 20 mL of saline solution containing 1 mg pClucf DNA at 2 mL/second. Repeat fluoroscopy demonstrated patent pancreatic ducts, indicating no injury from hydrodynamic injection (FIG. 64C). Blood was sampled prior to procedure, and then 15 minutes post-injection and 24 hours post-injection at harvest of pig pancreas. Data showed that amylase was elevated pancreatic injection, with no other abnormalities in serum chemistry (FIG. 65 (Table 1)). There was no elevation in leukocyte count after injection as well (FIG. 66 (Table 2)). Abdominal CT imaging at day 1 post-injection revealed no acute signs suggestive of pancreatitis, suggesting amylase may have been from leakage from pancreatic cells from the injection, without associated injury. Vital signs (heart rate, respiratory rate, pulse oximetry) were stable throughout the injection, indicating high tolerance from the procedure in pigs.

The pigs were harvested at day 1 post-injection to evaluate for gene expression, avoiding variables of plasmid DNA expression stability/silencing. Pig pancreas tissue was carefully dissected revealing the characteristic three lobe structure (FIG. 64A), lacking the rotation and fusion of the connecting and duodenal lobes seen in human patients, which forms the head of the pancreas. Pancreas weights were 112 g and 130 g, correlating with pig weight. Tissue was samples in all three pancreatic lobes, with catheter placement in the duodenal lobe. Histology from these sections revealed no obvious differences from a non-injected pig control as assessed by a board-certified pathologist.

The pCLucf plasmid encodes separate expression cassettes for firefly luciferase and GFPunder CMV and SV40 promoters, respectively. This should yield expression in a variety of cell types. PCR demonstrated the presence of pCLucf DNA in all three pancreatic lobes (FIG. 64D). Immunohistochemistry for FLuc and GFP yielded detectable expression in all three liver lobes, with expression noted in the pancreatic dutal epithelium and in acinar cells at the distal branches of the pancreatic duct (FIG. 64D). Expression was also detected in pancreatic islet cells as well. The results herein quantified levels of FLuc protein in the different pancreatic lobes, finding similar levels of FLuc protein, regardless of proximity to the injection site.

### Discussion

In this Example, it was shown for the first time that non-viral gene delivery into the pancreas of a large animal model was feasible, and the first publication to gene delivery into the pancreas of a human-sized animal model (50 kg vs 1.5 kg in previous AAV report). It was demonstrated that ERCP can readily access the pancreatic duct, whereafter hydrodynamic delivery can directly deliver DNA into multiple cell types.

The results herein successfully delivered DNA into pancreatic ductal epithelium, islet and acinar cells in all lobes of the pancreas proximal and distal to the injection site. Protein expression was confirmed using two different reporter proteins emphasizing the validity of findings. Importantly, only a very small level of DNA was injected into the pancreas in these proof-of-concept experiments, such that increasing the dose in the future may yield higher levels of protein expression. Further optimization of the injection parameters may also yield higher transfection efficiency.

A major concern of this approach is the potential for causing pancreatitis in human patients, which can occur during routine clinical ERCP procedures secondary to cannulation of the pancreatic duct and contrast injection. The data in pigs shows that the procedure was well-tolerated with only minimal elevation in amylase levels, with no other biochemistry hematological, histological, or imaging abnormalities. That said, the physiology and anatomy between pigs and humans is different, and future studies will have to evaluate tolerability in other animal models, and eventually in human patients. Additional studies will interrogate whether there are any long-term side effects from pancreatic injection, as well as confirm the stability of gene expression. Moreover, it will be important to test different pancreatic cell-specific promoters in order to explore targeting gene expression in intended cell types.

In conclusion, the data presented here demonstrated that gene delivery is possible and efficient into the pancreas of large animals, leveraging a common clinical procedure in ERCP routine today, as well as a power injector used in clinical practice. Given the employment of plasmid DNA as the gene vector, this greatly reduces cost of treatment and helps obviate potential immune responses to gene therapy. The scalability of plasmid DNA manufacturing may make pancreatic gene therapy possible for the million-plus patients with diabetes and other pancreatic diseases. Future studies will continue exploring the safety and persistence of gene expression, as well as the delivery of therapeutic genes in specific pig models of pancreatic disease. The demonstration of gene delivery in a human-sized animal model suggests the potential for clinical gene therapy of pancreatic diseases.

### Example 34: Demonstration of Cell-Specific Targeting with Cell-Specific Promoter after Hydrodynamic Injection Through Bile Ducts into Liver

This example demonstrates the ability to modify the promoter in the plasmid DNA injected in order to target one or more cell types of interest after hydrodynamic injection into tissues such as, for example, the liver, pancreas, or kidney. In an exemplary embodiment, endothelial cells were chosen in an exemplary target tissue, the liver. The endothelial cell specific promoter, intercellular adhesion molecule 2 (ICAM2) promoter, was chosen for this purpose (J Biol Chem. 1998 May 10 8; 273(19):11737-44.). The promoter was cloned in front of a GFP reporter gene. Pigs for this experiment were injected at 40 mL volume and 2 mL/sec by biliary hydrodynamic injection. The DNA dose was 8 mg of plasmid DNA.

The data obtained in this experiment is depicted in FIGS. 67A-F, which illustrate the ability to target protein expression specifically to endothelial cells after biliary hydrodynamic injection in the liver. Pigs were injected with a plasmid, pICAM2-GFP, that harbors an endothelial cell-specific promoter driving expression of green fluorescent protein (GFP). The data shows immunohistochemical staining for GFP protein in pig liver tissue. FIG. 67A depicts endothelial cells bordering a blood vessel specifically staining positive for GFP expression. FIG. 67B illustrates an isolated endothelial cell positive for GFP amidst negatively stained hepatocytes. FIGS. 67C, 67D, and 67E depict portal triads within the pig liver, demonstrating that only the endothelial cells (solid arrow) are positive for protein expression. For comparison, bile ducts (dashed arrow) are negative for any GFP expression, emphasizing the specificity of the cell-specific targeting. FIG. 67F depicts hepatocytes around a central vein in a lobule, which are all negative reflective of a lack of observed off-target cell expression. All references, including publications, patent applications, and patents, cited herein are hereby incorporated by reference to the same extent as if each reference were individually and specifically indicated to be incorporated by reference and were set forth in its entirety herein.

The use of the terms “a” and “an” and “the” and similar referents in the context of describing the disclosure (especially in the context of the following claims) are to be construed to cover both the singular and the plural, unless otherwise indicated herein or clearly contradicted by context. The terms “comprising,” “having,” “including,” and “containing” are to be construed as open-ended terms (i.e., meaning “including, but not limited to,”) unless otherwise noted. Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein, is intended merely to better illuminate the disclosure and does not pose a limitation on the scope of the disclosure unless otherwise claimed. No language in the specification should be construed as indicating any non-claimed element as essential to the practice of the disclosure.

Preferred embodiments of this disclosure are described herein, including the best mode known to the inventors for carrying out the disclosure. Variations of those preferred embodiments may become apparent to those of ordinary skill in the art upon reading the foregoing description. The inventors expect skilled artisans to employ such variations as appropriate, and the inventors intend for the disclosure to be practiced otherwise than as specifically described herein. Accordingly, this disclosure includes all modifications and equivalents of the subject matter recited in the claims appended hereto as permitted by applicable law. Moreover, any combination of the above-described elements in all possible variations thereof is encompassed by the disclosure unless otherwise indicated herein or otherwise clearly contradicted by context.

1. A method of treating a subject, comprising:
administering with a catheter an effective amount of a nucleic acid and/or protein solution at high-fluid pressure through the subject's biliary tree into liver,
wherein the catheter delivery portion is advanced through the biliary tract, common bile duct, upstream past the cystic duct, and located into the common hepatic duct at a position to avoid biliary rupture.

2. The method of claim 1 wherein the catheter tip is positioned at an extrahepatic location during delivery of the nucleic acids.

3. The method of claim 1 wherein the catheter tip is positioned within an intrahepatic position within the liver parenchyma during delivery of the nucleic acids.

4. The method of claim 1 wherein the catheter tip is placed within or proximate to the liver parenchyma.

5. The method of claim 1 wherein the catheter is positioned whereby the catheter balloon is inflated proximate to or downstream of the liver hilum within the common hepatic duct.

6. The method of claim 5 wherein the administered nucleic acid solution increases pressure within the liver.

7. The method of claim 1 wherein the catheter balloon is inflated inside the liver parenchyma to prevent retrograde flow of fluid into the common hepatic duct.

8. A method of treating a subject, comprising:
administering to the subject's nucleic acid and/or protein solution biliary tree, liver, kidney, or pancreas, 1) a first fluid composition that does not contain nucleic acid and/or protein and thereafter 2) an effective amount of a nucleic acid and/or protein solution.

9. A method of claim 8, wherein the first fluid injection serves to clear bile, urine, or pancreatic exocrine secretions from the biliary system, ureters and renal pelvis, or pancreatic ductal system, before the injection of the nucleic acid and/or protein solution.

10. A method of treating a subject, comprising:
administering a radiocontrast agent through the subject's biliary tree, liver, kidney or pancreas to verify catheter placement after balloon inflation, and thereafter
administering an effective amount of a nucleic acid and/or protein solution at high-fluid pressure through the subject's biliary tree, liver, kidney or pancreas.

11. The method of claim 10 wherein catheter position is selected and/or verified with visualization of the administered radiocontrast agent.

12. The method of claim 10 wherein the radiocontrast agent is administered through the subject's biliary tree into the subject's liver, through the subject's pancreatic ducts into the subject's pancreas, and through the subject's ureters into the subject's kidneys.

13. A method of placing the catheter into the common hepatic duct for biliary hydrodynamic injection, wherein the catheter placement includes the use of endoscopic retrograde cholangiopancreatography (ERCP) in order to insert a catheter into the common hepatic duct at a specified location of claim 1, or alternatively, the subject's pancreatic duct.

14. The method of claim 1 wherein the catheter is selectively advanced into the right hepatic bile duct whereby the nucleic acids is administered within right liver lobes.

15. The method of claim 1 wherein the catheter is selectively advanced into the left hepatic bile duct whereby the nucleic acids is administered within left liver lobes.

16. The method of claim 1 wherein one or more pharmacologic agents is injected into the pancreatic duct prior to or after the endoscopic retrograde cholangiopancreatography procedure to decrease the frequency and severity of post-procedure pancreatitis.

17. (canceled)

18. The method of claim 16 wherein the one or more pharmacologic agents comprise gabexate mesilate, nafamostat mesylate, ulinastatin, Camostat mesylate, Aprotinin, Pefabloc, Trasylol, and Urinary Trypsin Inhibitor, or enzyme suppressive agents, including somatostatin.

19. (canceled)

20. A method of placing the catheter into the common hepatic duct for biliary hydrodynamic injection, comprising steps of:
(a) advancing an endoscope/echoendoscope into the small intestine or stomach;
(b) inserting needle through the small intestine or stomach wall and into a bile duct or gallblader;
(c) optionally injecting fluid into a bile duct or the gallbladder in order to increase its diameter to allow easier entry of a guidewire and/or catheter
(d) passing a guidewire through the needle into a bile duct or gallbladder;
(e) advancing a catheter over the wire into the common hepatic duct and administering nucleic acid and/or protein via the catheter.

21. The method of claim 20 wherein sonographic guidance is used to locate the common bile duct, common hepatic duct, right hepatic duct, left hepatic duct, small intrahepatic ducts, or gallbladder.

22-42. (canceled)

43. The method of claim 1 further comprising selecting an amount of nucleic acid or protein to be administered to the subject based on the subject liver weight.

44-375. (canceled)
